(19) United States
(12) Patent Application Publication
(10) Pub. No.: US 2026/0265710 A1
(43) Pub. Date: Sep. 10, 2026

(54) METHODS AND COMPOSITIONS FOR PRODUCING A CAROTENOID OR TERPENOID

(71) Applicant: Allozymes Pte Ltd, Singapore (SG)

(72) Inventors: Akbar Khalfekandi Vahidi, Singapore (SG); Balaji Sundara Sekar, Singapore (SG); Tiago Resende, Singapore (SG); Jacky Tan, Singapore (SG); Pavan Dronamraju, Singapore (SG); Shelly Cheng, Singapore (SG); Pradeep Madhavankutty Nair, Singapore (SG)

(21) Appl. No.: 19/473,074

(22) PCT Filed: Apr. 10, 2024
(86) PCT No.: PCT/SG2024/050245

§ 371 Date: Oct. 6, 2025

Related U.S. Application Data
(60) Provisional application No. 63/495,349, filed on Apr. 11, 2023.

(51) Int. Cl. C12N 9/10; C12N 9/02; C12N 9/04; C12N 9/12; C12N 9/88; C12N 9/90; C12N 15/52; C12P 5/00; C12P 7/02; C12P 23/00

(52) U.S. Cl. CPC C12N 9/1085; C12N 9/0006; C12N 9/001; C12N 9/1022; C12N 9/1205; C12N 9/1229; C12N 9/88; C12N 9/90; C12N 15/52; C12P 5/007; C12P 7/02; C12P 23/00; C12Y 103/99029; C12Y 202/01007; C12Y 205/01029; C12Y 207/01036; C12Y 207/04002; C12Y 503/03002

(57) ABSTRACT

The invention relates to the production of industrially relevant carotenoids and terpenoids products such as phytoene, lycopene, and bisabolol using engineered microbes. More particularly, the invention relates to methods for biosynthesis of phytoene and bisabolol, and to nucleic acid sequences, enzymes, expression vectors, and transformed host cells for carrying out the methods.

### FIELD

The present invention is in the field of industrial biotechnology and relates to the biosynthesis of terpenes.

### BACKGROUND

Terpenes are a diverse class of naturally occurring chemical compounds composed of five-carbon isoprene units that are found widespread in nature, mainly in plants. Their building block is the hydrocarbon isoprene (C5H8) n. Examples of terpenes include isoprene (C5 hemiterpene), farnesene (C15 Sesquiterpenes), bisabolol (C15 Sesquiterpenes), and carotenoids (C40 Tetraterpenes). Bisabolol is commonly found in candeia trees and chamomile plants and is known for its fresh floral fragrance with a sweet hint of spice or citrus.

Carotenoids are organic compounds, that due to their chemical structure, result in distinct properties such as pigmentation, antioxidants and UV absorbance. They range in color from yellow to red that are naturally produced by certain organisms, including photosynthetic organisms (e.g., plants, algae, cyanobacteria), and some fungi. Examples of carotenoids are lycopene that is responsible for the red color found in tomatoes and β-carotene that is responsible for the orange color of carrots.

Due to these properties, terpenes (including bisabolol and carotenoids) are widely used in cosmetics, pharmaceuticals, and nutraceuticals. Carotenoids are also precursors for key components of cosmetics and pharmaceuticals, such as retinol.

In general, the biological systems that produce sesquiterpenes (including bisabolol) and tetraterpenes (including carotenoids) are industrially intractable and/or produce the compounds at such low levels that commercial scale isolation is not practical. Thus, many sesquiterpenes and tetraterpenes used in industry are produced by chemical synthesis. Microbial production of these compounds using a combination of engineered endogenous and heterologous enzymes have been reported, to circumvent the need to obtain the compounds from their natural sources. Some efforts have previously been made to genetically engineer certain bacteria or fungi to produce higher levels of terpenes (see, for example, Misawa et al., J. Biotechnol. 59:169, 1998; Visser et al., FEMS Yeast Research 4:221, 2003). However, improved systems, allowing higher levels of production and greater ease of isolation, are needed to produce carotenoids or terpenoids such as phytoene or bisabolol at commercially relevant volumes.

In microbes, terpenes are produced via one of two pathways and the overexpression of enzymes of the methyl erythritol 4-phosphate (MEP) pathway and the mevalonate (MVA) pathway. Overexpression of the enzymes in these pathways have been used to increase the production of the precursors for all terpenes, dimethylallyl diphosphate (DMAPP) and isopentenyl diphosphate (IPP), as well as for enhancing the production of carotenoids (including lycopene and β-carotene) and other terpenes (including bisabolol). Although the production of lycopene and β-carotene has been well described, the engineering of strains to produce phytoene as a final product has not. It is well-understood that producers of lycopene and β-carotene cannot be used to produce phytoene as it is a pathway intermediate.

Much work has focused on the modulation of certain enzymes in the relevant pathways, such as geranylgeranyl diphosphate synthase (GGPPS), which converts the natural precursors dimethylallyl diphosphate (DMAPP) and isopentenyl diphosphate (IPP) to geranylgeranyl Pyrophosphate (GGPP) which can be further converted to phytoene via a phytoene synthase (PYS), which can be further converted to lycopene by phytoene desaturase. The produced lycopene can be converted to β-carotene with additional overexpression of lycopene β-cyclase. Alternatively, farnesyl pyrophosphate synthase (FPPS) can also convert DMAPP and IPP to farnesyl diphosphate (FPP) which can be further converted to bisabolol using a α-bisabolol synthase (BBS).

### SUMMARY OF INVENTION

The present invention provides, inter alia, methods for the production of one or more terpenes and/or precursors thereof by microbial fermentation of a substrate, and recombinant microorganisms of use in such methods. In one embodiment, the microorganism contains various heterologous enzymes in the metabolic pathway for the production of terpenes, including bisabolol and phytoene, that improves metabolite production above that produced using known enzymes. In another embodiment, the microorganism contains the same enzymes which are optimized by genetic and/or protein engineering techniques, such as directed evolution or rational mutagenesis to optimize the enzymes for expression and activity in a host cell, which are known to those of ordinary skill in the art.

### DETAILED DESCRIPTION OF THE INVENTION

As used herein, the term “endogenous” refers to a substance or process that can occur naturally in a host cell. In contrast, the term “exogenous” refers to a substance or compound that originated outside an organism or cell. The exogenous substance or compound can retain its normal function or activity when introduced into an organism or host cell described herein.

The terms “modified,” “recombinant” and “engineered,” when used to modify a host cell described herein, refer to host cells or organisms that do not exist in nature, or express compounds, nucleic acids or proteins at levels that are not expressed by naturally occurring cells or organisms. Reference to an engineered enzyme, for example to an engineered HmgR enzyme is intended to include within the meaning any engineered HmgR enzyme described herein, whether it is explicitly described in that same embodiment, or in a different embodiment.

The term “heterologous compound” refers to the production of a compound by a cell that does not normally produce the compound, or to the production of a compound at a level at which it is not normally produced by the cell.

As used herein, the term “heterologous” refers to what is not normally found in nature. The term “heterologous compound” refers to the production of a compound by a cell that does not normally produce the compound, or to the production of a compound at a level not normally produced by the cell. For example, bisabolol or phytoene can be a heterologous compound.

As used herein, the phrase “heterologous enzyme” refers to an enzyme that is not normally found in a given cell in nature. The term encompasses an enzyme that is: (a) exogenous to a given cell (i.e., encoded by a nucleotide sequence that is not naturally present in the host cell or not naturally present in a given context in the host cell); (b) naturally found in the host cell (e.g., the enzyme is encoded by a nucleotide sequence that is endogenous to the cell) but that is produced in an unnatural amount (e.g., greater or lesser than that naturally found) in the host cell; or (c) an enzyme that may be endogenous to the host cell but which comprises one or more modifications, such as one or more substitutions, deletions of insertions such that the particular amino acid sequence is not found naturally in the given host cell or (d) an enzyme that is naturally found in the host cell, but positioned outside of its natural locus.

In some embodiments an enzyme expressed in a host cell is exogenous to a given cell (i.e., encoded by a nucleotide sequence that is not naturally present in the host cell or not naturally present in a given context in the host cell). In some embodiments an enzyme expressed in a host cell is naturally found in the host cell (e.g., the enzyme is encoded by a nucleotide sequence that is endogenous to the cell) but that is produced in an unnatural amount (e.g., greater or lesser than that naturally found) in the host cell. In some embodiments an enzyme expressed in a host cell is an enzyme that may be endogenous to the host cell but which comprises one or more modifications, such as one or more substitutions, deletions of insertions such that the particular amino acid sequence is not found naturally in the given host cell. In some embodiments an enzyme expressed in a host cell is an enzyme that is naturally found in the host cell, but positioned outside of its natural locus.

Provided herein are recombinant or modified host cells that are useful for producing a heterologous product, and methods of using the host cells. The recombinant or modified host cells comprise a heterologous genetic pathway that can be differentially regulated by one or more exogenous agents. The recombinant host cells provide the advantage of increasing expression of the heterologous product compared to the parent strain.

As used herein, the terms “MEP pathway” or “methyl erythritol 4-phosphate pathway” refers to the production of dimethylallyl diphosphate (DMAP) using the enzymes 1-deoxy-D-xylulose-5-phosphate synthase (DXS), 1-Deoxy-d-xylulose 5-phosphate reductoisomerase (IspC), 2-C-methyl-D-erythritol 4-phosphate cytidylyltransferase (IspD), 4-diphosphocytidyl-2-C-methyl-D-erythritol kinase (IspE), 2-C-methyl-D-erythritol 2,4-cyclodiphosphate synthase (IspF), 4-hydroxy-3-methylbut-2-en-1-yl diphosphate synthase (IspG), and 4-hydroxy-3-methylbut-2-enyl diphosphate reductase (IspH). In certain embodiments the genes within this pathway are native or endogenous to E. coli. In another embodiment the genes within this pathway are heterologous or not native to E. coli.

As used herein, the terms “MVA pathway” or “mevalonate pathway” refers to the production of isopentenyl diphosphate (IPP) and dimethylallyl diphosphate (DMAP) using the enzymes Acetyl-CoA C-acetyltransferase (HmgACT), 3-hydroxy-3-methylglutaryl-CoA (HMG-CoA) synthase (HmgS), 3-hydroxy-3-methylglutaryl coenzyme A (HMG-CoA) reductase (HmgR), mevalonate kinase (MVK), phosphomevalonate kinase (PMK), phosphomevalonate decarboxylase (PMD), and isopentenyl diphosphate dimethylallyl diphosphate isomerase (IDI). In certain embodiments the genes within this pathway are native or endogenous to Saccharomyces cerevisiae. In other embodiments the genes within this pathway are encoded by the polynucleotides from B. subtilis or E. coli.

The term “pyruvate” refers to the compound pyruvate including any stereoisomer of pyruvate. The chemical name of pyruvate is 2-oxopropanoic acid. In particular embodiments, the term refers to the compound according to the following structure:

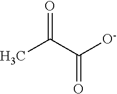

The term DXP refers to the compound DXP including any stereoisomer of DXP. The chemical name of DXP is 1-deoxy-D-xylulose 5-phosphate. In particular embodiments, the term refers to the compound according to the following structure:

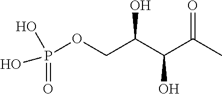

The term MEP refers to the compound MEP including any stereoisomer of MEP. The chemical name of DXP is methylerythritol 4-phosphate. In particular embodiments, the term refers to the compound according to the following structure:

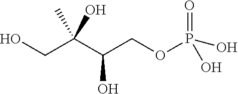

The term CDP-ME refers to the compound CDP-ME including any stereoisomer of CDP-ME. The chemical name of CDP-ME is 4-diphosphocytidyl-2-C-methylerythritol. In particular embodiments, the term refers to the compound according to the following structure:

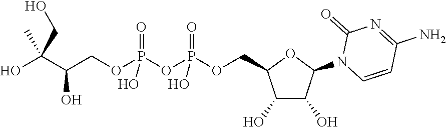 [SMILES: C[C@](O)(CO)[C@H](O)COP(=O)(O)OP(=O)(O)OC[C@H]1O[C@@H](n2ccc(N)nc2=O)[C@H](O)[C@@H]1O]

The term MEC refers to the compound MEC including any stereoisomer of MEC. The chemical name of MEC is 4-diphosphocytidyl-2-C-methylerythritol 2-phosphate. In particular embodiments, the term refers to the compound according to the following structure:

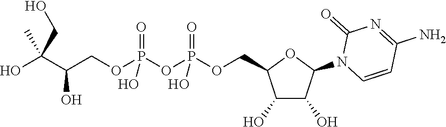 [SMILES: C[C@](O)(CO)[C@H](O)COP(=O)(O)OP(=O)(O)OC[C@H]1O[C@@H](n2ccc(N)nc2=O)[C@H](O)[C@@H]1O]

### HMBPP

The term HMBPP refers to the compound HMBPP including any stereoisomer of HMBPP. The chemical name of HMBPP is (E)-4-Hydroxy-3-methyl-but-2-enyl pyrophosphate. In particular embodiments, the term refers to the compound according to the following structure:

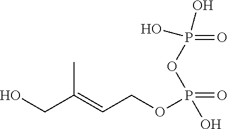

### Acetyl-coA

The term acetyl-coA refers to the compound acetyl-coA including any stereoisomer of acetyl-coA. In particular embodiments, the term refers to the compound according to the following structure:

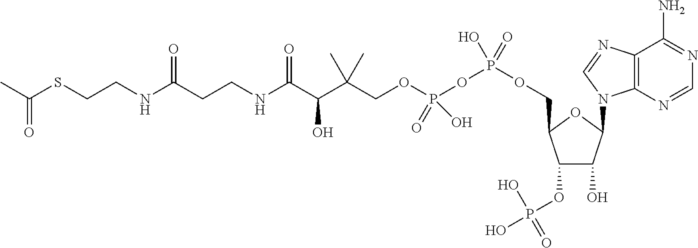 [SMILES: CC(=O)SCCNC(=O)CCNC(=O)[C@H](O)C(C)(C)COP(=O)(O)OP(=O)(O)OC[C@H]1O[C@@H](n2cnc3c(N)ncnc32)[C@H](O)[C@@H]1OP(=O)(O)O]

### Acetoacetyl-coA

The term acetoacetyl-coA refers to the compound acetoacetyl-coA including any stereoisomer of acetoacetyl-coA. The chemical name of acetoacetyl-coA is 3-oxoacyl-CoA. In particular embodiments, the term refers to the compound according to the following structure:

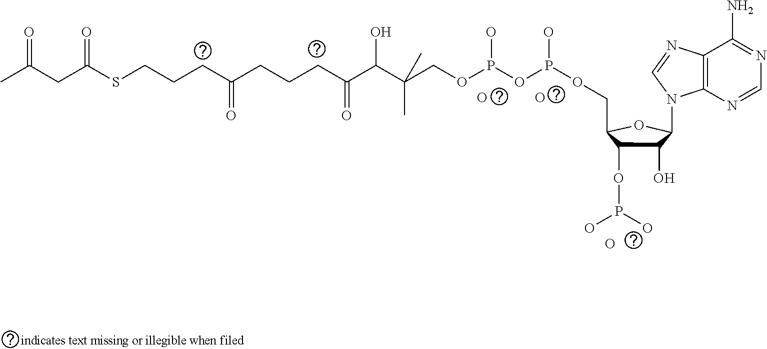

### HMG-CoA

The term HMG-CoA refers to the compound HMG-CoA including any stereoisomer of HMG-CoA. The chemical name of HMG-CoA is 3-hydroxy-3-methylglutaryl CoA. In particular embodiments, the term refers to the compound according to the following structure:

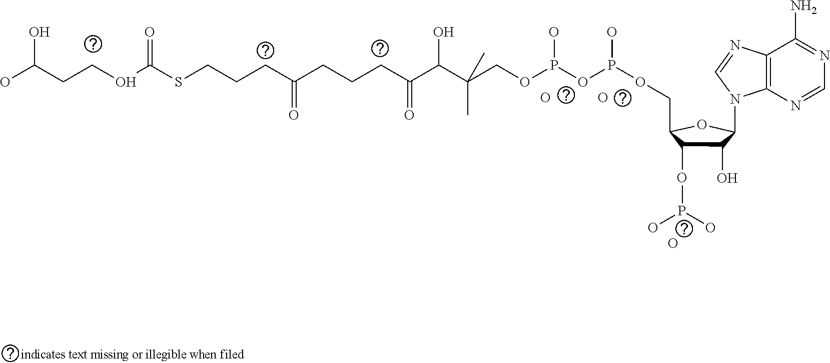

### MVA

The term MVA refers to the compound mevalonate including any stereoisomer of mevalonate. The chemical name of MVA is mevalonate. In particular embodiments, the term refers to the compound according to the following structure:

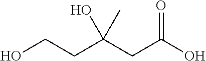

### MVAP

The term MVAP refers to the compound MVAP including any stereoisomer of MVAP. The chemical name of MVAP is mevalonate-5-phosphate. In particular embodiments, the term refers to the compound according to the following structure:

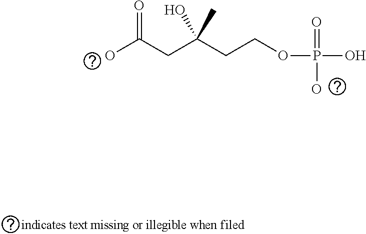

### MVAPP

The term MVAPP refers to the compound MVAPP including any stereoisomer of MVAPP. The chemical name of MVAP is mevalonate-5-pyrophosphate. In particular embodiments, the term refers to the compound according to the following structure:

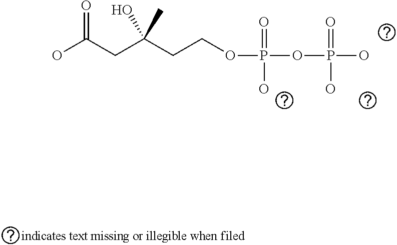 [SMILES: C[C@@](O)(CCOP(=O)(O)OP(=O)(O)O)CC(=O)O]

### IPP

The term IPP refers to the compound IPP including any stereoisomer of IPP. The chemical name of IPP is isopentyl pyrophosphate. In particular embodiments, the term refers to the compound according to the following structure:

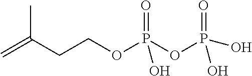

### DMAP

The term DMAP refers to the compound DMAP including any stereoisomer of DMAP. The chemical name of DMAP is 4-Dimethylaminopyridine. In particular embodiments, the term refers to the compound according to the following structure:

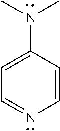

### FPP

The term FPP refers to the compound FPP including any stereoisomer of FPP. The chemical name of FPP is farnesyl diphosphate. In particular embodiments, the term refers to the compound according to the following structure:

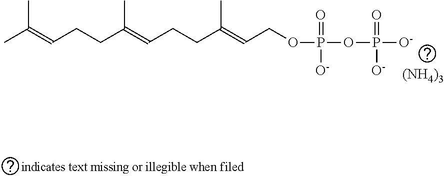

### GGPP

The term GGPP refers to the compound GGPP including any stereoisomer of GGPP. The chemical name of GGPP is Geranylgeranyl diphosphate. In particular embodiments, the term refers to the compound according to the following structure:

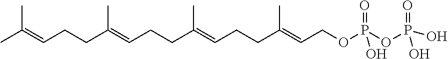 [SMILES: CC(C)=CCC/C(C)=C/CC/C(C)=C/CC/C(C)=C/COP(=O)(O)OP(=O)(O)O]

### Bisabolol

The term bisabolol refers to the compound bisabolol including any stereoisomer of bisabolol. The chemical name of α-(−)-bisabolol. In particular embodiments, the term refers to the compound according to the following structure:

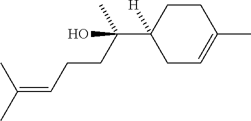 [SMILES: CC(C)=CCC[C@@](C)(O)[C@@H]1CC=C(C)CC1]

### Phytoene

The term phytoene refers to the compound phytoene including any stereoisomer of phytoene. The chemical name of phytoene is 15-cis-Phytoene. In particular embodiments, the term refers to the compound according to the following structure:

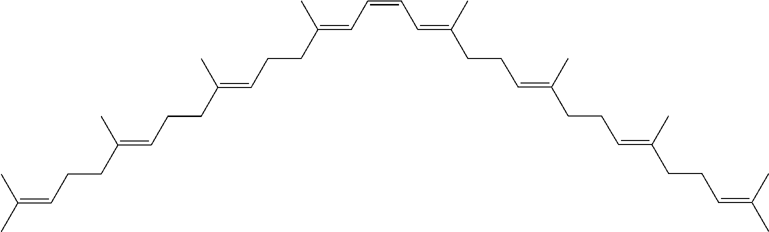 [SMILES: CC(C)=CCC/C(C)=C/CC/C(C)=C/CC/C(C)=C/C=C\C=C(/C)CC/C=C(\C)CC/C=C(\C)CCC=C(C)C]

The invention provides various enzymes, corresponding polynucleotides that encode said enzymes, metabolic pathways, engineered microbial cells, product producing cells, cell free systems and methods directed to the production of terpenoids such as bisabolol, and carotenoids such as phytoene, and in some instances to the further conversion of phytoene to lycopene or neurosporene. The various enzymes, corresponding polynucleotides that encode said enzymes, metabolic pathways, engineered microbial cells, product producing cells, and cell free systems can be used to produce any product that is derived from a terpenoid or a carotenoid. For example, in some embodiments, the various enzymes, corresponding polynucleotides that encode said enzymes, metabolic pathways, engineered microbial cells, product producing cells, cell free systems and methods are used for the production of retinol, which is produced from b-carotene.

The inventors have identified a number of novel sources and amino acid sequences of enzymes that function within these synthesis pathways. Each of these enzymes may be used in combination with known enzymes to improve the synthesis of terpenoids and carotenoids such as phytoene, lycopene, neurosporene or bisabolol. However, in some embodiments combinations of the enzymes of the invention described herein that function at different stages in the pathway are considered to be particularly advantageous.

Accordingly, the invention provides a selection of novel phytoene synthase (PYS) enzymes, geranylgeranyl diphosphate synthase enzymes (GGPPS), bisabolol synthase enzymes, 3-hydroxy-3-methylglutaryl-CoA reductase (HmgR) enzymes (including engineered HmgR enzymes), isopentenyl-diphosphate delta isomerase 1 (IDI) enzymes, 1-deoxy-D-xylulose-5-phosphate synthase (DXS) enzymes, phytoene desaturase (CrtI) enzymes, Acetoacetyl-CoA thiolase (HmgACT) enzymes, Hydroxymethylglutaryl-CoA synthase enzymes (HmgS), 3-hydroxy-3-methylglutaryl-CoA reductase (HmgR), Mevalonate kinase enzymes (MVK), Phosphomevalonate kinase enzymes (PMK), Phosphomevalonate decarboxylase enzymes (PMD), which can be used to improve production of terpenoids and carotenoids such as phytoene, lycopene, neurosporene and bisabolol, and other downstream products such as retinol.

FIG. 1 shows a schematic of the metabolic pathway from glucose and glycerol to bisabolol, phytoene, lycopene, neurosporene and b-carotene. Key enzymes in the production of phytoene are phytoene synthase (PYS) and geranylgeranyl diphosphate synthase (GGPPS). As described herein, novel PYS and GGPPS enzymes are able to improve production of phytoene. However, modulation of the upstream MVA and MEP pathways which drives the production of IPP and DMAPP is also shown herein to improve production of phytoene. Accordingly, improvement of each part of the pathway is, in itself, useful and an improvement on known methods. However, combining improvements in the MEP and MVA pathways with improved PYS and GGPPS enzymes improves phytoene production further.

The invention provides a number of different enzymes and polynucleotides encoding said enzymes, as described in detail below.

### Phytoene Synthase

The invention provides a phytoene synthase (PYS) enzyme or a polynucleotide encoding a PYS enzyme wherein the PYS enzyme has an amino acid sequence of a PYS enzyme from:
Enterobacter sp; Pantoea sp; Daucus sp; Synechocystis sp; Arthrospira sp; Cucumis sp; or Staphylococcus sp;
   or has an amino acid sequence with at least 80%, 85%, 90%, 92%, 94%, 96%, 98%, 99% or 100% similarity to a PYS enzyme from:
Enterobacter sp; Pantoea sp; Daucus sp; Synechocystis sp; Arthrospira sp; Cucumis sp; or Staphylococcus sp.

In some embodiments, the Enterobacter species is Enterobacter agglomerans; the Pantoea species is Pantoea ananatis; the Daucus species is Daucus carota; the Synechocystis species is selected from Synechocystis aquatilis, Synechocystis bourrellyi, Synechocystis fuscopigmentosa, Synechocystis limnetica, Synechocystis minuscula, Synechocystis nigrescens, Synechocystis pevalekii, Synechocystis salina, Synechocystis sp. (strain PCC 6803/Kazusa), and Synechocystis trididemni; the Arthrospira species is Arthrospira platensis; the Cucumis species is Cucumis melo; or the Staphylococcus species is Staphylococcus aureus strain bovine RF122/ET3-1).

In the same or different embodiments, the PYS enzyme has an amino acid sequence of any of SEQ ID NO: 1-7 or has an amino acid sequence with at least 80%, 85%, 90%, 92%, 94%, 96%, 98%, 99% or 100% similarity to any of SEQ ID NO: 1-7.

In some embodiments the PYS enzyme is from: Enterobacter agglomerans and has an amino acid sequence of SEQ ID NO: 3; Pantoea ananatis and has an amino acid sequence of SEQ ID NO: 1; Daucus carota and has an amino acid sequence of SEQ ID NO: 2; Synechocystis species has an amino acid sequence of SEQ ID NO: 4; Arthrospira platensis and has an amino acid sequence of SEQ ID NO: 5; Cucumis melo and has an amino acid sequence of SEQ ID NO: 6; or Staphylococcus aureus strain bovine RF122/ET3-1) and has an amino acid sequence of SEQ ID NO: 7;
or has an amino acid sequence with at least 80%, 85%, 90%, 92%, 94%, 96%, 98%, 99% or 100% similarity to any of the above sequences.

In preferred embodiments the PYS enzyme is from Enterobacter species, for example is from Enterobacter agglomerans. In preferred embodiments the PYS enzyme has an amino acid sequence of SEQ ID NO: 3 or has an amino acid sequence with at least 80%, 85%, 90%, 92%, 94%, 96%, 98%, 99% or 100% similarity to SEQ ID NO: 3.

In some embodiments the phytoene synthase activity is provided by enzyme expressed from CrtB_Aa from Agrobacterium aurantiacum (SEQ ID NO: 51), CrtB_Pa from Pantoea agglomerans (SEQ ID NO: 52), or an enzyme with at least 80%, 85%, 90%, 92%, 94%, 96%, 98%, 99% or 100% similarity to SEQ ID NO: 51 or 52.

In some embodiments where the host cell is S. cerevisiae, the phytoene synthase activity is provided by enzyme expressed from CrtB_Aa from Agrobacterium aurantiacum (SEQ ID NO: 51), CrtB_Pa from Pantoea agglomerans (SEQ ID NO: 52), or an enzyme with at least 80%, 85%, 90%, 92%, 94%, 96%, 98%, 99% or 100% similarity to SEQ ID NO: 51 or 52.

In some embodiments the phytoene synthase activity is a bifunctional lycopene cyclase/phytoene synthase (CrtYB), for example from CrtYB_MI from Mucor lusitanicus (SEQ ID NO: 53) or CrtYB_Xd from Xanthophyllomyces dendrorhous (SEQ ID NO: 54), or an enzyme with at least 80%, 85%, 90%, 92%, 94%, 96%, 98%, 99% or 100% similarity to SEQ ID NO: 53 or 54.

In some embodiments where the host cell is a bacterial cell such as an E. coli cell, the preferred phytoene synthase is SEQ ID NO: 3 or a sequence with an amino acid sequence with at least 80%, 85%, 90%, 92%, 94%, 96%, 98%, 99% or 100% similarity to SEQ ID NO: 3.

In some embodiments where the host cell is a yeast cell such as a S. cerevisiae cell, the preferred phytoene synthase is SEQ ID NO: 51 or a sequence with an amino acid sequence with at least 80%, 85%, 90%, 92%, 94%, 96%, 98%, 99% or 100% similarity to SEQ ID NO: 51.

### GGPPS

The invention also provides a geranylgeranyl diphosphate synthase (GGPPS) or a polynucleotide encoding a GGPPS, wherein the GGPPS enzyme has an amino acid sequence of a GGPPS enzyme from: Hevea sp; Saccharomyces sp; Capsicum sp; Abies sp; Penicillium sp; Mucor sp; Croton sp; Pseudescherichia sp; or Enterobacter sp;
or has an amino acid sequence with at least 80%, 85%, 90%, 92%, 94%, 96%, 98%, 99% or 100% similarity to a GGPPS enzyme from:
   Hevea sp; Saccharomyces sp; Capsicum sp; Abies sp; Penicillium sp; Mucor sp; Croton sp; Pseudescherichia sp; or Enterobacter sp.

In some embodiments: The Hevea sp is Hevea brasiliensis; The Saccharomyces sp is Saccharomyces cerevisiae; The Capsicum sp is Capsicum annuum; The Abies sp is Abies grandis; The Penicillium sp is Penicillium paxilli; The Mucor sp is Mucor lusitanicus; The Croton sp is Croton sublyratus; The Pseudescherichia sp is Pseudescherichia vulneris; or The Enterobacter sp is Enterobacter agglomerans.

In the same or different embodiments, the GGPPS enzyme has an amino acid sequence of any of SEQ ID NO: 8-16 or has an amino acid sequence with at least 80%, 85%, 90%, 92%, 94%, 96%, 98%, 99% or 100% similarity to any of SEQ ID NO: 8-16.

In some embodiments the GGPPS enzyme is from:
Hevea brasiliensis and has an amino acid sequence of SEQ ID NO: 14;
Saccharomyces cerevisiae and has an amino acid sequence of SEQ ID NO: 8;
Capsicum annuum and has an amino acid sequence of SEQ ID NO: 9;
Abies grandis and has an amino acid sequence of SEQ ID NO: 10;
Penicillium paxillin is and has an amino acid sequence of SEQ ID NO: 11;
Mucor lusitanicus and has an amino acid sequence of SEQ ID NO: 12;
Croton sublyratus and has an amino acid sequence of SEQ ID NO: 13;
Pseudescherichia vulneris and has an amino acid sequence of SEQ ID NO: 15; or
Enterobacter agglomerans and has an amino acid sequence of SEQ ID NO: 16;
or has an amino acid sequence with at least 80%, 85%, 90%, 92%, 94%, 96%, 98%, 99% or 100% similarity to any of the above sequences.

In preferred embodiments the GGPPS enzyme is from Hevea sp, preferably from Hevea brasiliensis, and preferably has an amino acid sequence of SEQ ID NO: 14 or has an amino acid sequence with at least 80%, 85%, 90%, 92%, 94%, 96%, 98%, 99% or 100% similarity to SEQ ID NO: 14.

In some embodiments, where the GGPPS enzyme is to be expressed in a eukaryote, for example expressed in yeast, for example expressed in S. cerevisiae, the GGPPS enzyme has an amino acid sequence of SEQ ID NO: 13 or 14, preferably SEQ ID NO: 14, or has an amino acid sequence with at least 80%, 85%, 90%, 92%, 94%, 96%, 98%, 99% or 100% similarity to SEQ ID NO: 13 or 14.

In some embodiments, where the GGPPS enzyme is to be expressed in a prokaryote, for example expressed in bacteria, for example expressed in E. coli, the GGPPS enzyme has an amino acid sequence of SEQ ID NO: 14, or has an amino acid sequence with at least 80%, 85%, 90%, 92%, 94%, 96%, 98%, 99% or 100% similarity to SEQ ID NO: 13 or 14.

In some embodiments the Geranylgeranyl diphosphate synthase activity is provided by the enzyme expressed from CrtE_Af from Archaeoglobus fulgidus (SEQ ID NO: 55), CrtE_Txm from Taxus x media (SEQ ID NO: 56), CrtE_Xd from Xanthophyllomyces dendrorhous (SEQ ID NO: 57), or CrtE_Bt from Blakeslea trispora (SEQ ID NO: 58).

In some embodiments where the host cell is a yeast cell such as S. cerevisiae, the Geranylgeranyl diphosphate synthase activity is provided by the enzyme expressed from CrtE_Af from Archaeoglobus fulgidus (SEQ ID NO: 55), CrtE_Txm from Taxus x media (SEQ ID NO: 56), CrtE_Xd from Xanthophyllomyces dendrorhous (SEQ ID NO: 57), or CrtE_Bt from Blakeslea trispora (SEQ ID NO: 58).

In embodiments where the host cell is a bacterial cell such as E. coli, the preferred GGPPS is SEQ ID NO: 14 or a sequence with an amino acid sequence with at least 80%, 85%, 90%, 92%, 94%, 96%, 98%, 99% or 100% similarity to SEQ ID NO: 14.

In embodiments where the host cell is a yeast cell such as S. cerevisiae, the preferred GGPPS is SEQ ID NO: 55, 56, 57 or 58 or a sequence with an amino acid sequence with at least 80%, 85%, 90%, 92%, 94%, 96%, 98%, 99% or 100% similarity to SEQ ID NO: 55, 56, 57 or 58.

### Bisabolol Synthase

The invention also provides a Bisabolol synthase enzyme or a polynucleotide encoding a Bisabolol synthase enzyme, wherein the Bisabolol synthase enzyme has an amino acid sequence of a Bisabolol synthase enzyme from:
Eremanthus sp; Cynara sp; Artemisia sp; Helianthus sp; or Matricaria sp;
   or has an amino acid sequence with at least 80%, 85%, 90%, 92%, 94%, 96%, 98%, 99% or 100% similarity to a Bisabolol synthase enzyme from:
Eremanthus sp; Cynara sp; Artemisia sp; Helianthus sp; or Matricaria sp.

In some embodiments: The Eremanthus species is Eremanthus erythropappus; The Cynara species is Cynara cardunculus; The Artemisia species is Artemisia annua;
The Helianthus species is Helianthus annuus; or The Matricaria species is Matricaria recutita.

In the same or different embodiments, the Bisabolol synthase enzyme has an amino acid sequence of any of SEQ ID NO: 25-30 or has an amino acid sequence with at least 80%, 85%, 90%, 92%, 94%, 96%, 98%, 99% or 100% similarity to any of SEQ ID NO: 25-30.

In some embodiments the Bisabolol synthase enzyme is from:
Eremanthus erythropappus and has an amino acid sequence of SEQ ID NO: 28;
Cynara cardunculus and has an amino acid sequence of SEQ ID NO: 25;
Artemisia annua and has an amino acid sequence of SEQ ID NO: 26 or 27;
Helianthus annuus and has an amino acid sequence of SEQ ID NO: 29; or
Matricaria recutita and has an amino acid sequence of SEQ ID NO: 30.

In some preferred embodiments, the Bisabolol synthase enzyme is:
a Bisabolol synthase enzyme with an amino acid sequence of SEQ ID NO: 30 or has an amino acid sequence with at least 80%, 85%, 90%, 92%, 94%, 96%, 98%, 99% or 100% similarity to SEQ ID NO: 28;
a Bisabolol synthase enzyme with an amino acid sequence of SEQ ID NO: 25 or has an amino acid sequence with at least 80%, 85%, 90%, 92%, 94%, 96%, 98%, 99% or 100% similarity to SEQ ID NO: 25;
a Bisabolol synthase enzyme with an amino acid sequence of SEQ ID NO: 30 or has an amino acid sequence with at least 80%, 85%, 90%, 92%, 94%, 96%, 98%, 99% or 100% similarity to SEQ ID NO: 30;
a Bisabolol synthase enzyme with an amino acid sequence of SEQ ID NO: 29 or has an amino acid sequence with at least 80%, 85%, 90%, 92%, 94%, 96%, 98%, 99% or 100% similarity to SEQ ID NO: 29.

In embodiments where the host cell is a bacterial cell such as E. coli, the Bisabolol synthase is SEQ ID 28 or a sequence with an amino acid sequence with at least 80%, 85%, 90%, 92%, 94%, 96%, 98%, 99% or 100% similarity to SEQ ID NO: 28.

In embodiments where the host cell is a yeast cell such as a S. cerevisiae cell, the preferred Bisabolol synthase may be SEQ ID NO: 28 or 25 or a sequence with an amino acid sequence with at least 80%, 85%, 90%, 92%, 94%, 96%, 98%, 99% or 100% similarity to SEQ ID NO: 25 or 28.

Preferably the Bisabolol synthase enzyme has an amino acid sequence of SEQ ID NO: 25 or has an amino acid sequence with at least 80%, 85%, 90%, 92%, 94%, 96%, 98%, 99% or 100% similarity to SEQ ID NO: 25.

### HmgR

The invention also provides a 3-hydroxy-3-methylglutaryl-CoA reductase (HmgR) enzyme or a polypeptide encoding a HmgR enzyme (including engineered HmgR enzymes described herein), wherein the enzyme has an amino acid sequence of a 3-hydroxy-3-methylglutaryl-CoA reductase enzyme from:
Streptococcus sp, Saccharomyces sp; or Enterococcus sp;
or has an amino acid sequence with at least 80%, 85%, 90%, 92%, 94%, 96%, 98%, 99% or 100% similarity to a 3-hydroxy-3-methylglutaryl-CoA reductase from: Streptococcus sp; Saccharomyces sp; or Enterococcus sp.

In some embodiments, the Streptococcus sp is Streptococcus pneumoniae; the Saccharomyces sp is Saccharomyces cerevisiae; or the Enterococcus sp is Enterococcus faecalis.

In the same or different embodiments, the 3-hydroxy-3-methylglutaryl-CoA reductase enzyme has an amino acid sequence of any of SEQ ID NO: 19-21 or has an amino acid sequence with at least 80%, 85%, 90%, 92%, 94%, 96%, 98%, 99% or 100% similarity to any of SEQ ID NO: 19-21.

Engineered 3-hydroxy-3-methylglutaryl-CoA reductase enzymes are also provided. The engineered 3-hydroxy-3-methylglutaryl-CoA reductase enzymes described herein comprise point mutations relative to the 3-hydroxy-3-methylglutaryl-CoA reductase protein sequence from S. pneumoniae with the sequence of SEQ ID NO: 19 (HmgR1), or relative to the 3-hydroxy-3-methylglutaryl-CoA reductase protein sequence from S. cerevisiae with the sequence of SEQ ID NO: 20, or relative to the 3-hydroxy-3-methylglutaryl-CoA reductase protein sequence from Enterococcus faecalis with the sequence of SEQ ID NO: 21. However, the skilled person will appreciate that corresponding point mutations may be made in other 3-hydroxy-3-methylglutaryl-CoA reductase enzyme sequences, for example from other species, with the same improved effect obtained. The corresponding amino acid positions for the mutations can be identified in other species using various tools that can identify regions of sequence and structure homology.

For example in one embodiment the 3-hydroxy-3-methylglutaryl-CoA reductase enzyme comprises a substitution at one or more positions selected from positions 102, 188, 236, or 343, wherein said positions are numbered with reference to SEQ ID No: 19 or 20 or 21.

In some embodiments the engineered 3-hydroxy-3-methylglutaryl-CoA reductase comprises a substitution at positions 188 and 236 with respect to SEQ ID NO: 19, or 20 or 21.

In some embodiments the engineered 3-hydroxy-3-methylglutaryl-CoA reductase comprises a substitution at positions 188 and 343 with respect to SEQ ID NO: 19, or 20 or 21.

In some embodiments the engineered 3-hydroxy-3-methylglutaryl-CoA reductase comprises a substitution at positions 236 and 343 with respect to SEQ ID NO: 19, or 20 or 21.

In some embodiments the engineered 3-hydroxy-3-methylglutaryl-CoA reductase comprises a substitution at positions 188, 236 and 343 with respect to SEQ ID NO: 19, or 20 or 21.

In some embodiments the engineered 3-hydroxy-3-methylglutaryl-CoA reductase comprises a substitution at positions 102, 188 and 236 with respect to SEQ ID NO: 19, or 20 or 21.

In some embodiments the engineered 3-hydroxy-3-methylglutaryl-CoA reductase comprises a substitution at positions 102, 188 and 343 with respect to SEQ ID NO: 19, or 20 or 21.

In some embodiments the engineered 3-hydroxy-3-methylglutaryl-CoA reductase comprises a substitution at positions 102, 236 and 343 with respect to SEQ ID NO: 19, or 20 or 21.

In some embodiments the engineered 3-hydroxy-3-methylglutaryl-CoA reductase comprises a substitution at positions 102, 188, 236 and 343 with respect to SEQ ID NO: 19, or 20 or 21.

In some embodiments the substitution at position 236 is a substitution to a histidine. For example where the HmgR enzyme is from S. pneumoniae and has a sequence of SEQ ID NO: 19, the mutation is a Q236H mutation (SEQ ID NO: 64); where the HmgR enzyme is from S. cerevisiae and has a sequence of SEQ ID NO: 20, the mutation is a W236H; or where the HmgR enzyme is from Enterococcus faecalis and has a sequence of SEQ ID NO: 21, the mutation is a S236H.

In some embodiments the substitution at position 188 is a substitution to a serine. For example where the HmgR enzyme is from S. pneumoniae and has a sequence of SEQ ID NO: 19, the mutation is a T188S mutation (SEQ ID NO: 63); where the HmgR enzyme is from S. cerevisiae and has a sequence of SEQ ID NO: 20, the mutation is a G188S; or where the HmgR enzyme is from Enterococcus faecalis and has a sequence of SEQ ID NO: 21, the mutation is a Q188S.

In some embodiments the substitution at position 343 is a substitution to an aspartic acid. For example where the HmgR enzyme is from S. pneumoniae and has a sequence of SEQ ID NO: 19, the mutation is a G343D mutation (SEQ ID NO: 65); where the HmgR enzyme is from S. cerevisiae and has a sequence of SEQ ID NO: 20, the mutation is a P343D; or where the HmgR enzyme is from Enterococcus faecalis and has a sequence of SEQ ID NO: 21, the mutation is a Q343D.

In some embodiments the substitution at position 102 is a substitution to an isoleucine. For example where the HmgR enzyme is from S. pneumoniae and has a sequence of SEQ ID NO: 19, the mutation is a T102I mutation (SEQ ID NO: 65); where the HmgR enzyme is from S. cerevisiae and has a sequence of SEQ ID NO: 20, the mutation is a K102I; or where the HmgR enzyme is from Enterococcus faecalis and has a sequence of SEQ ID NO: 21, the mutation is a F102I.

In some embodiments the engineered HmgR enzyme comprises or consists of a sequence of SEQ ID N: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80.

Preferably the engineered HmgR enzyme comprises or consists of a sequence of SEQ ID NO: 64.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 19, 20 or 21 provided that the enzyme comprises a substitution at one or more positions selected from positions 102, 188, 236, or 343, wherein said positions are numbered with reference to SEQ ID No: 19, 20 or 21.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 64, provided that the residue at position 236 is a histidine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 63, provided that the residue at position 188 is a serine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 65, provided that the residue at position 343 is an aspartic acid.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 76, provided that the residue at position 102 is an isoleucine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 79, provided that the residue at position 236 is a histidine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 78, provided that the residue at position 188 is a serine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 80, provided that the residue at position 343 is an aspartic acid.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 77, provided that the residue at position 102 is an isoleucine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 74, provided that the residue at position 236 is a histidine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 73, provided that the residue at position 188 is a serine.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 75, provided that the residue at position 343 is an aspartic acid.

In some embodiments the engineered 3-hydroxy-3-methylglutaryl-CoA reductase enzymes comprise a sequence that has at least 80%, 85%, 90%, 92%, 94%, 96%, 98%, 99% or 100% similarity to SEQ ID NO: 72, provided that the residue at position 102 is an isoleucine.

In some embodiments the engineered 3-hydroxy-3-methylglutaryl-CoA reductase comprises T188S and Q236H substitutions with respect to SEQ ID NO: 19.

In some embodiments the engineered 3-hydroxy-3-methylglutaryl-CoA reductase comprises T188S and G343D substitutions with respect to SEQ ID NO: 19.

In some embodiments the engineered 3-hydroxy-3-methylglutaryl-CoA reductase comprises Q236H and G343D substitutions with respect to SEQ ID NO: 19.

In some embodiments the engineered 3-hydroxy-3-methylglutaryl-CoA reductase comprises a substitution at positions T188S, Q236H and G343D substitutions with respect to SEQ ID NO: 19.

These engineered 3-hydroxy-3-methylglutaryl-CoA reductase enzymes have improved activity over the corresponding non-engineered enzymes, and are able to improve the production of terpenoid and carotene precursors. The engineered 3-hydroxy-3-methylglutaryl-CoA reductase enzymes described herein can be considered to provide means and methods for improved terpenoid and carotene production, for example improved phytoene, lycopene, neurosporene and bisabolol production.

### MvaK

The invention also provides a mevalonate kinase (MvaK) enzyme or a polypeptide encoding a MvaK enzyme (including engineered MvaK enzymes described herein), wherein the enzyme has an amino acid sequence of a mevalonate kinase enzyme from:
Streptococcus sp, Saccharomyces sp; or Enterococcus sp;
or has an amino acid sequence with at least 80%, 85%, 90%, 92%, 94%, 96%, 98%, 99% or 100% similarity to a mevalonate kinase from:

### Streptococcus sp; Saccharomyces sp; or Enterococcus sp.

In some embodiments, the:
Saccharomyces sp is Saccharomyces cerevisiae

In the same or different embodiments, the mevalonate kinase enzyme has an amino acid sequence of any of SEQ ID NO: 22 or has an amino acid sequence with at least 80%, 85%, 90%, 92%, 94%, 96%, 98%, 99% or 100% similarity to any of SEQ ID NO: 22.

Engineered mevalonate kinase enzymes are also provided. The engineered mevalonate kinase enzymes described herein comprise point mutations relative to the mevalonate kinase protein sequence from S. cerevisiae with the sequence of SEQ ID NO: 22. However, the skilled person will appreciate that corresponding point mutations may be made in other mevalonate kinase enzyme sequences, for example from other species, with the same improved effect obtained.

For example in one embodiment the mevalonate kinase enzyme comprises a substitution at one or more positions selected from positions E419, K402, E73, G393, L175, D74, V414, L413, L408, K409, Q426, K411, wherein said positions are numbered with reference to SEQ ID No: 22.

In some embodiments the substitution at position 419 is a substitution to a aspartic acid. For example where the MvaK enzyme is from S. cerevisiae and has a sequence of SEQ ID NO: 22, the mutation is a E419D mutation.

In some embodiments the substitution at position 402 is a substitution to a valine. For example where the MvaK enzyme is from S. cerevisiae and has a sequence of SEQ ID NO: 22, the mutation is a K402V mutation.

In some embodiments the substitution at position 73 is a substitution to a glycine. For example where the MvaK enzyme is from S. cerevisiae and has a sequence of SEQ ID NO: 22, the mutation is a E73G mutation.

In some embodiments the substitution at position 393 is a substitution to a valine. For example where the MvaK enzyme is from S. cerevisiae and has a sequence of SEQ ID NO: 22, the mutation is a G393V mutation.

In some embodiments the substitution at position 175 is a substitution to a valine. For example where the MvaK enzyme is from S. cerevisiae and has a sequence of SEQ ID NO: 22, the mutation is a L175V mutation.

In some embodiments the substitution at position 74 is a substitution to a glutamic acid. For example where the MvaK enzyme is from S. cerevisiae and has a sequence of SEQ ID NO: 22, the mutation is a D74E mutation.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, 414, 413, 408, 409, 426, 411, wherein said positions are numbered with reference to SEQ ID No: 12.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 419 is a aspartic acid.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 402 is a valine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 73 is a glycine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 393 is a valine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 175 is a valine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 74 is a glutamic acid.

These engineered mevalonate kinase enzymes have improved activity over the corresponding non-engineered enzymes, and are able to improve the production of terpenoid and carotene precursors. The engineered mevalonate kinase enzymes described herein can be considered to provide means and methods for improved terpenoid and carotene production, for example improved phytoene, lycopene, neurosporene and bisabolol production.

### IDI

The invention also provides an isopentenyl-diphosphate delta isomerase 1 (IDI) enzyme or a polynucleotide encoding an IDI enzyme, that in some embodiments is from B. subtilis or E. coli. In some embodiments the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41.

### DXS

The invention also provides a 1-deoxy-D-xylulose-5-phosphate synthase (DXS) enzyme or a polynucleotide encoding a DXS enzyme, that in some embodiments is from E. coli. In some embodiments the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

### CRTI

The invention also provides a phytoene desaturase (CrtI) enzyme or a polynucleotide encoding a CrtI enzyme, wherein the phytoene desaturase enzyme has an amino acid sequence of a phytoene desaturase enzyme from:
Pantoea sp; Paracoccus sp; Pseudescherichia sp; Rhodobacter sp; Synechocystis sp; or Streptomyces sp;
   or has an amino acid sequence with at least 80%, 85%, 90%, 92%, 94%, 96%, 98%, 99% or 100% similarity to a phytoene desaturase enzyme from:

### Pantoea sp; Paracoccus sp; Pseudescherichia sp; Rhodobacter sp; Synechocystis sp; or Streptomyces sp.

In some embodiments:
The Pantoea sp. is Pantoea ananatis;
The Paracoccus sp. is Paracoccus sp strain N81106/MBIC 01143;
The Pseudescherichia sp. is Pseudescherichia vulneris;
The Rhodobacter sp. is Rhodobacter capsulatus;
The Synechocystis sp. is Synechocystis sp strain PCC 6714; and
The Streptomyces sp. is Streptomyces griseus.

In some embodiments the phytoene desaturase enzyme has an amino acid sequence of any of SEQ ID NO: 33-38 or has an amino acid sequence with at least 80%, 85%, 90%, 92%, 94%, 96%, 98%, 99%98%, 99% or 100% similarity to any of SEQ ID NO: 33-38.

In some embodiments the phytoene desaturase enzyme is from:
Pantoea ananatis and has an amino acid sequence of SEQ ID NO: 33; Paracoccus sp strain N81106/MBIC 01143 and has an amino acid sequence of SEQ ID NO: 34;
Pseudescherichia vulneris and has an amino acid sequence of SEQ ID NO: 35; Rhodobacter capsulatus and has an amino acid sequence of SEQ ID NO: 36; Synechocystis sp strain PCC 6714 and has an amino acid sequence of SEQ ID NO: 37; or
Streptomyces griseus and has an amino acid sequence of SEQ ID NO: 38,
   or has an amino acid sequence with at least 80%, 85%, 90%, 92%, 94%, 96%, 98%, 99%98%, 99% or 100% similarity to any of the above sequences.

In some embodiments the phytoene desaturase enzyme converts phytoene to lycopene. In some embodiments the phytoene desaturase that converts phytoene to lycopene is:
is from Pantoea sp or from Pantoea ananatis; and/or has an amino acid sequence of SEQ ID NO: 33 or has an amino acid sequence with at least 80%, 85%, 90%, 92%, 94%, 96%, 98%, 99% or 100% similarity to SEQ ID NO: 33; or
is from Paracoccus sp; and/or has an amino acid sequence of SEQ ID NO: 34 or has an amino acid sequence with at least 80%, 85%, 90%, 92%, 94%, 96%, 98%, 99% or 100% similarity to SEQ ID NO: 34;
is from Pseudescherichia sp, or Pseudescherichia vulneris and/or has an amino acid sequence of SEQ ID NO: 35 or has an amino acid sequence with at least 80%, 85%, 90%, 92%, 94%, 96%, 98%, 99% or 100% similarity to SEQ ID NO: 35;
is from Synechocystis sp or Synechocystis sp strain PCC 6714 and/or has an amino acid sequence of SEQ ID NO: 37 or has an amino acid sequence with at least 80%, 85%, 90%, 92%, 94%, 96%, 98%, 99% or 100% similarity to SEQ ID NO: 37; or
is from Streptomyces sp or Streptomyces griseus and/or has an amino acid sequence of SEQ ID NO: 38 or has an amino acid sequence with at least 80%, 85%, 90%, 92%, 94%, 96%, 98%, 99% or 100% similarity to SEQ ID NO: 38.

In some embodiments the phytoene desaturase enzyme converts phytoene to neurosporene. In some embodiments the phytoene desaturase enzyme that converts phytoene to neurosporene:
is from Rhodobacter sp; optionally from Rhodobacter capsulatus; and/or
has an amino acid sequence of SEQ ID NO: 36 or has an amino acid sequence with at least 80%, 85%, 90%, 92%, 94%, 96%, 98%, 99%98%, 99% or 100% similarity to SEQ ID NO: 36.

In some preferred embodiments the phytoene desaturase enzyme:
a) is from Paracoccus sp;
b) is from Paracoccus sp strain N81106/MBIC 01143; and/or
c) has an amino acid sequence of SEQ ID NO: 34 or has an amino acid sequence with at least 80%, 85%, 90%, 92%, 94%, 96%, 98%, 99%98%, 99% or 100% similarity to SEQ ID NO: 34; or
d) is from Rhodobacter sp;
e) is from Rhodobacter capsulatus; and/or
f) has an amino acid sequence of SEQ ID NO: 36 or has an amino acid sequence with at least 80%, 85%, 90%, 92%, 94%, 96%, 98%, 99%98%, 99% or 100% similarity to SEQ ID NO: 36.

### Mevalonate Pathway Enzymes

The invention provides various new and improved enzymes that function in the mevalonate pathway.

In some embodiments the invention provides an Acetoacetyl-CoA thiolase (HmgACT), enzyme that is from Enterococcus faecalis. In some embodiments the HmgACT enzyme has an amino acid sequence of SEQ ID NO: 17 or has at least 80%, 85%, 90%, 92%, 94%, 96%, 98%, 99%98%, 99% or 100% similarity to SEQ ID NO: 17.

In some embodiments the invention provides a Hydroxymethylglutaryl-CoA synthase (HmgS) enzyme that is from Enterococcus faecalis. In some embodiments the HmgS enzyme has an amino acid sequence of SEQ ID NO: 18 or has at least 80%, 85%, 90%, 92%, 94%, 96%, 98%, 99% or 100% similarity to SEQ ID NO: 18.

In some embodiments the invention provides a 3-hydroxy-3-methylglutaryl-CoA reductase (HmgR) enzyme that is:
has at least 80%, 85%, 90%, 92%, 94%, 96%, 98%, 99% or 100% similarity to SEQ ID NO: 19, 20 or 21 provided that the enzyme comprises a substitution at one or more positions selected from positions 188, 236, or 343, wherein said positions are numbered with reference to SEQ ID No: 19, 20, or 21;
from S. pneumoniae and in some embodiments has an amino acid sequence of SEQ ID NO: 19 or has at least 80%, 85%, 90%, 92%, 94%, 96%, 98%, 99% or 100% similarity to SEQ ID NO: 19;
from S. cerevisiae and in some embodiments has an amino acid sequence of SEQ ID NO: 20 or has at least 80%, 85%, 90%, 92%, 94%, 96%, 98%, 99% or 100% similarity to SEQ ID NO: 20;
from Enterococcus faecalis and in some embodiments has an amino acid sequence of SEQ ID NO: 21 or has at least 80%, 85%, 90%, 92%, 94%, 96%, 98%, 99% or 100% similarity to SEQ ID NO: 21.

In some embodiments the invention provides a Mevalonate kinase (MVK) enzyme that is from S. cerevisiae. In some embodiments the MVK enzyme has an amino acid sequence of SEQ ID NO: 22 or has at least 80%, 85%, 90%, 92%, 94%, 96%, 98%, 99% or 100% similarity to SEQ ID NO: 22.

In some embodiments the mevalonate kinase enzyme comprises a substitution at one or more positions selected from positions 419, 402, 73, 393, 175, 74, 414, 413, 408, 409, 426, 411, wherein said positions are numbered with reference to SEQ ID No: 22.

In some embodiments the substitution at position 419 is a substitution to a aspartic acid. For example where the MvaK enzyme is from S. cerevisiae and has a sequence of SEQ ID NO: 22, the mutation is a E419D mutation [SEQ ID NO: 66].

In some embodiments the substitution at position 402 is a substitution to a valine. For example where the MvaK enzyme is from S. cerevisiae and has a sequence of SEQ ID NO: 22, the mutation is a K402V mutation [SEQ ID NO: 67].

In some embodiments the substitution at position 73 is a substitution to a glycine. For example where the MvaK enzyme is from S. cerevisiae and has a sequence of SEQ ID NO: 22, the mutation is a E73G mutation [SEQ ID NO: 68].

In some embodiments the substitution at position 393 is a substitution to a valine. For example where the MvaK enzyme is from S. cerevisiae and has a sequence of SEQ ID NO: 22, the mutation is a G393V mutation [SEQ ID NO: 69].

In some embodiments the substitution at position 175 is a substitution to a valine. For example where the MvaK enzyme is from S. cerevisiae and has a sequence of SEQ ID NO: 22, the mutation is a L175V mutation [SEQ ID NO: 70].

In some embodiments the substitution at position 74 is a substitution to a glutamic acid. For example where the MvaK enzyme is from S. cerevisiae and has a sequence of SEQ ID NO: 22, the mutation is a D74E mutation [SEQ ID NO: 71].

In some embodiments the engineered mevalonate kinase enzymes comprise a sequence that has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, 414, 413, 408, 409, 426, 411, wherein said positions are numbered with reference to SEQ ID No: 12.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 419 is a aspartic acid.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 402 is a valine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 73 is a glycine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 393 is a valine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 175 is a valine.

In some embodiments the engineered mevalonate kinase enzymes comprise a sequence that has at least 80%, 85%, 90%, 92%, 94%, 96%, 98%, 99% or 100% similarity to SEQ ID NO: 22, provided that the residue at position 74 is a glutamic acid. some embodiments the invention provides a Phosphomevalonate kinase (PMK) enzyme that is from S. cerevisiae. In some embodiments the PMK enzyme has an amino acid sequence of SEQ ID NO: 23 or has at least 80%, 85%, 90%, 92%, 94%, 96%, 98%, 99% or 100% similarity to SEQ ID NO: 23.

In some embodiments the invention provides a Phosphomevalonate decarboxylase (PMD) enzyme that is from S. cerevisiae. In some embodiments the PMD enzyme has an amino acid sequence of SEQ ID NO: 24 or has at least 80%, 85%, 90%, 92%, 94%, 96%, 98%, 99% or 100% similarity to SEQ ID NO: 24.

In some embodiments the invention provides a mevalonate pathway that comprises one or more of the above MVA pathway enzymes, for example comprises one or more or all of the Acetoacetyl-CoA thiolase (HmgACT), Hydroxymethylglutaryl-CoA synthase (HmgS), 3-hydroxy-3-methylglutaryl-CoA reductase (HmgR), Mevalonate kinase (MVK), Phosphomevalonate kinase (PMK), Phosphomevalonate decarboxylase (PMD) as defined herein.

In preferred embodiments, the mevalonate pathway comprises an engineered 3-hydroxy-3-methylglutaryl-CoA reductase (HmgR) as described herein, i.e.:
That has at least 80%, 85%, 90%, 92%, 94%, 96%, 98%, 99% or 100% similarity to SEQ ID NO: 19, 20 or 21 provided that the enzyme comprises a substitution at one or more positions selected from positions 102, 188, 236, or 343, wherein said positions are numbered with reference to SEQ ID No: 19, 20 or 21; or has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, a an aspartic acid at residue 343, and/or an isoleucine at position 102; or comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;

The invention also provides the necessary polynucleotides, vectors, host cells and systems that can be used to produce phytoene, lycopene, neurosporene and/or bisabolol.

### Polynucleotides

The invention provides polynucleotides that encode any one or more of the enzymes described herein, including: PYS, GGPPS, Bisabolol synthase, HmgR, IDI, DXS, CrtI, Acetoacetyl-CoA thiolase (HmgACT), Hydroxymethylglutaryl-CoA synthase (HmgS), 3-hydroxy-3-methylglutaryl-CoA reductase (HmgR) (including the engineered HmgR enzymes described herein), Mevalonate kinase (MVK), Phosphomevalonate kinase (PMK), Phosphomevalonate decarboxylase (PMD), acetoacetyl-CoA thiolase, Hmg-CoA synthase, or Hmg-CoA reductase as defined herein. The polynucleotides may be the naturally occurring polynucleotide, where appropriate. In some embodiments the polynucleotide is a polynucleotide that has been codon optimised to express the corresponding protein in a particular host cell. Codon optimisation is routine for the skilled person.

It will be clear then that the invention provides one or more polynucleotides that encode one or more of:
a) a phytoene synthase enzyme as defined herein;
b) a GGPPS enzyme as defined herein;
c) a Bisabololol synthase enzyme as defined herein;
d) a 3-hydroxy-3-methylglutaryl-CoA reductase enzyme as defined herein;
e) an isopentenyl-diphosphate delta isomerase enzyme as defined herein;
f) a 1-deoxy-D-xylulose-5-phosphate synthase enzyme as defined herein;
g) a phytoene desaturase enzyme as defined herein;
h) Acetoacetyl-CoA thiolase (HmgACT) enzyme as defined herein;
i) Hydroxymethylglutaryl-CoA synthase enzyme as defined herein;
j) 3-hydroxy-3-methylglutaryl-CoA reductase (HmgR) enzyme, including the engineered HmgR enzymes described herein as defined herein;
k) Mevalonate kinase (MVK) enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22;
l) Phosphomevalonate kinase (PMK) enzyme as defined herein;
m) Phosphomevalonate decarboxylase (PMD) enzyme as defined herein; and/or
n) 3-hydroxy-3-methylglutaryl-CoA reductase (HmgR) enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the Enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21:
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence.

The polynucleotide may be any polynucleotide, for example DNA or RNA. In some preferred embodiments, the polynucleotide is DNA.

In the same or different preferred embodiment the polynucleotide is operably linked to a promoter. In some embodiments the promoter is:
a) a constitutive promoter;
b) an inducible promoter;
c) a weak promoter; and/or
d) a strong promoter.

In the same or different embodiments the promoter may be selected from the group comprising or consisting of:
a) a eukaryotic promoter, optionally a promoter that is operable in a eukaryote, optionally a yeast cell, a mammalian cell, an insect cell, optionally wherein the yeast cell is a Saccharomyces cerevisiae cell;
b) a prokaryotic promoter, optionally a promoter that is operable in a prokaryote, optionally an Escherichia coli cell.

The skilled person will be aware of different strength promoters, and is able to optimise the most appropriate promoter or combination of promoters for the expression of a given set of genes or pathway. For example the pGal1/10 promoter is a strong bidirectional promoter induced by galactose, whilst the pGal3 and pGal7 are weak promoters induced by galactose.

The skilled person will appreciate that a single polynucleotide molecule may comprise a coding region for more than one polypeptide, for example more than one enzyme as described herein. Accordingly in some embodiments the polynucleotide of the invention encodes 2 or more of said polypeptides, optionally encodes 2, 3, 4, 5, 6, 7, 8, 9, 10, or more of said polypeptides. In some embodiments at least 2 of the said polypeptides are operably linked to the same promoter to form an operon.

The skilled person will appreciate that when used in a method of producing carotenoids and terpenoids, such as phytoene, lycopene, neurosporene and/or other downstream products such as retinol, in a cellular context, the polynucleotide(s) that encode the relevant polypeptides or enzymes may be integrated into the genome of the host cell, or may be maintained extra-chromosomally for example on a vector such as a plasmid. Where more than one polynucleotide is to be expressed in a cell, more than one polynucleotide can be integrated into the genome. The one or more polynucleotides may be integrated into the host genome at the same locus, or the one or more polynucleotides may be integrated into the host genome at different loci. A single host cell can comprise one or more genomically integrated polynucleotides as described here in combination with one or more extrachromosomally maintained polynucleotides described herein.

Accordingly in some embodiments, the polynucleotide(s) of the invention is located in the genomic nucleic acid of a host cell, for example located within a host chromosome of a host cell. Methods of integrating polynucleotides into host genomes are well known and include, for example, transformation followed by homologous recombination.

In other instances, the polynucleotide of the invention is part of a larger polynucleotide, for example part of a vector. In some instances the vector is a plasmid.

There may be some instances where it is appropriate for some of the enzymes required for the production of carotenoids and terpenoids, such as phytoene, lycopene, neurosporene and/or bisabolol synthesis to be located in the host genomic nucleic acid and some of the enzymes to be maintained extrachromosomally. The present invention contemplates such eventualities.

### Vectors

As discussed above, it is routine to express proteins of interest within a cell, such as the enzymes of the invention, from a vector such as a plasmid. Accordingly the invention also provides a vector comprising one or more polynucleotides of the invention as described elsewhere herein.

In some embodiments the vector is a plasmid. The skilled person will appreciate that one way to influence the expression level of a particular protein is to use a vector which has an appropriate copy number. Accordingly in some instances the plasmid is a high copy number plasmid or a medium copy plasmid or a low copy number plasmid. The skilled person will understand that various combinations of low, medium and high copy number plasmids can be deployed within the same cell to achieve appropriate expression levels of various proteins.

The skilled person will appreciate what is meant by low, medium and high copy number. In one embodiment a low copy number plasmid is defined as a plasmid that exists within a cell at less than 20 copies per cell; a medium copy number plasmid is defined as a plasmid that exists within a cell at between 2-40 copies per cell; and a high copy number plasmid is defined as a plasmid that exists within a cell at more than 40 copies per cell. The skilled person is readily able to identify the copy number per cell of a plasmid. For example the copy number can be determined by gel analysis (Held, D., Yaeger, K., and Novy, R. (2003) inNovations 18, 4-6).]

In specific embodiments the invention provides a vector that comprises a polynucleotide encoding one or more of:
a) a phytoene synthase enzyme as defined herein;
b) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52;
c) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
d) a GGPPS enzyme as defined herein;
e) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
f) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
g) a phytoene synthase enzyme as defined herein and a GGPPS enzyme as defined herein;
h) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
j) a phytoene synthase enzyme as defined herein and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
k) a phytoene synthase enzyme having an amino acid sequence of any of SEQ ID NO: 1-7 or having an amino acid sequence with at least 80%, 85%, 90%, 92%, 94%, 96%, 98%, 99% or 100% similarity to any of SEQ ID NO: 1-7 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
l) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
m) the mevalonate pathway expression system according to any of claims H1-H5;
n) a HmgACT enzyme, for example of SEQ ID NO: 17 or a protein with an amino acid sequence with at least 80%, 85%, 90%, 92%, 94%, 96%, 98%, 99% or 100% similarity to said sequence;
o) a HmgS enzyme, for example of SEQ ID NO: 18 or a protein with an amino acid sequence with at least 80%, 85%, 90%, 92%, 94%, 96%, 98%, 99% or 100% similarity to said sequence;
p) a HmgR enzyme (including engineered HmgR enzymes described herein), for example that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
q) a MVK enzyme, for example of SEQ ID NO: 22 or a protein with an amino acid sequence with at least 80%, 85%, 90%, 92%, 94%, 96%, 98%, 99% or 100% similarity to said sequence; or that
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22;
r) a PMK enzyme, for example of SEQ ID NO: 23 or a protein with an amino acid sequence with at least 80%, 85%, 90%, 92%, 94%, 96%, 98%, 99% or 100% similarity to said sequence;
s) a PMD enzyme, for example of SEQ ID NO: 24 or a protein with an amino acid sequence with at least 80%, 85%, 90%, 92%, 94%, 96%, 98%, 99% or 100% similarity to said sequence
t) a HmgACT enzyme, for example of SEQ ID NO: 17 or a protein with an amino acid sequence with at least 80%, 85%, 90%, 92%, 94%, 96%, 98%, 99% or 100% similarity to said sequence;
a HmgS enzyme, for example of SEQ ID NO: 18 or a protein with an amino acid sequence with at least 80%, 85%, 90%, 92%, 94%, 96%, 98%, 99% or 100% similarity to said sequence;
a HmgR enzyme (including engineered HmgR enzymes described herein), for example that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
A Mevalonate kinase (MVK) enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22;
a PMK enzyme, for example of SEQ ID NO: 23 or a protein with an amino acid sequence with at least 80%, 85%, 90%, 92%, 94%, 96%, 98%, 99% or 100% similarity to said sequence;
a PMD enzyme, for example of SEQ ID NO: 24 or a protein with an amino acid sequence with at least 80%, 85%, 90%, 92%, 94%, 96%, 98%, 99% or 100% similarity to said sequence; and
an isopentenyl-diphosphate delta isomerase 1 enzyme as described herein, for example wherein the IDI enzyme is from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41;
u) a phytoene desaturase enzyme as described herein, for example of a phytoene desaturase enzyme with an amino acid sequence of any of SEQ ID NO: 33-38 or has an amino acid sequence with at least 80%, 85%, 90%, 92% 94%, 96%, 98%, 99% or 100% similarity to any of SEQ ID NO: 33-38;
v) a bisabolol synthase enzyme as defined herein, for example a bisabolol synthase enzyme having an amino acid sequence of SEQ ID NO: 25-29 or having an amino acid sequence with at least 80%, 85%, 90%, 92%, 94%, 96%, 98%, 99% or 100% similarity to any of SEQ ID NO: 25-30;
w) an isopentenyl-diphosphate delta isomerase 1 enzyme as described herein, for example wherein the IDI enzyme is from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
x) a 1-deoxy-D-xylulose-5-phosphate synthase enzyme as described herein, optionally wherein the DXS enzyme is from E. coli, optionally wherein the 1-deoxy-D-xylulose-5 -phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32;
z) a phytoene synthase enzyme that is a CrtB enzyme with an amino acid sequence of SEQ ID NO: 52 or with an amino acid sequence with at least 80%, 85%, 90%, 92%, 94%, 96%, 98%, 99% or 100% similarity to SEQ ID NO: 52 and a GGPPS enzyme that is a CrtE enzyme having an amino acid sequence of SEQ ID NO: 55 or with an amino acid sequence with at least 80%, 85%, 90%, 92%, 94%, 96%, 98%, 99% or 100% similarity to SEQ ID NO: 55.

The invention also provides a vector that comprises a polynucleotide encoding all of:
i) a HmgACT enzyme;
ii) a HmgS enzyme;
iii) a HmgR enzyme (including engineered HmgR enzymes described herein);
iv) a MVK enzyme;
v) a PMK enzyme; and
vi) a PMD enzyme.

Collectively these 6 enzymes make up the MVA pathway. Accordingly, the invention provides an MVA pathway which comprises:
i) a HmgACT enzyme;
ii) a HmgS enzyme;
iii) a HmgR enzyme (including engineered HmgR enzymes described herein);
iv) a MVK enzyme;
v) a PMK enzyme; and
vi) a PMD enzyme;
   or one or more polynucleotides that encode the:
i) HmgACT enzyme;
ii) a HmgS enzyme;
iii) a HmgR enzyme (including engineered HmgR enzymes described herein);
iv) a MVK enzyme;
v) a PMK enzyme; and
vi) a PMD enzyme.

In some instances it is considered advantageous if the vector that comprises polynucleotides that encode the 6 MVA proteins above also encodes the IDI enzyme. Accordingly in some embodiments the vector comprises:
one or more polynucleotides that encode the:
   i) HmgACT enzyme;
   ii) a HmgS enzyme;
   iii) a HmgR enzyme (including engineered HmgR enzymes described herein);
   iv) a MVK enzyme;
   v) a PMK enzyme;
   vi) a PMD enzyme; and
   vii) an IDI enzyme

The Hmg ACT enzyme; HmgS enzyme; HmgR enzyme (including engineered HmgR enzymes described herein); MVK enzyme; PMK enzyme; PMD enzyme encoded by the vector or integrated in to the host genome may be any Hmg ACT enzyme; HmgS enzyme; HmgR enzyme (including engineered HmgR enzymes described herein); MVK enzyme; PMK enzyme; PMD enzyme. For example these enzymes may be autologous to a particular host cell which is intended to express the enzymes. For example S. cerevisiae natively contains the MVA pathway therefore overexpression of one or more of these genes may not be required. However, overexpression of one or more of these genes within the native hosts has been shown to improve production of the terpenoid precursors.

In other embodiments at least one of the enzymes is autologous to the particular host cell which is intended to express the enzymes. In other embodiments all of the enzymes are heterologous to the intended host cell.

In some embodiments each enzyme encoded by the vector is an enzyme as defined herein, i.e:
a HmgACT enzyme, for example of SEQ ID NO: 17 or a protein with an amino acid sequence with at least 80%, 85%, 90%, 92%, 94%, 96%, 98%, 99% or 100% similarity to said sequence;
a HmgS enzyme, for example of SEQ ID NO: 18 or a protein with an amino acid sequence with at least 80%, 85%, 90%, 92%, 94%, 96%, 98%, 99% or 100% similarity to said sequence;
a HmgR enzyme (including engineered HmgR enzymes described herein), for example that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102;
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
a Mevalonate kinase (MVK) enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22;
a PMK enzyme, for example of SEQ ID NO: 23 or a protein with an amino acid sequence with at least 80%, 85%, 90%, 92%, 94%, 96%, 98%, 99% or 100% similarity to said sequence; and
a PMD enzyme, for example of SEQ ID NO: 24 or a protein with an amino acid sequence with at least 80%, 85%, 90%, 92%, 94%, 96%, 98%, 99% or 100% similarity to said sequence.

In more specific embodiments, a vector of the invention comprises a polynucleotide encoding:
i) a HmgACT enzyme of SEQ ID NO 17;
ii) a HmgS enzyme of SEQ ID NO: 18;
iii) a HmgR enzyme (including engineered HmgR enzymes described herein) for example that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
iv) Mevalonate kinase (MVK) enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22;
v) a PMK enzyme of SEQ ID NO: 23;
vi) a PMD enzyme of SEQ ID NO: 24; and in some instances also includes:
vii) IDI enzyme of SEQ ID NO:31 or 41.

The invention also provides a vector that comprises a polynucleotide encoding both of:
a) an isopentenyl-diphosphate delta isomerase 1 enzyme; and
b) a 1-deoxy-D-xylulose-5-phosphate synthase enzyme.

These two enzymes are part of the MEP pathway. Accordingly, the invention provides an MEP pathway which comprises:
a) an isopentenyl-diphosphate delta isomerase 1 enzyme; and
b) a 1-deoxy-D-xylulose-5-phosphate synthase enzyme;
   or one or more polynucleotides that encode the:
a) isopentenyl-diphosphate delta isomerase 1 enzyme; and
b) 1-deoxy-D-xylulose-5-phosphate synthase enzyme.

The isopentenyl-diphosphate delta isomerase 1 enzyme and the 1-deoxy-D-xylulose-5-phosphate synthase enzyme may be any isopentenyl-diphosphate delta isomerase 1 enzyme and the 1-deoxy-D-xylulose-5-phosphate synthase enzyme. For example these enzymes may be autologous to a particular host cell which is intended to express the enzymes. In other embodiments at least one of the enzymes is autologous to the particular host cell which is intended to express the enzymes. In other embodiments all of the enzymes are heterologous to the intended host cell. In some embodiments each enzyme encoded by the vector is an enzyme as defined herein, i.e:
a) an isopentenyl-diphosphate delta isomerase 1 enzyme as described herein, for example wherein the IDI enzyme is from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
b) a 1-deoxy-D-xylulose-5-phosphate synthase enzyme as described herein, optionally wherein the DXS enzyme is from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

In more specific embodiments, a vector of the invention comprises a polynucleotide encoding:
a) an isopentenyl-diphosphate delta isomerase 1 enzyme of SEQ ID NO: 31 or 41; and
b) a 1-deoxy-D-xylulose-5-phosphate synthase enzyme of SEQ ID NO: 32.

It will be appreciated that various combinations of the polynucleotides and/or vectors of the invention can be combined with each other or, in some instances, with polynucleotides and/or vectors that express the native, autologous versions of some of the enzymes, to produce advantageous production systems for the production of phytoene, lycopene, neurosporene and/or bisabolol.

The phytoene, lycopene, neurosporene and bisabolol synthesis pathways are well studied and it will be clear to the skilled person as to what enzymes are required for the production of these products. FIG. 1 is a schematic showing the synthesis pathway of all of these products. The skilled person will readily understand that where the present disclosure presents, for example, a phytoene production system that comprises a first polynucleotide encoding phytoene synthase enzyme having an amino acid sequence of any of SEQ ID NO: 1-7, 51 or 52 the remaining enzymes in the pathway must also be present for phytoene production. The remaining enzymes may be any functional version of the enzyme, unless specified. For example where a phytoene production system comprise a first polynucleotide encoding phytoene synthase enzyme having an amino acid sequence of any of SEQ ID NO: 1-7, 51 or 52 the system may comprise any GGPPS enzyme, which may or may not be a GGPPS enzyme having an amino acid sequence of any of SEQ ID NO: 8-16, 55, 56, 57, or 58. The same premise applies to the production of each product.

For example, it will be appreciated that to produce any of phytoene, lycopene, neurosporene and bisabolol described herein, the relevant host cell (or cell-free system) must have a functional MVA or MEP pathway to produce the required precursors (unless, for example, intermediary components are provided to the cell or cell free system).

Applicable to all embodiments described herein, the skilled person will appreciate that the MVA and MEP pathways are metabolic pathways for the biosynthesis of isoprenoid precursors: IPP and DMAPP. Whereas plants use both the MVA and MEP pathway, most organisms only use one of the pathways for the biosynthesis of isoprenoid precursors. Bacteria typically use only the MEP pathway whereas yeast typically use only the MVA pathway.

Accordingly, some host cells have a functional native MVA and MEP pathway and engineering to cause expression of the relevant MVA and MEP proteins may not be needed. Some host cells do not have functional MVA or MEP pathway; and an intervention is required to engineer those cells so as to allow expression or overexpression of the relevant enzymatic pathways. The MVA and MEP pathway enzymes described herein can be introduced into a host cell to produce a functional MVA and/or MEP pathway within a given host cell.

Accordingly, any of the engineered cells described herein, for example engineered cells that express a phytoene synthase according to the invention, may also comprise the MVA and/or MEP pathways as described herein. Similarly, cell free production systems can also comprise the MEP and MVA polynucleotides and/or enzymes as described herein.

As described elsewhere, any polynucleotide of the invention may be present in a vector such as a plasmid or may be chromosomally integrated. Accordingly the polynucleotides that express components of the MVA or MEP pathway described herein may be present on a vector such as a plasmid, or may be integrated into a host cell genome.

As mentioned elsewhere, the invention provides various production systems for the production of phytoene, lycopene, neurosporene and/or bisabolol, and other downstream products such as retinol and phytofluene.

The invention provides a phytoene production system wherein the system comprises:
i) a first polynucleotide encoding phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and/or
ii) a second polynucleotide encoding a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58.

The first and second polynucleotide may be part of the same polynucleotide molecule, for example may both be incorporated into the same vector. As described elsewhere herein, and in relation to any of the polynucleotides that are introduced into a given host cell for expression, the polynucleotides may be expressed from one or more extrachromosomal vectors, or may be integrated into the genome of the host cell. Where more than one polynucleotide is to be expressed in a cell, more than one polynucleotide can be integrated into the genome. The one or more polynucleotides may be integrated into the host genome at the same locus, or the one or more polynucleotides may be integrated into the host genome at different loci. A single host cell can comprise one or more genomically integrated polynucleotides as described here in combination with one or more extrachromosomally maintained polynucleotides described herein.

The phytoene production system may also comprise a third or number of further polynucleotides, or further vectors that comprises the third or number of further polynucleotides, that encode components of the MVA (including IDI) and/or MEP pathway described herein.

In some embodiments the invention provides a phytoene production system that comprises:
i) a first polynucleotide encoding phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
ii) a second polynucleotide encoding a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58.

In a preferred embodiment, the invention provides a phytoene production system that comprises:
i) a first polynucleotide encoding phytoene synthase enzyme having an amino acid sequence of SEQ ID NO: 3 or 51 or having an amino acid sequence with at least 80%, 85%, 90%, 92%, 94%, 96%, 98%, 99% or 100% similarity to SEQ ID NO: 3 or 51; and
ii) a second polynucleotide encoding a GGPPS enzyme having an amino acid sequence of SEQ ID NO: 14, 55, 56, 57 or 58 or having an amino acid sequence with at least 80%, 85%, 90%, 92%, 94%, 96%, 98%, 99% or 100% similarity to any of SEQ ID NO: 14, 55, 56, 57 or 58.

In some embodiments where the phytoene production system is within a host cell that is a bacterial cell for example is an E. coli cell, the phytoene production system preferably comprises:
i) a first polynucleotide encoding phytoene synthase enzyme having an amino acid sequence of SEQ ID NO: 3 or having an amino acid sequence with at least 80%, 85%, 90%, 92%, 94%, 96%, 98%, 99% or 100% similarity to SEQ ID NO: 3; and
ii) a second polynucleotide encoding a GGPPS enzyme having an amino acid sequence of SEQ ID NO: 14 or having an amino acid sequence with at least 80%, 85%, 90%, 92%, 94%, 96%, 98%, 99% or 100% similarity to any of SEQ ID NO: 14.

In some embodiments where the phytoene production system is within a host cell that is a yeast cell for example is a S. cerevisiae cell, the phytoene production system preferably comprises:
i) a first polynucleotide encoding phytoene synthase enzyme having an amino acid sequence of SEQ ID NO: 51 or having an amino acid sequence with at least 80%, 85%, 90%, 92%, 94%, 96%, 98%, 99% or 100% similarity to SEQ ID NO: 51; and
ii) a second polynucleotide encoding a GGPPS enzyme having an amino acid sequence of SEQ ID NO: 55, 56, 57 or 58 or having an amino acid sequence with at least 80%, 85%, 90%, 92%, 94%, 96%, 98%, 99% or 100% similarity to any of SEQ ID NO: 55, 56, 57 or 58.

As used herein, the term “polynucleotide encoding” can be taken to also refer to the protein encoded by the polynucleotide. For example, it is possible to configure a phytoene production system comprising the relevant enzymes, rather than comprising the polynucleotides that encode the relevant enzymes. For example, in the use of a cell free or in vitro system.

The invention also provides a lycopene production system. The lycopene production system of the invention comprises the phytoene production system described above, and also comprises a polynucleotide that encodes a phytoene desaturase enzyme capable of converting phytoene to lycopene. In some instances the phytoene desaturase enzyme capable of converting phytoene to lycopene is a phytoene desaturase enzyme according to the invention. For example in some embodiments the phytoene desaturase enzyme:
is from Pantoea sp or from Pantoea ananatis; and/or
has an amino acid sequence of SEQ ID NO: 33 or has an amino acid sequence with at least 80%, 85%, 90%, 92%, 94%, 96%, 98%, 99% or 100% similarity to SEQ ID NO: 33; or
is from Paracoccus sp; and/or
has an amino acid sequence of SEQ ID NO: 34 or has an amino acid sequence with at least 80%, 85%, 90%, 92%, 94%, 96%, 98%, 99% or 100% similarity to SEQ ID NO: 34;
is from Pseudescherichia sp, or Pseudescherichia vulneris and has an amino acid sequence of SEQ ID NO: 35 or has an amino acid sequence with at least 80%, 85%, 90%, 92%, 94%, 96%, 98%, 99% or 100% similarity to SEQ ID NO: 35;
is from Synechocystis sp or Synechocystis sp strain PCC 6714 and has an amino acid sequence of SEQ ID NO: 37 or has an amino acid sequence with at least 80%, 85%, 90%, 92%, 94%, 96%, 98%, 99% or 100% similarity to SEQ ID NO: 37; or
is from Streptomyces sp or Streptomyces griseus and has an amino acid sequence of SEQ ID NO: 38 or has an amino acid sequence with at least 80%, 85%, 90%, 92%, 94%, 96%, 98%, 99% or 100% similarity to SEQ ID NO: 38.

As for the phytoene production system, the lycopene production system may comprise a combination of polynucleotides that encode the relevant enzymes and the enzymes themselves. Reference to a polynucleotide encoding in the context of the lycopene (and by reference, to the phytoene production system) production system can be taken to also refer to the protein encoded by the polynucleotide. For example it is possible to configure a lycopene production system comprising the relevant enzymes, rather than comprising the polynucleotides that encode the relevant enzymes. For example, in the use of a cell free or in vitro system.

Also as for the phytoene production system, the lycopene production system may also comprise one or more elements of the MVA (including IDI) or MEP pathway as described herein.

In some embodiments, the invention provides a retinol production system which comprises the lycopene production system described here. Lycopene is a precursor of b-carotene which itself is a precursor of retinol—it follows that a system which increases the production of lycopene will also increase the production of retinol.

Similarly, the invention also provides a neurosporene production system. In some embodiments, the neurosporene production system comprises the phytoene production system as described above, and a phytoene desaturase enzyme capable of converting phytoene to neurosporene.

In some embodiments the phytoene desaturase enzyme capable of converting phytoene to neurosporene is a phytoene desaturase enzyme according to the invention, for example is a phytoene desaturase enzyme having an amino acid sequence of SEQ ID NO: 36 or has an amino acid sequence with at least 80%, 85%, 90%, 92%, 94%, 96%, 98%, 99% or 100% similarity to SEQ ID NO: 36.

Also as for the phytoene production system, the neurosporene production system may also comprise one or more elements of the MVA (including IDI) or MEP pathway as described herein.

As described elsewhere, the inventors found novel enzymes that improve the production of bisabolol. Accordingly the invention also provides a bisabolol production system.

In one embodiment, the bisabolol production system comprises:
a HmgR enzyme (including engineered HmgR enzymes described herein) selected from:
   An engineered 3-hydroxy-3-methylglutaryl-CoA reductase enzyme that has an amino acid sequence with at least 80%, 85%, 90%, 92%, 94%, 96%, 98%, 99% or 100% similarity to SEQ ID NO: 19, provided that the enzyme comprises a substitution at one or more positions selected from positions 188, 236, or 343, wherein said positions are numbered with reference to SEQ ID No: 19; or a HmgR enzyme (including engineered HmgR enzymes described herein) of SEQ ID NO; 19, 20, 21 or a protein with an amino acid sequence with at least 80%, 85%, 90%, 92%, 94%, 96%, 98%, 99% or 100% similarity to said sequence.

In one embodiment, the bisabolol production system comprises:
a MVK enzyme (including engineered MVK enzymes described herein) that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22.

In the same or other embodiments the bisabolol production system comprises a bisabolol synthase of the invention as described herein.

In particularly advantageous embodiments, the bisabolol production system comprises the HmgR enzyme (including engineered HmgR enzymes described herein), the MVK enzyme, and the bisabolol synthase enzyme as described herein. For example in some embodiments the bisabolol production system comprises:
a HmgR enzyme (including engineered HmgR enzymes described herein) that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
and
a MVK enzyme (including engineered MVK enzymes described herein that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22; and
a bisabolol synthase of the invention as described herein.

For the production of bisabolol it is also considered to be necessary, in some embodiments, to express or increase expression of FPPS.

As mentioned elsewhere, any of the production systems disclosed herein, i.e. the phytoene, lycopene, neurosporene and bisabolol production systems, may also comprise an engineered MVA and/or MEP pathway. Such an approach is considered to increase the production of the FPP precursor necessary for the production of each of these products.

For example in some embodiments the phytoene, lycopene, neurosporene or the bisabolol production system may comprise any one or more of the following enzymes:
Acetoacetyl-CoA thiolase (HmgACT), optionally wherein the HmgACT protein is from Enterococcus faecalis, optionally has an amino acid sequence of SEQ ID NO: 17 or an amino acid sequence with at least 80%, 85%, 90%, 92%, 94%, 96%, 98%, 99% or 100% similarity to SEQ ID NO: 17;
a HmgR enzyme (including engineered HmgR enzymes described herein), for example that:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
Hydroxymethylglutaryl-CoA synthase (HmgS), optionally wherein the HmgS protein is from Enterococcus faecalis, optionally has a an amino acid sequence of SEQ ID NO: 18, or an amino acid sequence with at least 80%, 85%, 90%, 92%, 94%, 96%, 98%, 99% or 100% similarity to SEQ ID NO: 18;
   a MVK enzyme (including engineered MVK enzymes described herein) that: has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or comprises a E419D substitution, a K402V substitution, a E73G substitution, a G393V substitution, a L175V substitution, a D74E substitution, wherein said positions are numbered with reference to SEQ ID No: 22; or has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22; or comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74; or has a nucleotide sequence that encodes a protein with an amino acid sequence of SEQ ID NO: 22; or is from S. cerevisiae, optionally has a nucleotide sequence that encodes a protein with an amino acid sequence of SEQ ID NO: 22;
   Phosphomevalonate kinase (PMK), optionally wherein the PMK protein is from S. or an amino acid sequence with at least 80%, 85%, 90%, 92%, 94%, 96%, 98%, 99% or 100% similarity to SEQ ID NO: 23;
   Phosphomevalonate decarboxylase (PMD), optionally wherein the PMD protein is from S. cerevisiae, optionally has an amino acid sequence of SEQ ID NO: 24 or an amino acid sequence with at least 80%, 85%, 90%, 92%, 94%, 96%, 98%, 99% or 100% similarity to SEQ ID NO: 24;
   isopentenyl-diphosphate delta isomerase 1 (IDI), optionally wherein the IDI enzyme is from B. subtilis or E. coli, optionally wherein the isopentenyl-diphosphate delta isomerase enzyme has an amino acid sequence of SEQ ID NO: 31, or 41 or an amino acid sequence with at least 80%, 85%, 90%, 92%, 94%, 96%, 98%, 99% or 100% similarity to SEQ ID NO: 31 or 41; and/or
   1-deoxy-D-xylulose-5-phosphate synthase enzyme, that in some embodiments is from E. coli. In some embodiments the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

As described elsewhere, the production systems and methods described herein may comprise polynucleotides that encode the enzymes as described herein, or may comprise the enzymes themselves.

It will be apparent to the skilled person that the enzymes and polynucleotides of the invention will typically be expressed in a cell. The invention does also provide cell-free and in vitro systems, such as a cell-free or in vitro phytoene, lycopene, neurosporene or bisabolol production system, but preferably the enzymes are expressed in a cell.

Preferences for the preferred enzymes, and for the preferred enzymes in a bacterial or yeast context for each production system are set out elsewhere herein.

Accordingly, the invention provides an engineered cell that comprises any one or more of the enzymes and/or polynucleotides and/or production systems described herein.

In one embodiment the invention provides an engineered carotenoid production cell, wherein the engineered carotenoid production cell comprises:
a) a polynucleotide encoding a 3-hydroxy-3-methylglutaryl-CoA reductase enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
c) a polynucleotide encoding a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55 56, 57 or 58.
d) a polynucleotide encoding a mevalonate kinase enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
is from S. cerevisiae, optionally has a nucleotide sequence that encodes a protein with an amino acid sequence of SEQ ID NO: 22.

The engineered carotenoid production cell may be any cell, for example may be a bacterial cell or may be a eukaryotic cell such as a yeast. As set out elsewhere, the skilled person will appreciate that for the cell to produce a particular carotenoid, the cell must also comprise the other relevant enzymatic pathways necessary to provide upstream precursors and other downstream conversions.

Preferences for the preferred enzymes, and for the preferred enzymes in a bacterial or yeast context are set out elsewhere herein.

In one embodiment the engineered carotenoid production cell is for the production of phytoene and comprises:
a) a polynucleotide encoding a 3-hydroxy-3-methylglutaryl-CoA reductase enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
c) a polynucleotide encoding a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57, or 58;
d) a polynucleotide encoding a mevalonate kinase enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has a nucleotide sequence that encodes a protein with an amino acid sequence of SEQ ID NO: 22.

The engineered carotenoid production cell for the production of phytoene may be any cell, for example may be a bacterial cell or may be a eukaryotic cell such as a yeast. As set out elsewhere, the skilled person will appreciate that for the cell to produce phytoene, the cell must also comprise the other relevant enzymatic pathways necessary to provide upstream precursors and other downstream conversions.

In another embodiment the engineered carotenoid production cell is for the production of lycopene and comprises:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has
at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   or
b) a polynucleotide encoding a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
c) a polynucleotide encoding a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58
   and
d) a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38; and
e) a polynucleotide encoding a mevalonate kinase enzyme that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
has a nucleotide sequence that encodes a protein with an amino acid sequence of SEQ ID NO: 22;
is from S. cerevisiae, optionally has a nucleotide sequence that encodes a protein with an amino acid sequence of SEQ ID NO: 22.

The engineered carotenoid production cell for the production of lycopene may be any cell, for example may be a bacterial cell or may be a eukaryotic cell such as a yeast. As set out elsewhere, the skilled person will appreciate that for the cell to produce lycopene, the cell must also comprise the other relevant enzymatic pathways necessary to provide upstream precursors and other downstream conversions.

Preferences for the preferred enzymes, and for the preferred enzymes in a bacterial or yeast context are set out elsewhere herein.

In another embodiment the engineered carotenoid production cell is for the production of neurosporene and comprises:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
c) a polynucleotide encoding a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58,
   and
d) a phytoene desaturase enzyme that converts phytoene to neurosporene, optionally wherein the phytoene desaturase enzyme has an amino acid sequence of SEQ ID NO: 36, or has an amino acid sequence with at least 80%, 85%, 90%, 92%, 94%, 96%, 98%, 99% or 100% similarity to SEQ ID NO: 36;
e) a polynucleotide encoding a mevalonate kinase enzyme that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
has a nucleotide sequence that encodes a protein with an amino acid sequence of SEQ ID NO: 22;
is from S. cerevisiae, optionally has a nucleotide sequence that encodes a protein with an amino acid sequence of SEQ ID NO: 22.

The engineered carotenoid production cell for the production of neurosporene may be any cell, for example may be a bacterial cell or may be a eukaryotic cell such as a yeast. As set out elsewhere, the skilled person will appreciate that for the cell to produce neurosporene, the cell must also comprise the other relevant enzymatic pathways necessary to provide upstream precursors and other downstream conversions.

Preferences for the preferred enzymes, and for the preferred enzymes in a bacterial or yeast context are set out elsewhere herein.

The invention also provides an engineered terpenoid production cell wherein the cell comprises:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a mevalonate kinase enzyme that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
has a nucleotide sequence that encodes a protein with an amino acid sequence of SEQ ID NO: 22;
is from S. cerevisiae, optionally has a nucleotide sequence that encodes a protein with an amino acid sequence of SEQ ID NO: 22.

In some embodiments the engineered terpenoid production cell is for the production of bisabolol and the cell further comprises a bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30, optionally has an amino acid sequence of SEQ ID NO: 29 or 25 or with at least 80%, 85%, 90%, 92%, 94%, 96%, 98%, 99% or 100% similarity to any of SEQ ID NO: 29 or 25.

The engineered terpenoid production cell may be any cell, for example may be a bacterial cell or may be a eukaryotic cell such as a yeast. As set out elsewhere, the skilled person will appreciate that for the cell to produce a particular terpenoid, the cell must also comprise the other relevant enzymatic pathways necessary to provide upstream precursors and other downstream conversions.

Preferences for the preferred enzymes, and for the preferred enzymes in a bacterial or yeast context are set out elsewhere herein.

In another embodiment the engineered cell comprises the bisabolol production system of the invention.

The engineered cell may be derived from any cell. The skilled person will recognise that there are particular advantageous cell types that are routinely used for industrial processes.

In some embodiments the engineered cell is an engineered prokaryotic cell, optionally a bacterial cell, for example an Escherichia coli cell, Corynebacterium glutamicum, or a Bacillus sp cell. Other relevant industrial prokaryotic hosts will be apparent to the skilled person.

In one embodiment the bacterial cell is not a Deinococcus bacterial cell.

In other embodiments the engineered cell is an engineered eukaryotic cell, optionally a yeast cell, optionally a Saccharomyces cerevisiae cell, a Pichia pastoris cell or a Yarrowia lipolytica cell. Other relevant industrial eukaryotic hosts will be apparent to the skilled person.

The engineered cell may comprise a MVA pathway and/or MEP pathway as described herein. In some embodiments the cell comprises an engineered MVA and/or MEP pathway, by which we include the meaning that the MVA and/or MEP pathway comprised by the cell comprises at least one heterologous gene/protein, or at least one gene or protein that is mutated relative to the wild-type native gene or protein.

The engineered cells of the present invention are considered to be able to produce increased amounts of a product relative to the amount of product produced by the native, non-engineered cell, for example relative to a cell of the same species and same genetic background but which does not comprise the relevant polynucleotides or enzymes of the invention. In some embodiments the engineered cell of the invention (for example the phytoene production cell, the lycopene production cell, the neurosporene production cell and the bisabolol production cell of the invention as described herein) is able to produce larger amounts of the relevant product than a cell of the same species and genetic background that does not comprise the relevant polynucleotides or enzymes. In some embodiments a larger amount intended to include within the meaning that the cell is capable of producing at least 0.1%, 0.5%, 1%, 2%, 5%, 10%, 15%, 20%, 25%, 30%, 35%, 40%, 45%, 50%, 55%, 60%, 70%, 80%, 90%, 100% or more of the relevant product than a cell that does not comprise the relevant polynucleotides or enzymes.

In some embodiments the engineered cells of the invention are able to produce an increased amount of a product relative to the same cell that has been engineered only insofar as to incorporate the genes necessary for a functional MVA pathway, for example to have a functional pathway so as the allow the production of FPP. That is, some cells, such as bacteria such as E. coli, do not have a functional MVA pathway, and so the base cell has to be engineered to the extent necessary so as to allow general function of the pathways for the production of the products described herein. In these cases, an engineered cell of the invention has improved production of the target product over the base strain.

Examples show that point mutations in the HmgR enzyme increases the production of phytoene by around 20% or by around 25%; and that these point mutations can increase the activity of the enzyme itself by around 20%.

Other improvements achieved by the present invention include improved precursor availability, improved activity of key enzymes by overexpression or engineering, and/or improved carbon flux to direct carbon towards production formation.

Accordingly, in general the engineered cell of the invention is a cell that is able to produce farnesyl diphosphate (FPP). To produce FPP, the engineered cell may include either the MVA or the MEP pathway and/or components of each. The skilled person will appreciate that a bacterial cell contains a native MEP pathway and a yeast cell contains a native MVA pathway

The engineered cell of the invention may comprise any one or more polynucleotides that encode any one or more of:
a) a phytoene synthase enzyme as defined herein;
b) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52;
c) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
d) a GGPPS enzyme as defined herein;
e) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
f) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
g) a phytoene synthase enzyme as defined herein and a GGPPS enzyme as defined herein;
h) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 56 or 58 or having an amino acid sequence with at least 80%, 85%, 90%, 92%, 94%, 96%, 98%, 99% or 100% similarity to any of SEQ ID NO: 8-16, 55, 56, 57 or 58;
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
j) a phytoene synthase enzyme as defined herein and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
k) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
l) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
m) the mevalonate pathway expression system according to any of numbered embodiment paragraphs H1-H5 as set out below;
n) a HmgACT enzyme, for example of SEQ ID NO: 17 or a protein with an amino acid sequence with at least 80%, 85%, 90%, 92%, 94%, 96%, 98%, 99% or 100% similarity to said sequence;
o) a HmgS enzyme, for example of SEQ ID NO: 18 or a protein with an amino acid sequence with at least 80%, 85%, 90%, 92%, 94%, 96%, 98%, 99% or 100% similarity to said sequence;
p) a HmgR enzyme (including engineered HmgR enzymes described herein), for example that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
q) a MVK enzyme, for example that has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
encodes a protein with an amino acid sequence of SEQ ID NO: 22;
r) a PMK enzyme, for example of SEQ ID NO: 23 or a protein with an amino acid sequence with at least 80%, 85%, 90%, 92%, 94%, 96%, 98%, 99% or 100% similarity to said sequence;
s) a PMD enzyme, for example of SEQ ID NO: 24 or a protein with an amino acid sequence with at least 80%, 85%, 90%, 92%, 94%, 96%, 98%, 99% or 100% similarity to said sequence
t) a HmgACT enzyme, for example of SEQ ID NO: 17 or a protein with an amino acid sequence with at least 80%, 85%, 90%, 92%, 94%, 96%, 98%, 99% or 100% similarity to said sequence;
a HmgS enzyme, for example of SEQ ID NO: 18 or a protein with an amino acid sequence with at least 80%, 85%, 90%, 92%, 94%, 96%, 98%, 99% or 100% similarity to said sequence;
a HmgR enzyme (including engineered HmgR enzymes described herein), for example that:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
a MVK enzyme, for example that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following:
a aspartic acid at residue 419, a valine at residue 402, a glycine at residue 73, a valine at residue 393, a valine at residue 175, a glutamic acid at residue 74;
has an amino acid sequence of SEQ ID NO: 22;
a PMK enzyme, for example of SEQ ID NO: 23 or a protein with an amino acid sequence with at least 80%, 85%, 90%, 92%, 94%, 96%, 98%, 99% or 100% similarity to said sequence; and
a PMD enzyme, for example of SEQ ID NO: 24 or a protein with an amino acid sequence with at least 80%, 85%, 90%, 92%, 94%, 96%, 98%, 99% or 100% similarity to said sequence
u) a phytoene desaturase enzyme as described herein, for example of a phytoene desaturase enzyme with an amino acid sequence of any of SEQ ID NO: 33-38 or has an amino acid sequence with at least 80%, 85%, 90%, 92% 94%, 96%, 98%, 99% or 100% similarity to any of SEQ ID NO: 33-38;
v) a bisabolol synthase enzyme as defined herein, for example a bisabolol synthase enzyme having an amino acid sequence of SEQ ID NO: 25-29 or having an amino acid sequence with at least 80%, 85%, 90%, 92%, 94%, 96%, 98%, 99% or 100% similarity to any of SEQ ID NO: 25-30;
w) an Acetoacetyl-CoA thiolase enzyme as described herein, for example an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39;
x) a Hmg-CoA synthase enzyme as described herein, for example a Hmg-CoA synthase enzyme having an amino acid sequence of SEQ ID NO: 40 or having an amino acid sequence with at least 80%, 85%, 90%, 92%, 94%, 96%, 98%, 99% or 100% similarity to any of SEQ ID NO: 40;
y) an isopentenyl-diphosphate delta isomerase 1 enzyme as described herein, for example wherein the IDI enzyme is from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
z) a 1-deoxy-D-xylulose-5-phosphate synthase enzyme as described herein, optionally wherein the DXS enzyme is from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

Any one or more of the polynucleotides of the invention present in the cell may be present extra-chromosomally, for example may be present on a vector or a plasmid. In another or the same embodiment, any one or more of the polynucleotides of the invention present in the cell may be genomically integrated.

In some preferred embodiments, the engineered cell of the invention comprises one or more polynucleotides that encode all of the components of the MVA pathway as follows:
i) a HmgACT enzyme of SEQ ID NO 17 or having a nucleic acid sequence that encodes a protein with an amino acid sequence with at least 80%, 85%, 90%, 92%, 94%, 96%, 98%, 99% or 100% similarity to SEQ ID NO: 17;
ii) a HmgS enzyme of SEQ ID NO: 18 or having a nucleic acid sequence that encodes a protein with an amino acid sequence with at least 80%, 85%, 90%, 92%, 94%, 96%, 98%, 99% or 100% similarity to SEQ ID NO: 18;
iii) a HmgR enzyme (including engineered HmgR enzymes described herein) that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
iv) a MVK enzyme, for example that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has an amino acid sequence of SEQ ID NO: 22;
v) a PMK enzyme of SEQ ID NO: 23 or having a nucleic acid sequence that encodes a protein with an amino acid sequence with at least 80%, 85%, 90%, 92%, 94%, 96%, 98%, 99% or 100% similarity to SEQ ID NO: 23; and
vi) a PMD enzyme of SEQ ID NO: 24 or having a nucleic acid sequence that encodes a protein with an amino acid sequence with at least 80%, 85%, 90%, 92%, 94%, 96%, 98%, 99% or 100% similarity to SEQ ID NO: 24.

A cell comprising the above features (i)-(vi) is considered to comprise the components required for improved production of FPP which is a precursor for all of phytoene, lycopene, neurosporene and bisabolol production. Accordingly, a cell that comprises these features may be termed an engineered FPP production cell, and the invention provides an engineered FPP production cell wherein the cell comprises one or more polynucleotides that encode all of the features of (i)-(vi) above. As set out in detail elsewhere herein, the skilled person will appreciate that other components of the necessary pathways must be present to allow the production of various intermediate components. For example, for a cell comprising (i)-(vi) above to be able to produce FPP, the cell must also have a functional MEP pathway (See FIG. 1). The requirement for a functional MEP pathway, and whether this is endogenously present in the cell or is engineered into a cell that does not have an endogenous MEP pathway (or is in an improvement on an endogenous MEP pathway) to produce FPP and the other compounds described herein is detailed elsewhere herein.

Accordingly, In some embodiments the cell, for example the FPP production cell, also comprises one or more polynucleotides that encode the components of the MEP pathway as follows:
vii) an isopentenyl-diphosphate delta isomerase 1 enzyme as described herein, for example wherein the IDI enzyme is from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
viii) a 1-deoxy-D-xylulose-5-phosphate synthase enzyme as described herein, optionally wherein the DXS enzyme is from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

Inclusion of the DXS and IDI polynucleotides of (vii) and (viii) above are considered to further enhance the production of FPP.

Preferences for the cell are as described elsewhere. Preferably the cell is a S. cerevisiae cell or an E. coli cell. Preferences for the polynucleotides are as described elsewhere. For example the polynucleotide may be part of a vector such as a plasmid, or may be chromosomally integrated into the cell- or some polynucleotides may be present on vectors and some may be chromosomally located.

The engineered FPP production cell may be further engineered to drive the production of phytoene. For example, the engineered FPP production cell (which may or may not include features (vii) and (viii) above may be engineered to also include one or both of:
ix) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and/or
x) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58.

Preferences for the preferred enzymes, and for the preferred enzymes in a bacterial or yeast context are set out elsewhere herein.

Such an engineered cell, which also comprises features (ix) and (x) above may be termed an engineered phytoene production cell. Accordingly the invention provides an engineered phytoene production cell wherein the cell comprises one or more polynucleotides that encode features (i)-(vi) and (ix) and (x) and which in some embodiments include features (vii) and (viii) above.

In specific embodiments of the phytoene production cell the cell comprises both features (ix) and (x) as follows:
ix) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3; and
x) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14.

Preferences for the engineered cells are as described elsewhere. Preferably the cell is a S. cerevisiae cell or an E. coli cell. Preferences for the polynucleotides are as described elsewhere. For example the polynucleotide may be part of a vector such as a plasmid, or may be chromosomally integrated into the cell- or some polynucleotides may be present on vectors and some may be chromosomally located.

The engineered phytoene production cell may also feature (xi) one or more polynucleotides that encode a phytoene desaturase enzyme capable of converting phytoene to lycopene. In preferred embodiments feature (xi) is a polynucleotide that encodes a phytoene desaturase enzyme that:
is from Pantoea sp or from Pantoea ananatis; and/or
   has an amino acid sequence of SEQ ID NO: 33 or has an amino acid sequence with at least 80%, 85%, 90%, 92%, 94%, 96%, 98%, 99% or 100% similarity to SEQ ID NO: 33; or
   is from Paracoccus sp; and/or
   has an amino acid sequence of SEQ ID NO: 34 or has an amino acid sequence with at least 80%, 85%, 90%, 92%, 94%, 96%, 98%, 99% or 100% similarity to SEQ ID NO: 34;
   is from Pseudescherichia sp, or Pseudescherichia vulneris and has an amino acid sequence of SEQ ID NO: 35 or has an amino acid sequence with at least 80%, 85%, 90%, 92%, 94%, 96%, 98%, 99% or 100% similarity to SEQ ID NO: 35;
   is from Synechocystis sp or Synechocystis sp strain PCC 6714 and has an amino acid sequence of SEQ ID NO: 37 or has an amino acid sequence with at least 80%, 85%, 90%, 92%, 94%, 96%, 98%, 99% or 100% similarity to SEQ ID NO: 37; or
   is from Streptomyces sp or Streptomyces griseus and has an amino acid sequence of SEQ ID NO: 38 or has an amino acid sequence with at least 80%, 85%, 90%, 92%, 94%, 96%, 98%, 99% or 100% similarity to SEQ ID NO: 38.

Such an engineered cell, which comprises features (i)-(vi), (ix), (x) and (xi) and which may comprise in some embodiments features (vii) and (viii) above may be termed an engineered lycopene production cell. Accordingly the invention provides an engineered lycopene production cell wherein the cell comprises one or more polynucleotides that encode features (i)-(vi), (ix), (x) and (xi) and which may comprise in some embodiments features (vii) and (viii) above.

In other embodiments the engineered phytoene production cell may also comprise (xii) one or more polynucleotides that encode a phytoene desaturase enzyme capable of converting phytoene to neurosporene. In preferred embodiments feature (xii) is a polynucleotide that encodes a phytoene desaturase enzyme that:
Is from Rhodobacter sp or Rhodobacter capsulatus and has an amino acid sequence of SEQ ID NO: 36, or has an amino acid sequence with at least 80%, 85%, 90%, 92%, 94%, 96%, 98%, 99% or 100% similarity to SEQ ID NO: 36.

Such an engineered cell, which comprises features (i)-(vi), (ix), (x) and (xii) and which may comprise in some embodiments features (vii) and (viii) above may be termed an engineered neurosporene production cell. Accordingly the invention provides an engineered neurosporene production cell wherein the cell comprises one or more polynucleotides that encode features (i)-(vi), (ix), (x) and (xii) and which may comprise in some embodiments features (vii) and (viii) above.

In other embodiments the engineered FPP production cell may also feature (xiii) one or more polynucleotides that encode a bisabolol synthase enzyme. In preferred embodiments feature (xiii) is a polynucleotide that encodes a bisabolol synthase that is:
a Bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30, preferably of SEQ ID NO: 29 or 25 or with at least 80%, 85%, 90%, 92%, 94%, 96%, 98%, 99% or 100% similarity to any of SEQ ID NO: 29 or 25.

Such an engineered cell, which comprises features (i)-(vi) and (xiii), and which may comprise in some embodiments features (vii) and (viii) above, may be termed an engineered bisabolol production cell. Accordingly the invention provides an engineered bisabolol production cell wherein the cell comprises one or more polynucleotides that encode features (i)-(vi), and (xiii) and which may comprise in some embodiments features (vii) and (viii) above.

As mentioned elsewhere, the skilled person will appreciate that whilst the enzymes and polynucleotides of the invention lend themselves to being incorporated into and expressed within a cell, there are in some instances advantages associated with a cell free system. The skilled person will appreciate what is meant by a cell free system, and any one or more of the enzymes or polynucleotides of the invention described herein may be part of a cell free system of the invention.

Accordingly the invention provides a cell free FPP production system; a cell free phytoene production system; a cell free lycopene production system; a cell free neurosporene production system and a cell free bisabolol production system, wherein the cell free systems comprise the enzymes and/or polynucleotides encoding the enzymes required for engineered cell-based production systems described herein.

It will be clear to the skilled person that the various cells, cell free systems, enzymes and polynucleotides described herein can be used in methods of producing various products, including carotenoids and terpenoids, for example phytoene, lycopene, neurosporene and bisabolol.

The invention provides a method for producing a carotenoid or terpenoid wherein the method comprises culturing a cell as described herein.

In some embodiments the invention provides a method for producing a carotenoid or terpenoid wherein the method comprises culturing a recombinant cell such as a cell of the invention, wherein the cell:

Expresses or overexpresses one or more enzymes of the mevalonate pathway and/or methylerythritol pathway and wherein the cell expresses or overexpresses at least 3-hydroxy-3-methylglutaryl-CoA A reductase, wherein the 3-hydroxy-3-methylglutaryl-CoA reductase enzyme:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;

### And/or

a mevalonate kinase enzyme that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
has a nucleotide sequence that encodes a protein with an amino acid sequence of SEQ ID NO: 22;
is from S. cerevisiae, optionally has a nucleotide sequence that encodes a protein with an amino acid sequence of SEQ ID NO: 22;
   wherein said expression or overexpression results in increased production of:
i) phytoene;
ii) carotenoids;
iii) bisabolol;
iv) terpenoids; and/or
v) isopentenyl pyrophosphate (IPP) and dimethylallyl pyrophosphate (DMAPP)
   relative to a cell that does not express and/or overexpress said one or more enzymes of the mevalonate pathway and/or methylerythritol pathway, or
   relative to a cell that is able to produce
i) phytoene;
ii) carotenoids;
iii) bisabolol;
iv) terpenoids; and/or
v) isopentenyl pyrophosphate (IPP) and dimethylallyl pyrophosphate (DMAPP)
   but that does not express and/or overexpress said one or more enzymes of the mevalonate pathway and/or methylerythritol pathway.
   wherein said expression or overexpression results in increased production of isopentenyl pyrophosphate (IPP) and dimethylallyl pyrophosphate (DMAPP) relative to a cell that does not express or overexpress said one or more enzymes of the mevalonate pathway and/or methylerythritol pathway.

In some embodiments, for any of the enzymes described herein, the expression is an overexpression. Overexpression is well understood by the skilled person, and in one embodiment includes the meaning that the protein is expressed to a level that is higher than the level of expression of the protein in the native context. For example, proteins may be overexpressed by being expressed from high copy plasmids, or by being under the control of a strong promoter. The expression of the protein in these contexts is higher than if the protein was not expressed from a high copy plasmid or if the protein was not under the control of a strong promoter and was, for example, under the control of its own native promoter.

Expression or overexpression of the HmgR enzyme (including the engineered enzymes) is considered to be sufficient to increase the amount of the IPP and DMAPP precursors for the subsequent production of terpenoids and carotenoids. Overexpression of the other enzymes in the above pathways may be additionally advantageous.

In some embodiments, wherein the method is for producing a carotenoid, the cell further expresses or overexpresses:
a) phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
b) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58.

Where the carotenoid is phytoene, in some embodiments of the method the cell also expresses or overexpresses a) phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52; and
b) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58
   since expression or overexpression of these two enzymes is considered to lead to the direct production of phytoene.

For the production of downstream carotenoids, expression or overexpression of further enzymes is required. For example, where the carotenoid is lycopene, in some embodiments the cell further comprises a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38.

Where the carotenoid is neurosporene, in some embodiments the cell further comprises a phytoene desaturase enzyme that converts phytoene to neurosporene, optionally wherein the phytoene desaturase enzyme has an amino acid sequence of SEQ ID NO: 36, or has an amino acid sequence with at least 80%, 85%, 90%, 92%, 94%, 96%, 98%, 99% or 100% similarity to SEQ ID NO: 36.

In another embodiment, wherein the method is for producing the terpenoid bisabolol, the cell further expresses or overexpresses a Bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30.

Accordingly the invention also provides a method for the production of phytoene; a method for the production of lycopene; a method for the production of neurosporene; a method for the production of bisabolol; a method for the production of FPP; a method for the production of retinol.

In one embodiment the invention provides a method for the production of phytoene where the method comprises culturing an engineered phytoene production cell of the invention.

In one embodiment the invention provides a method for the production of lycopene where the method comprises culturing an engineered lycopene production cell of the invention.

In one embodiment the invention provides a method for the production of neurosporene where the method comprises culturing an engineered neurosporene production cell of the invention.

In one embodiment the invention provides a method for the production of bisabolol where the method comprises culturing an engineered bisabolol production cell of the invention.

In one embodiment the invention provides a method for the production of FPP where the method comprises culturing an engineered FPP production cell of the invention.

In one embodiment the invention provides a method for the production of retinol where the method comprises culturing an engineered retinol production cell of the invention.

As described elsewhere, the polynucleotides encoding the relevant enzyme(s) may be under the control of an inducible promoter. In some embodiments the polynucleotides encoding the relevant enzyme(s) are under the control of a galactose inducible promoter. In instances where one or more enzymes are under the control of an inducible promoter the method involves culturing the cell of the invention in the presence of one or more inducing agents.

In some embodiments the cells are cultured using fermentation. In some instances the fermentation is a batch fermentation. In some instances the fermentation is a fed-batch fermentation. In some embodiments the fermentation is a continuous fermentation.

As will be apparent to the skilled person, different cells are able to utilise different carbon sources. For example E. coli and S. cerevisiae can both metabolise hexose sugars such as glucose. In one embodiment the methods of producing the various products of the invention are cultured in a culture media that comprises:
a hexose sugar as a carbon source, optionally glucose;
a complex sugar as a carbon source;
crude glycerol as a carbon source; and/or glycerol as a carbon source.

The invention also provides various kits for putting the invention into practice.

### Sequences

| | | Seq ID | Species | |
|---|---|---|---|---|
| | Sequence | NO: | originated from | Blastp validation |
| PYS1 | MAVGSKSFATASKLFDAKTRRSVLMLYAWCRHCDDVIDDQTLGF | 1 | Pantoea | Select seq |
| | QARQPALQTPEQRLMQLEMKTRQAYAGSQMHEPAFAAFQEVAM | | ananatis | ref\|WP_041931046.1\| |
| | AHDIAPAYAFDHLEGFAMDVREAQYSQLDDTLRYCYHVAGVVGL | | | phytoene/squalene |
| | MMAQIMGVRDKATLDRACDLGLAFQLTNIARDIVDDAHAGRCYL | | | synthase family |
| | PASWLEHEGLNKENYAAPENRQALSRIARRLVQEAEPYYLSATAG | | | protein [Pantoea |
| | LARLPLRSAWAIATAKQVYRKIGVKVEQAGQQAWDQRQSTTTPE | | | ananatis] |
| | KLTLLLAASGQALTSRMRAHPPRPAHLWQRPL | | | |
| PYS2 | MACNFAVRVIYYPKEIHGVSVLNTNRSRKSRFSCRVMKLSTGVSA | 2 | Daucus | Select seq |
| | VAANPVRTSEERVYEVVLKQAALVREEKRSSRGLCLDTKRTGSKS | | carota | ref\|NP_001316106.1\| |
| | FDKSENDDAGMKSWNLLNEAYDRCGEVCAEYAKTFYLGTLLMTP | | | phytoene |
| | ERRRAVWAIYVWCRRTDELVDGPNASHITPKALDRWEKRLNDLF | | | synthase, chloroplastic |
| | DGQPYDMYDAALADTVSTYPVDIQPFKDMIDGMRMDLKKSRYQT | | | [Daucus carota subsp. |
| | FDELYLYCYYVAGTVGLMSVPVMGIAPESKATTESVYSAALALGIA | | | sativus] |
| | NQLTNILRDVGEDARRGRIYLPQEELKLAGITPEYIFKGKVTDKWR | | | |
| | SFMKGQIKRARMFFDEAEKGVAELSSASRWPVWASLLLYKQILD | | | |
| | AIEANDYDNFTKRAYVGKAKKLVSLPLAYSRALFAPSTVR | | | |
| PYS3 | MEVGSKSFATASKLFDAKTRRSVLMLYAWCRHCDDVIDDQVLGF | 3 | Enterobacter | Select seq |
| | SNDTPSLQSAEQRLAQLEMKTRQAYAGSQMHEPAFAAFQEVAMA | | agglomerans | ref\|WP_033773805.1\| |
| | HDILPAYAFDHLAGFAMDVHETRYQTLDDTLRYCYHVAGVVGLM | | | phytoene/ |
| | MAQIMGVRDNATLDRACDLGLAFQLTNIARDIVEDAEAGRCYLPA | | | squalene synthase family |
| | AWLAEEGLTRENLADPQNRKALSRVARRLVETAEPYYRSASAGLP | | | protein [Pantoea] |
| | GLPLRSAWAIATAQQVYRKIGMKVVQAGSQAWEQRQSTSTPEKL | | | |
| | ALLVAASGQAVTSRVARHAPRSADLWQRPV | | | |
| PYS4 | MANGQISPQRAVTKPQSWWLTSEPRPSLMLQLPKPSPSAKPCAS | 4 | Synechocystis | Select seq |
| | VEEAYEICRQVTAQYAKTFYLGTLLMPPAKRRAIWAIYLWCRRTDE | | sp. | ref\|WP_010872477.1\| |
| | LVDGPQAATTTPETLDHWERRLEGIFAGQPQDDADVALVDTLETF | | | phytoene |
| | PLDIQPFRDMIAGQRMDLYRSRYQTFEELDLYCYRVAGTVGLMSS | | | synthase [unclassified |
| | AVLGVDTGNGQAPWQPDAVYIPQEEAIALGVANQLTNILRDVGE | | | Synechocystis] |
| | DVERGRIYLPLEDLERFNYSEQDLLNGVNDDRWRSLMKFEIDRAR | | | |
| | HYFEDAERGIRALNRDARWPVWTALMLYKGILDVIEANNYNVFNR | | | |
| | RAYVPTPKKLLYLPVAWLRAQVL | | | |
| PYS5 | MLQLSKSPCMRTPATRSDSYELCRQITAQYAKTFYLGTQLMPLAK | 5 | Arthrospira | Select seq |
| | RQAIWAIYVWCRRTDELVDGPMASSTTLETLDHWEEQLESIFAG | | platensis | sp\|O07333.1\| |
| | HPIEPVDVALVDTLGRFPLDIQPFRDMIAGQRMDLSRNRYNTFDE | | | RecName: |
| | LNLYCYRVAGTVGLMSLAVMGTAEPDLSVPWNRDQSIYYPKEEAI | | | Full = Phytoene |
| | ALGIANQLTNILRDVGEDARRGRIYLPLDDLALFNYTEADLLNGKV | | | synthase [Arthrospira |
| | DERWRELMRFQIQRARKFYTLAEEGIAALHPDIRWPVWTALMLYR | | | platensis] |
| | QILDEIERNEYDVFNQRAYVPTWKKMMCLPLAQLRARVL | | | |
| PYS6 | MSLASSLVVSSNVELSPSSFGFLDSVRDGPQIPDSFRFSSRNRVP | 6 | Cucumismelo | Select seq |
| | NLINKKQKWGNHSHSTELKYPILHESGYGSVIVASMVANPAGEIA | | | sp\|P49293.1\| |
| | VSAEQKVYNVVMKQAALVKR7QLRTAGELDVKPDIVLPGTLSLLN | | | RecName: |
| | EAYDRCGEVCAEYA8KTFYLGTMLMTPERQKAIWAIYVWCRRTDE | | | Full = Phytoene |
| | LVDGPNASHITPTALDRWEARLEELFQGRPFDMLDAALADTVTKF | | | synthase, chloroplastic; |
| | PVDIQPFKDMIEGMRMDLRKSRYKNFDELYLYCYYVAGTVGLMSV | | | [Cucumis melo] |
| | PVMGIAPESQASTESVYNAALALGIANQAPPNILRDVGEDARRGR | | | |
| | IYLPQDELAQAGLSDEDIFAGRVTDKWRNFMKNQIKRARMFFDE | | | |
| | AEKGVLELNKASRWPVWASLLLYRQILDEIEANDYDNFTKRAYVS | | | |
| | KAKKILALPMAYGRALLGPS | | | |
| PYS7 | MTMMDMNFKYCHKIMKKHSKSFSYAFDLLPEDQRKAVWAIYAVC | 7 | Staphylococcus | Select seq |
| | RKIDDSIDVYGDIQFLNQIKEDIQSIEKYPYEHHHFQSDRRIMMAL | | aureus | ref\|WP_000178313.1\| |
| | QHVAQHKNIAFQSFYNLIDTVYKDQHFTMFETDAELFGYCYGVAG | | (strain | phytoene/ |
| | TVGEVLTPILSDHETHQTYDVARRLGESLQLINILRDVGEDFENER | | bovine | squalene synthase family |
| | IYFSKQRLKQYEVDIAEVYQNGVNNHYIDLWEYYAAIAEKDFRDV | | RF122 | protein |
| | MDQIKVFSIEAQPIIELAARIYIEILDEVRQASYTLHERVFVYKRKK | | ET3-1) | [Staphylococcus |
| | AKLFHEINSKYHRI | | | aureus] |
| GGPPS1 | MEAKIDELINNDPVWSSQNESLISKPYNHILLKPGKNFRLNLIVQI | 8 | Saccharomyces | Select seq pdb\|2DH4\|A |
| | NRVMNLPKDQLAIVSQIVELLHNSSLLIDDIEDNAPLRRGQTTSHL | | cerevisiae | Geranylgeranyl |
| | IFGVPSTINTANYMYFRAMQLVSQLTTKEPLYHNLITIFNEELINLH | | | pyrophosphate |
| | RGQGLDIYWRDFLPEIIPTQEMYLNMVMNKTGGLFRLTLRLMEAL | | | synthase |
| | SPSSHHGHSLVPFINLLGIIYQIRDDYLNLKDFQMSSEKGFAEDIT | | | [Saccharomyces |
| | EGKLSFPIVHALNFTKTKGQTEQHNEILRILLLRTSDKDIKLKLIQIL | | | cerevisiae] |
| | EFDTNSLAYTKNFINQLVNMIKNDNENKYLPDLASHSDTATNLHD | | | |
| | ELLYIIDHLSEL | | | |
| GGPPS2 | MRSMNLVDLWAQQACLVFNQTLSYKSFNGFMKIPLKNSKINPKL | 9 | Capsicum | Select seq |
| | NKKRPFSPLTVSAIATTKEDERIEAAQTEEPFNFKIYVTEKAISVNK | | annuum | ref\|NP_001385275.1\| |
| | ALDEAIIVKEPHVIHEAMRYSLLAGGKRVRPMLCLAACELVGGNQ | | | geranylgeranyl |
| | ENAMAAACAVEMIHTMSLIHDDLPCMDNDDLRRGKPTNHKIYGE | | | pyrophosphate |
| | DVAVLAGDSLLAFAFEHIVNSTAGVTPSRIVGAVAELAKSIGTEGL | | | synthase, chloroplastic |
| | VAGQVADIKCTGNASVSLETLEFIHVHKTAALLESSVVLGAILGGG | | | [Capsicum annuum] |
| | TNVEVEKLRRFARCIGLLFQVVDDILDVTKSSEELGKTAGKDLVV | | | |
| | DKTTYPKLLGLEKAKEFAAELNREAKQQLEGFDSRKAAPLIALADY | | | |
| | IAYRDN | | | |
| GGPPS3 | MAYSGMATSYHGLHFMNIATQECNLKRLSIPSRRFHGVSPSLWA | 10 | Abies | Select seq |
| | SNGFQGHLKRELSANSFLVSSSRYSNTIAKFTNLPEKVKEKVIEFD | | grandis | gb\|AAL17614.2\| |
| | FKEYLRSKAMAVNEALDRAVPLRYPERIHEAMRYSLLAGGKRVRP | | | geranylgeranyl |
| | VLCISACELVGGTEEVAMPTACAMEMIHTMSLIHDDLPCMDNDD | | | diphosphate synthase |
| | FRRGKPTNHKVFGEGTAILAGDALLSFAFEHIAVSTSKSVGTDRIL | | | [Abies grandis] |
| | RVVSELGRTIGSQGLVGGQVADITSEGDASVDLDTLEWIHIHKTA | | | |
| | VLLECSVMCGAIISGASDNEIERIQRYARSVGLLFQVVDDILDVTK | | | |
| | SSKELGKTAGKDLISDKATYPKLMGLEKAKQFASDLLIRAKEDLS | | | |
| | CFDPMKAAPLLGLADYIAFRQN | | | |
| GGPPS4 | MSYILAEALNFVRRGISHLNYWGASHSLSADNYWESNFQGFPRLL | 11 | Penicillium | Select seq |
| | SDSSKAPSTIRTVQVLEDDVDDIAIQYNKIVRGPLDYLLAIPGKDI | | paxilli | sp\|Q9C446.1\| |
| | RSKLIDSFNIWLQLPEEKLSIVKDIINLLHTASLLIDDIQDASRLRR | | | RecName: |
| | GKPVAHDVYGVAQTINSANYAYYLQQARLKEIGDPRAFEIFTRSLL | | | Full = Geranylgeranyl |
| | DLHLGQGMDLYWRDMVVCPTEEEYTRMVMYKTGGLFNLALDLM | | | pyrophosphate |
| | RIQSRKNTDFSKLVELLGVIFQIRDDYMNLQSGLYAEKKGLMEDL | | | synthase |
| | TEGKFSYPIIHSIRASPESSELLDILKQRTEDEAVKIRAVKIMESTG | | | paxG; [Penicillium |
| | SFQYTRETLSRLSAEARGYVKKLETSLGPNPGIHKILDLLEVEYPTN | | | paxilli] |
| | EKGRV | | | |
| GGPPS5 | MLNSHNRTEERSTEDIILEPYTYLISQPGKDIRAKLISAFDLWLHVP | 12 | Mucor | Select seq |
| | KDVLCVINKIIGMLHNASLMIDDVQDDSDLRRGVPVAHHIYGVPQ | | lusitanicus | sp\|Q9P885.1\| |
| | TINTANYVIFLALQEVMKLNIPSMMQVCTEELINLHRGQGIELYWR | | | RecName: |
| | DSLTCPTEEEYIDMVNNKTSGLLRLAVRLMQAASESDIDYTPLVNI | | | Full = Geranylgeranyl |
| | IGIHFQVRDDYMNLQSTSYTNNKGFCEDLTEGKFSFPIIHAIRKDP | | | pyrophosphate |
| | SNRQLLNIISQKPTSIEVKKYALEVIRKAGSFEYVREFLRQKEAESL | | | synthase; [Mucor |
| | KEIKRLGGNPLLEKYIETIRVEATND | | | lusitanicus] |
| GGPPS6 | MSSVNLGSWVHTSSVISQATRSRSKSKPLSFSPVSIPLFYRNSKR | 13 | Croton | Select seq |
| | SVSYVSAIVTKDEETIQEEQNKNSSSSLGFDFKSYMVQKASAINQ | | sublyratus | dbj\|BAA86284.1\| |
| | ALEAAVSLREPLKIHESMRYSLLAGGKRVRPALCLAACELVGGDE | | | geranylgeranyl |
| | SMAMPAACAVEMIHTMSLIHDDLPCMDNDDLRRGKPTNHIVFGE | | | pyrophosphate |
| | NVAVLAGDALLAFAFEHIAVSTLNVSPVRIVRGVGELAKAIGAEGL | | | Synthase [Croton |
| | VAGQVVDICSEGLSEVDLEKLEFIHIHKTAKLLEGAVVLGAIMGG | | | sublyratus] |
| | GTDEEVEKLRKYARDIGLLFQVVDDILDVTKSSQELGKTAGKDLV | | | |
| | ADKVTYPKLMGIEKSREFAEKLNKEAQEQLAGFDPEKAAPLIALAN | | | |
| | YIAYRQN | | | |
| GGPPS7 | MSSVNLGSWVHTSYVLNQATRSRSKSKSFSLPFNPLKSLAISFAY | 14 | Hevea | Select seq |
| | RKSERPISSVSAIITKEEETLQEEQNNPPPSFDFKSYMLQKGNSIN | | brasiliensis | sp\|Q94ID7.1\| |
| | QALEAAIPLQEPAKIHESMRYSLLAGGKRVRPALCLAACELVGGN | | | RecName: |
| | DSMAMPAACAVEMIHTMSLIHDDLPCMDNDDLRRGKPTNHIVFG | | | Full = Geranylgeranyl |
| | EDVAVLAGDALLAFAFEHIAVSTLNVSSARIVRAVGELAKAIGAEG | | | pyrophosphate |
| | LVAGQVVDINSEGSSEVDLEKLEFIHIHKTAKLLEGAVVLGAILGG | | | synthase, |
| | GTDEEVEKLRKYARDIGLLFQVVDDILDVTKSSQELGKTAGKDLV | | | chloroplastic; [Hevea |
| | ADKVTYPKLLGIEKSREFAEKLNKEAQEQLAGFDPEKAAPLIALAN | | | brasiliensis] |
| | YIAHRQN | | | |
| GGPPS8 | MVSGSKAGVSPHREIEVMRQSIDDHLAGLLPETDSQDIVSLAMR | 15 | Pseudescherichia | Select seq |
| | EGVMAPGKRIRPLLMLLAARDLRYQGSMPTLLDLACAVELTHTAS | | vulneris | sp\|P22873.1\| |
| | LMLDDMPCMDNAELRRGQPTTHKKFGESVAILASVGLLSKAFGLI | | | RecName: |
| | AATGDLPGERRAQAVNELSTAVGVQGLVLGQFRDLNDAALDRTP | | | Full = Geranylgeranyl |
| | DAILSTNHLKTGILFSAMLQIVAIASASSPSTRETLHAFALDFGQAF | | | diphosphate synthase; |
| | QLLDDLRDDHPETGKDRNKDAGKSTLVNRLGADAARQKLREHID | | | [Pseudescherichia |
| | SADKHLTFACPQGGAIRQFMHLWFGHHLADWSPVMKIA | | | vulneris] |
| GGPPS9 | MTVCAEQHVNFIHSDAASLLNDIEQRLDQLLPVESERDLVGAAMR | 16 | Enterobacter | Select seq |
| | DGALAPGKRIRPLLLLLAARDLGCNATSAGLLDLACAVEMVHAAS | | agglomerans | gb\|AFX61741.1\| |
| | LILDDMPCMDDAQLRRGRPTIHCQYGEHVAILAAVALLSKAFGVV | | | geranylgeranyl |
| | AAAEGLTATARADAVAELSHAVGMQGLVQGQFKDLSEGDKPRSA | | | diphosphate synthase |
| | DAILMTNHYKTSTLFCASMQMASIVAEASGEAREQLHRFSLNLGQ | | | [Pantoea agglomerans] |
| | AFQLLDDLTDGMADTGKDAHQDDGKSTLVNLLGPQAVETRLRDH | | | (Enterobacter |
| | LRCASEHLLSACQDGYATHHFVQAWFEKKLAAVS | | | agglomerans) |
| IDI | MTRAERKRQHINHALSIGQKRETGLDDITFVHVSLPDLALEQVDI | 31 | Bacillus | Select seq |
| | STKIGELSSSSPIFINAMTGGGGKLTYEINKSLARAASQAGIPLAV | | subtilis | ref\|WP_004399098.1\| |
| | GSQMSALKDPSERLSYEIVRKENPNGLIFANLGSEATAAQAKEAV | | | type 2 |
| | EMIGANALQIHLNVIQEIVMPEGDRSFSGALKRIEQICSRVSVPVI | | | isopentenyl- |
| | VKEVGFGMSKASAGKLYEAGAAAVDIGGYGGTNFSKIENLRRQR | | | diphosphate Delta- |
| | QISFFNSWGISTAASLAEIRSEFPASTMIASGGLQDALDVAKAIAL | | | isomerase [Bacillus] |
| | GASCTGMAGHFLKALTDSGEEGLLEEIQLILEELKLIMTVLGARTIA | | | |
| | DLQKAPLVIKGETHHWLTERGVNTSSYSVR | | | |
| IDI | MQTEHVILLNAQGVPTGTLEKYAAHTADTRLHLAFSSWLFNAKGQ | 41 | Escherichia | Select seq |
| | LLVTRRALSKKAWPGVWTNSVCGHPQLGESNEDAVIRRCRYELG | | coli | ref\|WP_001192820.1\| |
| | VEITPPESIYPDFRYRATDPSGIVENEVCPVFAARTTSALQINDDEV | | | isopentenyl- |
| | MDYQWCDLADVLHGIDATPWAFSPWMVMQATNREARKRLSAFT | | | diphosphate Delta- |
| | QLK | | | isomerase [Bacteria] |
| DXS | MSFDIAKYPTLALVDSTQELRLLPKESLPKLCDELRRYLLDSVSRS | 32 | Escherichia | Select seq |
| | SGHFASGLGTVELTVALHYVYNTPFDQLIWDVGHQAYPHKILTGR | | coli | ref\|WP_000006797.1\| |
| | RDKIGTIRQKGGLHPFPWRGESEYDVLSVGHSSTSISAGIGIAVA | | | 1-deoxy-D- |
| | AEKEGKNRRTVCVIGDGAITAGMAFEAMNHAGDIRPDMLVILND | | | xylulose-5-phosphate |
| | NEMSISENVGALNNHLAQLLSGKLYSSLREGGKKVFSGVPPIKEL | | | synthase |
| | LKRTEEHIKGMVVPGTLFEELGFNYIGPVDGHDVLGLITTLKNMRD | | | [Enterobacteriaceae] |
| | LKGPQFLHIMTKKGRGYEPAEKDPITFHAVPKFDPSSGCLPKSSG | | | (e. coli) |
| | GLPSYSKIFGDWLCETAAKDNKLMAITPAMREGSGMVEFSRKFP | | | |
| | DRYFDVAIAEQHAVTFAAGLAIGGYKPIVAIYSTFLQRAYDQVLHD | | | |
| | VAIQKLPVLFAIDRAGIVGADGQTHQGAFDLSYLRCIPEMVIMTPS | | | |
| | DENECRQMLYTGYHYNDGPSAVRYPRGNAVGVELTPLEKLPIGKG | | | |
| | IVKRRGEKLAILNFGTLMPEAAKVAESLNATLVDMRFVKPLDEALI | | | |
| | LEMAASHEALVTVEENAIMGGAGSGVNEVLMAHRKPVPVLNIGLP | | | |
| | DFFIPQGTQEEMRAELGLDAAGMEAKIKAWLA | | | |
| MVK | MSLPFLTSAPGKVIIFGEHSAVYNKPAVAASVSALRTYLLISESSAP | 22 | Saccharomyces | Select seq |
| | DTIELDFPDISFNHKWSINDFNAITEDQVNSQKLAKAQQATDGLS | | cerevisiae | ref\|NP_013935.1\| |
| | QELVSLLDPLLAQLSESFHYHAAFCFLYMFVCLCPHAKNIKFSLKS | | | mevalonate |
| | TLPIGAGLGSSASISVSLALAMAYLGGLIGSNDLEKLSENDKHIVN | | | kinase [Saccharomyces |
| | QWAFIGEKCIHGTPSGIDNAVATYGNALLFEKDSHNGTINTNNFK | | | cerevisiae S288C] |
| | FLDDFPAIPMILTYTRIPRSTKDLVARVRVLVTEKFPEVMKPILDAM | | | |
| | GECALQGLEIMTKLSKCKGTDDEAVETNNELYEQLLELIRINHGLL | | | |
| | VSIGVSHPGLELIKNLSDDLRIGSTKLTGAGGGGCSLTLLRRDITQ | | | |
| | EQIDSFKKKLQDDFSYETFETDLGGTGCCLLSAKNLNKDLKIKSLV | | | |
| | FQLFENKTTTKQQIDDLLLPGNTNLPWTS | | | |
| PMK | MSELRAFSAPGKALLAGGYLVLDTKYEAFVVGLSARMHAVAHPYG | 23 | Saccharomyces | Select seq |
| | SLQGSDKFEVRVKSKQFKDGEWLYHISPKSGFIPVSIGGSKNPFI | | cerevisiae | ref\|NP_013947.1\| |
| | EKVIANVFSYFKPNMDDYCNRNLFVIDIFSDDAYHSQEDSVTEHR | | | phosphomeval |
| | GNRRLSFHSHRIEEVPKTGLGSSAGLVTVLTTALASFFVSDLENNV | | | onate kinase |
| | DKYREVIHNLAQVAHCQAQGKIGSGFDVAAAAYGSIRYRRFPPAL | | | [Saccharomyces |
| | ISNLPDIGSATYGSKLAHLVDEEDWNITIKSNHLPSGLTLWMGDI | | | cerevisiae S288C] |
| | KNGSETVKLVQKVKNWYDSHMPESLKIYTELDHANSRFMDGLSK | | | |
| | LDRLHETHDDYSDQIFESLERNDCTCQKYPEITEVRDAVATIRRSF | | | |
| | RKITKESGADIEPPVQTSLLDDCQTLKGVLTCLIPGAGGYDAIAVI | | | |
| | TKQDVDLRAQTANDKRFSKVQWLDVTQADWGVRKEKDPETYLD | | | |
| | K | | | |
| PMD | MTVYTASVTAPVNIATLKYWGKRDTKLNLPTNSSISVTLSQDDLR | 24 | Saccharomyces | Select seq |
| | TLTSAATAPEFERDTLWLNGEPHSIDNERTQNCLRDLRQLRKEME | | cerevisiae | ref\|NP_014441.1\| |
| | SKDASLPTLSQWKLHIVSENNFPTAAGLASSAAGFAALVSAIAKLY | | | diphosphomev |
| | QLPQSTSEISRIARKGSGSACRSLFGGYVAWEMGKAEDGHDSM | | | alonate decarboxylase |
| | AVQIADSSDWPQMKACVLVVSDIKKDVSSTQGMQLTVATSELFK | | | MVD1 [Saccharomyces |
| | ERIEHVVPKRFEVMRKAIVEKDFATFAKETMMDSNSFHATCLDSF | | | cerevisiae S288C] |
| | PPIFYMNDTSKRIISWCHTINQFYGETIVAYTFDAGPNAVLYYLAE | | | |
| | NESKLFAFIYKLFGSVPGWDKKFTTEQLEAFNHQFESSNFTAREL | | | |
| | DLELQKDVARVILTQVGSGPQETNESLIDAKTGLPKE | | | |
| HmgACT | MKTVVIIDALRTPIGKYKGSLSQVSAVDLGTHVTTQLLKRHSTISE | 17 | Enterococcus | Select seq |
| | EIDQVIFGNVLQAGNGQNPARQIAINSGLSHEIPAMTVNEVCGSG | | faecalis | ref\|WP_071646686.1\| |
| | MKAVILAKQLIQLGEAEVLIAGGIENMSQAPKLQRFNYETESYDAP | | | 3-hydroxy-3- |
| | FSSMMYDGLTDAFSGQAMGLTAENVAEKYHVTREEQDQFSVHS | | | methylglutaryl-CoA |
| | QLKAAQAQAEGIFADEIAPLEVSGTLVEKDEGIRPNSSVEKLGTLK | | | reductase, degradative |
| | TVFKEDGTVTAGNASTINDGASALIIASQEYAEAHGLPYLAIIRDS | | | [Enterococcus faecalis] |
| | VEVGIDPAYMGISPIKAIQKLLARNQLTTEEIDLYEINEAFAATSIV | | | |
| | VQRELALPEEKVNIYGGGISLGHAIGATGARLLTSLSYQLNQKEKK | | | |
| | YGVASLCIGGGLGLAMLLERPQQKKNSRFYQMSPEERLASLLNEG | | | |
| | QISADTKKEFENTALSSQIANHMIENQISETEVPMGVGLHLTVDE | | | |
| | TDYLVPMATEEPSVIAALSNGAKIAQGFKTVSQQRLMRGQIVFYN | | | |
| | VADPESLIDKLQVREAEIFQQAELSYPSIVKRGGGLRDLQYRAFDE | | | |
| | SFVSVDFLVDVKDAMGANIVNAMLEGVAELFREWFAEQKILFSIL | | | |
| | SNYATESVVTMKTAIPVSRLSKGSNGREIAEKIVLASRYASLDPYR | | | |
| | AVTHNKGIMNGIEAVVLATGNDTRAVSASCHAFAVKEGRYQGLT | | | |
| | SWTLDGEQLIGEISVPLALATVGGATKVLPKSQAAADLLAVTDAK | | | |
| | ELSRVVAAVGLAQNLAALRALVSEGIQKGHMALQARSLAMTVGA | | | |
| | TGKEVEAVAQQLKRQKTMNQDRALAILNDLRKQ | | | |
| HmgS | MTIGIDKISFFVPPYYIDMTALAEARNVDPGKFHIGIGQDQMAVNP | 18 | Enterococcus | Select seq pdb |
| | ISQDIVTFAANAAEAILTKEDKEAIDMVIVGTESSIDESKAAAVVL | | faecalis | \|2HDB\|A |
| | HRLMGIQPFARSFEIKEGCYGATAGLQLAKNHVALHPDKKVLVVA | | | Chain A, HMG- |
| | ADIAKYGLNSGGEPTQGAGAVAMLVASEPRILALKEDNVMLTQDI | | | CoA synthase |
| | YDFWRPTGHPYPMVDGPLSNETYIQSFAQVWDEHKKRTGLDFAD | | | [Enterococcus faecalis] |
| | YDALAFHIPYTKMGKKALLAKISDQTEAEQERILARYEESIIYSRRV | | | |
| | GNLYTGSLYLGLISLLENATTLTAGNQIGLFSYGSGAVAEFFTGEL | | | |
| | VAGYQNHLQKETHLALLDNRTELSIAEYEAMFAETLDTDIDQTLED | | | |
| | ELKYSISAINNTVRSYRN | | | |
| HmgR1 | MKISWNGFSKKSYQERLELLKAQALLSPERQASLEKDEQMSVTV | 19 | S. | Select seq |
| | ADQLSENVVGTFSLPYSLVPEVLVNGQEYTVPYVTEEPSVVAAASY | | pneumoniae | ref\|WP_000704388.1\| |
| | ASKIIKRAGGFTAQVHQRQMIGQVALYQVANPKLAQEKIASKKAE | | | 3-hydroxy-3- |
| | LLELANQAYPSIVKRGGGARDLHVEQIKGEPDFLVVYIHVDTQEA | | | methylglutaryl-CoA |
| | MGANMLNTMLEALKPVLEELSQGQSLMGILSNYATDSLVTASCRI | | | reductase, degradative |
| | AFRYLSRQKDQGREIAEKIALASQFAQADPYRAATHNKGIFNGID | | | [Streptococcus |
| | AILIATGNDWRAIEAGAHAFASRDGRYQGLSCWTLDLEREELVGE | | | pneumoniae] |
| | MTLPMPVATKGGSIGLNPRVALSHDLLGNPSARELAQIIVSIGLAQ | | | |
| | NFAALKALVSTGIQQGHMKLQAKSLALLAGASESEVAPLVERLIS | | | |
| | DKTFNLETAQRYLENLRS | | | |
| HmgR2 | MDQLVKTEVTKKSFTAPVQKASTPVLTNKTVISGSKVKSLSSAQS | 20 | Saccharomyces | Select seq |
| | SSSGPSSSSEEDDSRDIESLDKKIRPLEELEALLSSGNTKQLKNKE | | cerevisiae | gb\|AJS96703.1\| |
| | VAALVIHGKLPLYALEKKLGDTTRAVAVRRKALSILAEAPVLASDR | | | Hmg1p |
| | LPYKNYDYDRVFGACCENVIGYMPLPVGVIGPLVIDGTSYHIPMAT | | | [Saccharomyces |
| | TEGCLVASAMRGCKAINAGGGATTVLTKDGMTRGPVVRFPTLKR | | | cerevisiae YJM1463] |
| | SGACKIWLDSEEGQNAIKKAFNSTSRFARLQHIQTCLAGDLLFMR | | | |
| | FRTTTGDAMGMNMISKGVEYSLKQMVEEYGWEDMEVVSVSGNY | | | |
| | CTDKKPAAINWIEGRGKSVVAEATIPGDVVRKVLKSDVSALVELN | | | |
| | IAKNLVGSAMAGSVGGFNAHAANLVTAVFLALGQDPAQNVESSN | | | |
| | CITLMKEVDGDLRISVSMPSIEVGTIGGGTVLEPQGAMLDLLGVR | | | |
| | GPHATAPGTNARQLARIVACAVLAGELSLCAALAAGHLVQSHMTH | | | |
| | NRKPAEPTKPNNLDATDINRLKDGSVTCIKS | | | |
| HmgR3 | MSPEERLASLLNEGQISADTKKEFENTALSSQIANHMIENQISETE | 21 | Enterococcus | Select seq |
| | VPMGVGLHLTVDETDYLVPMATEEPSVIAALSNGAKIAQGFKTVN | | faecalis | ref\|WP_010820440.1\| |
| | QQRLMRGQIVFYDVADAESLIDELQVRETEIFQQAELSYPSIVKRG | | | 3-hydroxy-3- |
| | GGLRDLQYRAFDESFVSVDFLVDVKDAMGANIVNAMLEGVAELF | | | methylglutaryl-CoA |
| | REWFAEQKILFSILSNYATESVVTMKTAIPVSRLSKGSNGREIAEK | | | reductase, degradative |
| | IVLASRYASLDPYRAVTHNKGIMNGIEAVVLATGNDTRAVSASCH | | | [Enterococcus faecalis] |
| | AFAVKEGRYQGLTSWTLDGEQLIGEISVPLALATVGGATKVLPKS | | | |
| | QAAADLLAVTDAKELSRVVAAVGLAQNLAALRALVSEGIQKGHM | | | |
| | ALQARSLAMTVGATGKEVEAVAQQLKRQKTMNQDRALAILNDLR | | | |
| | KQ | | | |
| CrtI1 | MKPTTVIGAGFGGLALAIRLQAAGIPVLLLEQRDKPGGRAYVYED | 33 | Pantoea | Select seq |
| | QGFTFDAGPTVITDPSAIEELFALAGKQLKEYVELLPVTPFYRLCWE | | ananatis | ref\|WP_176017230.1\| |
| | SGKVFNYDNDQTRLEAQIQQFNPRDVEGYRQFLDYSRAVFKEGY | | | phytoene |
| | LKLGTVPFLSFRDMLRAAPQLAKLQAWRSVYSKVASYIEDEHLRQ | | | desaturase [Pantoea |
| | AFSFHSLLVGGNPFATSSIYTLIHALEREWGVWFPRGGTGALVQG | | | ananatis] |
| | MIKLFQDLGGEVVLNARVSHMETTGNKIEAVHLEDGRRFLTQAVA | | | |
| | SNADVVHTYRDLLSQHPAAVKQSNKLQTKRMSNSLFVLYFGLNH | | | |
| | HHDQLAHHTVCFGPRYRELIDEIFNHDGLAEDFSLYLHAPCVTDS | | | |
| | SLAPEGCGSYYVLAPVPHLGTANLDWTVEGPKLRDRIFAYLEQHY | | | |
| | MPGLRSQLVTHRMFTPFDFRDQLNAYHGSAFSVEPVLTQSAWFR | | | |
| | PHNRDKTITNLYLVGAGTHPGAGIPGVIGSAKATAGLMLEDLI | | | |
| CrtI2 | MNAHSPAAKTAIVIGAGFGGLALAIRLQSAGIATTLVEARDKPGG | 34 | Paracoccus | Select seq |
| | RAYVWHDQGHLFDAGPTVITDPDALKELWALTGQDMARDVTLM | | sp. | sp\|P54978.2\| |
| | PVSPFYRLMWPGGKVFDYVNEADQLERQIAQFNPDDLEGYRRFR | | | RecName: |
| | DYAEEVYQEGYVKLGTVPFLKLGQMLKAAPALMKLEAYKSVHAKV | | | Full = Phytoene |
| | ATFIKDPYLRQAFSYHTLLVGGNPFSTSSIYALIHALERRGGVWFA | | | desaturase (lycopene- |
| | KGGTNQLVAGMVALFERLGGQMLLNAKVARIDTDGPRATGVTLA | | | forming); [Paracoccus |
| | DGRALTADMVASNGDVMHNYRDLLGHTARGQSRAKSLNAKRW | | | sp. N81106] |
| | SMSLFVLHFGLREAPKDVAHHTILFGPRYKELVNEIFKGPKLAEDF | | | |
| | SLYLHSPCTTDPEMAPPGMSTHYVLAPVPHLGRADIDWAVEGPRY | | | |
| | ADRILASLEERLIPNLRANLTTTRIFTPSDFASELNAHHGSAFSVEP | | | |
| | ILTQSAWFRPHNRDKTIRNFYLVGAGTHPGAGIPGVVGSAKATAQ | | | |
| | VMLSDLASA | | | |
| CrtI3 | MKKTVVIGAGFGGLALAIRLQAAGIPTVLLEQRDKPGGRAYVWHD | 35 | Pseudescherichia | Select seq |
| | QGFTFDAGPTVITDPTALEALFTLAGRRMEDYVRLLPVKPFYRLCW | | vulneris | ref\|WP_020503291.1\| |
| | ESGKTLDYANDSAELEAQITQFNPRDVEGYRRFLAYSQAVFQEGY | | | phytoene |
| | LRLGSVPFLSFRDMLRAGPQLLKLQAWQSVYQSVSRFIEDEHLRQ | | | desaturase |
| | AFSFHSLLVGGNPFTTSSIYTLIHALEREWGVWFPEGGTGALVNG | | | [Lamprocystis |
| | MVKLFTDLGGEIELNARVEELVVADNRVSQVRLADGRIFDTDAVA | | | purpurea] |
| | SNADVVNTYKKLLGHHPVGQKRAAALERKSMSNSLFVLYFGLNQ | | | |
| | PHSQLAHHTICFGPRYRELIDEIFTGSALADDFSLYLHSPCVTDPSL | | | |
| | APPGCASFYVLAPVPHLGNAPLDWAQEGPKLRDRIFDYLEERYMP | | | |
| | GLRSQLVTQRIFTPADFHDTLDAHLGSAFSIEPLLTQSAWFRPHN | | | |
| | RDSDIANLYLVGAGTHPGAGIPGVVASAKATASLMIEDLQ | | | |
| CrtI4 | MSKNTEGMGRAVVIGAGLGGLAAAMRLGAKGYKVTVVDRLDRP | 36 | Rhodobacter | Select seq |
| | GGRGSSITKGGHRFDLGPTIVTVPDRLRELWADCGRDFDKDVSL | | capsulatus | ref\|WP_013066423.1\| |
| | VPMEPFYTIDFPDGEKYTAYGDDAKVKAEVARISPGDVEGFRHFM | | | phytoene |
| | WDAKARYEFGYENLGRKPMSKLWDLIKVLPTFGWLRADRSVYGH | | | desaturase |
| | AKKMVKDDHLRFALSFHPLFIGGDPFHVTSMYILVSQLEKKFGVH | | | [Rhodobacter |
| | YAIGGVQAIADAMAKVITDQGGEMRLNTEVDEILVSRDGKATGIR | | | capsulatus] |
| | LMDGTELPAQVVVSNADAGHTYKRLLRNRDRWRWTDEKLDKKR | | | |
| | WSMGLFVWYFGTKGTAKMWKDVGHHTVVVGPRYKEHVQDIFIK | | | |
| | GELAEDMSLYVHRPSVTDPTAAPKGDDTFYVLSPVPNLGFDNGV | | | |
| | DWSVEAEKYKAKVLKVIEERLLPGVAEKITEEVVFTPETFRDRYLS | | | |
| | PLGAGFSLEPRILQSAWFRPHNASEEVDGLYLVGAGTHPGAGVPS | | | |
| | VIGSGELVAQMIPDAPKPETPAAAAPKARTPRAKAAQ | | | |
| CrtI5 | MLPAVGVDRPVGRRLQLHARVQQGIGVRSIAWSRVQAVALLAVV | 37 | Synechocystis | Select seq |
| | RDRQAAVQTPRVGQHQAVVQRNRRRHVRRARQGVADGRQHLG | | sp. | sp\|P21134.1\| |
| | HARRAERDDLCVQEGGGDIEIQALVRAFEQDGVECELDPLALGLA | | | RecName: |
| | DVSAAVGVIGQTGRRLQLLEFIGEDVHRQAKTVRRPTQPGLIGGD | | | Full = Phytoene |
| | GFGIERQGLGLGEDRGAVEAAAAIAGPRRDEDVQVRGKGVFGRD | | | dehydrogenase; |
| | PTRHVVVLEGAVPDEGIGRGEGLGEGRDAPRVQVVVAPAEGRLV | | | [Synechocystis sp. PCC |
| | AGVTQTARQGQLVGQVIVDLAEHGEAVARLLVQVVVLGPGRIAG | | | 6714] |
| | PVGVEVDIVRDVEGAHDPVQTTVVVVAQTHFQRGLVQRLIGIEAR | | | |
| | GRIGRAVAVALHRASRALDVADHRQIQVIAQTMLQRRRHAIDVD | | | |
| | AMGRILRRIELVRVVVLARIGDIAVQRRRDTVVLARVAPVAAAEQL | | | |
| | HGQVGVRREIEGDSGPAGEQILVAETFTAARIDDGRNLGLGHAG | | | |
| | DDRLEARQVARARPGHEAAPCSASTASRTPTVSLRGPPTAASIAR | | | |
| CrtI6 | MITVKGPVDHVVVVGAGLAGLAAALHLLGAGRRVTVVEREDVPG | 38 | Streptomyces | Select seq |
| | GRAGLLESGGFRIDTGPTVLTMPDLVEDAFAAVGERMADRLELIR | | griseus | sp\|P54981.1\| |
| | LAPAYRARFADGSQLDVHTDGAAMEAAVEEFAGARQAVGYRRLR | | | RecName: |
| | IWLERLYRVQMRRFIDTNFDSPLQLAHPDLARLAALGGFGRLDAR | | | Full = Phytoene |
| | IGHFVSDERLRRVFSFQALYAGVPPARALAAYAVIAYMDTVAGVYF | | | dehydrogenase; |
| | PRGGMHALPRAMADAASDAGAVLRYGQRVTRLERSGDRVTAVV | | | [Streptomyces griseus] |
| | TDQEHIPCDAVVLTPDLPVSYRLLGRTPHRPLPLRHSPSAVILHTG | | | |
| | TDRTWPDLAHHTISFGAAWKNTFHELTRTGRLMSDPSLLITRPTA | | | |
| | TDPSLAPPGKHLHYVLAPCPNTEVGPGVREWRELGPRYRDELLAE | | | |
| | LERREMPGLGAAIEEEGLVTPVDWTAQGHAAGTPFSVAHTFPQTG | | | |
| | PFRPRNLVRGTVNAVLAGCGTTPGVGVPTVLISGKLAAERITGPRI | | | |
| | ARAPRSAPGGSSA | | | |
| BBS1 | MSNFLVSTCSSPLALDENNSTKQDHVIRNTVTFHSSIWGDQFLTY | 25 | Cynara | Select seq |
| | DEKDDLVAEKQLAEELIEETRKELIITTSSHEPIQHMKLIQLIDAVQ | | cardunculus | ref\|XP_024994640.1\| |
| | RLGVAYHFEKEIEDALQHVYRTYGHQGIHNNNDLQSISLWFRILR | | | (E)-beta- |
| | QQGFNVSSEIFKNHMDEKGNLFSNDVQSMLALYEASYMRVEGEK | | | farnesene synthase- |
| | VLDDALEFTKTHLAIIAKDPSCDSSLRTQIQDALRQPLRKRLPRLE | | | like [Cynara |
| | AVRYIPIYQQQSSHNQILLKLAKLDFNMLQTMHKKELSEICKWWK | | | cardunculus var. |
| | DLDMQNKLPFVRDRLIEGYFWILGIYFEPHHSRSRMFLIKSCMWL | | | scolymus] |
| | VVIDDTFDNYGTYEELEIFTEAVERWSINCLDMLPEYMKLIYKELVI | | | |
| | VHQEMEETLEKEGKAYHIHHVKELAKECTRSLLVEAKWLKEGYMP | | | |
| | TLDEYISNSLITCAYAVMIARSYVGGDDKLVNEDSFKWVATHPPL | | | |
| | VKASCLILRLMDDIATHKEEQERGHVASSIECYIKETGATEEEARE | | | |
| | HFSKQVEDAWKVVNRESLRPTAVAFPLVMPAINLARMCDALYKG | | | |
| | NHDGYNHAGKEVIQYIKSLLVHPLI | | | |
| BBS2 | MSTLSVSSPSFSSFPLSSADNTHSTKKDVIRNSVIFHDSIWGDQF | 26 | Artemisia | Select seq |
| | LEHDEKFNVATEKQLIEELKEEVRKELMIRASNEASQDIKLIQLIDE | | annua | gb\|PWA60940.1\| |
| | VQRLGVAYHFEKEIEESLQHIYVTYGDNWIGNNIESVSLWFQLLR | | | sesquiterpene |
| | QHGFNISSDIFKNYKDEKGKIQESLCDDPQGMLALYEATYLRVEG | | | synthase 1 [Artemisia |
| | EEVLDDANEFTKINLGIMSKDPFCDSSLRTEIQQALKQPLRRRLPR | | | annua] |
| | LEAVRYIPIYQQKASHNEVLLKLAKLDFNVLQDMHKHELSLICKW | | | |
| | WKDLDIRNKLPFVRDRLIEGYFWIMGIYFEPQHSRTRIFLMKTCM | | | |
| | WLVVLDDTFDNYGTYEELEMFTQAVERWSITCLDELPEYMKLIYQ | | | |
| | EQLSVHQEMEESLEKEGKAYQIHYIKEMAKEGTRSLLLEAKWLRE | | | |
| | GYMPTLEEYISNSLVTCGYAVMTARSYVARDDDIVSEDAFKWVAT | | | |
| | HPPLVKATCKILRLMDDIATHKEEQERGHIASSIECYRKETGASEE | | | |
| | EACEFFSKQVEDAWKVINRESLRPTDVPFPLLIPAINLARVSDTLY | | | |
| | KYNDGYNHAGKEVISHIKSIFVNPMIV | | | |
| BBS3 | MSTLPISSVSSSSSTSPIVVDDKDSTKPDVIRHTTNFNASIWGDQ | 27 | Artemisia | Select seq |
| | FLTYDEPEDLVMKKQLVEELKEEVKKELITIKGSNEPMQHVKLIELI | | annua | gb\|QKE59890.1\| beta- |
| | DAVQRLGIAYHFEEEIEEALQHIHVTYGEQWVDKENLQSISLWFR | | | farnesene synthase |
| | LLRQQGFNVSSGVFKDFMDEKGKFKESLCNDAQGILALYEAAFM | | | [synthetic construct] |
| | RVEDETILDNALEFSKVHLDIIAKDPSCDSSLRTQIHQALKQPLRR | | | |
| | RLARIEALHYMPIYQQETSHDEVLLKLAKLDFSVLQSMHKKELSHI | | | |
| | CKWWKDLDLQNKLPFVRDRVVEGYFWILSIYYEPQHARTRMFLM | | | |
| | KSCMWLVVLDDTFDNYGTYEELEIFTQAVEKWSISCLDMLPEYMK | | | |
| | LIYQELVNLHVEMEESLEKEGKAYQIHYVKEMAKELVRNYLVEAR | | | |
| | WLKEGYMPTLEEYMSVSMVTGTYGLMTARSYVGRGDIVNEDTFK | | | |
| | WVSSYPPIVKASCVIIRLMDDIVSHKEEQERGHVASSIECYSKES | | | |
| | GASEEEACEYISRKVEDAWKVINRESLRPTAVPFPLLMPAINLARM | | | |
| | CEVLYSVNDGFTHAEGDMKSYMKSFFVHPMVV | | | |
| BBS4 | MSTAFPVAALVSASASFSSSEPLPSPPPTTTATKQRNTTNHHSSI | 28 | Eremanthus | Select seq |
| | WGDQFLTYHESEDILKEKLLVDELKEQVRKELISASVDDHLELIDA | | erythropappus | gb\|AYJ71561.1\| |
| | IQRLGVAYYYENEIEEALQHIYVTYGDHWIHTDNLQSISLWFRLLR | | | bisabolol |
| | QQGFNVSSGIFKNHMNDKGNFKESISNDVQGMLELYEAAYMRVE | | | synthase [Eremanthus |
| | GEEILDYALEFTKTNLGILANDPSCDSSLRSQIQQALKQPLRKRLP | | | erythropappus] |
| | RLEAVRYIPIYKQQACHNDVLLKLAKLDFNVLQSMHKKELSHICK | | | |
| | WWKDLNMQEKLPYVRDRLVEGYFWILGIYFEPHHARTRMFLIKTC | | | |
| | MWLVVIDDTFDNYGAYEELEQFTEAVERWSISCLDELPEYMKLVY | | | |
| | QELVNVHQEMEESLEKEGKAFQIHYVKEMAKECTRSLLLEAKWLK | | | |
| | DGYMPTLEEYLSNSLITCAYAVMVARSYVGREDVMIREETFKWVA | | | |
| | THPPLVKASCLILRLMDDIATHKEEQERGHVASSIECYIRENVGAT | | | |
| | EEEACELFSKQIEDAWKVINRECLRPMDVPFLLVMPAINLARICDA | | | |
| | LYSKGNDGFNHAGEEVINYIKSLVVHPLVV | | | |
| BBS5 | MSNFQLSSVSFSSSILPSAVDDNSDTKLDVIRNTMNFSASIWGD | 29 | Helianthus | Select seq |
| | QFLTFEEPDDLEMEKKVVEVLKEEVRKELVIKGSSNESLQHMKLIE | | annuus | ref\|XP_022017387.1\| |
| | LIDVVQRLGVAYHFEEEIEEALKHIYVTYGEKWVDLNNLHNLSLW | | | (E)-beta- |
| | FRLLRQQGFNVSSGIFKYHMCEKGNFKESLCEDAQGLLALYEASY | | | farnesene synthase |
| | MRVEGEKVLDDALEFTKTHLAIIAKDPSCDSLLRAQIQEALKQPLR | | | [Helianthus annuus] |
| | KRLPRLEAVRYIPIYQQDVSHNEVLLKLAKSDFNVLQSMHKEELS | | | |
| | QICKWWKDLDMQKKLPYVRDRLIEGYFWILGVYFEPQHSHTRIFL | | | |
| | MKTSMWLIVLDDTYDNYGTYEELKIFTDAVQRWSMSCLDLLPEY | | | |
| | MKLIFQELLNHHQEMEESLEKEGKTYQIHYVIEMAKEVLENNLVEA | | | |
| | KWLKEGYMPTLEEYMSVSMKTCTYGLMIARSFVGRVDNMVTEDT | | | |
| | FKWVATYPPIVKAACLVLRLMDDITTHKEEQERGHVASSIECYQK | | | |
| | ETGASEKEACEFISNMVEDAWKVINRESLRPTDIPFPLLPSTINFA | | | |
| | RACDVIYKVNDSYTHARKEMINHIKSLLVHPLAI | | | |
| BBS6 | MSTLSVSTPSFSSSPLSSVNKNSTKQHVTRNSVIFHDSIWGDQFL | 30 | Matricaria | Select seq |
| | EYKEKFNVATEKQLIEELKEEVRNELMIRACNEASRYIKLIQLIDVV | | recutita | gb\|AIG92846.1\| |
| | ERLGLAYHFEKEIEESLQHIYVTYGHKWTNYNNIESLSLWFRLLRQ | | | sesquiterpene |
| | NGFNVSSDIFENHIDEKGNFQESLCNDPQGMLALYEAAYMRVEG | | | synthase 1 [Matricaria |
| | EIILDKALEFTKLHLGIISNDPSCDSSLRTEIKQALKQPLRRRLPRL | | | chamomilla var. |
| | EAVRYIAIYQQKASHSEVLLKLAKLDFNVLQEMHKDELSQICKWW | | | recutita] |
| | KDLDIRNKLPYVRDRLIEGYFWILGIYFEPQHSRTRMFLMKTCMW | | | |
| | LIVLDDTFDNYGTYEELEIFTQAVERWSITCLDELPEYMKLIYHEQF | | | |
| | RVHQEMEESLEKEGKAYQIHYIKEMAKEGTRSLLLEAKWLKEGYM | | | |
| | PTLDEYLSNSLVTCGYALMTARSYVARDDGIVTEDAFKWVATHPP | | | |
| | IVKAACKILRLMDDIATHKEEQERGHIASSIECYRKETGASEEEAC | | | |
| | MDFLKQVEDGWKVINQESLMPTDVPFPLLIPAINLARVSDTLYKD | | | |
| | NDGYNHADKEVIGYIKSLFVHPMIV | | | |
| FPPS | MDFPQQLEACVKQANQALSRFIAPLPFQNTPVVETMQYGALLGGK | 42 | Escherichia | Select seq |
| | RLRPFLVYATGHMFGVSTNTLDAPAAAVECIHAYSLIHDDLPAMD | | coli | ref\|WP_175283368.1\| |
| | DDDLRRGLPTCHVKFGEANAVLAGDALQTLAFSILSDADMPEVSD | | | (2E,6E)-farnesyl |
| | RDRISMISELASASGIAGMCGGQALDLDAEGKHVPLDALERIHRH | | | diphosphate synthase |
| | KTGALIRAAVRLGALSAGDKGRRALPVLDKYAESIGLAFQVQDDI | | | [Escherichia coli] |
| | LDVVGDTATLGKRQGADQQLGKSTYPALLGLEQARKKARDLIDD | | | |
| | ARQSLKQLAEQSLDTSALEALADYIIQRNK | | | |
| MVK | MIEVTTPGKLFIAGEYAVVEPGHPAIIVAVDQFVTVTVEETTDEGSIQ | 48 | Enterococcus | |
| | SAQYSSLPIRWTRRNGELVLDIRENPFHYVLAAIHLTEKYAQEQNK | | faecalis | |
| | ELSFYHLKVTSELDSSNGRKYGLGSSGAVTVGTVKALNIFYDLGLE | | | |
| | NEEIFKLSALAHLAVQGNGSCGDIAASCYGGWIAFSTFDHDWVNQ | | | |
| | KVATETLTDLLAMDWPELMIFPLKVPKQLRLLIGWTGSPASTSDLV | | | |
| | DQVHQSKEEKQAAYEQFLMKSRLCVETMINGENTGKISVIQKQITK | | | |
| | NRQLLAELSSLTGVVIETEALKNLCDLAESYTGAAKSSGAGGGDCG | | | |
| | IVIFRQKSGILPLMTAWEKDGITPLPLHVYTYGQKECKEKHESKR | | | |
| PMD | MLSGKARAHTNIALIKYWGKANEEYILPMNSSLSLTLDAFYTETTVIF | 49 | Enterococcus | |
| | DAHYSEDVFILDGILQNEKQTKKVKEFLNLVRQQADCTWFAKVESQ | | faecalis | |
| | NFVPTAAGLASSASGLAALAGACNVALGLNLSAKDLSRLARRGSG | | | |
| | SACRSIFGGFAQWNKGHSDETSFAENIPANNWENELAMLFILINDG | | | |
| | EKDVSSRDGMKRTVETSSFYQGWLDNVEKDLSQVHEAIKTKDFPR | | | |
| | LGEIIEANGLRMHGTTLGAVPPFTYWSPGSLQAMALVRQARAKGIP | | | |
| | CYFTMDAGPNVKVLVEKKNLEALKTFLSEHFSKEQLVPAFAGPGIE | | | |
| | LFETKGMDK | | | |
| | Blank sequence | 50 | | |
| CrtB_Aa | MSDLVLTSTEAITQGSQSFATAAKLMPPGIRDDTVMLYAWCRHADDVIDGQ | 51 | Agrobacterium | |
| | ALGSRPEAVNDPQARLDGLRADTLAALQGDGPVTPPFAALRAVARRHDFPQ | | aurantiacum | |
| | AWPMDLIEGFAMDVEARDYRYLDDVLEYSYHVAGIVGVMMARVMGVRDDPV | | | |
| | LDRACDLGLAFQLTNIARDVIDDARIGRCYLPGDWLDQAGARVDGPVPSPE | | | |
| | LYTVILRLLDAAELYYASARVGLADLPPRCAWSIAAALRIYRAIGLRIRKG | | | |
| | GPEAYRQRISTSKAAKIGLLGIGGWDVARSRLPGAGVSRQGLWTRPHHA | | | |
| CrtB_Pa | MSQPPLLDHATQTMANGSKSFATAAKLFDPATRRSVLMLYTWCRHCDDVID | 52 | Pantoea | |
| | DQTHGFASEAAAEEEATQRLARLRTLTLAAFEGAEMQDPAFAAFQEVALTH | | agglomerans | |
| | GITPRMALDHLDGFAMDVAQTRYVTFEDTLRYCYHVAGVVGLMMARVMGVR | | | |
| | DERVLDRACDLGLAFQLTNIARDIIDDAAIDRCYLPAEWLQDAGLTPENYA | | | |
| | ARENRAALARVAERLIDAAEPYYISSQAGLHDLPPRCAWAIATARSVYREI | | | |
| | GIKVKAAGGSAWDRRQHTSKGEKIAMLMAAPGQVIRAKTTRVTPRPAGLWQ | | | |
| | RPV | | | |
| CrtYB_ | MLLTYMEVHLYYTLPVLGVLSWLSRPYYTATDALKFKFLTLVAFTTASAWDNYIVYHK | 53 | Mucor | |
| MI | AWSYCPTCVTAVIGYVPLEEYMFFIIMTLLTVAFTNLVMRWHLHSFFIRPETPVMQS | | lusitanicus | |
| | VLVRLVPITALLITAYKAWHLAVPGKPLFYGSCILWYACPVLALLWFGAGEYMMRRP | | | |
| | LAVLVSIALPTLFLCWVDVVAIGAGTWDISLATSTGKFVVPHLPVEEFMFFALINTVLV | | | |
| | FGTCAIDRTMAILHLFKNKSPYQRPYQHSKSFLHQILEMTWAFCLPDQVLHSDTFHD | | | |
| | LSVSWDILRKASKSFYTASAVFPGDVRQELGVLYAFCRATDDLCDNEQVPVQTRKEQ | | | |
| | LILTHQFVSDLFGQKTSAPTAIDWDFYNDQLPASCISAFKSFTRLRHVLEAGAIKELLD | | | |
| | GYKWDLERRSIRDQEDLRYYSACVASSVGEMCTRIILAHADKPASRQQTQWHIIQRAR | | | |
| | EMGLVLQYTNIARDIVTDSEELGRCYLPQDWLTEKEVALIQGGLAREIGEERLLSLSHR | | | |
| | LIYQADELMVVANKGIDKLPSHCQGGVRAACNVYASIGTKLKSYKHHYPSRAHVGN | | | |
| | SKRVEIALLSVYNLYTAPIATSSTTHCRQGKMRNLNTI | | | |
| CrtYB_ | MTALAYYQIHLIYTLPILGLLGLLTSPILTKFDIYKISILVFIAFSATTPW | 54 | Xanthophyl | |
| Xd | DSWIIRNGAWTYPSAESGQ | | lomyces | |
| | GVFGTFLDVPYEEYAFFVIQTVITGLVYVLATRHLLPSLALPKTRSSA | | dendrorhous | |
| | LSLALKALIPLPITYLFTAHPS | | | |
| | PSPDPLVTDHYFYMRALSLLITPPTMLLAALSGEYAFDWKSGRAKS | | | |
| | TIAAIMIPTVYLIWVDYVAVGQDS | | | |
| | WSINDEKIVGWRLGGVLPIEEAMFFLLTNLMIVLGLSACDHTQALYL | | | |
| | LHGRTIYGNKKMPSSFPLITPPV | | | |
| | LSLFFSSRPYSSQPKRDLELAVKLLEEKSRSFFVASAGFPSEVRER | | | |
| | LVGLYAFCRVTDDLIDSPEVSSNP | | | |
| | HATIDMVSDFLTLLFGPPLHPSQPDKILSSPLLPPSHPSRPTGMYPL | | | |
| | PPPPSLSPAELVQFLTERVPVQY | | | |
| | HFAFRLLAKLQGLIPRYPLDELLRGYTTDLIFPLSTEAVQARKTPIET | | | |
| | TADLLDYGLCVAGSVAELLVYV | | | |
| | SWASAPSQVPATIEEREAVLVASREMGTALQLVNIARDIKGDATEG | | | |
| | RFYLPLSFFGLRDESKLAIPTDWT | | | |
| | EPRPQDFDKLLSLSPSSTLPSSNASESFRFEWKTYSLPLVAYAEDL | | | |
| | AKHSYKGIDRLPTEVQAGMRAACA | | | |
| | SYLLIGREIKVVWKGDVGERRTVAGWRRVRKVLSVVMSGWEGQ | | | |
| CrtE_ | MLKEEIAKRAEIINKAIEELLPEREPIGLYKAARHLIKAGGKRLRPVISLL | 55 | Archaeoglobus | |
| Af | AVEALGKDYRKIIPAAVSIETIHNFTLVHDDIMDRDEMRRGVPTVHRVYGE | | fulgidus | |
| | ATAILAGDTLFAEAFKLLTKCDVESEGIRKATEMLSDVCIKICEGQYYDMS | | | |
| | FEKKESVSEEEYLRMVELKTGVLIAASAALPAVLEGESEEIVKALWDYGVL | | | |
| | SGIGFQIQDDLLDLTEETGKDWGSDLLKGKKTLIVIKAFEKGVKLKTFGKE | | | |
| | KADVSEIRDDIEKLRECGAIDYAASMARKMAEEAKRKLEVLPESKAKETLL | | | |
| | ELTDFLVTRKK | | | |
| CrtE_ | MAYTAMAAGTQSLQLRTVASYQECNSMRSCFKLTPFKSFHGVNF | 56 | Taxus X | |
| Txm | NVPSLGAANCEIMGHLKLGSLPYKQCSVSSKSTKTMAQLVDLAET | | media | |
| | EKAEGKDIEFDFNEYMKSKAVAVDAALDKAIPLEYPEKIHESMRYSL | | | |
| | LAGGKRVRPALCIAACELVGGSQDLAMPTACAMEMIHTMSLIHDDL | | | |
| | PCMDNDDFRRGKPTNHKVFGEDTAVLAGDALLSFAFEHIAVATSK | | | |
| | TVPSDRTLRVISELGKTIGSQGLVGGQVVDITSEGDANVDLKTLEWI | | | |
| | HIHKTAVLLECSVVSGGILGGATEDEIARIRRYARCVGLLFQVVDDIL | | | |
| | DVTKSSEELGKTAGKDLLTDKATYPKLMGLEKAKEFAAELATRAKE | | | |
| | ELSSFDQIKAAPLLGLADYIAFRQN | | | |
| CrtE_ | MDYANILTAIPLEFTPQDDIVLLEPYHYLGKNPGKEIRSQLIEAFNYWLDVKKEDLEVI | 57 | Xanthophyl | |
| Xd | QNVVGMLHTASLLMDDVEDSSVLRRGSPVAHLIYGIPQTINTANYVYFLAYQEIFKLR | | lomyces | |
| | PTPIPMPVIPPSSASLQSSVSSASSSSSASSENGGTSTPNSQIPFSKDTYLDKVITDEML | | dendrorhous | |
| | SLHRGQGLELFWRDSLTCPSEEEYVKMVLGKTGGLFRIAVRLMMAKSECDIDFVQL | | | |
| | VNLISTYFQIRDDYMNLQSSEYAHNKNFAEDLTEGKFSFPTIHSIHANPSSRLVINTLQK | | | |
| | KSTSPEILHHCVNYMRTETHSFEYTQEVLNTLSGALERELGRLQGEFAEANSKIDLGD | | | |
| | VESEGRTGKNVKLEAILKKLADIPL | | | |
| CrtE_ | MLTSSKSIESFPKNVQPYGKHYQNGLEPVGKSQEDILLEPFHYLCS | 58 | Blakeslea | |
| Bt | NPGKDVRTKMIEAFNAWLKVPKDDLIVITRVIEMLHSASLLIDDVED | | trispora | |
| | DSVLRRGVPAAHHIYGTPQTINCANYVYFLALKEIAKLNKPNMITIYT | | | |
| | DELINLHRGQGMELFWRDTLTCPTEKEFLDMVNDKTGGLLRLAVK | | | |
| | LMQEASQSGTDYTGLVSKIGIHFQVRDDYMNLQSKNYADNKGFCE | | | |
| | DLTEGKFSFPIIHSIRSDPSNRQLLNILKQRSSSIELKQFALQLLENT | | | |
| | NTFQYCRDFLRVLEKEAREEIKLLGGNIMLEKIMDVLSVNE | | | |

| | SEQ | | |
|---|---|---|---|
| Name/ | ID | | |
| Type | NO | DNA Sequence | Organism |
| gene | 43 | atgagttttgatattgccaaatacccgaccctggcactggtcgactccacccaggagttacga | Escherichia |
| | | ctgttgccgaaagagagtttaccgaaactctgcgacgaactgcgccgctatttactcgacag | coli |
| | | cgtgagccgttccagcgggcacttcgcctccgggctgggcacggtcgaactgaccgtggcg | |
| | | ctgcactatgtctacaacaccccgtttgaccaattgatttgggatgtggggcatcaggcttatc | |
| | | cgcataaaattttgaccggacgccgcgacaaaatcggcaccatccgtcagaaaggcggtct | |
| | | gcacccgttcccgtggcgcggcgaaagcgaatatgacgtattaagcgtcgggcattcatca | |
| | | acctccatcagtgccggaattggtattgcggttgctgccgaaaaagaaggcaaaaatcgcc | |
| | | gcaccgtctgtgtcattggcgatggcgcgattaccgcaggcatggcgtttgaagcgatgaat | |
| | | cacgcgggcgatatccgtcctgatatgctggtgattctcaacgacaatgaaatgtcgatttcc | |
| | | gaaaatgtcggcgcgctcaacaaccatctggcacagctgctttccggtaagctttactcttca | |
| | | ctgcgcgaaggcgggaaaaaagttttctctggcgtgccgccaattaaagagctgctcaaac | |
| | | gcaccgaagaacatattaaaggcatggtagtgcctggcacgttgtttgaagagctgggcttt | |
| | | aactacatcggcccggtggacggtcacgatgtgctggggcttatcaccacgctaaagaaca | |
| | | tgcgcgacctgaaaggcccgcagttcctgcatatcatgaccaaaaaaggtcgtggttatga | |
| | | accggcagaaaaagacccgatcactttccacgccgtgcctaaatttgatccctccagcggtt | |
| | | gtttgccgaaaagtagcggcggtttgccgagctattcaaaaatctttggcgactggttgtgcg | |
| | | aaacggcagcgaaagacaacaagctgatggcgattactccggcgatgcgtgaaggttccg | |
| | | gcatggtcgagttttcacgtaaattcccggatcgctacttcgacgtggcaattgccgagcaac | |
| | | acgcggtgacctttgctgcgggtctggcgattggtgggtacaaacccattgtcgcgatttact | |
| | | ccactttcctgcaacgcgcctatgatcaggtgctgcatgacgtggcgattcaaaagcttccgg | |
| | | tcctgttcgccatcgaccgcgcgggcattgttggtgctgacggtcaaacccatcagggtgctt | |
| | | ttgatctctcttacctgcgctgcataccggaaatggtcattatgaccccgagcgatgaaaacg | |
| | | aatgtcgccagatgctctataccggctatcactataacgatggcccgtcagcggtgcgctac | |
| | | ccgcgtggcaacgcggtcggcgtggaactgacgccgctggaaaaactaccaattggcaaa | |
| | | ggcattgtgaagcgtcgtggcgagaaactggcgatccttaactttggtacgctgatgccaga | |
| | | agcggcgaaagtcgccgaatcgctgaacgccacgctggtcgatatgcgttttgtgaaaccg | |
| | | cttgatgaagcgttaattctggaaatggccgccagccatgaagcgctggtcaccgtagaag | |
| | | aaaacgccattatgggcggcgcaggcagcggcgtgaacgaagtgctgatggcccatcgta | |
| | | aaccagtacccgtgctgaacattggcctgccggacttctttattccgcaaggaactcaggaa | |
| | | gaaatgcgcgccgaactcggcctcgatgccgctggtatggaagccaaaatcaaggcctgg | |
| | | ctggcataa | |
| gene | 44 | atgcaaacggaacacgtcattttattgaatgcacagggagttcccacgggtacgctggaaa | Escherichia |
| | | agtatgccgcacacacggcagacacccgcttacatctcgcgttctccagttggctgtttaatg | coli |
| | | ccaaaggacaattattagttacccgccgcgcactgagcaaaaaagcatggcctggcgtgtg | |
| | | gactaactcggtttgtgggcacccacaactgggagaaagcaacgaagacgcagtgatccg | |
| | | ccgttgccgttatgagcttggcgtggaaattacgcctcctgaatctatctatcctgactttcgct | |
| | | accgcgccaccgatccgagtggcattgtggaaaatgaagtgtgtccggtatttgccgcacg | |
| | | caccactagtgcgttacagatcaatgatgatgaagtgatggattatcaatggtgtgatttagc | |
| | | agatgtattacacggtattgatgccacgccgtgggcgttcagtccgtggatggtgatgcagg | |
| | | cgacaaatcgcgaagccagaaaacgattatctgcatttacccagcttaaataa | |
| Plasmid | 45 | accggtgtcattccgctgttatggccgcgtttgtctcattccacgcctgacactcagttccgggt | Escherichia |
| | | aggcagttcgctccaagctggactgtatgcacgaaccccccgttcagtccgaccgctgcgcc | coli |
| | | ttatccggtaactatcgtcttgagtccaacccggaaagacatgcaaaagcaccactggcagc | |
| | | agccactggtaattgatttagaggagttagtcttgaagtcatgcgccggttaaggctaaact | |
| | | gaaaggacaagttttggtgactgcgctcctccaagccagttacctcggttcaaagagttggt | |
| | | agctcagagaaccttcgaaaaaccgccctgcaaggcggttttttcgttttcagagcaagaga | |
| | | ttacgcgcagaccaaaacgatctcaagaagatcatcttattaatcagataaaatatttctaga | |
| | | tttcagtgcaatttatctcttcaaatgtagcacctgaagtcagccccatacgatataagttgta | |
| | | attctcatgttagtcatgccccgcgcccaccggaaggagctgactgggttgaaggctctcaa | |
| | | gggcatcggtcgagatcccggtgcctaatgagtgagctaacttacattaattgcgttgcgctc | |
| | | actgcccgctttccagtcgggaaacctgtcgtgccagctgcattaatgaatcggccaacgcg | |
| | | cggggagaggcggtttgcgtattgggcgccagggtggtttttcttttcaccagtgagacggg | |
| | | caacagctgattgcccttcaccgcctggccctgagagagttgcagcaagcggtccacgctg | |
| | | gtttgccccagcaggcgaaaatcctgtttgatggtggttaacggcgggatataacatgagct | |
| | | gtcttcggtatcgtcgtatcccactaccgagatgtccgcaccaacgcgcagcccggactcgg | |
| | | taatggcgcgcattgcgcccagcgccatctgatcgttggcaaccagcatcgcagtgggaac | |
| | | gatgccctcattcagcatttgcatggtttgttgaaaaccggacatggcactccagtcgccttcc | |
| | | cgttccgctatcggctgaatttgattgcgagtgagatatttatgccagccagccagacgcag | |
| | | acgcgccgagacagaacttaatgggcccgctaacagcgcgatttgctggtgacccaatgcg | |
| | | accagatgctccacgcccagtcgcgtaccgtcttcatgggagaaaataatactgttgatggg | |
| | | tgtctggtcagagacatcaagaaataacgccggaacattagtgcaggcagcttccacagca | |
| | | atggcatcctggtcatccagcggatagttaatgatcagcccactgacgcgttgcgcgagaa | |
| | | gattgtgcaccgccgctttacaggcttcgacgccgcttcgttctaccatcgacaccaccacgc | |
| | | tggcacccagttgatcggcgcgagatttaatcgccgcgacaatttgcgacggcgcgtgcag | |
| | | ggccagactggaggtggcaacgccaatcagcaacgactgtttgcccgccagttgttgtgcc | |
| | | acgcggttgggaatgtaattcagctccgccatcgccgcttccactttttcccgcgttttcgcag | |
| | | aaacgtggctggcctggttcaccacgcgggaaacggtctgataagagacaccggcatactc | |
| | | tgcgacatcgtataacgttactggtttcacattcaccaccctgaattgactctcttccgggcgct | |
| | | atcatgccataccgcgaaaggttttgcgccattcgatggtgtccgggatctcgacgctctccc | |
| | | ttatgcgactcctgcattaggaaattaatacgactcactataggggaattgtgagcggataa | |
| | | caattcccctgtagaaataattttgtttaactttaataaggagatataccatgggcagcagcc | |
| | | atcaccatcatcaccacagccaggatccgaattcgagctcaccaaacgccaaaggagcatc | |
| | | cagcatgagttttgatattgccaaatacccgaccctggcactggtcgactccacccaggagtt | |
| | | acgactgttgccgaaagagagtttaccgaaactctgcgacgaactgcgccgctatttactcg | |
| | | acagcgtgagccgttccagcgggcacttcgcctccgggctgggcacggtcgaactgaccgt | |
| | | ggcgctgcactatgtctacaacaccccgtttgaccaattgatttgggatgtggggcatcagg | |
| | | cttatccgcataaaattttgaccggacgccgcgacaaaatcggcaccatccgtcagaaagg | |
| | | cggtctgcacccgttcccgtggcgcggcgaaagcgaatatgacgtattaagcgtcgggcat | |
| | | tcatcaacctccatcagtgccggaattggtattgcggttgctgccgaaaaagaaggcaaaa | |
| | | atcgccgcaccgtctgtgtcattggcgatggcgcgattaccgcaggcatggcgtttgaagcg | |
| | | atgaatcacgcgggcgatatccgtcctgatatgctggtgattctcaacgacaatgaaatgtc | |
| | | gatttccgaaaatgtcggcgcgctcaacaaccatctggcacagctgctttccggtaagcttta | |
| | | ctcttcactgcgcgaaggcgggaaaaaagttttctctggcgtgccgccaattaaagagctgc | |
| | | tcaaacgcaccgaagaacatattaaaggcatggtagtgcctggcacgttgtttgaagagct | |
| | | gggctttaactacatcggcccggtggacggtcacgatgtgctggggcttatcaccacgctaa | |
| | | agaacatgcgcgacctgaaaggcccgcagttcctgcatatcatgaccaaaaaaggtcgtg | |
| | | gttatgaaccggcagaaaaagacccgatcactttccacgccgtgcctaaatttgatccctcca | |
| | | gcggttgtttgccgaaaagtagcggcggtttgccgagctattcaaaaatctttggcgactggt | |
| | | tgtgcgaaacggcagcgaaagacaacaagctgatggcgattactccggcgatgcgtgaag | |
| | | gttccggcatggtcgagttttcacgtaaattcccggatcgctacttcgacgtggcaattgccg | |
| | | agcaacacgcggtgacctttgctgcgggtctggcgattggtgggtacaaacccattgtcgcg | |
| | | atttactccactttcctgcaacgcgcctatgatcaggtgctgcatgacgtggcgattcaaaag | |
| | | cttccggtcctgttcgccatcgaccgcgcgggcattgttggtgctgacggtcaaacccatcag | |
| | | ggtgcttttgatctctcttacctgcgctgcataccggaaatggtcattatgaccccgagcgatg | |
| | | aaaacgaatgtcgccagatgctctataccggctatcactataacgatggcccgtcagcggtg | |
| | | cgctacccgcgtggcaacgcggtcggcgtggaactgacgccgctggaaaaactaccaatt | |
| | | ggcaaaggcattgtgaagcgtcgtggcgagaaactggcgatccttaactttggtacgctga | |
| | | tgccagaagcggcgaaagtcgccgaatcgctgaacgccacgctggtcgatatgcgttttgt | |
| | | gaaaccgcttgatgaagcgttaattctggaaatggccgccagccatgaagcgctggtcacc | |
| | | gtagaagaaaacgccattatgggcggcgcaggcagcggcgtgaacgaagtgctgatggc | |
| | | ccatcgtaaaccagtacccgtgctgaacattggcctgccggacttctttattccgcaaggaac | |
| | | tcaggaagaaatgcgcgccgaactcggcctcgatgccgctggtatggaagccaaaatcaa | |
| | | ggcctggctggcataactgcaggtcgacaagcttgcggccgcataatgcttaagtcgaaca | |
| | | gaaagtaatcgtattgtacacggccgcataatcgaaattaatacgactcactataggggaat | |
| | | tgtgagcggataacaattccccatcttagtatattagttaagtataagaaggagatatacata | |
| | | tggcagatctcaattggatatcggccggccacgcgatcgctgacgtcggtaccgaaaaaat | |
| | | caaaaggagcatcgcctatgcaaacggaacacgtcattttattgaatgcacagggagttcc | |
| | | cacgggtacgctggaaaagtatgccgcacacacggcagacacccgcttacatctcgcgttc | |
| | | tccagttggctgtttaatgccaaaggacaattattagttacccgccgcgcactgagcaaaaa | |
| | | agcatggcctggcgtgtggactaactcggtttgtgggcacccacaactgggagaaagcaac | |
| | | gaagacgcagtgatccgccgttgccgttatgagcttggcgtggaaattacgcctcctgaatc | |
| | | tatctatcctgactttcgctaccgcgccaccgatccgagtggcattgtggaaaatgaagtgtg | |
| | | tccggtatttgccgcacgcaccactagtgcgttacagatcaatgatgatgaagtgatggatt | |
| | | atcaatggtgtgatttagcagatgtattacacggtattgatgccacgccgtgggcgttcagtc | |
| | | cgtggatggtgatgcaggcgacaaatcgcgaagccagaaaacgattatctgcatttaccca | |
| | | gcttaaataactcgagtctggtaaagaaaccgctgctgcgaaatttgaacgccagcacatg | |
| | | gactcgtctactagcgcagcttaattaacctaggctgctgccaccgctgagcaataactagc | |
| | | ataaccccttggggcctctaaacgggtcttgaggggttttttgctgaaacctcaggcatttga | |
| | | gaagcacacggtcacactgcttccggtagtcaataaaccggtaaaccagcaatagacataa | |
| | | gcggctatttaacgaccctgccctgaaccgacgaccgggtcgaatttgctttcgaatttctgc | |
| | | cattcatccgcttattatcacttattcaggcgtagcaccaggcgtttaagggcaccaataact | |
| | | gccttaaaaaaattacgccccgccctgccactcatcgcagtactgttgtaattcattaagcatt | |
| | | ctgccgacatggaagccatcacagacggcatgatgaacctgaatcgccagcggcatcagc | |
| | | accttgtcgccttgcgtataatatttgcccatagtgaaaacgggggcgaagaagttgtccat | |
| | | attggccacgtttaaatcaaaactggtgaaactcacccagggattggctgagacgaaaaac | |
| | | atattctcaataaaccctttagggaaataggccaggttttcaccgtaacacgccacatcttgc | |
| | | gaatatatgtgtagaaactgccggaaatcgtcgtggtattcactccagagcgatgaaaacgt | |
| | | ttcagtttgctcatggaaaacggtgtaacaagggtgaacactatcccatatcaccagctcac | |
| | | cgtctttcattgccatacggaactccggatgagcattcatcagggggcaagaatgtgaata | |
| | | aaggccggataaaacttgtgcttatttttctttacggtctttaaaaaggccgtaatatccagct | |
| | | gaacggtctggttataggtacattgagcaactgactgaaatgcctcaaaatgttctttacgat | |
| | | gccattgggatatatcaacggtggtatatccagtgatttttttctccattttagcttccttagctc | |
| | | ctgaaaatctcgataactcaaaaaatacgcccggtagtgatcttatttcattatggtgaaagt | |
| | | tggaacctcttacgtgccgatcaacgtctcattttcgccaaaagttggcccagggcttcccgg | |
| | | tatcaacagggacaccaggatttatttattctgcgaagtgatcttccgtcacaggtatttattc | |
| | | ggcgcaaagtgcgtcgggtgatgctgccaacttactgatttagtgtatgatggtgtttttgag | |
| | | gtgctccagtggcttctgtttctatcagctgtccctcctgttcagctactgacggggtggtgcgt | |
| | | aacggcaaaagcaccgccggacatcagcgctagcggagtgtatactggcttactatgttgg | |
| | | cactgatgagggtgtcagtgaagtgcttcatgtggcaggagaaaaaaggctgcaccggtg | |
| | | cgtcagcagaatatgtgatacaggatatattccgcttcctcgctcactgactcgctacgctcg | |
| | | gtcgttcgactgcggcgagcggaaatggcttacgaacggggcggagatttcctggaagat | |
| | | gccaggaagatacttaacagggaagtgagagggccgcggcaaagccgtttttccataggc | |
| | | tccgcccccctgacaagcatcacgaaatctgacgctcaaatcagtggtggcgaaacccgac | |
| | | aggactataaagataccaggcgtttcccctggcggctccctcgtgcgctctcctgttcctgcct | |
| | | ttcggttt | |
| plasmid | 46 | gatccccaccgtttccggcgggtcgctccctcttgcgctctcctgttccgaccctgccgtttacc | Escherichia |
| | | ggatacctgttccgcctttctcccttacgggaagtgtggcgctttctcatagctcacacactgg | coli |
| | | tatctcggctcggtgtaggtcgttcgctccaagctgggctgtaagcaagaactccccgttcag | |
| | | cccgactgctgcgccttatccggtaactgttcacttgagtccaacccggaaaagcacggtaa | |
| | | aacgccactggcagcagccattggtaactgggagttcgcagaggatttgtttagctaaacac | |
| | | gcggttgctcttgaagtgtgcgccaaagtccggctacactggaaggacagatttggttgctg | |
| | | tgctctgcgaaagccagttaccacggttaagcagttccccaactgacttaaccttcgatcaaa | |
| | | ccacctccccaggtggttttttcgtttacagggcaaaagattacgcgcagaaaaaaaggatc | |
| | | tcaagaagatcctttgatcttttctactgaaccgctctagatttcagtgcaatttatctcttcaaa | |
| | | tgtagcacctgaagtcagccccatacgatataagttgtaattctcatgttagtcatgccccgc | |
| | | gcccaccggaaggagctgactgggttgaaggctctcaagggcatcggtcgagatcccggt | |
| | | gcctaatgagtgagctaacttacattaattgcgttgcgctcactgcccgctttccagtcggga | |
| | | aacctgtcgtgccagctgcattaatgaatcggccaacgcgcggggagaggcggtttgcgta | |
| | | ttgggcgccagggtggtttttcttttcaccagtgagacgggcaacagctgattgcccttcacc | |
| | | gcctggccctgagagagttgcagcaagcggtccacgctggtttgccccagcaggcgaaaat | |
| | | cctgtttgatggtggttaacggcgggatataacatgagctgtcttcggtatcgtcgtatcccac | |
| | | taccgagatgtccgcaccaacgcgcagcccggactcggtaatggcgcgcattgcgcccagc | |
| | | gccatctgatcgttggcaaccagcatcgcagtgggaacgatgccctcattcagcatttgcat | |
| | | ggtttgttgaaaaccggacatggcactccagtcgccttcccgttccgctatcggctgaatttg | |
| | | attgcgagtgagatatttatgccagccagccagacgcagacgcgccgagacagaacttaat | |
| | | gggcccgctaacagcgcgatttgctggtgacccaatgcgaccagatgctccacgcccagtc | |
| | | gcgtaccgtcttcatgggagaaaataatactgttgatgggtgtctggtcagagacatcaaga | |
| | | aataacgccggaacattagtgcaggcagcttccacagcaatggcatcctggtcatccagcg | |
| | | gatagttaatgatcagcccactgacgcgttgcgcgagaagattgtgcaccgccgctttacag | |
| | | gcttcgacgccgcttcgttctaccatcgacaccaccacgctggcacccagttgatcggcgcg | |
| | | agatttaatcgccgcgacaatttgcgacggcgcgtgcagggccagactggaggtggcaac | |
| | | gccaatcagcaacgactgtttgcccgccagttgttgtgccacgcggttgggaatgtaattca | |
| | | gctccgccatcgccgcttccactttttcccgcgttttcgcagaaacgtggctggcctggttcac | |
| | | cacgcgggaaacggtctgataagagacaccggcatactctgcgacatcgtataacgttact | |
| | | ggtttcacattcaccaccctgaattgactctcttccgggcgctatcatgccataccgcgaaag | |
| | | gttttgcgccattcgatggtgtccgggatctcgacgctctcccttatgcgactcctgcattagg | |
| | | aaattaatacgactcactataggggaattgtgagcggataacaattcccctgtagaaataat | |
| | | tttgtttaactttaataaggagatataccatgggcagcagccatcaccatcatcaccacagcc | |
| | | agGATCCGATGGAAGTGGGTAGCAAGAGCTTCGCGACCGCGAGCAAG | |
| | | CTGTTTGATGCGAAAACCCGTCGTAGCGTTCTGATGCTGTACGCGTGG | |
| | | TGCCGTCACTGCGACGATGTGATCGACGATCAAGTTCTGGGTTTCAGC | |
| | | AACGATACCCCGAGCCTGCAGAGCGCGGAGCAACGTCTGGCGCAGCT | |
| | | GGAAATGAAAACCCGTCAAGCGTATGCGGGCAGCCAGATGCATGAGC | |
| | | CGGCGTTCGCGGCGTTTCAAGAAGTGGCGATGGCGCATGACATCCTG | |
| | | CCGGCGTACGCGTTTGATCACCTGGCGGGCTTTGCGATGGACGTTCA | |
| | | CGAGACCCGTTATCAGACCCTGGACGATACCCTGCGTTACTGCTATCA | |
| | | CGTGGCGGGTGTGGTTGGCCTGATGATGGCGCAAATTATGGGTGTTC | |
| | | GTGATAACGCGACCCTGGACCGTGCGTGCGATCTGGGCCTGGCGTTT | |
| | | CAGCTGACCAACATCGCGCGTGACATTGTGGAGGATGCGGAAGCGGG | |
| | | TCGTTGCTACCTGCCGGCGGCGTGGCTGGCGGAGGAAGGCCTGACCC | |
| | | GTGAAAACCTGGCGGACCCGCAGAACCGTAAGGCGCTGAGCCGTGTG | |
| | | GCGCGTCGTCTGGTTGAGACCGCGGAACCGTACTATCGTAGCGCGAG | |
| | | CGCGGGTCTGCCGGGTCTGCCGCTGCGTAGCGCGTGGGCGATCGCG | |
| | | ACCGCGCAGCAAGTTTATCGTAAGATTGGTATGAAAGTGGTGCAGGC | |
| | | GGGCAGCCAGGCGTGGGAGCAGCGTCAAAGCACCAGCACCCCGGAA | |
| | | AAACTGGCGCTGCTGGTTGCGGCGAGCGGTCAAGCGGTTACCAGCCG | |
| | | TGTTGCGCGTCATGCGCCGCGTAGCGCGGATCTGTGGCAGCGTCCGG | |
| | | TTTAAGaattcgagctcggcgcgcctgcaggtcgacaagcttgcggccgcataatgctta | |
| | | agtcgaacagaaagtaatcgtattgtacacggccgcataatcgaaattaatacgactcacta | |
| | | taggggaattgtgagcggataacaattccccatcttagtatattagttaagtataagaagga | |
| | | gatatacaTATGAGCAGCGTGAACCTGGGCAGCTGGGTGCACACCAGCT | |
| | | ACGTTCTGAACCAGGCGACCCGTAGCCGTAGCAAGAGCAAGAGCTTC | |
| | | AGCCTGCCGTTTAACCCGCTGAAGAGCCTGGCGATTAGCTTCGCGTAT | |
| | | CGTAAAAGCGAGCGTCCGATCAGCAGCGTTAGCGCGATCATTACCAA | |
| | | GGAAGAGGAGACCCTGCAGGAAGAGCAAAACAACCCGCCGCCGAGCT | |
| | | TCGACTTTAAGAGCTACATGCTGCAGAAGGGTAACAGCATCAACCAAG | |
| | | CGCTGGAAGCGGCGATTCCGCTGCAAGAACCGGCGAAGATCCACGAG | |
| | | AGCATGCGTTATAGCCTGCTGGCGGGTGGCAAACGTGTGCGTCCGGC | |
| | | GCTGTGCCTGGCGGCGTGCGAACTGGTTGGTGGCAACGACAGCATGG | |
| | | CGATGCCGGCGGCGTGCGCGGTGGAGATGATCCACACCATGAGCCTG | |
| | | ATTCACGACGATCTGCCGTGCATGGATAACGACGATCTGCGTCGTGGC | |
| | | AAACCGACCAACCACATTGTTTTTGGCGAAGACGTTGCGGTTCTGGCG | |
| | | GGTGATGCGCTGCTGGCGTTTGCGTTTGAGCACATCGCTGTGAGCAC | |
| | | CCTGAACGTTAGCAGCGCGCGTATTGTGCGTGCGGTTGGTGAACTGG | |
| | | CGAAAGCGATTGGTGCGGAGGGTCTGGTGGCGGGCCAGGTGGTTGA | |
| | | CATTAACAGCGAAGGTAGCAGCGAGGTTGATCTGGAAAAACTGGAGTT | |
| | | CATCCACATTCACAAGACCGCGAAACTGCTGGAAGGCGCGGTGGTTCT | |
| | | GGGTGCGATCCTGGGTGGTGGCACCGACGAAGAGGTGGAGAAGCTG | |
| | | CGTAAATACGCGCGTGATATTGGTCTGCTGTTTCAGGTGGTTGACGAC | |
| | | ATCCTGGACGTTACCAAGAGCAGCCAAGAACTGGGCAAGACCGCGGG | |
| | | TAAAGACCTGGTGGCGGATAAGGTTACCTATCCGAAACTGCTGGGCAT | |
| | | CGAGAAGAGCCGTGAGTTCGCGGAGAAGCTGAACAAAGAAGCGCAG | |
| | | GAGCAACTGGCGGGTTTTGATCCGGAAAAAGCGGCGCCGCTGATTGC | |
| | | GCTGGCGAACTACATTGCGCACCGTCAAAACTAAAgatctcaattggatatc | |
| | | ggccggccacgcgatcgctgacgtcggtaccctcgagtctggtaaagaaaccgctgctgcg | |
| | | aaatttgaacgccagcacatggactcgtctactagcgcagcttaattaacctaggctgctgcc | |
| | | accgctgagcaataactagcataaccccttggggcctctaaacgggtcttgaggggttttttg | |
| | | ctgaaacctcaggcatttgagaagcacacggtcacactgcttccggtagtcaataaaccggt | |
| | | aaaccagcaatagacataagcggctatttaacgaccctgccctgaaccgacgaccgggtca | |
| | | tcgtggccggatcttgcggcccctcggcttgaacgaattgttagacattatttgccgactacct | |
| | | tggtgatctcgcctttcacgtagtggacaaattcttccaactgatctgcgcgcgaggccaagc | |
| | | gatcttcttcttgtccaagataagcctgtctagcttcaagtatgacgggctgatactgggccg | |
| | | gcaggcgctccattgcccagtcggcagcgacatccttcggcgcgattttgccggttactgcgc | |
| | | tgtaccaaatgcgggacaacgtaagcactacatttcgctcatcgccagcccagtcgggcgg | |
| | | cgagttccatagcgttaaggtttcatttagcgcctcaaatagatcctgttcaggaaccggatc | |
| | | aaagagttcctccgccgctggacctaccaaggcaacgctatgttctcttgcttttgtcagcaa | |
| | | gatagccagatcaatgtcgatcgtggctggctcgaagatacctgcaagaatgtcattgcgct | |
| | | gccattctccaaattgcagttcgcgcttagctggataacgccacggaatgatgtcgtcgtgca | |
| | | caacaatggtgacttctacagcgcggagaatctcgctctctccaggggaagccgaagtttcc | |
| | | aaaaggtcgttgatcaaagctcgccgcgttgtttcatcaagccttacggtcaccgtaaccagc | |
| | | aaatcaatatcactgtgtggcttcaggccgccatccactgcggagccgtacaaatgtacggc | |
| | | cagcaacgtcggttcgagatggcgctcgatgacgccaactacctctgatagttgagtcgata | |
| | | cttcggcgatcaccgcttccctcatactcttcctttttcaatattattgaagcatttatcagggtt | |
| | | attgtctcatgagcggatacatatttgaatgtatttagaaaaataaacaaatagctagctcac | |
| | | tcggtcgctacgctccgggcgtgagactgcggcgggcgctgcggacacatacaaagttacc | |
| | | cacagattccgtggataagcaggggactaacatgtgaggcaaaacagcagggccgcgcc | |
| | | ggtggcgtttttccataggctccgccctcctgccagagttcacataaacagacgcttttccggt | |
| | | gcatctgtgggagccgtgaggctcaaccatgaatctgacagtacgggcgaaacccgacag | |
| | | gacttaaa | |
| plasmid | 47 | atttcagtgcaatttatctcttcaaatgtagcacctgaagtcagccccatacgatataagttgt | Escherichia |
| | | aattctcatgttagtcatgccccgcgcccaccggaaggagctgactgggttgaaggctctca | coli |
| | | agggcatcggtcgagatcccggtgcctaatgagtgagctaacttacattaattgcgttgcgct | |
| | | cactgcccgctttccagtcgggaaacctgtcgtgccagctgcattaatgaatcggccaacgc | |
| | | gcggggagaggcggtttgcgtattgggcgccagggtggtttttcttttcaccagtgagacgg | |
| | | gcaacagctgattgcccttcaccgcctggccctgagagagttgcagcaagcggtccacgct | |
| | | ggtttgccccagcaggcgaaaatcctgtttgatggtggttaacggcgggatataacatgag | |
| | | ctgtcttcggtatcgtcgtatcccactaccgagatgtccgcaccaacgcgcagcccggactcg | |
| | | gtaatggcgcgcattgcgcccagcgccatctgatcgttggcaaccagcatcgcagtgggaa | |
| | | cgatgccctcattcagcatttgcatggtttgttgaaaaccggacatggcactccagtcgccttc | |
| | | ccgttccgctatcggctgaatttgattgcgagtgagatatttatgccagccagccagacgca | |
| | | gacgcgccgagacagaacttaatgggcccgctaacagcgcgatttgctggtgacccaatgc | |
| | | gaccagatgctccacgcccagtcgcgtaccgtcttcatgggagaaaataatactgttgatgg | |
| | | gtgtctggtcagagacatcaagaaataacgccggaacattagtgcaggcagcttccacagc | |
| | | aatggcatcctggtcatccagcggatagttaatgatcagcccactgacgcgttgcgcgaga | |
| | | agattgtgcaccgccgctttacaggcttcgacgccgcttcgttctaccatcgacaccaccacg | |
| | | ctggcacccagttgatcggcgcgagatttaatcgccgcgacaatttgcgacggcgcgtgca | |
| | | gggccagactggaggtggcaacgccaatcagcaacgactgtttgcccgccagttgttgtgc | |
| | | cacgcggttgggaatgtaattcagctccgccatcgccgcttccactttttcccgcgttttcgca | |
| | | gaaacgtggctggcctggttcaccacgcgggaaacggtctgataagagacaccggcatac | |
| | | tctgcgacatcgtataacgttactggtttcacattcaccaccctgaattgactctcttccgggcg | |
| | | ctatcatgccataccgcgaaaggttttgcgccattcgatggtgtccgggatctcgacgctctc | |
| | | ccttatgcgactcctgcattaggaaatttgacaattaatcatcggctcgtataatgtgtggaat | |
| | | tgtgagcgctcacaattcccctgtagaaataattttgtttaactttaataaggagatataccat | |
| | | ggcaatgacccgtgcggagcgtaagcgtcagcacatcaaccacgcgctgagcattggtca | |
| | | aaaacgtgagaccggcctggacgacatcaccttcgtgcacgttagcctgccggacctggcg | |
| | | ctggaacaggtggacatcagcaccaagattggcgagctgagcagcagcagcccgatcttt | |
| | | attaacgcgatgaccggtggcggtggcaagctgacctacgaaatcaacaagagcctggcg | |
| | | cgtgcggcgagccaggcgggtattccgctggcggtgggtagccaaatgagcgcgctgaag | |
| | | gacccgagcgagcgtctgagctatgaaatcgttcgtaaagagaacccgaacggtctgatttt | |
| | | cgcgaacctgggtagcgaggcgaccgcggcgcaggcgaaagaggcggttgaaatgatc | |
| | | ggtgcgaacgcgctgcaaattcacctgaacgtgatccaagaaattgttatgccggagggtg | |
| | | accgtagcttcagcggcgcgctgaagcgtatcgaacaaatttgcagccgtgtgagcgttcc | |
| | | ggtgatcgttaaagaggtgggtttcggcatgagcaaggcgagcgcgggtaaactgtatga | |
| | | agcgggtgcggcggcggtggacatcggtggttacggtggcaccaacttcagcaagattga | |
| | | gaacctgcgtcgtcagcgtcaaatcagcttctttaacagctggggtattagcaccgcggcga | |
| | | gcctggcggagatccgtagcgaatttccggcgagcaccatgattgcgagcggtggcctgca | |
| | | agacgcgctggatgtggcgaaagcgattgcgctgggtgcgagctgcaccggtatggcggg | |
| | | ccactttctgaaagcgctgaccgacagcggcgaggaaggcctgctggaggaaatccagct | |
| | | gattctggaggaactgaaactgatcatgaccgtgctgggtgcgcgtaccattgcggatctgc | |
| | | aaaaggcgccgctggttattaaaggtgaaacccaccactggctgaccgagcgtggcgtga | |
| | | acaccagcagctacagcgttcgttaaggatccgaattcgaagtcagattaaaggaggatcg | |
| | | cgcatgagcctgccgttcctgaccagcgcgccgggcaaagttatcatttttggcgagcacag | |
| | | cgcggtgtacaacaaaccggcggttgcggcgagcgtgagcgcgctgcgtacctatctgctg | |
| | | atcagcgagagcagcgcgccggacaccatcgaactggacttcccggatattagctttaacc | |
| | | acaaatggagcatcaacgactttaacgcgattaccgaagatcaggttaacagccaaaaact | |
| | | ggcgaaggcgcagcaagcgaccgacggtctgagccaagagctggtgagcctgctggacc | |
| | | cgctgctggcgcaactgagcgagagcttccactaccacgcggcgttctgctttctgtatatgtt | |
| | | tgtttgcctgtgcccgcacgcgaaaaacatcaagttcagcctgaaaagcaccctgccgattg | |
| | | gtgcgggtctgggtagcagcgcgagcatcagcgtgagcctggcgctggcgatggcgtacc | |
| | | tgggtggcctgattggcagcaacgacctggagaaactgagcgaaaacgataagcacatcg | |
| | | ttaaccagtgggcgtttatcggcgagaagtgcattcacggcaccccgagcggtattgacaa | |
| | | cgcggtggcgacctacggcaacgcgctgctgtttgaaaaagatagccacaacggcaccat | |
| | | caacaccaacaacttcaagtttctggacgatttcccggcgatcccgatgattctgacctatac | |
| | | ccgtattccgcgtagcaccaaagacctggttgcgcgtgtgcgtgttctggtgaccgagaaat | |
| | | tcccggaagtgatgaagccgattctggatgcgatgggcgagtgcgcgctgcagggtctgg | |
| | | aaatcatgaccaagctgagcaaatgcaagggcaccgacgatgaggcggttgaaaccaac | |
| | | aacgagctgtacgaacaactgctggagctgatccgtattaaccacggtctgctggttagcat | |
| | | cggcgtgagccacccgggtctggaactgatcaaaaacctgagcgacgatctgcgtattggc | |
| | | agcaccaagctgaccggtgcgggtggcggtggctgcagcctgaccctgctgcgtcgtgaca | |
| | | tcacccaggagcaaattgatagcttcaagaaaaagctgcaggacgatttcagctatgagac | |
| | | ctttgaaaccgacctgggtggcaccggttgctgcctgctgagcgcgaaaaacctgaacaag | |
| | | gatctgaaaattaagagcctggtgttccaactgtttgaaaacaaaaccaccaccaagcagc | |
| | | aaatcgacgatctgctgctgccgggtaacaccaacctgccgtggaccagctaacaatgacg | |
| | | ttaaaggagaatctgtcatgagcgagctgcgtgcgtttagcgcgccgggtaaagcgctgct | |
| | | ggcgggtggctacctggttctggacaccaagtatgaagcgtttgtggttggtctgagcgcgc | |
| | | gtatgcatgcggttgcgcacccgtacggtagcctgcagggcagcgacaagttcgaagtgcg | |
| | | tgttaagagcaagcaattcaaggatggtgaatggctgtatcacatcagcccgaagagcggc | |
| | | ttcatcccggttagcattggtggcagcaagaacccgtttatcgagaaagttattgcgaacgt | |
| | | gttcagctactttaaaccgaacatggacgattattgcaaccgtaacctgttcgttatcgacatt | |
| | | tttagcgacgatgcgtaccacagccaggaagacagcgtgaccgaacaccgtggtaaccgt | |
| | | cgtctgagcttccacagccaccgtatcgaggaagttccgaaaaccggtctgggcagcagcg | |
| | | cgggtctggtgaccgttctgaccaccgcgctggcgagcttctttgtgagcgacctggagaac | |
| | | aacgttgataaataccgtgaagtgattcacaacctggcgcaagttgcgcactgccaggcgc | |
| | | aaggcaagattggtagcggttttgatgtggcggcggcggcgtacggtagcattcgttatcgt | |
| | | cgttttccgccggcgctgatcagcaacctgccggatattggtagcgcgacctatggcagcaa | |
| | | actggcgcacctggtggacgaggaagattggaacatcaccattaagagcaaccacctgcc | |
| | | gagcggtctgaccctgtggatgggtgacatcaagaacggcagcgagaccgtgaaactggt | |
| | | tcagaaggtgaaaaactggtacgacagccacatgccggagagcctgaaaatctacaccga | |
| | | actggatcacgcgaacagccgtttcatggacggcctgagcaagctggatcgtctgcacgaa | |
| | | acccacgacgattacagcgaccagattttcgagagcctggaacgtaacgattgcacctgcc | |
| | | aaaaatatccggagattaccgaagttcgtgacgcggtggcgaccatccgtcgtagcttccgt | |
| | | aagattaccaaagagagcggtgcggacatcgaaccgccggttcagaccagcctgctggac | |
| | | gattgccaaaccctgaaaggtgtgctgacctgcctgattccgggtgcgggtggctacgatgc | |
| | | gattgcggttattaccaagcaggacgtggatctgcgtgcgcaaaccgcgaacgataagcgt | |
| | | tttagcaaagttcagtggctggacgtgacccaagcggattggggtgttcgtaaggagaaag | |
| | | acccggaaacctatctggataagtaacctatcgcttaaaggagcatcacgaatgaccgttta | |
| | | caccgcgagcgtgaccgcgccggttaacatcgcgaccctgaagtattggggtaaacgtgat | |
| | | accaagctgaacctgccgaccaacagcagcattagcgtgaccctgagccaggacgatctg | |
| | | cgtaccctgaccagcgcggcgaccgcgccggagttcgaacgtgataccctgtggctgaacg | |
| | | gcgagccgcacagcatcgacaacgaacgtacccagaactgcctgcgtgatctgcgtcaact | |
| | | gcgtaaagagatggaaagcaaagatgcgagcctgccgaccctgagccaatggaaactgc | |
| | | acatcgttagcgagaacaacttcccgaccgcggcgggtctggcgagcagcgcgggggct | |
| | | ttgcggcgctggttagcgcgattgcgaaactgtaccagctgccgcaaagcaccagcgaaat | |
| | | cagccgtattgcgcgtaagggtagcggcagcgcgtgccgtagcctgtttggtggctatgttg | |
| | | cgtgggagatgggtaaagcggaagacggccacgatagcatggcggtgcagatcgcggac | |
| | | agcagcgattggccgcaaatgaaggcgtgcgttctggtggtgtctgacattaagaaagatg | |
| | | ttagcagcacccagggtatgcaactgaccgtggcgaccagcgagctgttcaaagagcgtat | |
| | | cgaacacgtggttccgaagcgttttgaggtgatgcgtaaagcgattgttgaaaaggatttcg | |
| | | cgacctttgcgaaagaaaccatgatggacagcaacagcttccacgcgacctgcctggatag | |
| | | cttcccgccgatcttttacatgaacgacaccagcaagcgtatcattagctggtgccacaccat | |
| | | caaccagttctacggcgagaccattgttgcgtatacctttgatgcgggcccgaacgcggtgc | |
| | | tgtactatctggcggagaacgaaagcaaactgttcgcgtttatttataagctgttcggtagcg | |
| | | ttccgggctgggacaagaaatttaccaccgagcagctggaagcgttcaaccaccaatttga | |
| | | gagcagcaactttaccgcgcgtgagctggacctggaactgcagaaggatgtggcgcgtgtt | |
| | | atcctgacccaagtgggtagcggcccgcaagagaccaacgaaagcctgattgacgcgaaa | |
| | | accggcctgccgaaggaataaaagcttgcggccgcataatgcttaagtcgaacagaaagt | |
| | | aatcgtattgtacacggccgcataatcgaaatttgacaattaatcatcggctcgtataatgtgt | |
| | | ggaattgtgagcgctcacaattccccatcttagtatattagttaagtataagaaggagatata | |
| | | catatggcagatctacaacacaattaaaggagcatcttgtatgaagaccgtggttatcattg | |
| | | atgcgctgcgtaccccgatcggcaagtacaaaggtagcctgagccaagtgagcgcggttg | |
| | | acctgggcacccacgttaccacccagctgctgaaacgtcacagcaccattagcgaggaaat | |
| | | cgatcaagtgattttcggcaacgttctgcaaggggtaacggtcaaaacccggcgcgtcag | |
| | | atcgcgattaacagcggtctgagccacgagatcccggcgatgaccgtgaacgaagtttgcg | |
| | | gcagcggtatgaaggcggttatcctggcgaaacagctgattcaactgggtgaagcggaag | |
| | | tgctgattgcgggtggcattgagaacatgagccaagcgccgaagctgcagcgtttcaacta | |
| | | cgagaccgaaagctatgatgcgccgtttagcagcatgatgtacgacggtctgaccgatgcg | |
| | | ttcagcggccaagcgatgggtctgaccgcggagaacgtggcggaaaaatatcacgttacc | |
| | | cgtgaggaacaggaccaattcagcgttcacagccaactgaaggcggcgcaggcgcaagc | |
| | | ggaaggcatctttgcggatgagattgcgccgctggaagtgagcggcaccctggttgagaa | |
| | | agacgaaggtatccgtccgaacagcagcgtggagaagctgggcaccctgaagaccgtgtt | |
| | | taaagaagatggcaccgttaccgcgggtaacgcgagcaccattaacgacggcgcgagcg | |
| | | cgctgatcattgcgagccaggagtacgcggaagcgcacggtctgccgtatctggcgatcat | |
| | | tcgtgacagcgttgaggtgggtatcgatccggcgtacatgggtatcagcccgattaaggcg | |
| | | atccaaaaactgctggcgcgtaaccagctgaccaccgaggaaatcgacctgtatgagatta | |
| | | acgaagcgttcgcggcgaccagcatcgtggttcagcgtgaactggcgctgccggaggaaa | |
| | | aggttaacatctacggtggcggtattagcctgggtcatgcgattggtgcgaccggtgcgcgt | |
| | | ctgctgaccagcctgagctaccagctgaaccaaaaagagaagaaatatggtgtggcgagc | |
| | | ctgtgcatcggcggtggcctgggtctggcgatgctgctggaacgtccgcagcaaaagaaa | |
| | | aacagccgtttctatcaaatgagcccggaggaacgtctggcgagcctgctgaacgagggtc | |
| | | agatcagcgcggataccaagaaagagtttgaaaacaccgcgctgagcagccaaattgcg | |
| | | aaccacatgatcgagaaccagattagcgagaccgaagtgccgatgggcgttggtctgcac | |
| | | ctgaccgtggacgaaaccgattacctggttccgatggcgaccgaggaaccgagcgttatcg | |
| | | cggcgctgagcaacggcgcgaagattgcgcaaggtttcaaaaccgtgaaccagcaacgtc | |
| | | tgatgcgtggtcagatcgtgttttatgacgttgcggatgcggagagcctgatcgacgaactg | |
| | | caagttcgtgagaccgaaattttccagcaagcggagctgagctacccgagcattgtgaaac | |
| | | gtggtggcggtctgcgtgacctgcagtatcgtgcgttcgatgagagcttcgtgagcgttgac | |
| | | tttctggtggacgttaaggatgcgatgggcgcgaacatcgtgaacgcgatgctggagggtg | |
| | | ttgcggaactgttccgtgagtggtttgcggaacagaaaatcctgttcagcattctgagcaact | |
| | | acgcgaccgagagcgtggttaccatgaagaccgcgattccggtgagccgtctgagcaaag | |
| | | gcagcaacggtcgtgagatcgcggaaaagattgttctggcgagccgttatgcgagcctgga | |
| | | cccgtatcgtgcggtgacccacaacaagggcatcatgaacggtattgaggcggtggttctg | |
| | | gcgaccggtaacgatacccgtgcggttagcgcgagctgccatgcgtttgcggttaaagaag | |
| | | gccgttatcaaggtctgaccagctggaccctggacggcgagcagctgattggtgaaatcag | |
| | | cgttccgctggcgctggcgaccgtgggcggtgcgaccaaggttctgccgaaaagccaggc | |
| | | ggcggcggacctgctggcggtgaccgatgcgaaggagctgagccgtgtggttgcggcggt | |
| | | tggtctggcgcagaacctggcggcgctgcgtgcgctggttagcgaaggcatccaaaaagg | |
| | | tcacatggcgctgcaagcgcgtagcctggcgatgaccgtgggtgcgaccggtaaagaggt | |
| | | ggaagcggttgcgcagcaactgaagcgtcaaaaaaccatgaaccaggatcgtgcgctggc | |
| | | gatcctgaacgacctgcgtaaacagtaaacatgtcatgaaaggagcatcttccatgaccatc | |
| | | ggtattgataagatcagcttctttgtgccgccgtactatattgatatgaccgcgctggcggaa | |
| | | gcgcgtaacgttgacccgggcaaattccacatcggtattggccaggatcaaatggcggtga | |
| | | acccgatcagccaggacattgttaccttcgcggcgaacgcggcggaggcgatcctgaccaa | |
| | | ggaagataaagaggcgatcgacatggtgattgttggcaccgaaagcagcattgacgaga | |
| | | gcaaggctgcggcggtggttctgcaccgtctgatgggtatccagccgttcgcgcgtagcttt | |
| | | gaaattaaagagggttgctatggtgcgaccgcgggtctgcaactggcgaaaaaccacgtg | |
| | | gcgctgcacccggataagaaagttctggtggttgcggcggacatcgcgaagtatggtctga | |
| | | acagcggtggcgaaccgacccagggtgcgggtgcggtggcgatgctggttgcgagcgaa | |
| | | ccgcgtatcctggcgctgaaagaggataacgtgatgctgacccaagatatttacgacttctg | |
| | | gcgtccgaccggtcacccgtatccgatggtggacggtccgctgagcaacgaaacctacatc | |
| | | cagagctttgcgcaagtttgggatgagcacaagaaacgtaccggcctggacttcgcggact | |
| | | acgatgcgctggcgtttcacatcccgtataccaagatgggtaagaaagcgctgctggcgaa | |
| | | aatcagcgatcagaccgaggcggaacaagagcgtattctggcgcgttacgaggaaagcat | |
| | | catttatagccgtcgtgttggtaacctgtacaccggcagcctgtatctgggtctgatcagcctg | |
| | | ctggaaaacgcgaccaccctgaccgcgggtaaccagattggcctgttcagctacggtagcg | |
| | | gtgcggtggcggagttcttcaccggcgagctggttgcgggttatcagaaccacctgcaaaa | |
| | | ggaaacccacctggcgctgctggataaccgtaccgagctgagcatcgcggaatacgaggc | |
| | | gatgtttgcggagaccctggacaccgatattgaccaaaccctggaagacgagctgaaatac | |
| | | agcatcagcgcgattaacaacaccgtgcgtagctatcgtaactaagacgtcggtaccacca | |
| | | aacgccaaaggagcatccagcatgagttttgatattgccaaatacccgaccctggcactggt | |
| | | cgactccacccaggagttacgactgttgccgaaagagagtttaccgaaactctgcgacgaa | |
| | | ctgcgccgctatttactcgacagcgtgagccgttccagcgggcacttcgcctccgggctggg | |
| | | cacggtcgaactgaccgtggcgctgcactatgtctacaacaccccgtttgaccaattgatttg | |
| | | ggatgtggggcatcaggcttatccgcataaaattttgaccggacgccgcgacaaaatcggc | |
| | | accatccgtcagaaaggcggtctgcacccgttcccgtggcgcggcgaaagcgaatatgac | |
| | | gtattaagcgtcgggcattcatcaacctccatcagtgccggaattggtattgcggttgctgcc | |
| | | gagaaagaaggcaaaaatcgccgcaccgtctgtgtaatcggcgatggtgcgattaccgca | |
| | | ggcatggcgtttgaagcgatgaatcacgcgggcgatatccgtcctgatatgctggtggttct | |
| | | caacgacaatgaaatgtcgatttccgaaaatgtcggcgcgctcaacaaccatctggcgcag | |
| | | ctgctttccggtaagctttactcttcactgcgcgaaggcgggaaaaaagttttctctggcgttc | |
| | | cgccaattaaagagctgctcaaacgcaccgaagaacatattaaaggcatggtagtgcctgg | |
| | | cacgttgtttgaagagctgggctttaactacatcggcccggttgacggtcacgatgtgctgg | |
| | | ggcttatcaccacgcttaagaacatgcgcgacctgaaaggcccgcagttcctgcatatcatg | |
| | | accaaaaaaggtcgtggttatgaaccggcagaaaaagacccaatcactttccacgccgtgc | |
| | | ctaaatttgatccctccagcggttgtttgccgaaaagtagcggcggtttgccgagctattcaa | |
| | | aaatctttggcgactggttgtgtgaaacggcagcgaaagacaacaagctgatggcgattac | |
| | | tccggcaatgcgtgaaggttccggcatggtcgagttttcacgtaaattcccggatcgctactt | |
| | | cgacgtggcaattgccgagcaacacgcagtgacctttgccgccggtctggcgattggtggg | |
| | | tacaaacccattgtcgcgatttactccactttcctgcaacgcgcctatgatcaggtgctgcatg | |
| | | acgtggcgattcaaaagctaccggtcctgttcgccattgaccgcgcgggcattgttggtgct | |
| | | gacggtcaaactcaccagggcgcttttgatctctcttacctgcgctgcataccagaaatggtc | |
| | | attatgaccccgagcgatgaaaacgaatgtcgccagatgctctataccggctatcactataa | |
| | | cgatggcccgtccgcggtgcgctacccgcgcggcaacgcggttggcgtggaactgacgcc | |
| | | gctggaaaaactaccaattggcaaaggcattgtgaagcgtcgtggcgagaaactggcgat | |
| | | ccttaactttggtacgctgatgccagaagcggcgaaagtcgccgaatcgctgaacgccacg | |
| | | ctggtcgatatgcgttttgtgaaaccgcttgatgaagcgttaattctggaaatggccaccagc | |
| | | catgaagcgctggtcaccgtagaagaaaacgccattatgggcggcgcaggtagcggagt | |
| | | gaacgaagtgctgatggcccatcgtaaaccagtacccgtgctgaacattggtctgccggact | |
| | | tctttattccgcaaggaactcaggaagaaatgcgcgccgaactcggcctcgatgccgccgg | |
| | | tatggaagccaaaatcaaggcctggctggcataactcgagtctggtaaagaaaccgctgct | |
| | | gcgaaatttgaacgccagcacatggactcgtctactagcgcagcttaattaacctaggctgc | |
| | | tgccaccgctgagcaataactagcataaccccttggggccaggcatcaaataaaacgaaa | |
| | | ggctcagtcgaaagactgggcctttcgttttatctgttgtttgtcggtgaacgctctccctctaa | |
| | | acgggtcttgaggggttttttgctgaaacctcaggcatttgagaagcacacggtcacactgct | |
| | | tccggtagtcaataaaccggtaaaccagcaatagacataagcggctatttaacgaccctgc | |
| | | cctgaaccgacgacaagctgacgaccgggtctccgcaagtggcacttttcggggaaatgtg | |
| | | cgcggaacccctatttgtttatttttctaaatacattcaaatatgtatccgctcatgaattaattc | |
| | | ttagaaaaactcatcgagcatcaaatgaaactgcaatttattcatatcaggattatcaatacc | |
| | | atatttttgaaaaagccgtttctgtaatgaaggagaaaactcaccgaggcagttccatagga | |
| | | tggcaagatcctggtatcggtctgcgattccgactcgtccaacatcaatacaacctattaattt | |
| | | cccctcgtcaaaaataaggttatcaagtgagaaatcaccatgagtgacgactgaatccggt | |
| | | gagaatggcaaaagtttatgcatttctttccagacttgttcaacaggccagccattacgctcgt | |
| | | catcaaaatcactcgcatcaaccaaaccgttattcattcgtgattgcgcctgagcgagacga | |
| | | aatacgcggtcgctgttaaaaggacaattacaaacaggaatcgaatgcaaccggcgcagg | |
| | | aacactgccagcgcatcaacaatattttcacctgaatcaggatattcttctaatacctggaat | |
| | | gctgttttcccggggatcgcagtggtgagtaaccatgcatcatcaggagtacggataaaatg | |
| | | cttgatggtcggaagaggcataaattccgtcagccagtttagtctgaccatctcatctgtaac | |
| | | atcattggcaacgctacctttgccatgtttcagaaacaactctggcgcatcgggcttcccatac | |
| | | aatcgatagattgtcgcacctgattgcccgacattatcgcgagcccatttatacccatataaat | |
| | | cagcatccatgttggaatttaatcgcggcctagagcaagacgtttcccgttgaatatggctca | |
| | | tactcttcctttttcaatattattgaagcatttatcagggttattgtctcatgagcggatacata | |
| | | tttgaatgtatttagaaaaataaacaaataggcatgcagcgctcttccgcttcctcgctcactga | |
| | | ctcgctacgctcggtcgttcgactgcggcgagcggtgtcagctcactcaaaagcggtaatac | |
| | | ggttatccacagaatcaggggataaagccggaaagaacatgtgagcaaaaagcaaagca | |
| | | ccggaagaagccaacgccgcaggcgtttttccataggctccgcccccctgacgagcatcac | |
| | | aaaaatcgacgctcaagccagaggtggcgaaacccgacaggactataaagataccaggc | |
| | | gtttccccctggaagctccctcgtgcgctctcctgttccgaccctgccgcttaccggatacctgt | |
| | | ccgcctttctcccttcgggaagcgtggcgctttctcatagctcacgctgttggtatctcagttcg | |
| | | gtgtaggtcgttcgctccaagctgggctgtgtgcacgaaccccccgttcagcccgaccgctg | |
| | | cgccttatccggtaactatcgtcttgagtccaacccggtaagacacgacttatcgccactggc | |
| | | agcagccattggtaactgatttagaggactttgtcttgaagttatgcacctgttaaggctaaa | |
| | | ctgaaagaacagattttggtgagtgcggtcctccaacccacttaccttggttcaaagagttgg | |
| | | tagctcagcgaaccttgagaaaaccaccgttggtagcggtggtttttctttatttatgagatga | |
| | | tgaatcaatcggtctatcaagtcaacgaacagctattccgttactctag | |
| Plasmid | 59 | TCGCGCGTTTCGGTGATGACGGTGAAAACCTCTGACACATGCAGCTCC | E. coli/ |
| | | CGGAGACGGTCACAGCTTGTCTGTAAGCGGATGCCGGGAGCAGACAA | S. |
| | | GCCCGTCAGGGCGCGTCAGCGGGTGTTGGCGGGTGTCGGGGCTGGC | cerevisiae |
| | | TTAACTATGCGGCATCAGAGCAGATTGTACTGAGAGTGCACCATACCA | |
| | | CCGTTTTAAGAGCTTGGTGAGCGCTAGGAGTCACTGCCAGGTATCGTT | |
| | | TGAACACGGCATTAGTCAGGGAAGTCATAACACAGTCCTTTCCCGCAA | |
| | | TTTTCTTTTTCTATTACTCTTGGCCTCCTCTAGTACACTCTATATTTTTTT | |
| | | ATGCCTCGGTAATGATTTTCATTTTTTTTTTTCCCCTAGCGGATGACTCT | |
| | | TTTTTTTTCTTAGCGATTGGCATTATCACATAATGAATTATACATTATAT | |
| | | AAAGTAATGTGATTTCTTCGAAGAATATACTAAAAAATGAGCAGGCAA | |
| | | GATAAACGAAGGCAAAGATGACAGAGCAGAAAGCCCTAGTAAAGCGT | |
| | | ATTACAAATGAAACCAAGATTCAGATTGCGATCTCTTTAAAGGGTGGTC | |
| | | CCCTAGCGATAGAGCACTCGATCTTCCCAGAAAAAGAGGCAGAAGCA | |
| | | GTAGCAGAACAGGCCACACAATCGCAAGTGATTAACGTCCACACAGGT | |
| | | ATAGGGTTTCTGGACCATATGATACATGCTCTGGCCAAGCATTCCGGC | |
| | | TGGTCGCTAATCGTTGAGTGCATTGGTGACTTACACATAGACGACCAT | |
| | | CACACCACTGAAGACTGCGGGATTGCTCTCGGTCAAGCTTTTAAAGAG | |
| | | GCCCTACTGGCGCGTGGAGTAAAAAGGTTTGGATCAGGATTTGCGCCT | |
| | | TTGGATGAGGCACTTTCCAGAGCGGTGGTAGATCTTTCGAACAGGCCG | |
| | | TACGCAGTTGTCGAACTTGGTTTGCAAAGGGAGAAAGTAGGAGATCTC | |
| | | TCTTGCGAGATGATCCCGCATTTTCTTGAAAGCTITGCAGAGGCTAGC | |
| | | AGAATTACCCTCCACGTTGATTGTCTGCGAGGCAAGAATGATCATCAC | |
| | | CGTAGTGAGAGTGCGTTCAAGGCTCTTGCGGTTGCCATAAGAGAAGC | |
| | | CACCTCGCCCAATGGTACCAACGATGTTCCCTCCACCAAAGGTGTTCT | |
| | | TATGTAGTGACACCGATTATTTAAAGCTGCAGCATACGATATATATACA | |
| | | TGTGTATATATGTATACCTATGAATGTCAGTAAGTATGTATACGAACAG | |
| | | TATGATACTGAAGATGACAAGGTAATGCATCATTCTATACGTGTCATTC | |
| | | TGAACGAGGCGCGCTTTCCTTTTTTCTTTTTGCTTTTTCTTTTTTTTTCT | |
| | | CTTGAACTCGATATGCGGTGTGAAATACCGCACAGATGCGTAAGGAGA | |
| | | AAATACCGCATCAGGAAATTGTAAACGTTAATATTTTGTTAAAATTCGC | |
| | | GTTAAATTTTTGTTAAATCAGCTCATTTTTTAACCAATAGGCCGAAATC | |
| | | GGCAAAATCCCTTATAAATCAAAAGAATAGACCGAGATAGGGTTGAGT | |
| | | GTTGTTCCAGTTTGGAACAAGAGTCCACTATTAAAGAACGTGGACTCC | |
| | | AACGTCAAAGGGCGAAAAACCGTCTATCAGGGCGATGGCCCACTACG | |
| | | TGAACCATCACCCTAATCAAGTTTTTTGGGGTCGAGGTGCCGTAAAGC | |
| | | ACTAAATCGGAACCCTAAAGGGAGCCCCCGATTTAGAGCTTGACGGG | |
| | | GAAAGCCGGCGAACGTGGCGAGAAAGGAAGGGAAGAAAGCGAAAGG | |
| | | AGCGGGCGCTAGGGCGCTGGCAAGTGTAGCGGTCACGCTGCGCGTA | |
| | | ACCACCACACCCGCCGCGCTTAATGCGCCGCTACAGGGCGCGTCCAT | |
| | | TCGCCATTCAGGCTGCGCAACTGTTGGGAAGGGCGATCGGTGCGGGC | |
| | | CTCTTCGCTATTACGCCAGCTGAATTGGAGCGACCTCATGCTATACCT | |
| | | GAGAAAGCAACCTGACCTACAGGAAAGAGTTACTCAAGAATAAGAATT | |
| | | TTCGTTTTAAAACCTAAGAGTCACTTTAAAATTTGTATACACTTATTTTT | |
| | | TTTATAACTTATTTAATAATAAAAATCATAAATCATAAGAAATTCGCTTA | |
| | | TTTAGAAGTGTCAACAACGTATCTACCAACGATTTGACCCTTTTCCATC | |
| | | TTTTCGTAAATTTCTGGCAAGGTAGACAAGCCGACAACCTTGATTGGA | |
| | | GACTTGACCAAACCTCTGGCGAAGAATTGTTAATTAAGAGCTCTTATAA | |
| | | TGGTCTTTGCCACAAATGAGCTGGTCTTGGTGGATGTGCTCTCATTCTT | |
| | | GAAGTCAATGCTTGACCAGATGCAGCCAACAACAATGTCAATTTTTCTG | |
| | | GAGTTGTAGTAGATTGTCTTTGATCCCATGCTTGTTGACCTGCTTGTTC | |
| | | AACTTTAACACCGATCTTTCTGTAAACTTGCTTAGCTGTTGCGATAGCC | |
| | | CATGCTGATCTCAATGGTAATCTAGCTAAACCAGCAGTTGCAGACAAG | |
| | | TAATATGGTTCTGCTTCTTGAACTAATCTTCTAGCAATTCTTGACAATGC | |
| | | TTGTCTATTTTCTGGTGCAGCGTAGTTTTCCTTATTCAAACCTTCATGTT | |
| | | CCAACCAAGAAGCTGGCAAATAACATCTACCAGCATGTGCATCATCAA | |
| | | CAATATCTCTAGCGATGTTTGTCAATTGGAATGCCAAACCCAAATCACA | |
| | | AGCTCTATCCAAAGTTGCTTTATCTCTAACACCCATAATTTGAGCCATC | |
| | | ATTAAACCAACAACACCTGCAACATGGTAACAGTATCTCAAAGTATCAT | |
| | | CCAATTGTGAGTATTGAGCTTCTCTAACATCCATTGCAAAACCTTCCAA | |
| | | ATGATCAAAAGCATAAGCTGGAGCAATATCATGTGCCATAGCAACTTC | |
| | | TTGAAATGCAGCAAAAGCTGGTTCATGCATTTGAGAACCAGCATAGGC | |
| | | TTGTCTTGTCTTCATTTCCAATTGCATCAATCTTTGTTCTGGAGTTTGTA | |
| | | AAGCTGGTTGTCTTGCTTGAAAACCCAATGTTTGATCATCGATAACATC | |
| | | ATCACAATGTCTACACCAAGCATACAACATCAAAACTGATCTTCTAGTC | |
| | | TTTGCATCGAACAATTTAGATGCTGTAGCAAATGATTTAGAACCAACAG | |
| | | CCATGAATTCGAATTTTCAAAAATTCTTACTTTTTTTTTGGATGGACGCA | |
| | | AAGAAGTTTAATAATCATATTACATGGCATTACCACCATATACATATCC | |
| | | ATATACATATCCATATCTAATCTTACTTATATGTTGTGGAAATGTAAAGA | |
| | | GCCCCATTATCTTAGCCTAAAAAAACCTTCTCTTTGGAACTTTCAGTAA | |
| | | TACGCTTAACTGCTCATTGCTATATTGAAGTACGGATTAGAAGCCGCC | |
| | | GAGCGGGTGACAGCCCTCCGAAGGAAGACTCTCCTCCGTGCGTCCTC | |
| | | GTCTTCACCGGTCGCGTTCCTGAAACGCAGATGTGCCTCGCGCCGCAC | |
| | | TGCTCCGAACAATAAAGATTCTACAATACTAGCTTTTATGGTTATGAAG | |
| | | AGGAAAAATTGGCAGTAACCTGGCCCCACAAACCTTCAAATGAACGAA | |
| | | TCAAATTAACAACCATAGGATGATAATGCGATTAGTTTTTTAGCCTTAT | |
| | | TTCTGGGGTAATTAATCAGCGAAGCGATGATTTTTGATCTATTAACAGA | |
| | | TATATAAATGCAAAAACTGCATAACCACTTTAACTAATACTTTCAACATT | |
| | | TTCGGTTTGTATTACTTCTTATTCAAATGTAATAAAAGTATCAACAAAAA | |
| | | ATTGTTAATATACCTCTATACTTTAACGTCAAGGAGAAAAAACCCCGGA | |
| | | TCCGTAATACGACTCACTATAGGGCCCGGGCGTCGACATGGAACAGA | |
| | | AGTTGATTTCCGAAGAAGACCTCGAGTAAGCTTGGTACCGCGGCTAGC | |
| | | TAAGATCCGCTCTAACCGAAAAGGAAGGAGTTAGACAACCTGAAGTCT | |
| | | AGGTCCCTATTTATTTTTTTATAGTTATGTTAGTATTAAGAACGTTATTT | |
| | | ATATTTCAAATTTTTCTTTTTTTTCTGTACAGACGCGTGTACGCATGTAA | |
| | | CATTATACTGAAAACCTTGCTTGAGAAGGTTTTGGGACGCTCGAAGAT | |
| | | CCAGCTGCATTAATGAATCGGCCAACGCGCGGGGAGAGGCGGTTTGC | |
| | | GTATTGGGCGCTCTTCCGCTTCCTCGCTCACTGACTCGCTGCGCTCGG | |
| | | TCGTTCGGCTGCGGCGAGCGGTATCAGCTCACTCAAAGGCGGTAATA | |
| | | CGGTTATCCACAGAATCAGGGGATAACGCAGGAAAGAACATGTGAGC | |
| | | AAAAGGCCAGCAAAAGGCCAGGAACCGTAAAAAGGCCGCGTTGCTGG | |
| | | CGTTTTTCCATAGGCTCCGCCCCCCTGACGAGCATCACAAAAATCGAC | |
| | | GCTCAAGTCAGAGGTGGCGAAACCCGACAGGACTATAAAGATACCAG | |
| | | GCGTTTCCCCCTGGAAGCTCCCTCGTGCGCTCTCCTGTTCCGACCCTG | |
| | | CCGCTTACCGGATACCTGTCCGCCTTTCTCCCTTCGGGAAGCGTGGCG | |
| | | CTTTCTCATAGCTCACGCTGTAGGTATCTCAGTTCGGTGTAGGTCGTTC | |
| | | GCTCCAAGCTGGGCTGTGTGCACGAACCCCCCGTTCAGCCCGACCGC | |
| | | TGCGCCTTATCCGGTAACTATCGTCTTGAGTCCAACCCGGTAAGACAC | |
| | | GACTTATCGCCACTGGCAGCAGCCACTGGTAACAGGATTAGCAGAGC | |
| | | GAGGTATGTAGGCGGTGCTACAGAGTTCTTGAAGTGGTGGCCTAACTA | |
| | | CGGCTACACTAGAAGGACAGTATTTGGTATCTGCGCTCTGCTGAAGCC | |
| | | AGTTACCTTCGGAAAAAGAGTTGGTAGCTCTTGATCCGGCAAACAAAC | |
| | | CACCGCTGGTAGCGGTGGTTTTTTTGTTTGCAAGCAGCAGATTACGCG | |
| | | CAGAAAAAAAGGATCTCAAGAAGATCCTTTGATCTTTTCTACGGGGTCT | |
| | | GACGCTCAGTGGAACGAAAACTCACGTTAAGGGATTTTGGTCATGAGA | |
| | | TTATCAAAAAGGATCTTCACCTAGATCCTITTAAATTAAAAATGAAGTTT | |
| | | TAAATCAATCTAAAGTATATATGAGTAAACTTGGTCTGACAGTTACCAA | |
| | | TGCTTAATCAGTGAGGCACCTATCTCAGCGATCTGTCTATTTCGTTCAT | |
| | | CCATAGTTGCCTGACTCCCCGTCGTGTAGATAACTACGATACGGGAGG | |
| | | GCTTACCATCTGGCCCCAGTGCTGCAATGATACCGCGAGACCCACGCT | |
| | | CACCGGCTCCAGATTTATCAGCAATAAACCAGCCAGCCGGAAGGGCC | |
| | | GAGCGCAGAAGTGGTCCTGCAACTTTATCCGCCTCCATCCAGTCTATT | |
| | | AATTGTTGCCGGGAAGCTAGAGTAAGTAGTTCGCCAGTTAATAGTTTG | |
| | | CGCAACGTTGTTGCCATTGCTACAGGCATCGTGGTGTCACGCTCGTCG | |
| | | TTTGGTATGGCTTCATTCAGCTCCGGTTCCCAACGATCAAGGCGAGTT | |
| | | ACATGATCCCCCATGTTGTGCAAAAAAGCGGTTAGCTCCTTCGGTCCT | |
| | | CCGATCGTTGTCAGAAGTAAGTTGGCCGCAGTGTTATCACTCATGGTT | |
| | | ATGGCAGCACTGCATAATTCTCTTACTGTCATGCCATCCGTAAGATGCT | |
| | | TTTCTGTGACTGGTGAGTACTCAACCAAGTCATTCTGAGAATAGTGTAT | |
| | | GCGGCGACCGAGTTGCTCTTGCCCGGCGTCAATACGGGATAATACCG | |
| | | CGCCACATAGCAGAACTTTAAAAGTGCTCATCATTGGAAAACGTTCTTC | |
| | | GGGGCGAAAACTCTCAAGGATCTTACCGCTGTTGAGATCCAGTTCGAT | |
| | | GTAACCCACTCGTGCACCCAACTGATCTTCAGCATCTTITACTTTCACC | |
| | | AGCGTTTCTGGGTGAGCAAAAACAGGAAGGCAAAATGCCGCAAAAAA | |
| | | GGGAATAAGGGCGACACGGAAATGTTGAATACTCATACTCTTCCTTTT | |
| | | TCAATATTATTGAAGCATTTATCAGGGTTATTGTCTCATGAGCGGATAC | |
| | | ATATTTGAATGTATTTAGAAAAATAAACAAATAGGGGTTCCGCGCACAT | |
| | | TTCCCCGAAAAGTGCCACCTGAACGAAGCATCTGTGCTTCATTTTGTA | |
| | | GAACAAAAATGCAACGCGAGAGCGCTAATTTTTCAAACAAAGAATCTG | |
| | | AGCTGCATTTTTACAGAACAGAAATGCAACGCGAAAGCGCTATTTTAC | |
| | | CAACGAAGAATCTGTGCTTCATTTTTGTAAAACAAAAATGCAACGCGA | |
| | | GAGCGCTAATTTTTCAAACAAAGAATCTGAGCTGCATTTTTACAGAACA | |
| | | GAAATGCAACGCGAGAGCGCTATTTTACCAACAAAGAATCTATACTTCT | |
| | | TTTTTGTTCTACAAAAATGCATCCCGAGAGCGCTATTTTTCTAACAAAG | |
| | | CATCTTAGATTACTTTTTTTCTCCTTTGTGCGCTCTATAATGCAGTCTCT | |
| | | TGATAACTTTTTGCACTGTAGGTCCGTTAAGGTTAGAAGAAGGCTACTT | |
| | | TGGTGTCTATTTTCTCTTCCATAAAAAAAGCCTGACTCCACTTCCCGCG | |
| | | TTTACTGATTACTAGCGAAGCTGCGGGTGCATTTTTTCAAGATAAAGG | |
| | | CATCCCCGATTATATTCTATACCGATGTGGATTGCGCATACTTTGTGAA | |
| | | CAGAAAGTGATAGCGTTGATGATTCTTCATTGGTCAGAAAATTATGAAC | |
| | | GGTTTCTTCTATTTTGTCTCTATATACTACGTATAGGAAATGTTTACATT | |
| | | TTCGTATTGTTTTCGATTCACTCTATGAATAGTTCTTACTACAATTTTTTT | |
| | | GTCTAAAGAGTAATACTAGAGATAAACATAAAAAATGTAGAGGTCGAG | |
| | | TTTAGATGCAAGTTCAAGGAGCGAAAGGTGGATGGGTAGGTTATATAG | |
| | | GGATATAGCACAGAGATATATAGCAAAGAGATACTTTTGAGCAATGTT | |
| | | TGTGGAAGCGGTATTCGCAATATTTTAGTAGCTCGTTACAGTCCGGTG | |
| | | CGTTTTTGGTTTTTTGAAAGTGCGTCTTCAGAGCGCTTTTGGTTTTCAA | |
| | | AAGCGCTCTGAAGTTCCTATACTTTCTAGAGAATAGGAACTTCGGAATA | |
| | | GGAACTTCAAAGCGTTTCCGAAAACGAGCGCTTCCGAAAATGCAACGC | |
| | | GAGCTGCGCACATACAGCTCACTGTTCACGTCGCACCTATATCTGCGT | |
| | | GTTGCCTGTATATATATATACATGAGAAGAACGGCATAGTGCGTGTTTA | |
| | | TGCTTAAATGCGTACTTATATGCGTCTATTTATGTAGGATGAAAGGTAG | |
| | | TCTAGTACCTCCTGTGATATTATCCCATTCCATGCGGGGTATCGTATGC | |
| | | TTCCTTCAGCACTACCCTTTAGCTGTTCTATATGCTGCCACTCCTCAATT | |
| | | GGATTAGTCTCATCCTTCAATGCTATCATTTCCTTTGATATTGGATCATA | |
| | | CTAAGAAACCATTATTATCATGACATTAACCTATAAAAATAGGCGTATC | |
| | | ACGAGGCCCTTTCGTC | |
| Plasmid | 60 | tcgcgcgtttcggtgatgacggtgaaaacctctgacacatgcagctcccggagacggtcac | E. coli/S. |
| | | agcttgtctgtaagcggatgccgggagcagacaagcccgtcagggcgcgtcagcgggtgt | cerevisiae |
| | | tggcgggtgtcggggctggcttaactatgcggcatcagagcagattgtactgagagtgcac | |
| | | cataccacagcttttcaattcaattcatcattttttttttattcttttttttgatttcggtttct | |
| | | ttgaaatttttttgattcggtaatctccgaacagaaggaagaacgaaggaaggagcacagacttag | |
| | | attggtatatatacgcatatgtagtgttgaagaaacatgaaattgcccagtattcttaaccca | |
| | | actgcacagaacaaaaacctgcaggaaacgaagataaatcatgtcgaaagctacatataa | |
| | | ggaacgtgctgctactcatcctagtcctgttgctgccaagctatttaatatcatgcacgaaaa | |
| | | gcaaacaaacttgtgtgcttcattggatgttcgtaccaccaaggaattactggagttagttga | |
| | | agcattaggtcccaaaatttgtttactaaaaacacatgtggatatcttgactgatttttccatg | |
| | | gagggcacagttaagccgctaaaggcattatccgccaagtacaattttttactcttcgaaga | |
| | | cagaaaatttgctgacattggtaatacagtcaaattgcagtactctgcgggtgtatacagaa | |
| | | tagcagaatgggcagacattacgaatgcacacggtgtggtgggcccaggtattgttagcgg | |
| | | tttgaagcaggcggcagaagaagtaacaaaggaacctagaggccttttgatgttagcaga | |
| | | attgtcatgcaagggctccctatctactggagaatatactaagggtactgttgacattgcgaa | |
| | | gagcgacaaagattttgttatcggctttattgctcaaagagacatgggtggaagagatgaa | |
| | | ggttacgattggttgattatgacacccggtgtgggtttagatgacaagggagacgcattggg | |
| | | tcaacagtatagaaccgtggatgatgtggtctctacaggatctgacattattattgttggaag | |
| | | aggactatttgcaaagggaagggatgctaaggtagagggtgaacgttacagaaaagcag | |
| | | gctgggaagcatatttgagaagatgcggccagcaaaactaaaaaactgtattataagtaa | |
| | | atgcatgtatactaaactcacaaattagagcttcaatttaattatatcagttattaccctatgcg | |
| | | gtgtgaaataccgcacagatgcgtaaggagaaaataccgcatcaggaaattgtaaacgtt | |
| | | aatattttgttaaaattcgcgttaaatttttgttaaatcagctcattttttaaccaataggccgaa | |
| | | atcggcaaaatcccttataaatcaaaagaatagaccgagatagggttgagtgttgttccagt | |
| | | ttggaacaagagtccactattaaagaacgtggactccaacgtcaaagggcgaaaaaccgt | |
| | | ctatcagggcgatggcccactacgtgaaccatcaccctaatcaagttttttggggtcgaggt | |
| | | gccgtaaagcactaaatcggaaccctaaagggagcccccgatttagagcttgacggggaa | |
| | | agccggcgaacgtggcgagaaaggaagggaagaaagcgaaaggagcgggcgctagg | |
| | | gcgctggcaagtgtagcggtcacgctgcgcgtaaccaccacacccgccgcgcttaatgcgc | |
| | | cgctacagggcgcgtccattcgccattcaggctgcgcaactgttgggaagggcgatcggtg | |
| | | cgggcctcttcgctattacgccagctgaattggagcgacctcatgctatacctgagaaagca | |
| | | acctgacctacaggaaagagttactcaagaataagaattttcgttttaaaacctaagagtca | |
| | | ctttaaaatttgtatacacttattttttttataacttatttaataataaaaatcataaatcataag | |
| | | aaattcgcttatttagaagtgtcaacaacgtatctaccaacgatttgacccttttccatcttttcg | |
| | | taaatttctggcaaggtagacaagccgacaaccttgattggagacttgaccaaacctctggc | |
| | | gaagaattgttaattaagagctcagatcttatcgtcgtcatccttgtaatccatcgatactagt | |
| | | gcttaggatttaatgcaggtgacggacccatctttcaaacgatttatatcagtggcgtccaaa | |
| | | ttgttaggttttgttggttcagcaggtttcctgttgtgggtcatatgactttgaaccaaatggcc | |
| | | ggctgctagggcagcacataaggataattcacctgccaagacggcacaggcaactattctt | |
| | | gctaattgacgtgcgttggtaccaggagcggtagcatgcgggcctcttacacctaataagtc | |
| | | caacatggcaccttgtggttctagaacagtaccaccaccgatggtacctacttcgatggatg | |
| | | gcatggatacggaaattctcaaatcaccgtccacttctttcatcaatgttatacagttggaact | |
| | | ttcaacattttgtgcaggatcttgtcctaatgccaagaaaacagctgtcactaaattagctgc | |
| | | atgtgcgttaaatccaccaacagacccagccattgcagatccaaccaaattcttagcaatgtt | |
| | | caactcaaccaatgcggaaacatcactttttaacacttttctgacaacatcaccaggaatagt | |
| | | agcttctgcgacgacactcttaccacgaccttcgatccagttgatggcagctggttttttgtcg | |
| | | gtacagtagttaccagaaacggagacaacctccatatcttcccagccatactcttctaccattt | |
| | | gctttaatgagtattcgacacctttagaaatcatattcatacccattgcgtcaccagtagttgtt | |
| | | ctaaatctcatgaagagtaaatctcctgctagacaagtttgaatatgttgcagacgtgcaaat | |
| | | cttgatgtagagttaaaagcttttttaattgcgttttgtccctcttctgagtctaaccatatctta | |
| | | caggcaccagatcttttcaaagttgggaaacggactactgggcctcttgtcataccatccttag | |
| | | ttaaaacagttgttgcaccaccgccagcattgattgccttacagccacgcatggcagaagct | |
| | | accaaacaaccctctgtagttgccattggtatatgataagatgtaccatcgataaccaaggg | |
| | | gcctataacaccaacgggcaaaggcatgtaacctataacattttcacaacaagcgccaaat | |
| | | acgcggtcgtagtcataatttttatatggtaaacgatcagatgctaatacaggagcttctgcc | |
| | | aaaattgaaagagccttcctacgtaccgcaaccgctctcgtagtatcacctaattttttctcca | |
| | | aagcgtacaaaggtaacttaccgtgaataaccaaggcagcgacctctttgttcttcaattgtt | |
| | | ttgtatttccactacttaataatgcttctaattcttctaaaggacgtattttcttatccaagcttt | |
| | | caatatcgcgggaatcatcttcctcactagatgatgaaggtcctgatgagctcgattgcgcag | |
| | | atgataaacttttgactttcgatccagaaatgactgttttattggttaaaactggtgtagaagc | |
| | | cttttgtacaggagcagtaaaagacttcttggtgacttcagttttcaccaattggtccatggcc | |
| | | gccctttagtgagggttgaattcgaattttcaaaaattcttactttttttttggatggacgcaaa | |
| | | gaagtttaataatcatattacatggcattaccaccatatacatatccatatacatatccatatct | |
| | | aatcttacttatatgttgtggaaatgtaaagagccccattatcttagcctaaaaaaaccttctc | |
| | | tttggaactttcagtaatacgcttaactgctcattgctatattgaagtacggattagaagccgc | |
| | | cgagcgggtgacagccctccgaaggaagactctcctccgtgcgtcctcgtcttcaccggtcg | |
| | | cgttcctgaaacgcagatgtgcctcgcgccgcactgctccgaacaataaagattctacaata | |
| | | ctagcttttatggttatgaagaggaaaaattggcagtaacctggccccacaaaccttcaaat | |
| | | gaacgaatcaaattaacaaccataggatgataatgcgattagttttttagccttatttctggg | |
| | | gtaattaatcagcgaagcgatgatttttgatctattaacagatatataaatgcaaaaactgca | |
| | | taaccactttaactaatactttcaacattttcggtttgtattacttcttattcaaatgtaataaaa | |
| | | gtatcaacaaaaaattgttaatatacctctatactttaacgtcaaggagaaaaaaccccgga | |
| | | tccgtaatacgactcactatagggcccgggcgtcgacatggaacagaagttgatttccgaa | |
| | | gaagacctcgagtaagcttggtaccgcggctagctaagatccgctctaaccgaaaaggaa | |
| | | ggagttagacaacctgaagtctaggtccctatttatttttttatagttatgttagtattaagaac | |
| | | gttatttatatttcaaatttttcttttttttctgtacagacgcgtgtacgcatgtaacattatact | |
| | | gaaaaccttgcttgagaaggttttgggacgctcgaagatccagctgcattaatgaatcggcca | |
| | | acgcgcggggagaggcggtttgcgtattgggcgctcttccgcttcctcgctcactgactcgct | |
| | | gcgctcggtcgttcggctgcggcgagcggtatcagctcactcaaaggcggtaatacggttat | |
| | | ccacagaatcaggggataacgcaggaaagaacatgtgagcaaaaggccagcaaaaggc | |
| | | caggaaccgtaaaaaggccgcgttgctggcgtttttccataggctccgcccccctgacgagc | |
| | | atcacaaaaatcgacgctcaagtcagaggtggcgaaacccgacaggactataaagatacc | |
| | | aggcgtttccccctggaagctccctcgtgcgctctcctgttccgaccctgccgcttaccggata | |
| | | cctgtccgcctttctcccttcgggaagcgtggcgctttctcatagctcacgctgtaggtatctca | |
| | | gttcggtgtaggtcgttcgctccaagctgggctgtgtgcacgaaccccccgttcagcccgacc | |
| | | gctgcgccttatccggtaactatcgtcttgagtccaacccggtaagacacgacttatcgccac | |
| | | tggcagcagccactggtaacaggattagcagagcgaggtatgtaggcggtgctacagagt | |
| | | tcttgaagtggtggcctaactacggctacactagaaggacagtatttggtatctgcgctctgc | |
| | | tgaagccagttaccttcggaaaaagagttggtagctcttgatccggcaaacaaaccaccgct | |
| | | ggtagcggtggtttttttgtttgcaagcagcagattacgcgcagaaaaaaaggatctcaaga | |
| | | agatcctttgatcttttctacggggtctgacgctcagtggaacgaaaactcacgttaagggat | |
| | | tttggtcatgagattatcaaaaaggatcttcacctagatccttttaaattaaaaatgaagtttt | |
| | | aaatcaatctaaagtatatatgagtaaacttggtctgacagttaccaatgcttaatcagtgag | |
| | | gcacctatctcagcgatctgtctatttcgttcatccatagttgcctgactccccgtcgtgtagat | |
| | | aactacgatacgggagggcttaccatctggccccagtgctgcaatgataccgcgagaccca | |
| | | cgctcaccggctccagatttatcagcaataaaccagccagccggaagggccgagcgcaga | |
| | | agtggtcctgcaactttatccgcctccatccagtctattaattgttgccgggaagctagagtaa | |
| | | gtagttcgccagttaatagtttgcgcaacgttgttgccattgctacaggcatcgtggtgtcacg | |
| | | ctcgtcgtttggtatggcttcattcagctccggttcccaacgatcaaggcgagttacatgatcc | |
| | | cccatgttgtgcaaaaaagcggttagctccttcggtcctccgatcgttgtcagaagtaagttg | |
| | | gccgcagtgttatcactcatggttatggcagcactgcataattctcttactgtcatgccatccgt | |
| | | aagatgcttttctgtgactggtgagtactcaaccaagtcattctgagaatagtgtatgcggcg | |
| | | accgagttgctcttgcccggcgtcaatacgggataataccgcgccacatagcagaactttaa | |
| | | aagtgctcatcattggaaaacgttcttcggggcgaaaactctcaaggatcttaccgctgttga | |
| | | gatccagttcgatgtaacccactcgtgcacccaactgatcttcagcatcttttactttcaccagc | |
| | | gtttctgggtgagcaaaaacaggaaggcaaaatgccgcaaaaaagggaataagggcga | |
| | | cacggaaatgttgaatactcatactcttcctttttcaatattattgaagcatttatcagggttatt | |
| | | gtctcatgagcggatacatatttgaatgtatttagaaaaataaacaaataggggttccgcgc | |
| | | acatttccccgaaaagtgccacctgaacgaagcatctgtgcttcattttgtagaacaaaaatg | |
| | | caacgcgagagcgctaatttttcaaacaaagaatctgagctgcatttttacagaacagaaat | |
| | | gcaacgcgaaagcgctattttaccaacgaagaatctgtgcttcatttttgtaaaacaaaaatg | |
| | | caacgcgagagcgctaatttttcaaacaaagaatctgagctgcatttttacagaacagaaat | |
| | | gcaacgcgagagcgctattttaccaacaaagaatctatacttcttttttgttctacaaaaatgc | |
| | | atcccgagagcgctatttttctaacaaagcatcttagattactttttttctcctttgtgcgctcta | |
| | | taatgcagtctcttgataactttttgcactgtaggtccgttaaggttagaagaaggctactttgg | |
| | | tgtctattttctcttccataaaaaaagcctgactccacttcccgcgtttactgattactagcgaa | |
| | | gctgcgggtgcattttttcaagataaaggcatccccgattatattctataccgatgtggattgc | |
| | | gcatactttgtgaacagaaagtgatagcgttgatgattcttcattggtcagaaaattatgaac | |
| | | ggtttcttctattttgtctctatatactacgtataggaaatgtttacattttcgtattgttttcga | |
| | | ttcactctatgaatagttcttactacaatttttttgtctaaagagtaatactagagataaacataa | |
| | | aaaatgtagaggtcgagtttagatgcaagttcaaggagcgaaaggtggatgggtaggtta | |
| | | tatagggatatagcacagagatatatagcaaagagatacttttgagcaatgtttgtggaagc | |
| | | ggtattcgcaatattttagtagctcgttacagtccggtgcgtttttggttttttgaaagtgcgtct | |
| | | tcagagcgcttttggttttcaaaagcgctctgaagttcctatactttctagagaataggaactt | |
| | | cggaataggaacttcaaagcgtttccgaaaacgagcgcttccgaaaatgcaacgcgagct | |
| | | gcgcacatacagctcactgttcacgtcgcacctatatctgcgtgttgcctgtatatatatatac | |
| | | atgagaagaacggcatagtgcgtgtttatgcttaaatgcgtacttatatgcgtctatttatgta | |
| | | ggatgaaaggtagtctagtacctcctgtgatattatcccattccatgcggggtatcgtatgctt | |
| | | ccttcagcactaccctttagctgttctatatgctgccactcctcaattggattagtctcatccttc | |
| | | aatgctatcatttcctttgatattggatcatactaagaaaccattattatcatgacattaaccta | |
| | | taaaaataggcgtatcacgaggccctttcgtc | |
| Plasmid | 61 | tcgcgcgtttcggtgatgacggtgaaaacctctgacacatgcagctcccggagacggtcac | E. coli/S. |
| | | agcttgtctgtaagcggatgccgggagcagacaagcccgtcagggcgcgtcagcgggtgt | cerevisiae |
| | | tggcgggtgtcggggctggcttaactatgcggcatcagagcagattgtactgagagtgcac | |
| | | cataaattcccgttttaagagcttggtgagcgctaggagtcactgccaggtatcgtttgaaca | |
| | | cggcattagtcagggaagtcataacacagtcctttcccgcaattttctttttctattactcttggc | |
| | | ctcctctagtacactctatatttttttatgcctcggtaatgattttcatttttttttttcccctag | |
| | | cggatgactctttttttttcttagcgattggcattatcacataatgaattatacattatataaagt | |
| | | aatgtgatttcttcgaagaatatactaaaaaatgagcaggcaagataaacgaaggcaaagatg | |
| | | acagagcagaaagccctagtaaagcgtattacaaatgaaaccaagattcagattgcgatct | |
| | | ctttaaagggtggtcccctagcgatagagcactcgatcttcccagaaaaagaggcagaagc | |
| | | agtagcagaacaggccacacaatcgcaagtgattaacgtccacacaggtatagggtttctg | |
| | | gaccatatgatacatgctctggccaagcattccggctggtcgctaatcgttgagtgcattggt | |
| | | gacttacacatagacgaccatcacaccactgaagactgcgggattgctctcggtcaagctttt | |
| | | aaagaggccctactggcgcgtggagtaaaaaggtttggatcaggatttgcgcctttggatg | |
| | | aggcactttccagagcggtggtagatctttcgaacaggccgtacgcagttgtcgaacttggt | |
| | | ttgcaaagggagaaagtaggagatctctcttgcgagatgatcccgcattttcttgaaagcttt | |
| | | gcagaggctagcagaattaccctccacgttgattgtctgcgaggcaagaatgatcatcacc | |
| | | gtagtgagagtgcgttcaaggctcttgcggttgccataagagaagccacctcgcccaatggt | |
| | | accaacgatgttccctccaccaaaggtgttcttatgtagtgacaccgattatttaaagctgca | |
| | | gcatacgatatatatacatgtgtatatatgtatacctatgaatgtcagtaagtatgtatacgaa | |
| | | cagtatgatactgaagatgacaaggtaatgcatcattctatacgtgtcattctgaacgaggc | |
| | | gcgctttccttttttctttttgctttttctttttttttctcttgaactcgacggatctatgcggtg | |
| | | tgaaataccgcacagatgcgtaaggagaaaataccgcatcaggaaattgtaaacgttaatatttt | |
| | | gttaaaattcgcgttaaatttttgttaaatcagctcattttttaaccaataggccgaaatcggc | |
| | | aaaatcccttataaatcaaaagaatagaccgagatagggttgagtgttgttccagtttggaa | |
| | | caagagtccactattaaagaacgtggactccaacgtcaaagggcgaaaaaccgtctatca | |
| | | gggcgatggcccactacgtgaaccatcaccctaatcaagttttttggggtcgaggtgccgta | |
| | | aagcactaaatcggaaccctaaagggagcccccgatttagagcttgacggggaaagccgg | |
| | | cgaacgtggcgagaaaggaagggaagaaagcgaaaggagcgggcgctagggcgctgg | |
| | | caagtgtagcggtcacgctgcgcgtaaccaccacacccgccgcgcttaatgcgccgctaca | |
| | | gggcgcgtcgcgccattcgccattcaggctgcgcaactgttgggaagggcgatcggtgcgg | |
| | | gcctcttcgctattacgccagctggcgaaagggggatgtgctgcaaggcgattaagttgggt | |
| | | aacgccagggttttcccagtcacgacgttgtaaaacgacggccagtgagcgcgcgtaatac | |
| | | gactcactatagggcgaattgggtaccgggcccccccagcaacaggcgcgttggactttta | |
| | | attttcgaggaccgcgaatccttacatcacacccaatcccccacaagtgatcccccacacacc | |
| | | atagcttcaaaatgtttctactccttttttactcttccagattttctcggactccgcgcatcgccg | |
| | | taccacttcaaaacacccaagcacagcatactaaatttcccctctttcttcctctagggtgtcgtt | |
| | | aattacccgtactaaaggtttggaaaagaaaaaagagaccgcctcgtttctttttcttcgtcg | |
| | | aaaaaggcaataaaaatttttatcacgtttctttttcttgaaaatttttttttttgatttttttct | |
| | | ctttcgatgacctcccattgatatttaagttaataaacggtcttcaatttctcaagtttcagtttc | |
| | | atttttcttgttctattacaactttttttacttcttgctcattagaaagaaagcatagcaatctaa | |
| | | tctaagttttaattacaaaatgtcaactttgtcagtttctacaccatctttttcttcttctccatt | |
| | | gtcttctgttaacaaaaattcaactaagcaacatgttacaagaaattcagttatcttccatgattc | |
| | | tatttggggtgatcaattcttggaatacaaggaaaagttcaatgttgctactgaaaaacaattaat | |
| | | tgaagaattgaaagaagaagttagaaatgaattaatgatcagagcttgtaacgaagcatcaa | |
| | | gatacatcaagttgatccaattgatcgatgttgttgaaagattgggtttggcataccatttcga | |
| | | aaaggaaatcgaagaatctttgcaacatatctacgttacttacggtcataagtggacaaact | |
| | | acaacaacatcgaatcattgtctttgtggttcagattgttgagacaaaatggtttcaacgtttc | |
| | | atctgatattttcgaaaatcatattgatgaaaagggtaattttcaagaatctttgtgtaatgat | |
| | | ccacaaggcatgttagctttgtatgaagctgcatacatgagagttgaaggtgaaatcatctt | |
| | | ggataaggcattggaatttactaagttgcatttgggtatcatctcaaacgatccatcttgtgat | |
| | | tcatctttgagaacagaaatcaagcaagctttgaaacaaccattgagaagaagattgccaa | |
| | | gattggaagctgttagatacatcgcaatctaccaacaaaaagcttcacattctgaagttttgtt | |
| | | gaagttggcaaagttggatttcaatgttttgcaagaaatgcataaggatgaattatcacaaa | |
| | | tctgtaagtggtggaaggatttggatattagaaacaagttgccatacgttagagatagattg | |
| | | atcgaaggttacttctggatcttgggtatctacttcgaaccacaacattctagaactagaatgt | |
| | | tcttgatgaagacatgtatgtggttgatcgttttggatgatactttcgataactatggtacatac | |
| | | gaagaattagaaatcttcactcaagctgttgaaagatggtctatcacatgtttggatgaattg | |
| | | ccagaatacatgaagttgatctaccatgaacaattcagagttcatcaagaaatggaagaat | |
| | | cattggaaaaagaaggtaaagcttaccaaatccattacatcaaggaaatggcaaaagaag | |
| | | gtactagatctttgttattggaagctaaatggttgaaagaaggttatatgccaacattggatg | |
| | | aatacttgtcaaattctttggttacttgtggttacgctttaatgacagcaagatcatacgttgca | |
| | | agagatgatggtatcgttactgaagatgctttcaagtgggttgcaacacatccaccaatcgtt | |
| | | aaggctgcttgtaagatcttgagattgatggatgatattgctactcataaagaagaacaaga | |
| | | aagaggtcatatcgcatcatctatcgaatgttacagaaaagaaacaggtgcttctgaagaa | |
| | | gaagcttgtatggatttcttgaaacaagttgaagatggttggaaggttatcaaccaagaatc | |
| | | attgatgccaactgatgttccattcccattgttgatcccagctatcaacttggcaagagtttctg | |
| | | atacattgtacaaggataatgatggttacaatcatgctgataaagaagttattggttacatta | |
| | | aatctttatttgttcatccaatgattgtttaagcgaatttcttatgatttatgatttttattatta | |
| | | aataagttataaaaaaaataagtgtatacaaattttaaagtgactcttaggttttaaaacgaaaa | |
| | | ttcttattcttgagtaactctttcctgtaggtcaggttgctttctcaggtatagcatgaggtcgct | |
| | | gatccactagttctagagcggccgccaccgcggtggagctccagcttttgttccctttagtga | |
| | | gggttaattgcgcgcttggcgtaatcatggtcatagctgtttcctgtgtgaaattgttatccgct | |
| | | cacaattccacacaacataggagccggaagcataaagtgtaaagcctggggtgcctaatg | |
| | | agtgaggtaactcacattaattgcgttgcgctcactgcccgctttccagtcgggaaacctgtc | |
| | | gtgccagctgcattaatgaatcggccaacgcgcggggagaggcggtttgcgtattgggcgc | |
| | | tcttccgcttcctcgctcactgactcgctgcgctcggtcgttcggctgcggcgagcggtatcag | |
| | | ctcactcaaaggcggtaatacggttatccacagaatcaggggataacgcaggaaagaaca | |
| | | tgtgagcaaaaggccagcaaaaggccaggaaccgtaaaaaggccgcgttgctggcgtttt | |
| | | tccataggctccgcccccctgacgagcatcacaaaaatcgacgctcaagtcagaggtggcg | |
| | | aaacccgacaggactataaagataccaggcgtttccccctggaagctccctcgtgcgctctc | |
| | | ctgttccgaccctgccgcttaccggatacctgtccgcctttctcccttcgggaagcgtggcgct | |
| | | ttctcatagctcacgctgtaggtatctcagttcggtgtaggtcgttcgctccaagctgggctgt | |
| | | gtgcacgaaccccccgttcagcccgaccgctgcgccttatccggtaactatcgtcttgagtcc | |
| | | aacccggtaagacacgacttatcgccactggcagcagccactggtaacaggattagcaga | |
| | | gcgaggtatgtaggcggtgctacagagttcttgaagtggtggcctaactacggctacacta | |
| | | gaaggacagtatttggtatctgcgctctgctgaagccagttaccttcggaaaaagagttggt | |
| | | agctcttgatccggcaaacaaaccaccgctggtagcggtggtttttttgtttgcaagcagcag | |
| | | attacgcgcagaaaaaaaggatctcaagaagatcctttgatcttttctacggggtctgacgct | |
| | | cagtggaacgaaaactcacgttaagggattttggtcatgagattatcaaaaaggatcttcac | |
| | | ctagatccttttaaattaaaaatgaagttttaaatcaatctaaagtatatatgagtaaacttgg | |
| | | tctgacagttaccaatgcttaatcagtgaggcacctatctcagcgatctgtctatttcgttcatc | |
| | | catagttgcctgactccccgtcgtgtagataactacgatacgggagggcttaccatctggccc | |
| | | cagtgctgcaatgataccgcgagacccacgctcaccggctccagatttatcagcaataaacc | |
| | | agccagccggaagggccgagcgcagaagtggtcctgcaactttatccgcctccatccagtc | |
| | | tattaattgttgccgggaagctagagtaagtagttcgccagttaatagtttgcgcaacgttgtt | |
| | | gccattgctacaggcatcgtggtgtcacgctcgtcgtttggtatggcttcattcagctccggtt | |
| | | cccaacgatcaaggcgagttacatgatcccccatgttgtgcaaaaaagcggttagctccttc | |
| | | ggtcctccgatcgttgtcagaagtaagttggccgcagtgttatcactcatggttatggcagca | |
| | | ctgcataattctcttactgtcatgccatccgtaagatgcttttctgtgactggtgagtactcaac | |
| | | caagtcattctgagaatagtgtatgcggcgaccgagttgctcttgcccggcgtcaatacggg | |
| | | ataataccgcgccacatagcagaactttaaaagtgctcatcattggaaaacgttcttcgggg | |
| | | cgaaaactctcaaggatcttaccgctgttgagatccagttcgatgtaacccactcgtgcaccc | |
| | | aactgatcttcagcatcttttactttcaccagcgtttctgggtgagcaaaaacaggaaggcaa | |
| | | aatgccgcaaaaaagggaataagggcgacacggaaatgttgaatactcatactcttcctttt | |
| | | tcaatattattgaagcatttatcagggttattgtctcatgagcggatacatatttgaatgtattt | |
| | | agaaaaataaacaaataggggttccgcgcacatttccccgaaaagtgccacctgaacgaa | |
| | | gcatctgtgcttcattttgtagaacaaaaatgcaacgcgagagcgctaatttttcaaacaaa | |
| | | gaatctgagctgcatttttacagaacagaaatgcaacgcgaaagcgctattttaccaacgaa | |
| | | gaatctgtgcttcatttttgtaaaacaaaaatgcaacgcgagagcgctaatttttcaaacaaa | |
| | | gaatctgagctgcatttttacagaacagaaatgcaacgcgagagcgctattttaccaacaaa | |
| | | gaatctatacttcttttttgttctacaaaaatgcatcccgagagcgctatttttctaacaaagca | |
| | | tcttagattactttttttctcctttgtgcgctctataatgcagtctcttgataactttttgcact | |
| | | gtaggtccgttaaggttagaagaaggctactttggtgtctattttctcttccataaaaaaagcct | |
| | | gactccacttcccgcgtttactgattactagcgaagctgcgggtgcattttttcaagataaaggca | |
| | | tccccgattatattctataccgatgtggattgcgcatactttgtgaacagaaagtgatagcgtt | |
| | | gatgattcttcattggtcagaaaattatgaacggtttcttctattttgtctctatatactacgtat | |
| | | aggaaatgtttacattttcgtattgttttcgattcactctatgaatagttcttactacaatttttt | |
| | | tgtctaaagagtaatactagagataaacataaaaaatgtagaggtcgagtttagatgcaagtt | |
| | | caaggagcgaaaggtggatgggtaggttatatagggatatagcacagagatatatagcaa | |
| | | agagatacttttgagcaatgtttgtggaagcggtattcgcaatattttagtagctcgttacagt | |
| | | ccggtgcgtttttggttttttgaaagtgcgtcttcagagcgcttttggttttcaaaagcgctctg | |
| | | aagttcctatactttctagagaataggaacttcggaataggaacttcaaagcgtttccgaaa | |
| | | acgagcgcttccgaaaatgcaacgcgagctgcgcacatacagctcactgttcacgtcgcac | |
| | | ctatatctgcgtgttgcctgtatatatatatacatgagaagaacggcatagtgcgtgtttatgc | |
| | | ttaaatgcgtacttatatgcgtctatttatgtaggatgaaaggtagtctagtacctcctgtgat | |
| | | attatcccattccatgcggggtatcgtatgcttccttcagcactaccctttagctgttctatatgc | |
| | | tgccactcctcaattggattagtctcatccttcaatgctatcatttcctttgatattggatcatct | |
| | | aagaaaccattattatcatgacattaacctataaaaataggcgtatcacgaggccctttcgtc | |
| Plasmid | 62 | tgcttgcgccctatggatgtaccttttctgcttgtgatgccagccattaatctggcacgtatctg | Escherichia |
| | | tgacgctttgtattcgaaaggtaatgatgggttcaaccacgctggagaagaagtgatcaact | coli |
| | | acatcaaaagcctcgtagtccatccgctcgtggtgtaagagctcggcgcgcctgcaggtcg | |
| | | acaagcttgcggccgcataatgcttaagtcgaacagaaagtaatcgtattgtacacggccg | |
| | | cataatcgaaatttgacaattaatcatcggctcgtataatgtgtggaattgtgagcgctcaca | |
| | | attccccatcttagtatattagttaagtataagaaggagatatacatatggcagatctcaatt | |
| | | ggatatcgatggactttccgcagcaactcgaagcctgcgttaagcaggccaaccaggcgct | |
| | | gagccgttttatcgccccactgccctttcagaacactcccgtggtcgaaaccatgcagtatgg | |
| | | cgcattattaggtggtaagcgcctgcgacctttcctggtttatgccaccggtcatatgttcggc | |
| | | gttagcacaaacacgctggacgcacccgctgccgccgttgagtgtatccacgcttactcatta | |
| | | attcatgatgatttaccggcaatggatgatgacgatctgcgtcgcggtttgccaacctgccat | |
| | | gtgaagtttggcgaagcaaacgcgattctcgctggcgacgctttacaaacgctggcgttctc | |
| | | gattttaagcgatgccgatatgccggaagtgtcggaccgcgacagaatttcgatgatttctg | |
| | | aactggcgagcgccagtggtattgccggaatgtgcggtggtcaggcattagatttagacgc | |
| | | ggaaggcaaacacgtacctctggacgcgcttgagcgtattcatcgtcataaaaccggcgca | |
| | | ttgattcgcgccgccgttcgccttggtgcattaagcgccggagataaaggacgtcgtgctct | |
| | | gccggtactcgacaagtatgcagagagcatcggccttgccttccaggttcaggatgacatcc | |
| | | tggatgtggtgggagatactgcaacgttgggaaaacgccagggtgccgaccagcaacttg | |
| | | gtaaaagtacctaccctgcacttctgggtcttgagcaagcccggaagaaagcccgggatct | |
| | | gatcgacgatgcccgtcagtcgctgaaacaactggctgaacagtcactcgatacctcggca | |
| | | ctggaagcgctagcggactacatcatccagcgtaataaataactcgagtctggtaaagaaa | |
| | | ccgctgctgcgaaatttgaacgccagcacatggactcgtctactagcgcagcttaattaacct | |
| | | aggctgctgccaccgctgagcaataactagcataaccccttggggccaggcatcaaataaa | |
| | | acgaaaggctcagtcgaaagactgggcctttcgttttatctgttgtttgtcggtgaacgctctc | |
| | | cctctaaacgggtcttgaggggttttttgctgaaacctcaggcatttgagaagcacacggtca | |
| | | cactgcttccggtagtcaataaaccggtaaaccagcaatagacataagcggctatttaacg | |
| | | accctgccctgaaccgacgaccgggtcatcgtggccggatcttgcggcccctcggcttgaac | |
| | | gaattgttagacattatttgccgactaccttggtgatctcgcctttcacgtagtggacaaattct | |
| | | tccaactgatctgcgcgcgaggccaagcgatcttcttcttgtccaagataagcctgtctagctt | |
| | | caagtatgacgggctgatactgggccggcaggcgctccattgcccagtcggcagcgacatc | |
| | | cttcggcgcgattttgccggttactgcgctgtaccaaatgcgggacaacgtaagcactacatt | |
| | | tcgctcatcgccagcccagtcgggcggcgagttccatagcgttaaggtttcatttagcgcctc | |
| | | aaatagatcctgttcaggaaccggatcaaagagttcctccgccgctggacctaccaaggca | |
| | | acgctatgttctcttgcttttgtcagcaagatagccagatcaatgtcgatcgtggctggctcga | |
| | | agatacctgcaagaatgtcattgcgctgccattctccaaattgcagttcgcgcttagctggat | |
| | | aacgccacggaatgatgtcgtcgtgcacaacaatggtgacttctacagcgcggagaatctc | |
| | | gctctctccaggggaagccgaagtttccaaaaggtcgttgatcaaagctcgccgcgttgtttc | |
| | | atcaagccttacggtcaccgtaaccagcaaatcaatatcactgtgtggcttcaggccgccat | |
| | | ccactgcggagccgtacaaatgtacggccagcaacgtcggttcgagatggcgctcgatgac | |
| | | gccaactacctctgatagttgagtcgatacttcggcgatcaccgcttccctcatactcttcctttt | |
| | | tcaatattattgaagcatttatcagggttattgtctcatgagcggatacatatttgaatgtattt | |
| | | agaaaaataaacaaatagctagctcactcggtcgctacgctccgggcgtgagactgcggc | |
| | | gggcgctgcggacacatacaaagttacccacagattccgtggataagcaggggactaaca | |
| | | tgtgaggcaaaacagcagggccgcgccggtggcgtttttccataggctccgccctcctgcca | |
| | | gagttcacataaacagacgcttttccggtgcatctgtgggagccgtgaggctcaaccatgaa | |
| | | tctgacagtacgggcgaaacccgacaggacttaaagatccccaccgtttccggcgggtcgc | |
| | | tccctcttgcgctctcctgttccgaccctgccgtttaccggatacctgttccgcctttctccctta | |
| | | cgggaagtgtggcgctttctcatagctcacacactggtatctcggctcggtgtaggtcgttcgc | |
| | | tccaagctgggctgtaagcaagaactccccgttcagcccgactgctgcgccttatccggtaa | |
| | | ctgttcacttgagtccaacccggaaaagcacggtaaaacgccactggcagcagccattggt | |
| | | aactgggagttcgcagaggatttgtttagctaaacacgcggttgctcttgaagtgtgcgcca | |
| | | aagtccggctacactggaaggacagatttggttgctgtgctctgcgaaagccagttaccacg | |
| | | gttaagcagttccccaactgacttaaccttcgatcaaaccacctccccaggtggttttttcgttt | |
| | | acagggcaaaagattacgcgcagaaaaaaaggatctcaagaagatcctttgatcttttcta | |
| | | ctgaaccgctctagatttcagtgcaatttatctcttcaaatgtagcacctgaagtcagccccat | |
| | | acgatataagttgtaattctcatgttagtcatgccccgcgcccaccggaaggagctgactgg | |
| | | gttgaaggctctcaagggcatcggtcgagatcccggtgcctaatgagtgagctaacttacat | |
| | | taattgcgttgcgctcactgcccgctttccagtcgggaaacctgtcgtgccagctgcattaatg | |
| | | aatcggccaacgcgcggggagaggcggtttgcgtattgggcgccagggtggtttttcttttc | |
| | | accagtgagacgggcaacagctgattgcccttcaccgcctggccctgagagagttgcagca | |
| | | agcggtccacgctggtttgccccagcaggcgaaaatcctgtttgatggtggttaacggcgg | |
| | | gatataacatgagctgtcttcggtatcgtcgtatcccactaccgagatgtccgcaccaacgcg | |
| | | cagcccggactcggtaatggcgcgcattgcgcccagcgccatctgatcgttggcaaccagc | |
| | | atcgcagtgggaacgatgccctcattcagcatttgcatggtttgttgaaaaccggacatggc | |
| | | actccagtcgccttcccgttccgctatcggctgaatttgattgcgagtgagatatttatgccag | |
| | | ccagccagacgcagacgcgccgagacagaacttaatgggcccgctaacagcgcgatttgc | |
| | | tggtgacccaatgcgaccagatgctccacgcccagtcgcgtaccgtcttcatgggagaaaat | |
| | | aatactgttgatgggtgtctggtcagagacatcaagaaataacgccggaacattagtgcag | |
| | | gcagcttccacagcaatggcatcctggtcatccagcggatagttaatgatcagcccactgac | |
| | | gcgttgcgcgagaagattgtgcaccgccgctttacaggcttcgacgccgcttcgttctaccat | |
| | | cgacaccaccacgctggcacccagttgatcggcgcgagatttaatcgccgcgacaatttgc | |
| | | gacggcgcgtgcagggccagactggaggtggcaacgccaatcagcaacgactgtttgccc | |
| | | gccagttgttgtgccacgcggttgggaatgtaattcagctccgccatcgccgcttccacttttt | |
| | | cccgcgttttcgcagaaacgtggctggcctggttcaccacgcgggaaacggtctgataaga | |
| | | gacaccggcatactctgcgacatcgtataacgttactggtttcacattcaccaccctgaattg | |
| | | actctcttccgggcgctatcatgccataccgcgaaaggttttgcgccattcgatggtgtccgg | |
| | | gatctcgacgctctcccttatgcgactcctgcattaggaaatttgacaattaatcatcggctcg | |
| | | tataatgtgtggaattgtgagcgctcacaattcccctgtagaaataattttgtttaactttaata | |
| | | aggagatataccatggcaatgtcaaccgcttttccggtggcggcattagtaagtgccagcgc | |
| | | atcattttcctcttcagaaccccttccttctccaccaccgactacgactgcgacgaaacaacgc | |
| | | aacactacaaaccatcacagttctatctggggtgatcagtttctgacctaccacgaaagtga | |
| | | agacatcctgaaagaaaaactgctggttgacgaactgaaagaacaggtccgtaaagagct | |
| | | gatttcggcgtctgttgacgatcatctggaactgattgatgcgattcagagactgggggtgg | |
| | | cttactattatgaaaatgagatcgaggaggctctacagcacatctatgtaacctacggtgatc | |
| | | attggatacacactgacaacctgcaaagcatcagtctatggtttcgtttgttacgccagcaag | |
| | | gcttcaatgtttcgtcgggcatctttaagaaccatatgaacgacaaggggaacttcaaggag | |
| | | tctattagcaatgacgtccagggcatgttggaactgtatgaagccgcctatatgcgcgtcga | |
| | | gggtgaagaaatcttggactacgctctggaattcaccaaaaccaacttaggcattttagcga | |
| | | acgatccgtcatgcgatagcagtttacgttcgcagatccaacaagcactgaaacaaccactg | |
| | | cgaaaaagattgccgcgtttggaagccgtcaggtatatcccgatatataaacaacaggcgt | |
| | | gtcacaacgacgttctgctgaaactggcaaaacttgatttcaacgtgctccagtccatgcaca | |
| | | aaaaggagctttcccacatctgcaaatggtggaaggacttaaacatgcaggaaaaactgc | |
| | | cgtatgtgcgtgatcgactggtggaaggctacttctggatcttaggtatctactttgaacccca | |
| | | tcatgcgcgcacgcgcatgtttctgatcaaaacctgtatgtggctcgtagttatcgatgatac | |
| | | gttcgacaattacggtgcctatgaagaactggaacagtttaccgaggcggtagaacgttgg | |
| | | tcgatttcttgcttggatgaactcccggagtatatgaaactggtttatcaggagctggtcaac | |
| | | gttcatcaagagatggaggagagcctcgaaaaggaaggcaaggcgtttcagattcactac | |
| | | gtgaaggaaatggcgaaagaatgcacccgcagcttactgctggaagcgaaatggctgaa | |
| | | agatggctatatgccgacgctggaagaatacctcagcaatagcctgattacatgcgcctatg | |
| | | cagttatggtggcgcggtcatatgtgggacgtgaagatgttatgattcgtgaggagacgttt | |
| | | aaatgggtggcaacacatccgccgcttgtcaaagccagttgcctaattctgcgcctgatgga | |
| | | tgatattgcaacccataaagaagagcaggaacgcggccatgtggccagctccattgaatgt | |
| | | tacattcgcgagaatgtcggtgcgaccgaagaagaagcgtgtgaacttttctccaaacaga | |
| | | tcgaggatgcctggaaagttatcaatcgggaa | |
| Engine- | 63 | MKISWNGFSKKSYQERLELLKAQALLSPERQASLEKDEQMSVTVADQLS | |
| ered | | ENVVGTFSLPYSLVPEVLVNGQEYTVPYVTEEPSVVAAASYASKIIKRAGG | |
| S. | | FTAQVHQRQMIGQVALYQVANPKLAQEKIASKKAELLELANQAYPSIVKR | |
| pneum- | | GGGARDLHVEQIKGEPDFLVVYIHVDTQEAMGANMLNSMLEALKPVLEE | |
| oniae | | LSQGQSLMGILSNYATDSLVTASCRIAFRYLSRQKDQGREIAEKIALASQ | |
| HmgR | | FAQADPYRAATHNKGIFNGIDAILIATGNDWRAIEAGAHAFASRDGRYQ | |
| T188S | | GLSCWTLDLEREELVGEMTLPMPVATKGGSIGLNPRVALSHDLLGNPSA | |
| | | RELAQIIVSIGLAQNFAALKALVSTGIQQGHMKLQAKSLALLAGASESEV | |
| | | APLVERLISDKTFNLETAQRYLENLRS | |
| Engine- | 64 | MKISWNGFSKKSYQERLELLKAQALLSPERQASLEKDEQMSVTVADQLS | |
| ered | | ENVVGTFSLPYSLVPEVLVNGQEYTVPYVTEEPSVVAAASYASKIIKRAGG | |
| S. | | FTAQVHQRQMIGQVALYQVANPKLAQEKIASKKAELLELANQAYPSIVKR | |
| pneum- | | GGGARDLHVEQIKGEPDFLVVYIHVDTQEAMGANMLNTMLEALKPVLEE | |
| oniae | | LSQGQSLMGILSNYATDSLVTASCRIAFRYLSRQKDHGREIAEKIALASQ | |
| HmgR | | FAQADPYRAATHNKGIFNGIDAILIATGNDWRAIEAGAHAFASRDGRYQ | |
| Q236H | | GLSCWTLDLEREELVGEMTLPMPVATKGGSIGLNPRVALSHDLLGNPSA | |
| | | RELAQIIVSIGLAQNFAALKALVSTGIQQGHMKLQAKSLALLAGASESEV | |
| | | APLVERLISDKTFNLETAQRYLENLRS | |
| Engine- | 65 | MKISWNGFSKKSYQERLELLKAQALLSPERQASLEKDEQMSVTVADQLS | |
| ered | | ENVVGTFSLPYSLVPEVLVNGQEYTVPYVTEEPSVVAAASYASKIIKRAGG | |
| S. | | FTAQVHQRQMIGQVALYQVANPKLAQEKIASKKAELLELANQAYPSIVKR | |
| pneum- | | GGGARDLHVEQIKGEPDFLVVYIHVDTQEAMGANMLNTMLEALKPVLEE | |
| oniae | | LSQGQSLMGILSNYATDSLVTASCRIAFRYLSRQKDQGREIAEKIALASQ | |
| HmgR | | FAQADPYRAATHNKGIFNGIDAILIATGNDWRAIEAGAHAFASRDGRYQ | |
| G343D | | GLSCWTLDLEREELVGEMTLPMPVATKGGSIGLNPRVALSHDLLDNPSA | |
| | | RELAQIIVSIGLAQNFAALKALVSTGIQQGHMKLQAKSLALLAGASESEV | |
| | | APLVERLISDKTFNLETAQRYLENLRS | |
| Engine- | 66 | MSLPFLTSAPGKVIIFGEHSAVYNKPAVAASVSALRTYLLISESSAPDTIELDFPDISF | |
| ered | | NHKWSINDFNAITEDQVNSQKLAKAQQATDGLSQELVSLLDPLLAQLSESFHYHA | |
| S. | | AFCFLYMFVCLCPHAKNIKFSLKSTLPIGAGLGSSASISVSLALAMAYLGGLIGSNDL | |
| cere- | | EKLSENDKHIVNQWAFIGEKCIHGTPSGIDNAVATYGNALLFEKDSHNGTINTNNF | |
| visiae | | KFLDDFPAIPMILTYTRIPRSTKDLVARVRVLVTEKFPEVMKPILDAMGECALQGLEI | |
| MVK | | MTKLSKCKGTDDEAVETNNELYEQLLELIRINHGLLVSIGVSHPGLELIKNLSDDLRI | |
| E419D | | GSTKLTGAGGGGCSLTLLRRDITQEQIDSFKKKLQDDFSYETFETDLGGTGCCLLS | |
| | | AKNLNKDLKIKSLVFQLFDNKTTTKQQIDDLLLPGNTNLPWTS | |
| Engine- | 67 | MSLPFLTSAPGKVIIFGEHSAVYNKPAVAASVSALRTYLLISESSAPDTIELDFPDISF | |
| ered | | NHKWSINDFNAITEDQVNSQKLAKAQQATDGLSQELVSLLDPLLAQLSESFHYHA | |
| S. | | AFCFLYMFVCLCPHAKNIKFSLKSTLPIGAGLGSSASISVSLALAMAYLGGLIGSNDL | |
| cere- | | EKLSENDKHIVNQWAFIGEKCIHGTPSGIDNAVATYGNALLFEKDSHNGTINTNNF | |
| visiae | | KFLDDFPAIPMILTYTRIPRSTKDLVARVRVLVTEKFPEVMKPILDAMGECALQGLEI | |
| MVK | | MTKLSKCKGTDDEAVETNNELYEQLLELIRINHGLLVSIGVSHPGLELIKNLSDDLRI | |
| K402V | | GSTKLTGAGGGGCSLTLLRRDITQEQIDSFKKKLQDDFSYETFETDLGGTGCCLLS | |
| | | AVNLNKDLKIKSLVFQLFENKTTTKQQIDDLLLPGNTNLPWTS | |
| Engine- | 68 | MSLPFLTSAPGKVIIFGEHSAVYNKPAVAASVSALRTYLLISESSAPDTIELDFPDISF | |
| ered | | NHKWSINDFNAITGDQVNSQKLAKAQQATDGLSQELVSLLDPLLAQLSESFHYHA | |
| S. | | AFCFLYMFVCLCPHAKNIKFSLKSTLPIGAGLGSSASISVSLALAMAYLGGLIGSNDL | |
| cere- | | EKLSENDKHIVNQWAFIGEKCIHGTPSGIDNAVATYGNALLFEKDSHNGTINTNNF | |
| visiae | | KFLDDFPAIPMILTYTRIPRSTKDLVARVRVLVTEKFPEVMKPILDAMGECALQGLEI | |
| MVK | | MTKLSKCKGTDDEAVETNNELYEQLLELIRINHGLLVSIGVSHPGLELIKNLSDDLRI | |
| E73G | | GSTKLTGAGGGGCSLTLLRRDITQEQIDSFKKKLQDDFSYETFETDLGGTGCCLLS | |
| | | AKNLNKDLKIKSLVFQLFENKTTTKQQIDDLLLPGNTNLPWTS | |
| Engine- | 69 | MSLPFLTSAPGKVIIFGEHSAVYNKPAVAASVSALRTYLLISESSAPDTIELDFPDISF | |
| ered | | NHKWSINDFNAITEDQVNSQKLAKAQQATDGLSQELVSLLDPLLAQLSESFHYHA | |
| S. | | AFCFLYMFVCLCPHAKNIKFSLKSTLPIGAGLGSSASISVSLALAMAYLGGLIGSNDL | |
| cere- | | EKLSENDKHIVNQWAFIGEKCIHGTPSGIDNAVATYGNALLFEKDSHNGTINTNNF | |
| visiae | | KFLDDFPAIPMILTYTRIPRSTKDLVARVRVLVTEKFPEVMKPILDAMGECALQGLEI | |
| MVK | | MTKLSKCKGTDDEAVETNNELYEQLLELIRINHGLLVSIGVSHPGLELIKNLSDDLRI | |
| G393V | | GSTKLTGAGGGGCSLTLLRRDITQEQIDSFKKKLQDDFSYETFETDLGVTGCCLLS | |
| | | AKNLNKDLKIKSLVFQLFENKTTTKQQIDDLLLPGNTNLPWTS | |
| Engine- | 70 | MSLPFLTSAPGKVIIFGEHSAVYNKPAVAASVSALRTYLLISESSAPDTIELDFPDISF | |
| ered | | NHKWSINDFNAITEDQVNSQKLAKAQQATDGLSQELVSLLDPLLAQLSESFHYHA | |
| S. | | AFCFLYMFVCLCPHAKNIKFSLKSTLPIGAGLGSSASISVSLALAMAYLGGLIGSNDL | |
| cere- | | EKVSENDKHIVNQWAFIGEKCIHGTPSGIDNAVATYGNALLFEKDSHNGTINTNNF | |
| visiae | | KFLDDFPAIPMILTYTRIPRSTKDLVARVRVLVTEKFPEVMKPILDAMGECALQGLEI | |
| MVK | | MTKLSKCKGTDDEAVETNNELYEQLLELIRINHGLLVSIGVSHPGLELIKNLSDDLRI | |
| L175V | | GSTKLTGAGGGGCSLTLLRRDITQEQIDSFKKKLQDDFSYETFETDLGGTGCCLLS | |
| | | AKNLNKDLKIKSLVFQLFENKTTTKQQIDDLLLPGNTNLPWTS | |
| Engine- | 71 | MSLPFLTSAPGKVIIFGEHSAVYNKPAVAASVSALRTYLLISESSAPDTIELDFPDISF | |
| ered | | NHKWSINDFNAITEEQVNSQKLAKAQQATDGLSQELVSLLDPLLAQLSESFHYHAA | |
| S. | | FCFLYMFVCLCPHAKNIKFSLKSTLPIGAGLGSSASISVSLALAMAYLGGLIGSNDLE | |
| cere- | | KLSENDKHIVNQWAFIGEKCIHGTPSGIDNAVATYGNALLFEKDSHNGTINTNNFK | |
| visiae | | FLDDFPAIPMILTYTRIPRSTKDLVARVRVLVTEKFPEVMKPILDAMGECALQGLEIM | |
| MVK | | TKLSKCKGTDDEAVETNNELYEQLLELIRINHGLLVSIGVSHPGLELIKNLSDDLRIG | |
| D74E | | STKLTGAGGGGCSLTLLRRDITQEQIDSFKKKLQDDFSYETFETDLGGTGCCLLSA | |
| | | KNLNKDLKIKSLVFQLFENKTTTKQQIDDLLLPGNTNLPWTS | |
| HmgR3 | 72 | MSPEERLASLLNEGQISADTKKEFENTALSSQIANHMIENQISETEVPMGVGLHLTV | |
| F102I | | DETDYLVPMATEEPSVIAALSNGAKIAQGFKTVNQQRLMRGQIVIYDVADAESLID | |
| | | ELQVRETEIFQQAELSYPSIVKRGGGLRDLQYRAFDESFVSVDFLVDVKDAMGANI | |
| | | VNAMLEGVAELFREWFAEQKILFSILSNYATESVVTMKTAIPVSRLSKGSNGREIAE | |
| | | KIVLASRYASLDPYRAVTHNKGIMNGIEAVVLATGNDTRAVSASCHAFAVKEGRYQ | |
| | | GLTSWTLDGEQLIGEISVPLALATVGGATKVLPKSQAAADLLAVTDAKELSRVVAA | |
| | | VGLAQNLAALRALVSEGIQKGHMALQARSLAMTVGATGKEVEAVAQQLKRQKTM | |
| | | NQDRALAILNDLRKQ | |
| HmgR3 | 73 | MSPEERLASLLNEGQISADTKKEFENTALSSQIANHMIENQISETEVPMGVGLHLTV | |
| Q188S | | DETDYLVPMATEEPSVIAALSNGAKIAQGFKTVNQQRLMRGQIVFYDVADAESLID | |
| | | ELQVRETEIFQQAELSYPSIVKRGGGLRDLQYRAFDESFVSVDFLVDVKDAMGANI | |
| | | VNAMLEGVAELFREWFAESKILFSILSNYATESVVTMKTAIPVSRLSKGSNGREIAE | |
| | | KIVLASRYASLDPYRAVTHNKGIMNGIEAVVLATGNDTRAVSASCHAFAVKEGRYQ | |
| | | GLTSWTLDGEQLIGEISVPLALATVGGATKVLPKSQAAADLLAVTDAKELSRVVAA | |
| | | VGLAQNLAALRALVSEGIQKGHMALQARSLAMTVGATGKEVEAVAQQLKRQKTM | |
| | | NQDRALAILNDLRKQ | |
| HmgR3 | 74 | MSPEERLASLLNEGQISADTKKEFENTALSSQIANHMIENQISETEVPMGVGLHLTV | |
| S236H | | DETDYLVPMATEEPSVIAALSNGAKIAQGFKTVNQQRLMRGQIVFYDVADAESLID | |
| | | ELQVRETEIFQQAELSYPSIVKRGGGLRDLQYRAFDESFVSVDFLVDVKDAMGANI | |
| | | VNAMLEGVAELFREWFAEQKILFSILSNYATESVVTMKTAIPVSRLSKGSNGREIAE | |
| | | KIVLASRYAHLDPYRAVTHNKGIMNGIEAVVLATGNDTRAVSASCHAFAVKEGRYQ | |
| | | GLTSWTLDGEQLIGEISVPLALATVGGATKVLPKSQAAADLLAVTDAKELSRVVAA | |
| | | VGLAQNLAALRALVSEGIQKGHMALQARSLAMTVGATGKEVEAVAQQLKRQKTM | |
| | | NQDRALAILNDLRKQ | |
| HmgR3 | 75 | MSPEERLASLLNEGQISADTKKEFENTALSSQIANHMIENQISETEVPMGVGLHLTV | |
| Q343D | | DETDYLVPMATEEPSVIAALSNGAKIAQGFKTVNQQRLMRGQIVFYDVADAESLID | |
| | | ELQVRETEIFQQAELSYPSIVKRGGGLRDLQYRAFDESFVSVDFLVDVKDAMGANI | |
| | | VNAMLEGVAELFREWFAEQKILFSILSNYATESVVTMKTAIPVSRLSKGSNGREIAE | |
| | | KIVLASRYASLDPYRAVTHNKGIMNGIEAVVLATGNDTRAVSASCHAFAVKEGRYQ | |
| | | GLTSWTLDGEQLIGEISVPLALATVGGATKVLPKSQAAADLLAVTDAKELSRVVAA | |
| | | VGLADNLAALRALVSEGIQKGHMALQARSLAMTVGATGKEVEAVAQQLKRQKTM | |
| | | NQDRALAILNDLRKQ | |
| HmgR1 | 76 | MKISWNGFSKKSYQERLELLKAQALLSPERQASLEKDEQMSVTVADQLSENVVGT | |
| T102I | | FSLPYSLVPEVLVNGQEYTVPYVTEEPSVVAAASYASKIIKRAGGFIAQVHQRQMIG | |
| | | QVALYQVANPKLAQEKIASKKAELLELANQAYPSIVKRGGGARDLHVEQIKGEPDF | |
| | | LVVYIHVDTQEAMGANMLNTMLEALKPVLEELSQGQSLMGILSNYATDSLVTASCR | |
| | | IAFRYLSRQKDQGREIAEKIALASQFAQADPYRAATHNKGIFNGIDAILIATGNDWR | |
| | | AIEAGAHAFASRDGRYQGLSCWTLDLEREELVGEMTLPMPVATKGGSIGLNPRVAL | |
| | | SHDLLGNPSARELAQIIVSIGLAQNFAALKALVSTGIQQGHMKLQAKSLALLAGASE | |
| | | SEVAPLVERLISDKTFNLETAQRYLENLRS | |
| HmgR2 | 77 | MAADQLVKTEVTKKSFTAPVQKASTPVLTNKTVISGSKVKSLSSAQSSSSGPSSSS | |
| K102I | | EEDDSRDIESLDKKIRPLEELEALLSSGNTKQLKNKEVAALVIHGILPLYALEKKLGD | |
| | | TTRAVAVRRKALSILAEAPVLASDRLPYKNYDYDRVFGACCENVIGYMPLPVGVIGP | |
| | | LVIDGTSYHIPMATTEGCLVASAMRGCKAINAGGGATTVLTKDGMTRGPVVRFPTL | |
| | | KRSGACKIWLDSEEGQNAIKKAFNSTSRFARLQHIQTCLAGDLLFMRFRTTTGDA | |
| | | MGMNMISKGVEYSLKQMVEEYGWEDMEVVSVSGNYCTDKKPAAINWIEGRGKS | |
| | | VVAEATIPGDVVRKVLKSDVSALVELNIAKNLVGSAMAGSVGGFNAHAANLVTAVF | |
| | | LALGQDPAQNVESSNCITLMKEVDGDLRISVSMPSIEVGTIGGGTVLEPQGAMLDL | |
| | | LGVRGPHATAPGTNARQLARIVACAVLAGELSLCAALAAGHLVQSHMTHNRKPAEP | |
| | | TKPNNLDATDINRLKDGSVTCIKS | |
| HmgR2 | 78 | MAADQLVKTEVTKKSFTAPVQKASTPVLTNKTVISGSKVKSLSSAQSSSSGPSSSS | |
| G188S | | EEDDSRDIESLDKKIRPLEELEALLSSGNTKQLKNKEVAALVIHGKLPLYALEKKLG | |
| | | DTTRAVAVRRKALSILAEAPVLASDRLPYKNYDYDRVFGACCENVIGYMPLPVGVIG | |
| | | PLVIDGTSYHIPMATTESCLVASAMRGCKAINAGGGATTVLTKDGMTRGPVVRFPT | |
| | | LKRSGACKIWLDSEEGQNAIKKAFNSTSRFARLQHIQTCLAGDLLFMRFRTTTGDA | |
| | | MGMNMISKGVEYSLKQMVEEYGWEDMEVVSVSGNYCTDKKPAAINWIEGRGKS | |
| | | VVAEATIPGDVVRKVLKSDVSALVELNIAKNLVGSAMAGSVGGFNAHAANLVTAVF | |
| | | LALGQDPAQNVESSNCITLMKEVDGDLRISVSMPSIEVGTIGGGTVLEPQGAMLDL | |
| | | LGVRGPHATAPGTNARQLARIVACAVLAGELSLCAALAAGHLVQSHMTHNRKPAEP | |
| | | TKPNNLDATDINRLKDGSVTCIKS | |
| HmgR2 | 79 | MAADQLVKTEVTKKSFTAPVQKASTPVLTNKTVISGSKVKSLSSAQSSSSGPSSSS | |
| W236H | | EEDDSRDIESLDKKIRPLEELEALLSSGNTKQLKNKEVAALVIHGKLPLYALEKKLG | |
| | | DTTRAVAVRRKALSILAEAPVLASDRLPYKNYDYDRVFGACCENVIGYMPLPVGVIG | |
| | | PLVIDGTSYHIPMATTEGCLVASAMRGCKAINAGGGATTVLTKDGMTRGPVVRFPT | |
| | | LKRSGACKIHLDSEEGQNAIKKAFNSTSRFARLQHIQTCLAGDLLFMRFRTTTGDA | |
| | | MGMNMISKGVEYSLKQMVEEYGWEDMEVVSVSGNYCTDKKPAAINWIEGRGKS | |
| | | VVAEATIPGDVVRKVLKSDVSALVELNIAKNLVGSAMAGSVGGFNAHAANLVTAVF | |
| | | LALGQDPAQNVESSNCITLMKEVDGDLRISVSMPSIEVGTIGGGTVLEPQGAMLDL | |
| | | LGVRGPHATAPGTNARQLARIVACAVLAGELSLCAALAAGHLVQSHMTHNRKPAEP | |
| | | TKPNNLDATDINRLKDGSVTCIKS | |
| HmgR2 | 80 | MAADQLVKTEVTKKSFTAPVQKASTPVLTNKTVISGSKVKSLSSAQSSSSGPSSSS | |
| P343D | | EEDDSRDIESLDKKIRPLEELEALLSSGNTKQLKNKEVAALVIHGKLPLYALEKKLG | |
| | | DTTRAVAVRRKALSILAEAPVLASDRLPYKNYDYDRVFGACCENVIGYMPLPVGVIG | |
| | | PLVIDGTSYHIPMATTEGCLVASAMRGCKAINAGGGATTVLTKDGMTRGPVVRFPT | |
| | | LKRSGACKIWLDSEEGQNAIKKAFNSTSRFARLQHIQTCLAGDLLFMRFRTTTGDA | |
| | | MGMNMISKGVEYSLKQMVEEYGWEDMEVVSVSGNYCTDKKPAAINWIEGRGKS | |
| | | VVAEATIDGDVVRKVLKSDVSALVELNIAKNLVGSAMAGSVGGFNAHAANLVTAV | |
| | | FLALGQDPAQNVESSNCITLMKEVDGDLRISVSMPSIEVGTIGGGTVLEPQGAMLD | |
| | | LLGVRGPHATAPGTNARQLARIVACAVLAGELSLCAALAAGHLVQSHMTHNRKPAE | |
| | | PTKPNNLDATDINRLKDGSVTCIKS | |
| aceto- | 39 | MSQNVYIVSTARTPIGSFQGSLSSKTAVELGAVALKGALAKVPELDASKDFDEIIFG | |
| acetyl- | | NVLSANLGQAPARQVALAAGLSNHIVASTVNKVCASAMKAIILGAQSIKCGNADV | |
| CoA | | VVAGGCESMTNAPYYMPAARAGAKFGQTVLVDGVERDGLNDAYDGLAMGVHAEK | |
| thio- | | CARDWDITREQQDNFAIESYQKSQKSQKEGKFDNEIVPVTIKGFRGKPDTQVTKD | |
| lase, | | EEPARLHVEKLRSARTVFQKENGTVTAANASPINDGAAAVILVSEKVLKEKNLKPLA | |
| S. | | IIKGWGEAAHQPADFTWAPSLAVPKALKHAGIEDINSVDYFEFNEAFSVVGLVNTK | |
| cere- | | ILKLDPSKVNVYGGAVALGHPLGCSGARVVVTLLSILQQEGGKIGVAAICNGGGGA | |
| visiae | | SSIVIEKI | |
| Hmg-CoA | 40 | MKLSTKLCWCGIKGRLRPQKQQQLHNTNLQMTELKKQKTAEQKTRPQNVGIKGIQ | |
| synthase | | IYIPTQCVNQSELEKFDGVSQGKYTIGLGQTNMSFVNDREDIYSMSLTVLSKLIKSY | |
| S. | | NIDTNKIGRLEVGTETLIDKSKSVKSVLMQLFGENTDVEGIDTLNACYGGTNALFN | |
| cere- | | SLNWIESNAWDGRDAIVVCGDIAIYDKGAARPTGGAGTVAMWIGPDAPIVFDSVR | |
| visiae | | ASYMEHAYDFYKPDFTSEYPYVDGHFSLTCYVKALDQVYKSYSKKAISKGLVSDPA | |
| | | GSDALNVLKYFDYNVFHVPTCKLVTKSYGRLLYNDFRANPQLFPEVDAELATRDYD | |
| | | ESLTDKNIEKTFVNVAKPFHKERVAQSLIVPTNTGNMYTASVYAAFASLLNYVGSD | |
| | | DLQGKRVGLFSYGSGLAASLYSCKIVGDVQHIIKELDITNKLAKRITETPKDYEAAIE | |
| | | LRENAHLKKNFKPQGSIEHLQSGVYYLTNIDDKFRRSYDVKK | |

The invention also provides the following numbered embodiment paragraphs:

### A—Phytoene Synthase:

A1. A phytoene synthase (PYS) enzyme wherein the PYS enzyme has an amino acid sequence of a PYS enzyme from:
Pantoea sp; Daucus sp; Enterobacter sp; Synechocystis sp; Arthrospira sp; Cucumis sp; or Staphylococcus sp;
   or has an amino acid sequence with at least 80%, 85%, 90%, 92%, 94%, 96%, 98%, 99% or 100% similarity to a PYS enzyme from:
Pantoea sp; Daucus sp; Enterobacter sp; Synechocystis sp; Arthrospira sp; Cucumis sp; or Staphylococcus sp.

A2. The PYS enzyme of embodiment A1 wherein:
The Pantoea species is Pantoea ananatis;
The Daucus species is Daucus carota;
The Enterobacter species is Enterobacter agglomerans;
The Synechocystis species is selected from Synechocystis aquatilis, Synechocystis bourrellyi, Synechocystis fuscopigmentosa, Synechocystis limnetica, Synechocystis minuscula, Synechocystis nigrescens, Synechocystis pevalekii, Synechocystis salina, Synechocystis sp. (strain PCC 6803/Kazusa), and Synechocystis trididemni;
The Arthrospira species is Arthrospira platensis;
The Cucumis species is Cucumis melo; or
The Staphylococcus species is Staphylococcus aureus strain bovine RF122/ET3-1).

A3. The PYS enzyme of any of embodiments A1 and A2 wherein the PYS enzyme has an amino acid sequence of any of SEQ ID NO: 1-7 or has an amino acid sequence with at least 80%, 85%, 90%, 92%, 94%, 96%, 98%, 99% or 100% similarity to any of SEQ ID NO: 1-7.

A4. The PYS enzyme of any of embodiments A1-A3 wherein the PYS enzyme is from Enterobacter species, optionally from Enterobacter agglomerans, optionally has an amino acid sequence of SEQ ID NO: 3 or has an amino acid sequence with at least 80%, 85%, 90%, 92%, 94%, 96%, 98%, 99% or 100% similarity to SEQ ID NO: 3.

A5. The PYS enzyme of any of embodiments A1-A4 wherein the PYS enzyme is from:
Pantoea ananatis and has an amino acid sequence of SEQ ID NO: 1;
Daucus carota and has an amino acid sequence of SEQ ID NO: 2;
Enterobacter agglomerans and has an amino acid sequence of SEQ ID NO: 3;
Synechocystis species has an amino acid sequence of SEQ ID NO: 4;
Arthrospira platensis and has an amino acid sequence of SEQ ID NO: 5;
Cucumis melo and has an amino acid sequence of SEQ ID NO: 6; or
Staphylococcus aureus strain bovine RF122/ET3-1) and has an amino acid sequence of SEQ ID NO: 7;
   or has an amino acid sequence with at least 80%, 85%, 90%, 92%, 94%, 96%, 98%, 99% or 100% similarity to any of the above sequences.

A6. A phytoene synthase enzyme (CrtB) wherein the CrtB has an amino acid sequence from Agrobacterium aurantiacum or Pantoea agglomerans.

A7. The phytoene synthase enzyme of embodiment A6 wherein the CrtB from Agrobacterium aurantiacum has a sequence of SEQ ID NO: 51, and/or the CrtB from Pantoea agglomerans has a sequence of SEQ ID NO: 52, or has a sequence with at least 80%, 85%, 90%, 92%, 94%, 96%, 98%, 99% or 100% similarity to SEQ ID NO: 51 or 52.

### B—GGPPS

B1. A geranylgeranyl diphosphate synthase (GGPPS) wherein the GGPPS enzyme has an amino acid sequence of a GGPPS enzyme from:
Saccharomyces sp; Capsicum sp; Abies sp; Penicillium sp; Mucor sp; Croton sp; Hevea sp; Pseudescherichia sp; or Enterobacter sp;
   or has an amino acid sequence with at least 80%, 85%, 90%, 92%, 94%, 96%, 98%, 99% or 100% similarity to a GGPPS enzyme from:
Saccharomyces sp; Capsicum sp; Abies sp; Penicillium sp; Mucor sp; Croton sp; Hevea sp; Pseudescherichia sp; or Enterobacter sp.

B2. The GGPPS enzyme of embodiment B1 wherein:
The Saccharomyces sp is Saccharomyces cerevisiae;
The Capsicum sp is Capsicum annuum;
The Abies sp is Abies grandis;
The Penicillium sp is Penicillium paxilli;
The Mucor sp is Mucor lusitanicus;
The Croton sp is Croton sublyratus;
The Hevea sp is Hevea brasiliensis;
The Pseudescherichia sp is Pseudescherichia vulneris; or
The Enterobacter sp is Enterobacter agglomerans.

B3. The GGPPS enzyme of any of embodiments B1 or B2 wherein the GGPPS enzyme has an amino acid sequence of any of SEQ ID NO: 8-16 or has an amino acid sequence with at least 80%, 85%, 90%, 92%, 94%, 96%, 98%, 99% or 100% similarity to any of SEQ ID NO: 8-16.

B4. The GGPPS enzyme of any of embodiments B1-B3 wherein the GGPPS enzyme is from Hevea sp, optionally is from Hevea brasiliensis, optionally has an amino acid sequence of SEQ ID NO: 14 or has an amino acid sequence with at least 80%, 85%, 90%, 92%, 94%, 96%, 98%, 99% or 100% similarity to SEQ ID NO: 14.

B5. The GGPPS enzyme of any of embodiments B1 or B2 wherein the GGPPS enzyme is from:
Saccharomyces cerevisiae and has an amino acid sequence of SEQ ID NO: 8;
Capsicum annuum and has an amino acid sequence of SEQ ID NO: 9;
Abies grandis and has an amino acid sequence of SEQ ID NO: 10;
Penicillium paxillin is and has an amino acid sequence of SEQ ID NO: 11;
Mucor lusitanicus and has an amino acid sequence of SEQ ID NO: 12;
Croton sublyratus and has an amino acid sequence of SEQ ID NO: 13; or
Hevea brasiliensis and has an amino acid sequence of SEQ ID NO: 14;
Pseudescherichia vulneris and has an amino acid sequence of SEQ ID NO: 15;
Enterobacter agglomerans and has an amino acid sequence of SEQ ID NO: 16;
   or has an amino acid sequence with at least 80%, 85%, 90%, 92%, 94%, 96%, 98%, 99% or 100% similarity to any of the above sequences.

B6. A Geranylgeranyl diphosphate synthase enzyme wherein the enzyme is a CrtE enzyme from Archaeoglobus fulgidus, a CrtE enzyme from Taxus x media, a CrtE enzyme Xanthophyllomyces dendrorhous, or a CrtE enzyme from Blakeslea trispora.

B7. The Geranylgeranyl diphosphate synthase enzyme of embodiment B6 wherein the CrtE enzyme from Archaeoglobus fulgidus has a sequence of SEQ ID NO: 55, the CrtE enzyme from Taxus x media has a sequence of SEQ ID NO: 56, the CrtE enzyme Xanthophyllomyces dendrorhous has a sequence of SEQ ID NO: 57, or the CrtE enzyme from Blakeslea trispora has a sequence of SEQ ID NO: 58.

### C—Bisabolol Synthase

C1. A Bisabolol synthase enzyme wherein the Bisabolol synthase enzyme has an amino acid sequence of a Bisabolol synthase enzyme from:
Cynara sp; Artemisia sp; Eremanthus sp; Helianthus sp; or Matricaria sp;
   or has an amino acid sequence with at least 80%, 85%, 90%, 92%, 94%, 96%, 98%, 99% or 100% similarity to a Bisabolol synthase enzyme from:
Cynara sp; Artemisia sp; Eremanthus sp; Helianthus sp; or Matricaria sp.

C2. The Bisabolol synthase enzyme of embodiment C1 wherein:
The Cynara species is Cynara cardunculus;
The Artemisia species is Artemisia annua;
The Eremanthus species is Eremanthus erythropappus;
The Helianthus species is Helianthus annuus; or
The Matricaria species is Matricaria recutita.

C3. The Bisabolol synthase enzyme of any of embodiments C1 and C2 wherein the Bisabolol synthase enzyme has an amino acid sequence of any of SEQ ID NO: 25-30 or has an amino acid sequence with at least 80%, 85%, 90%, 92%, 94%, 96%, 98%, 99% or 100% similarity to any of SEQ ID NO: 25-30.

C4. The Bisabolol synthase enzyme of any of embodiments C1 and C2 wherein the Bisabolol synthase enzyme is:
a) from Cynara sp;
b) from Cynara cardunculus; and/or
c) has an amino acid sequence of SEQ ID NO: 25 or has an amino acid sequence with at least 80%, 85%, 90%, 92%, 94%, 96%, 98%, 99% or 100% similarity to SEQ ID NO: 25; or
d) from Helianthus sp;
e) from Helianthus annuus; and/or
f) has an amino acid sequence of SEQ ID NO: 29 or has an amino acid sequence with at least 80%, 85%, 90%, 92%, 94%, 96%, 98%, 99% or 100% similarity to SEQ ID NO: 29.

C5. The Bisabolol synthase enzyme of any of embodiments C1 and C2 wherein the Bisabolol synthase enzyme is from:
Cynara cardunculus and has an amino acid sequence of SEQ ID NO: 25;
Artemisia annua and has an amino acid sequence of SEQ ID NO: 26 or 27;
Eremanthus erythropappus and has an amino acid sequence of SEQ ID NO: 28;
Helianthus annuus and has an amino acid sequence of SEQ ID NO: 29; or
Matricaria recutita and has an amino acid sequence of SEQ ID NO: 30.
   or has an amino acid sequence with at least 80%, 85%, 90%, 92%, 94%, 96%, 98%, 99% or 100% similarity to any of the above sequences.

### D—HMGR Enzymes (Including Engineered HmgR Enzymes Described Herein)

D1. A 3-hydroxy-3-methylglutaryl-CoA reductase enzyme wherein the enzyme has an amino acid sequence:
of a 3-hydroxy-3-methylglutaryl-CoA reductase enzyme from Streptococcus sp, Saccharomyces sp; or Enterococcus sp;
has an amino acid sequence with at least 80%, 85%, 90%, 92%, 94%, 96%, 98%, 99% or 100% similarity to a 3-hydroxy-3-methylglutaryl-CoA reductase from: Streptococcus sp; Saccharomyces sp; or Enterococcus sp;

D2. The -hydroxy-3-methylglutaryl-CoA reductase enzyme according to embodiment D1 wherein the Streptococcus sp is Streptococcus pneumoniae; Saccharomyces sp is Saccharomyces cerevisiae; or Enterococcus sp is Enterococcus faecalis.

D3. The 3-hydroxy-3-methylglutaryl-CoA reductase enzyme of any of embodiments D1 and D2 wherein the 3-hydroxy-3-methylglutaryl-CoA reductase has an amino acid sequence of any of SEQ ID NO: 19-21 or has an amino acid sequence with at least 80%, 85%, 90%, 92%, 94%, 96%, 98%, 99% or 100% similarity to any of SEQ ID NO: 19-21.

D4. The 3-hydroxy-3-methylglutaryl-CoA reductase enzyme of any of embodiments D1-D3 wherein the enzyme has at least 80%, 85%, 90%, 92%, 94%, 96%, 98%, 99% or 100% similarity to SEQ ID NO: 19, provided that the enzyme comprises a substitution at one or more positions selected from positions 188, 236, or 343, wherein said positions are numbered with reference to SEQ ID No: 19.

D5. The hydroxy-3-methylglutaryl-CoA reductase enzyme of embodiment D4 wherein the enzyme;
a) comprises a T188S substitution, a Q236H substitution, a T102I and/or a G343D substitution wherein said positions are numbered with reference to SEQ ID No: 19;
b) comprises a K102I, a G188S, a W236H and/or a P343D substitution wherein said positions are numbered with reference to SEQ ID NO 20;
c) comprises a F102I, a Q188S, a S236H and/or a Q343D substitution wherein said positions are numbered with reference to SEQ ID NO 21;
d) comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80.

### E—IDI

E1. An isopentenyl-diphosphate delta isomerase 1 enzyme, optionally wherein the IDI enzyme is from B. subtilis or E. coli, optionally wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41.

### F—DXS

1. A 1-deoxy-D-xylulose-5-phosphate synthase enzyme, optionally wherein the DXS enzyme is from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

### G—CRTI

G1. A phytoene desaturase enzyme wherein the phytoene desaturase enzyme has an amino acid sequence of a phytoene desaturase enzyme from:
Pantoea sp; Paracoccus sp; Pseudescherichia sp; Rhodobacter sp; Synechocystis sp; or Streptomyces sp;
or has an amino acid sequence with at least 80%, 85%, 90%, 92%, 94%, 96%, 98%, 99% or 100% similarity to a phytoene desaturase enzyme from:
Pantoea sp; Paracoccus sp; Pseudescherichia sp; Rhodobacter sp; Synechocystis sp; or Streptomyces sp.

G2. A phytoene desaturase enzyme wherein the phytoene desaturase enzyme converts phytoene to lycopene, optionally wherein the phytoene desaturase enzyme:
is from Pantoea sp or from Pantoea ananatis; and/or
has an amino acid sequence of SEQ ID NO: 33 or has an amino acid sequence with at least 80%, 85%, 90%, 92%, 94%, 96%, 98%, 99% or 100% similarity to SEQ ID NO: 33; or
is from Paracoccus sp; and/or
has an amino acid sequence of SEQ ID NO: 34 or has an amino acid sequence with at least 80%, 85%, 90%, 92%, 94%, 96%, 98%, 99% or 100% similarity to SEQ ID NO: 34;
is from Pseudescherichia sp, or Pseudescherichia vulneris and has an amino acid sequence of SEQ ID NO: 35 or has an amino acid sequence with at least 80%, 85%, 90%, 92%, 94%, 96%, 98%, 99% or 100% similarity to SEQ ID NO: 35;
is from Synechocystis sp or Synechocystis sp strain PCC 6714 and has an amino acid sequence of SEQ ID NO: 37 or has an amino acid sequence with at least 80%, 85%, 90%, 92%, 94%, 96%, 98%, 99% or 100% similarity to SEQ ID NO: 37; or
is from Streptomyces sp or Streptomyces griseus and has an amino acid sequence of SEQ ID NO: 38 or has an amino acid sequence with at least 80%, 85%, 90%, 92%, 94%, 96%, 98%, 99% or 100% similarity to SEQ ID NO: 38.

G3. A phytoene desaturase enzyme wherein the phytoene desaturase enzyme converts phytoene to neurosporene, optionally wherein the phytoene desaturase enzyme:
is from Rhodobacter sp; optionally from Rhodobacter capsulatus; and/or
has an amino acid sequence of SEQ ID NO: 36 or has an amino acid sequence with at least 80%, 85%, 90%, 92%, 94%, 96%, 98%, 99% or 100% similarity to SEQ ID NO: 36.

G4. The phytoene desaturase enzyme of any of embodiments G1 wherein:
The Pantoea sp. is Pantoea ananatis;
The Paracoccus sp. is Paracoccus sp strain N81106/MBIC 01143;
The Pseudescherichia sp. is Pseudescherichia vulneris;
The Rhodobacter sp. is Rhodobacter capsulatus;
The Synechocystis sp. is Synechocystis sp strain PCC 6714; and
The Streptomyces sp. is Streptomyces griseus.

G5. The phytoene desaturase enzyme of any of embodiments G1-G4 wherein the phytoene desaturase enzyme has an amino acid sequence of any of SEQ ID NO: 33-38 or has an amino acid sequence with at least 80%, 85%, 90%, 92%, 94%, 96%, 98%, 99% or 100% similarity to any of SEQ ID NO: 33-38.

G6. The phytoene desaturase enzyme of any of embodiments G1-G5 wherein the phytoene desaturase enzyme:
a) is from Paracoccus sp;
b) is from Paracoccus sp strain N81106/MBIC 01143; and/or
c) has an amino acid sequence of SEQ ID NO: 34 or has an amino acid sequence with at least 80%, 85%, 90%, 92%, 94%, 96%, 98%, 99% or 100% similarity to SEQ ID NO: 34; or
d) is from Rhodobacter sp;
e) is from Rhodobacter capsulatus; and/or
f) has an amino acid sequence of SEQ ID NO: 36 or has an amino acid sequence with at least 80%, 85%, 90%, 92%, 94%, 96%, 98%, 99% or 100% similarity to SEQ ID NO: 36.

G7. The phytoene desaturase enzyme of any of embodiments G1-G6 wherein the phytoene desaturase enzyme is from:
Pantoea ananatis and has an amino acid sequence of SEQ ID NO: 33; Paracoccus sp strain N81106/MBIC 01143 and has an amino acid sequence of SEQ ID NO: 34;
Pseudescherichia vulneris and has an amino acid sequence of SEQ ID NO: 35; Rhodobacter capsulatus and has an amino acid sequence of SEQ ID NO: 36; Synechocystis sp strain PCC 6714 and has an amino acid sequence of SEQ ID NO: 37; or
Streptomyces griseus and has an amino acid sequence of SEQ ID NO: 38, or has an amino acid sequence with at least 80%, 85%, 90%, 92%, 94%, 96%, 98%, 99% or 100% similarity to any of the above sequences.

### H—Mevalonate Pathway

H1. A mevalonate pathway expression system comprising one or more nucleic acids comprising any one or more or all of the following genes:

Acetoacetyl-CoA thiolase (HmgACT), Hydroxymethylglutaryl-CoA synthase (HmgS), 3-hydroxy-3-methylglutaryl-CoA reductase (HmgR), Mevalonate kinase (MVK), Phosphomevalonate kinase (PMK), Phosphomevalonate decarboxylase (PMD).

H2. The mevalonate pathway expression system of embodiment H1 wherein each of the genes are located on the same nucleic acid molecule, optionally located on the same plasmid.

H3. The mevalonate pathway expression system of embodiment H1 wherein at least one the genes is located on a different nucleic acid molecule to the rest of the genes, optionally wherein at least one gene is located on a first plasmid and at least a second gene is located on a second plasmid.

H4. The mevalonate pathway expression system of embodiment H1 to H3 wherein at least one of the genes is located in the nucleic acid of a host cell, optionally is located in a chromosome of a host cell.

H5. The mevalonate pathway expression system of any of embodiments H1-H4 wherein the:
HmgACT gene is from Enterococcus faecalis, optionally has a nucleotide sequence that encodes a protein with an amino acid sequence of SEQ ID NO: 17;
HmgS gene is from Enterococcus faecalis, optionally has a nucleotide sequence that encodes a protein with an amino acid sequence of SEQ ID NO: 18; HmgR gene encodes a protein that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
MVK gene is from S. cerevisiae, optionally has a nucleotide sequence that encodes a protein with an amino acid sequence of SEQ ID NO: 22; or:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   has an amino acid sequence of SEQ ID NO: 22;
PMK gene is from S. cerevisiae, optionally has a nucleotide sequence that encodes a protein with an amino acid sequence of SEQ ID NO: 23;
PMD gene is from S. cerevisiae, optionally has a nucleotide sequence that encodes a protein with an amino acid sequence of SEQ ID NO: 24;
or has a nucleic acid sequence that encodes a protein with an amino acid sequence with at least 80%, 85%, 90%, 92%, 94%, 96%, 98%, 99% or 100% similarity to any of the above sequences.

### I—Yeast Acetoacetyl-CoA Thiolase

I1. An acetoacetyl-CoA thiolase enzyme, optionally wherein the acetoacetyl-CoA thiolase enzyme has an amino acid sequence of an acetoacetyl-CoA thiolase enzyme from Saccharomyces cerevisiae, optionally has an amino acid sequence of SEQ ID NO: 39, or has an amino acid sequence with at least 80%, 85%, 90%, 92%, 94%, 96%, 98%, 99% or 100% similarity to SEQ ID NO: 39.

### J—Hmg-CoA Synthase

J1. A Hmg-CoA synthase enzyme, optionally wherein the Hmg-CoA synthase enzyme has an amino acid sequence of a Hmg-CoA synthase enzyme from Saccharomyces cerevisiae, optionally has an amino acid sequence of SEQ ID NO: 40, or has an amino acid sequence with at least 80%, 85%, 90%, 92%, 94%, 96%, 98%, 99% or 100% similarity to SEQ ID NO: 40.

### K—Nucleic Acids

K1. A polynucleotide encoding one or more proteins selected from the group comprising or consisting of:
a) a phytoene synthase enzyme according to any of embodiments A1-7;
b) a GGPPS enzyme according to any of embodiments B1-7;
c) a Bisabololol synthase enzyme according to any of embodiments C1-5;
d) a 3-hydroxy-3-methylglutaryl-CoA reductase enzyme according to any of embodiments D1-5;
e) an isopentenyl-diphosphate delta isomerase enzyme according to embodiment E1;
f) a 1-deoxy-D-xylulose-5-phosphate synthase enzyme according to embodiment F1;
g) a phytoene desaturase enzyme according to any of embodiments G1-G7;
h) a mevalonate pathway expression system according to any of embodiments H1-H5;
i) an acetoacetyl-CoA thiolase enzyme according to embodiment I1; and/or j) a Hmg-CoA synthase enzyme according to embodiment J1.

K2. The polynucleotide according to embodiment K1 wherein the polynucleotide is operably linked to a promoter, optionally wherein the promoter is:
a) a constitutive promoter;
b) an inducible promoter, optionally a galactose inducible promoter;
c) a weak promoter; and/or
d) a strong promoter.

K3. The polynucleotide according to embodiment K2 wherein the promoter is selected from the group comprising or consisting of:
a) a eukaryotic promoter, optionally a promoter that is operable in a eukaryote, optionally a yeast cell, a mammalian cell, an insect cell, optionally wherein the yeast cell is a Saccharomyces cerevisiae cell;
b) a prokaryotic promoter, optionally a promoter that is operable in a prokaryote, optionally an E. coli cell.

K4. The polynucleotide according to any of embodiments K1-K3 wherein the polynucleotide encodes 2 or more of said proteins, optionally encodes 2, 3, 4, 5, 6, 7, 8, 9, 10, or more of said proteins, optionally wherein at least 2 of the said proteins are operably linked to the same promoter to form an operon.

K5. The polynucleotide according to any of embodiments K1-K4 wherein the polynucleotide is located in the nucleic acid of a host cell, optionally located within a host chromosome of a host cell.

K6. The polynucleotide according to any of embodiments K1-K4 wherein the polynucleotide is part of a larger polynucleotide, optionally part of a vector, optionally wherein the vector is a plasmid.

K7. A polynucleotide encoding any one or more of:
a) a phytoene synthase enzyme according to any of embodiments A1-A7
b) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52;
c) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
d) a GGPPS enzyme according to any of embodiments B1-B7
e) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
f) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
g) a phytoene synthase enzyme according to any of embodiments A1-5 and a GGPPS enzyme according to any of embodiments B1-B7;
h) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
j) a phytoene synthase enzyme according to any of embodiments A1-A7 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
k) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
l) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
m) the mevalonate pathway expression system according to any of embodiments H1-H5;
n)
   i) a HmgACT enzyme;
   ii) a HmgS enzyme;
   iii) a HmgR enzyme (including engineered HmgR enzymes described herein);
   iv) a MVK enzyme (including engineered MVK enzymes described herein);
   v) a PMK enzyme; and
   vi) a PMD enzyme;
o)
   i) a HmgACT enzyme of SEQ ID NO 17;
   ii) a HmgS enzyme of SEQ ID NO: 18;
   iii) a HmgR enzyme (including engineered HmgR enzymes described herein) that:
      has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
      has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
      comprises a Q236H substitution, a T188S substitution a G343D and/or a T102I substitution wherein said positions are numbered with reference to SEQ ID No: 19;
      comprises a K102I substitution, a G188S substitution, a W236H substitution and/or a P343D substitution wherein said positions are numbered with reference to SEQ ID NO: 20;
      comprises a F102I substitution, a Q188S substitution, a S236H substitution and/or a Q343D substitution wherein said positions are numbered with reference to SEQ ID NO: 21;
      comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
      is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
         is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
         is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   iv) a MVK enzyme of SEQ ID NO: 22; or:
      has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
      comprises a E419D substitution, a K402V substitution, a E73G substitution, a G393V substitution, a L175V substitution, a D74E substitution, wherein said positions are numbered with reference to SEQ ID No: 22;
      has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
      has a nucleotide sequence that encodes a protein with an amino acid sequence of SEQ ID NO: 22;
         is from S. cerevisiae, optionally has a nucleotide sequence that encodes a protein with an amino acid sequence of SEQ ID NO: 22.
   v) a PMK enzyme of SEQ ID NO: 23; and
   vi) a PMD enzyme of SEQ ID NO: 24;
      or a protein with an amino acid sequence with at least 80%, 85%, 90%, 92%, 94%, 96%, 98%, 99% or 100% similarity to any of the above sequences;
p) a phytoene desaturase enzyme according to any of embodiments G1-G7;
q) a phytoene desaturase enzyme having an amino acid sequence of SEQ ID NO: 33-38 or having an amino acid sequence with at least 80%, 85%, 90%, 92%, 94%, 96%, 98%, 99% or 100% similarity to any of SEQ ID NO: 33-38;
r) a bisabolol synthase enzyme according to any of embodiments C1-C5;
s) a bisabolol synthase enzyme having an amino acid sequence of SEQ ID NO: 25-29 or having an amino acid sequence with at least 80%, 85%, 90%, 92%, 94%, 96%, 98%, 99% or 100% similarity to any of SEQ ID NO: 25-30;
t) an Acetoacetyl-CoA thiolase enzyme;
u) an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39;
v) a Hmg-CoA reductase enzyme;
w) a Hmg-CoA synthase enzyme, for example having an amino acid sequence of SEQ ID NO: 40 or having an amino acid sequence with at least 80%, 85%, 90%, 92%, 94%, 96%, 98%, 99% or 100% similarity to any of SEQ ID NO: 40;
x) an isopentenyl-diphosphate delta isomerase 1 enzyme, optionally wherein the IDI enzyme is from B. subtilis or E. coli, optionally wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
y) a 1-deoxy-D-xylulose-5-phosphate synthase enzyme, optionally wherein the DXS enzyme is from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

### L—Vectors

L1. A vector comprising one or more polynucleotides according to any of embodiments K1-K6.

L2. The vector according to embodiment L1 wherein the vector is a plasmid, optionally wherein the plasmid is a high copy number plasmid or a medium copy plasmid or a low copy number plasmid.

L3. The vector according to any of embodiments L1 or L2 wherein the vector comprises a polynucleotide encoding:
a) a phytoene synthase enzyme according to any of embodiments A1-A7
b) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52;
c) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
d) a GGPPS enzyme according to any of embodiments B1-B7
e) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
f) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
g) a phytoene synthase enzyme according to any of embodiments A1-5 and a GGPPS enzyme according to any of embodiments B1-B7;
h) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
j) a phytoene synthase enzyme according to any of embodiments A1-A7 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
k) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
l) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
m) the mevalonate pathway expression system according to any of embodiments H1-H5;
n)
   i) a HmgACT enzyme;
   ii) a HmgS enzyme;
   iii) a HmgR enzyme (including engineered HmgR enzymes described herein);
   iv) a MVK enzyme (including engineered MVK enzymes described herein);
   v) a PMK enzyme; and
   vi) a PMD enzyme;
o) i) a HmgACT enzyme of SEQ ID NO 17;
   ii) a HmgS enzyme of SEQ ID NO: 18;
   iii) a HmgR enzyme (including engineered HmgR enzymes described herein) that:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   v) a PMK enzyme of SEQ ID NO: 23; and
   vi) a PMD enzyme of SEQ ID NO: 24;
   or a protein with an amino acid sequence with at least 80%, 85%, 90%, 92%, 94%, 96%, 98%, 99% or 100% similarity to any of the above sequences;
p) a phytoene desaturase enzyme according to any of embodiments G1-G7;
q) a phytoene desaturase enzyme having an amino acid sequence of SEQ ID NO: 33-38 or having an amino acid sequence with at least 80%, 85%, 90%, 92%, 94%, 96%, 98%, 99% or 100% similarity to any of SEQ ID NO: 33-38;
r) a bisabolol synthase enzyme according to any of embodiments C1-C5;
s) a bisabolol synthase enzyme having an amino acid sequence of SEQ ID NO: 25-29 or having an amino acid sequence with at least 80%, 85%, 90%, 92%, 94%, 96%, 98%, 99% or 100% similarity to any of SEQ ID NO: 25-30;
t) an Acetoacetyl-CoA thiolase enzyme;
u) an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39;
v) a Hmg-CoA reductase enzyme;
w) a Hmg-CoA synthase enzyme, for example having an amino acid sequence of SEQ ID NO: 40 or having an amino acid sequence with at least 80%, 85%, 90%, 92%, 94%, 96%, 98%, 99% or 100% similarity to any of SEQ ID NO: 40;
x) an isopentenyl-diphosphate delta isomerase 1 enzyme, optionally wherein the IDI enzyme is from B. subtilis or E. coli, optionally wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
y) a 1-deoxy-D-xylulose-5-phosphate synthase enzyme, optionally wherein the DXS enzyme is from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

L4. The vector according to embodiment L1 or L2 wherein the vector comprises a polynucleotide encoding:
a) phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58; or b)
   i) a HmgACT enzyme;
   ii) a HmgS enzyme;
   iii) a HmgR enzyme (including engineered HmgR enzymes described herein);
   iv) a MVK enzyme (including engineered MVK enzymes described herein);
   v) a PMK enzyme;
   vi) a PMD enzyme; and
   vii) an isopentenyl-diphosphate delta isomerase 1 enzyme
      optionally wherein the HmgR enzyme:
      has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
      has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
      comprises a Q236H substitution, a T188S substitution a G343D and/or a T102I substitution wherein said positions are numbered with reference to SEQ ID No: 19;
      comprises a K102I substitution, a G188S substitution, a W236H substitution and/or a P343D substitution wherein said positions are numbered with reference to SEQ ID NO: 20;
      comprises a F102I substitution, a Q188S substitution, a S236H substitution and/or a Q343D substitution wherein said positions are numbered with reference to SEQ ID NO: 21;
      comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
      is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   optionally where the MVK enzyme:
is from S. cerevisiae, optionally has a nucleotide sequence that encodes a protein with an amino acid sequence of SEQ ID NO: 22;
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22; or
   has an amino acid sequence of SEQ ID NO: 22;
L3. The vector according to L1 or L2 wherein the vector comprises a polynucleotide encoding:
i) a HmgACT enzyme of SEQ ID NO 17;
ii) a HmgS enzyme of SEQ ID NO: 18;
iii) a HmgR enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID No: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
      is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
      is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
iv) a MVK enzyme that:
   is from S. cerevisiae, optionally has a nucleotide sequence that encodes a protein with an amino acid sequence of SEQ ID NO: 22; has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   has an amino acid sequence of SEQ ID NO: 22;
v) a PMK enzyme of SEQ ID NO: 23;
vi) a PMD enzyme of SEQ ID NO: 24;
vii) and an isopentenyl-diphosphate delta isomerase 1 enzyme, optionally wherein the isopentenyl-diphosphate delta isomerase 1 enzyme has an amino acid sequence with at least 80%, 85%, 90%, 92%, 94%, 96%, 98%, 99% or 100% similarity to any of the above sequences.

L4. A Phytoene expression system wherein the system comprises:
a) a first vector that comprises a polynucleotide encoding:
   i) phytoene synthase enzyme having an amino acid sequence of any of SEQ ID NO: 1-7, 51 o 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58; and
b) a second vector that comprises a polynucleotide encoding:
1)
   i) a HmgACT enzyme;
   ii) a HmgS enzyme;
   iii) a HmgR enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   iv) a MVK enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;
   v) a PMK enzyme;
   vi) a PMD enzyme; and
   vii) an isopentenyl-diphosphate delta isomerase 1 enzyme;
      or
2)
   i) a HmgACT enzyme of SEQ ID NO 17;
   ii) a HmgS enzyme of SEQ ID NO: 18;
   iii) a HmgR enzyme (including engineered HmgR enzymes described herein) of SEQ ID NO: 19, 20 or 21;
   iv) a MVK enzyme (including engineered MVK enzymes described herein) of SEQ ID NO: 22;
   v) a PMK enzyme of SEQ ID NO: 23;
   vi) a PMD enzyme of SEQ ID NO: 24;
   vii) and an isopentenyl-diphosphate delta isomerase 1 enzyme, optionally wherein the isopentenyl-diphosphate delta isomerase 1 enzyme has an amino acid sequence with at least 80%, 85%, 90%, 92%, 94%, 96%, 98%, 99% or 100% similarity to any of the above sequences.

### M—Phytoene Production System

M1. A phytoene production system comprising:
a) a phytoene synthase enzyme according to any of embodiments A1-A7; and
b) a GGPPS enzyme according to any of embodiments B1-B7.

M2. The phytoene production system according to embodiment M1 wherein the system further comprises:
c) a HmgACT enzyme, a HmgS enzyme, a HmgR enzyme (including engineered HmgR enzymes described herein), a MVK enzyme, a PMK enzyme, and a PMD enzyme.

M3. The phytoene production system according to any of embodiments M1 or M2 wherein the phytoene synthase enzyme has an amino acid sequence of SEQ ID NO: 3 or has an amino acid sequence with at least 80%, 85%, 90%, 92%, 94%, 96%, 98%, 99% or 100% similarity to any of SEQ ID NO: 3, and the GGPPS enzyme has an amino acid sequence of SEQ ID NO: 14 or has an amino acid sequence with at least 80%, 85%, 90%, 92%, 94%, 96%, 98%, 99% or 100% similarity to any of SEQ ID NO: 14.

M4. The phytoene production system according to any of embodiments M2 or M3 wherein:
the HmgACT is from Enterococcus faecalis, optionally has a nucleotide sequence that encodes a protein with an amino acid sequence of SEQ ID NO: 17;
the HmgS is from Enterococcus faecalis, optionally has a nucleotide sequence that encodes a protein with an amino acid sequence of SEQ ID NO: 18;
the HmgR:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
the MVK:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;
the PMK is from S. cerevisiae, optionally has a nucleotide sequence that encodes a protein with an amino acid sequence of SEQ ID NO: 23; and/or
the PMD is from S. cerevisiae, optionally has a nucleotide sequence that encodes a protein with an amino acid sequence of SEQ ID NO: 24;
   or has a nucleic acid sequence that encodes a protein with an amino acid sequence with at least 80%, 85%, 90%, 92%, 94%, 96%, 98%, 99% or 100% similarity to any of the above sequences.

M5. A phytoene production system comprising:
a) a polynucleotide encoding a phytoene synthase enzyme according to any of embodiments A1-A7, optionally wherein the polynucleotide is a polynucleotide according to any of embodiments K1-K7 or a vector according to any of L1-L3; and
b) a polynucleotide encoding a GGPPS enzyme according to any of embodiments B1-B7, optionally wherein the polynucleotide is a polynucleotide according to any of embodiments K1-K7.

M6. The phytoene production system according to embodiment M5 wherein the polynucleotide encoding a phytoene synthase encodes a phytoene synthase having an amino acid sequence of SEQ ID NO: 3 or has an amino acid sequence with at least 80%, 85%, 90%, 92%, 94%, 96%, 98%, 99% or 100% similarity to any of SEQ ID NO: 3, and wherein the polynucleotide encoding the GGPPS enzyme encodes a GGPPS enzyme having an amino acid sequence of SEQ ID NO: 14 or has an amino acid sequence with at least 80%, 85%, 90%, 92%, 94%, 96%, 98%, 99% or 100% similarity to any of SEQ ID NO: 14.

M7. The phytoene production system according to embodiment M1 to M5 wherein the system further comprises one or more polynucleotides that encode:
a HmgACT enzyme, a HmgS enzyme, a HmgR enzyme (including engineered HmgR enzymes described herein), a MVK enzyme, a PMK enzyme, and a PMD enzyme, optionally wherein the system comprises the mevalonate pathway expression system of any of embodiments H1-H5.

### N—Lycopene Production System

N1. A lycopene production system comprising:
a) a phytoene synthase enzyme according to any of embodiments A1-A7; and
b) a GGPPS enzyme according to any of embodiments B1-B7; and
c) a phytoene desaturase enzyme capable of converting phytoene to lycopene.

N2. The lycopene production system according to embodiment N1 wherein the system further comprises:
d) a HmgACT enzyme, a HmgS enzyme, a HmgR enzyme (including engineered HmgR enzymes described herein), a MVK enzyme, a PMK enzyme, and a PMD enzyme.

N3. The lycopene production system according to any of embodiments N1 or N2 wherein the phytoene synthase enzyme has an amino acid sequence of SEQ ID NO: 3 or has an amino acid sequence with at least 80%, 85%, 90%, 92%, 94%, 96%, 98%, 99% or 100% similarity to any of SEQ ID NO: 3, and the GGPPS enzyme has an amino acid sequence of SEQ ID NO: 14 or has an amino acid sequence with at least 80%, 85%, 90%, 92%, 94%, 96%, 98%, 99% or 100% similarity to any of SEQ ID NO: 14.

N4. The lycopene production system according to any of embodiments N2 or N3 wherein:
the HmgACT is from Enterococcus faecalis, optionally has a nucleotide sequence that encodes a protein with an amino acid sequence of SEQ ID NO: 17;
the HmgS is from Enterococcus faecalis, optionally has a nucleotide sequence that encodes a protein with an amino acid sequence of SEQ ID NO: 18;
the HmgR:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
the MVK:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;
the PMK is from S. cerevisiae, optionally has a nucleotide sequence that encodes a protein with an amino acid sequence of SEQ ID NO: 23; and/or
the PMD is from S. cerevisiae, optionally has a nucleotide sequence that encodes a protein with an amino acid sequence of SEQ ID NO: 24;
   or has a nucleic acid sequence that encodes a protein with an amino acid sequence with at least 80%, 85%, 90%, 92%, 94%, 96%, 98%, 99% or 100% similarity to any of the above sequences.

N5. The lycopene production system of any of embodiments N1-N4 wherein the phytoene desaturase enzyme capable of converting phytoene to lycopene is a phytoene desaturase enzyme according to any of embodiments G1-G7, optionally
is from Pantoea sp or from Pantoea ananatis; and/or
   has an amino acid sequence of SEQ ID NO: 33 or has an amino acid sequence with at least 80%, 85%, 90%, 92%, 94%, 96%, 98%, 99% or 100% similarity to SEQ ID NO: 33; or
   is from Paracoccus sp; and/or
   has an amino acid sequence of SEQ ID NO: 34 or has an amino acid sequence with at least 80%, 85%, 90%, 92%, 94%, 96%, 98%, 99% or 100% similarity to SEQ ID NO: 34;
   is from Pseudescherichia sp, or Pseudescherichia vulneris and has an amino acid sequence of SEQ ID NO: 35 or has an amino acid sequence with at least 80%, 85%, 90%, 92%, 94%, 96%, 98%, 99% or 100% similarity to SEQ ID NO: 35;
   is from Synechocystis sp or Synechocystis sp strain PCC 6714 and has an amino acid sequence of SEQ ID NO: 37 or has an amino acid sequence with at least 80%, 85%, 90%, 92%, 94%, 96%, 98%, 99% or 100% similarity to SEQ ID NO: 37; or
   is from Streptomyces sp or Streptomyces griseus and has an amino acid sequence of SEQ ID NO: 38 or has an amino acid sequence with at least 80%, 85%, 90%, 92%, 94%, 96%, 98%, 99% or 100% similarity to SEQ ID NO: 38.

N6. A lycopene production system comprising:
a) a polynucleotide encoding a phytoene synthase enzyme according to any of embodiments A1-A7, optionally wherein the polynucleotide is a polynucleotide according to any of embodiments K1-K7 or a vector according to any of L1-L3;
b) a polynucleotide encoding a GGPPS enzyme according to any of embodiments B1-B7, optionally wherein the polynucleotide is a polynucleotide according to any of embodiments K1-K7; and
c) a polynucleotide encoding a phytoene desaturase enzyme capable of converting phytoene to lycopene.

N7. The lycopene production system according to embodiment N6 wherein the polynucleotide encoding a phytoene synthase encodes a phytoene synthase having an amino acid sequence of SEQ ID NO: 3 or has an amino acid sequence with at least 80%, 85%, 90%, 92%, 94%, 96%, 98%, 99% or 100% similarity to any of SEQ ID NO: 3, and wherein the nucleic acid encoding the GGPPS enzyme encodes a GGPPS enzyme having an amino acid sequence of SEQ ID NO: 14 or has an amino acid sequence with at least 80%, 85%, 90%, 92%, 94%, 96%, 98%, 99% or 100% similarity to any of SEQ ID NO: 14.

N8. The lycopene production system according to embodiment N6 or N7 wherein the system further comprises one or more polynucleotides that encode:
a HmgACT enzyme, a HmgS enzyme, a HmgR enzyme (including engineered HmgR enzymes described herein), a MVK enzyme, a PMK enzyme, and a PMD enzyme, optionally wherein the system comprises the mevalonate pathway expression system of any of embodiments H1-H5.

N9. The lycopene production system according to any of embodiments N6-N8 wherein the phytoene desaturase enzyme that is capable of converting phytoene to lycopene is a phytoene desaturase enzyme according to any of embodiments G1-G7, optionally
is from Pantoea sp or from Pantoea ananatis; and/or
has an amino acid sequence of SEQ ID NO: 33 or has an amino acid sequence with at least 80%, 85%, 90%, 92%, 94%, 96%, 98%, 99% or 100% similarity to SEQ ID NO: 33; or
is from Paracoccus sp; and/or
has an amino acid sequence of SEQ ID NO: 34 or has an amino acid sequence with at least 80%, 85%, 90%, 92%, 94%, 96%, 98%, 99% or 100% similarity to SEQ ID NO: 34;
is from Pseudescherichia sp, or Pseudescherichia vulneris and has an amino acid sequence of SEQ ID NO: 35 or has an amino acid sequence with at least 80%, 85%, 90%, 92%, 94%, 96%, 98%, 99% or 100% similarity to SEQ ID NO: 35;
is from Synechocystis sp or Synechocystis sp strain PCC 6714 and has an amino acid sequence of SEQ ID NO: 37 or has an amino acid sequence with at least 80%, 85%, 90%, 92%, 94%, 96%, 98%, 99% or 100% similarity to SEQ ID NO: 37; or
is from Streptomyces sp or Streptomyces griseus and has an amino acid sequence of SEQ ID NO: 38 or has an amino acid sequence with at least 80%, 85%, 90%, 92%, 94%, 96%, 98%, 99% or 100% similarity to SEQ ID NO: 38.

### O—Neurosporene Production System

O1. A neurosporene production system comprising:
a) a phytoene synthase enzyme according to any of embodiments A1-A7; and
b) a GGPPS enzyme according to any of embodiments B1-B7; and
c) a phytoene desaturase enzyme capable of converting phytoene to neurosporene.

O2. The neurosporene production system according to embodiment O1 wherein the system further comprises:
d) a HmgACT enzyme, a HmgS enzyme, a HmgR enzyme (including engineered HmgR enzymes described herein), a MVK enzyme, a PMK enzyme, and a PMD enzyme.

O3. The neurosporene production system according to any of embodiments O1 or O2 wherein the phytoene synthase enzyme has an amino acid sequence of SEQ ID NO: 3 or has an amino acid sequence with at least 80%, 85%, 90%, 92%, 94%, 96%, 98%, 99% or 100% similarity to any of SEQ ID NO: 3, and the GGPPS enzyme has an amino acid sequence of SEQ ID NO: 14 or has an amino acid sequence with at least 80%, 85%, 90%, 92%, 94%, 96%, 98%, 99% or 100% similarity to any of SEQ ID NO: 14.

O4. The neurosporene production system according to any of embodiments O2 or 03 wherein:
the HmgACT is from Enterococcus faecalis, optionally has a nucleotide sequence that encodes a protein with an amino acid sequence of SEQ ID NO: 17;
the HmgS is from Enterococcus faecalis, optionally has a nucleotide sequence that encodes a protein with an amino acid sequence of SEQ ID NO: 18;
the HmgR:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
the MVK:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;
the PMK is from S. cerevisiae, optionally has a nucleotide sequence that encodes a protein with an amino acid sequence of SEQ ID NO: 23; and/or
the PMD is from S. cerevisiae, optionally has a nucleotide sequence that encodes a protein with an amino acid sequence of SEQ ID NO: 24;
   or has a nucleic acid sequence that encodes a protein with an amino acid sequence with at least 80%, 85%, 90%, 92%, 94%, 96%, 98%, 99% or 100% similarity to any of the above sequences.

O5. The neurosporene production system of any of embodiments O1-O4 wherein the phytoene desaturase enzyme capable of converting phytoene to neurosporene is a phytoene desaturase enzyme according to any of embodiments G1-G7, optionally is a phytoene desaturase enzyme having an amino acid sequence of SEQ ID NO: 36 or has an amino acid sequence with at least 80%, 85%, 90%, 92%, 94%, 96%, 98%, 99% or 100% similarity to SEQ ID NO: 36.

O6. A neurosporene production system comprising:
a) a polynucleotide encoding a phytoene synthase enzyme according to any of embodiments A1-A7, optionally wherein the polynucleotide is a polynucleotide according to any of embodiments K1-K7 or a vector of any of embodiments L1-L3;
b) a polynucleotide encoding a GGPPS enzyme according to any of embodiments B1-B7, optionally wherein the polynucleotide is a polynucleotide according to any of embodiments K1-K7 or a vector of any of embodiments L1-L3; and
c) a polynucleotide encoding a phytoene desaturase enzyme capable of converting phytoene to lycopene.

O7. The neurosporene production system according to embodiment O6 wherein the polynucleotide encoding a phytoene synthase encodes a phytoene synthase having an amino acid sequence of SEQ ID NO: 3 or has an amino acid sequence with at least 80%, 85%, 90%, 92%, 94%, 96%, 98%, 99% or 100% similarity to any of SEQ ID NO: 3, and wherein the polynucleotide encoding the GGPPS enzyme encodes a GGPPS enzyme having an amino acid sequence of SEQ ID NO: 14 or has an amino acid sequence with at least 80%, 85%, 90%, 92%, 94%, 96%, 98%, 99% or 100% similarity to any of SEQ ID NO: 14.

O8. The neurosporene production system according to embodiment O6 or O7 wherein the system further comprises one or more polynucleotides that encode:
a HmgACT enzyme, a HmgS enzyme, a HmgR enzyme (including engineered HmgR enzymes described herein), a MVK enzyme, a PMK enzyme, and a PMD enzyme, optionally wherein the system comprises the mevalonate pathway expression system of any of embodiments H1-H5.

O9. The neurosporene production system according to any of embodiments O6-O8 wherein the phytoene desaturase enzyme that is capable of converting phytoene to lycopene is a phytoene desaturase enzyme according to any of embodiments G1-G7, optionally is a phytoene desaturase enzyme having an amino acid sequence of SEQ ID NO: 36, or has an amino acid sequence with at least 80%, 85%, 90%, 92%, 94%, 96%, 98%, 99% or 100% similarity to SEQ ID NO: 36.

### P—Bisabolol Production System

P1. A bisabolol production system comprising a HmgR enzyme (including engineered HmgR enzymes described herein):
is from S. pneumoniae, optionally has a nucleotide sequence that encodes a protein with an amino acid sequence of SEQ ID NO: 19;
is from S. cerevisiae, optionally has a nucleotide sequence that encodes a protein with an amino acid sequence of SEQ ID NO: 20; or
is from Enterococcus faecalis, optionally has a nucleotide sequence that encodes a protein with an amino acid sequence of SEQ ID NO: 21;
has at least 80%, 85%, 90%, 92%, 94%, 96%, 98%, 99% or 100% similarity to SEQ ID NO: 19, provided that the enzyme comprises a substitution at one or more positions selected from positions 188, 236, or 343, wherein said positions are numbered with reference to SEQ ID No: 19;
comprises a T188S substitution, a Q236H substitution and/or a G343D substitution wherein said positions are numbered with reference to SEQ ID No: 19;
has at least 80%, 85%, 90%, 92%, 94%, 96%, 98%, 99% or 100% similarity to SEQ ID NO: 19, provided that the enzyme comprises at least one of the following: a serine at residue 188, a histidine at residue 236 and/or an aspartic acid at residue 343; or comprises or consists of a sequence of SEQ ID NO: 63, 64 or 65.

P2. A bisabolol production system comprising a bisabolol synthase selected from any one of:
A) a Bisabolol synthase enzyme from Cynara sp; Artemisia sp; Eremanthus sp; Helianthus sp; or Matricaria sp;
or has an amino acid sequence with at least 80%, 85%, 90%, 92%, 94%, 96%, 98%, 99% or 100% similarity to a Bisabolol synthase enzyme from:
   Cynara sp; Artemisia sp; Eremanthus sp; Helianthus sp; or Matricaria sp;
   Optionally wherein:
      The Cynara species is Cynara cardunculus;
      The Artemisia species is Artemisia annua;
   The Eremanthus species is Eremanthus erythropappus;
   The Helianthus species is Helianthus annuus; or
   The Matricaria species is Matricaria recutita;
B) a Bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30;
C) a Bisabolol synthase enzyme with an amino acid sequence of SEQ ID NO: 29 or has an amino acid sequence with at least 80%, 85%, 90%, 92%, 94%, 96%, 98%, 99% or 100% similarity to SEQ ID NO: 29;
Or
D) a Bisabolol synthase enzyme with an amino acid sequence of SEQ ID NO: 25 or has an amino acid sequence with at least 80%, 85%, 90%, 92%, 94%, 96%, 98%, 99% or 100% similarity to SEQ ID NO: 25.

P3. A bisabolol production system comprising a MVK enzyme (including engineered MVK enzymes described herein) that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
has a nucleotide sequence that encodes a protein with an amino acid sequence of SEQ ID NO: 22;
is from S. cerevisiae, optionally has an amino acid sequence of SEQ ID NO: 22;

P4. A bisabolol production system comprising:
A HmgR enzyme (including engineered HmgR enzymes described herein) according to paragraph P1, the bisabolol synthase according to claim P2, and the MVK enzyme according to paragraph P3.

P5. The bisabolol production system according to any of claims P1-P4 further comprising the following enzymes:
Acetoacetyl-CoA thiolase (HmgACT), optionally wherein the HmgACT protein is from Enterococcus faecalis, optionally has an amino acid sequence of SEQ ID NO: 17;
Hydroxymethylglutaryl-CoA synthase (HmgS), optionally wherein the HmgS protein is from Enterococcus faecalis, optionally has an amino acid sequence of SEQ ID NO: 18;
Phosphomevalonate kinase (PMK), optionally wherein the PMK protein is from S. cerevisiae, optionally has an amino acid sequence of SEQ ID NO: 23;
Phosphomevalonate decarboxylase (PMD), optionally wherein the PMD protein is from S. cerevisiae, optionally has an amino acid sequence of SEQ ID NO: 24;
isopentenyl-diphosphate delta isomerase 1 (IDI), optionally wherein the IDI enzyme is from B. subtilis or E. coli, optionally wherein the isopentenyl-diphosphate delta isomerase enzyme has an amino acid sequence of SEQ ID NO: 31, or 41; and
FPP synthase from E. coli, for example of SEQ ID NO: 42;
or an amino acid sequence with at least 80%, 85%, 90%, 92%, 94%, 96%, 98%, 99% or 100% similarity to any of the above sequences.

P6. A bisabolol production system comprising:
a) A nucleic acid that encodes a HmgR enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) A nucleic acid that encodes a bisabolol synthase selected from any one of:
i) a Bisabolol synthase enzyme from Cynara sp; Artemisia sp; Eremanthus sp; Helianthus sp; or Matricaria sp or that has an amino acid sequence with at least 80%, 85%, 90%, 92%, 94%, 96%, 98%, 99% or 100% similarity to a Bisabolol synthase enzyme from Cynara sp; Artemisia sp; Eremanthus sp; Helianthus sp; or Matricaria sp;
ii) a Bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30;
optionally
a Bisabolol synthase enzyme with an amino acid sequence of SEQ ID NO: 29 or has an amino acid sequence with at least 80%, 85%, 90%, 92%, 94%, 96%, 98%, 99% or 100% similarity to SEQ ID NO: 29; or or
a Bisabolol synthase enzyme with an amino acid sequence of SEQ ID NO: 25 or has an amino acid sequence with at least 80%, 85%, 90%, 92%, 94%, 96%, 98%, 99% or 100% similarity to SEQ ID NO: 25.
c) A nucleic acid that encodes a mevalonate kinase enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;

P7. The bisabolol production system according to embodiment P6 wherein the nucleic acid encoding a phytoene synthase encodes a phytoene synthase having an amino acid sequence of SEQ ID NO: 3 or has an amino acid sequence with at least 80%, 85%, 90%, 92%, 94%, 96%, 98%, 99% or 100% similarity to any of SEQ ID NO: 3, and wherein the nucleic acid encoding the GGPPS enzyme encodes a GGPPS enzyme having an amino acid sequence of SEQ ID NO:14 or has an amino acid sequence with at least 80%, 85%, 90%, 92%, 94%, 96%, 98%, 99% or 100% similarity to any of SEQ ID NO: 14.

P8. The bisabolol production system according to embodiment P6 or P7 wherein the system further comprises nucleic acids that encode:
a HmgACT enzyme, a HmgS enzyme, a HmgR enzyme (including engineered HmgR enzymes described herein), a MVK enzyme, a PMK enzyme, and a PMD enzyme, optionally wherein the system comprises the mevalonate pathway expression system of any of embodiments H1-H5.

P9. The bisabolol production system according to any of embodiments P6-P8 wherein the bisabolol synthase is a bisabolol synthase according to any of embodiments C1-C5, optionally is a bisabolol synthase having an amino acid sequence of SEQ ID NO: 25-30, or has an amino acid sequence with at least 80%, 85%, 90%, 92%, 94%, 96%, 98%, 99% or 100% similarity to SEQ ID NO: 25-30.

### Q—Cells

Q1. An engineered cell comprising one or more of the following:
a) the phytoene synthase according to any of embodiments A1-A7;
b) the GGPPS enzyme according to any of embodiments B1-B7;
c) the bisabolol synthase according to any of embodiments C1-C5;
d) the 3-hydroxy-3-methylglutaryl-CoA reductase enzyme according to any of embodiments D1-D5;
e) the isopentenyl-diphosphate delta isomerase enzyme according to any of embodiments E1;
f) the 1-deoxy-D-xylulose-5-phosphate synthase enzyme according to any of claims F1;
g) the phytoene desaturase enzyme according to any of embodiments G1-G7;
h) the mevalonate pathway expression system of any of embodiments H1-H5;
i) one or more polynucleotides according to any of embodiments K1-K&;
j) one or more vectors according to any of embodiments L1-L3;
k) the phytoene production system of any of embodiments M1-M7;
l) the lycopene production system of any of embodiments N1-N9;
m) the neurosporene production system of any of embodiments O1-O9; and/or
n) the bisabolol production system of any of embodiments P1-X.

Q2. The engineered cell according to embodiment Q1, wherein the cell is a prokaryotic cell, optionally a bacterial cell, optionally an Escherichia coli cell.

Q3. The engineered cell according to embodiment Q1, wherein the cell is a eukaryotic cell, optionally a yeast cell, optionally a Saccharomyces cerevisiae cell.

Q4. The engineered cell according to any of embodiments Q1-Q3 wherein the cell comprises a mevalonate pathway expression system, optionally the mevalonate pathway expression system according to any of embodiments H1-H5.

Q5. The engineered cell according to any of embodiments Q1-Q4 wherein the engineered cell is able to produce farnesyl diphosphate (FPP).

Q6. The engineered cell according to any of embodiments Q1-Q5 wherein the cell comprises: a polynucleotide encoding:
a) a phytoene synthase enzyme according to any of embodiments A1-A7;
b) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52;
c) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
d) a GGPPS enzyme according to any of embodiments B1-B7;
e) a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
f) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
g) a phytoene synthase enzyme according to any of embodiments A1-A7 and a GGPPS enzyme according to any of embodiments B1-B7;
h) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58;
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
j) a phytoene synthase enzyme according to any of embodiments A1-A7 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
k) a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
l) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
m) the mevalonate pathway expression system according to any of embodiments H1-H5;
n)
   i) a HmgACT enzyme;
   ii) a HmgS enzyme;
   iii) a HmgR enzyme (including engineered HmgR enzymes described herein), for example that:
      has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
      has at least 80%, 85%, 90%, 92%, 94%, 96%, 98%, 99% or 100% similarity to SEQ ID NO: 19, 20 or 21 provided that the enzyme
      comprises at least one of the following: a histidine at residue 236, a serine at residue 188, an aspartic acid at residue 343, and/or an isoleucine at position 102; or
      comprises a Q236H substitution, a T188S substitution a G343D and/or a T102I substitution wherein said positions are numbered with reference to SEQ ID No: 19;
      comprises a K102I substitution, a G188S substitution, a W236H substitution and/or a P343D substitution wherein said positions are numbered with reference to SEQ ID NO: 20;
      comprises a F102I substitution, a Q188S substitution, a S236H substitution and/or a Q343D substitution wherein said positions are numbered with reference to SEQ ID NO: 21;
      comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
      is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
      is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
      is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   iv) a MVK enzyme that:
      has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
      comprises a E419D substitution, a K402V substitution, a E73G substitution, a G393V substitution, a L175V substitution, a D74E substitution, wherein said positions are numbered with reference to SEQ ID No: 22;
      has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
      comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74; has a nucleotide sequence that encodes a protein with an amino acid sequence of SEQ ID NO: 22;
      is from S. cerevisiae, optionally has an amino acid sequence of SEQ ID NO: 22;
   v) a PMK enzyme; and
   vi) a PMD enzyme;
o) i) a HmgACT enzyme of SEQ ID NO 17;
   ii) a HmgS enzyme of SEQ ID NO: 18;
   iii) a HmgR enzyme that:
      has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
      has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
      comprises a Q236H substitution, a T188S substitution a G343D and/or a T102I substitution wherein said positions are numbered with reference to SEQ ID No: 19;
      comprises a K102I substitution, a G188S substitution, a W236H substitution and/or a P343D substitution wherein said positions are numbered with reference to SEQ ID NO: 20;
      comprises a F102I substitution, a Q188S substitution, a S236H substitution and/or a Q343D substitution wherein said positions are numbered with reference to SEQ ID NO: 21;
      comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
      is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
      is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
      is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   iv) a MVK enzyme that:
      has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
      comprises a E419D substitution, a K402V substitution, a E73G substitution, a G393V substitution, a L175V substitution, a D74E substitution, wherein said positions are numbered with reference to SEQ ID No: 22;
      has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
      comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74; has a nucleotide sequence that encodes a protein with an amino acid sequence of SEQ ID NO: 22;
      is from S. cerevisiae, optionally has an amino acid sequence of SEQ ID NO: 22;
   v) a PMK enzyme of SEQ ID NO: 23; and
   vi) a PMD enzyme of SEQ ID NO: 24;
   or has a nucleic acid sequence that encodes a protein with an amino acid sequence with at least 80%, 85%, 90%, 92%, 94%, 96%, 98%, 99% or 100% similarity to any of the above sequences;
p) a phytoene desaturase enzyme according to any of embodiments G1-G7;
q) a phytoene desaturase enzyme having an amino acid sequence of SEQ ID NO: 33-38 or having an amino acid sequence with at least 80%, 85%, 90%, 92%, 94%, 96%, 98%, 99% or 100% similarity to any of SEQ ID NO: 33-38;
r) a bisabolol synthase enzyme according to any of embodiments C1-C5;
s) a bisabolol synthase enzyme having an amino acid sequence of SEQ ID NO: 25-29 or having an amino acid sequence with at least 80%, 85%, 90%, 92%, 94%, 96%, 98%, 99% or 100% similarity to any of SEQ ID NO: 25-30;
t) an Acetoacetyl-CoA thiolase enzyme;
u) an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39;
v) a Hmg-CoA reductase enzyme;
w) a Hmg-CoA synthase enzyme, for example having an amino acid sequence of SEQ ID NO: 40 or having an amino acid sequence with at least 80%, 85%, 90%, 92%, 94%, 96%, 98%, 99% or 100% similarity to any of SEQ ID NO: 40.

Q7. The engineered cell according to any of embodiments Q1-Q6 wherein the cell comprises one or more polynucleotides that encode:
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
ii) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
iii) a HmgACT enzyme of SEQ ID NO 17 or having a nucleic acid sequence that encodes a protein with an amino acid sequence with at least 80%, 85%, 90%, 92%, 94%, 96%, 98%, 99% or 100% similarity to SEQ ID NO: 17;
iv) a HmgS enzyme of SEQ ID NO: 18 or having a nucleic acid sequence that encodes a protein with an amino acid sequence with at least 80%, 85%, 90%, 92%, 94%, 96%, 98%, 99% or 100% similarity to SEQ ID NO: 18;
v) a HmgR enzyme that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
vi) a MVK enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;
vii) a PMK enzyme of SEQ ID NO: 23 or having a nucleic acid sequence that encodes a protein with an amino acid sequence with at least 80%, 85%, 90%, 92%, 94%, 96%, 98%, 99% or 100% similarity to SEQ ID NO: 23; and
viii) a PMD enzyme of SEQ ID NO: 24 or having a nucleic acid sequence that encodes a protein with an amino acid sequence with at least 80%, 85%, 90%, 92%, 94%, 96%, 98%, 99% or 100% similarity to SEQ ID NO: 24;
x) an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39; and
xi) a Hmg-CoA synthase enzyme having an amino acid sequence of SEQ ID NO: 40 or having an amino acid sequence with at least 80%, 85%, 90%, 92%, 94%, 96%, 98%, 99% or 100% similarity to any of SEQ ID NO: 40.

Q8. The cell according to embodiment Q7 further comprising a polynucleotide that encodes a phytoene desaturase enzyme having an amino acid sequence of SEQ ID NO: 33-38 or having an amino acid sequence with at least 80%, 85%, 90%, 92%, 94%, 96%, 98%, 99% or 100% similarity to any of SEQ ID NO: 33-38; optionally
wherein the phytoene desaturase enzyme is capable of converting phytoene to lycopene, and optionally is a phytoene desaturase enzyme having an amino acid sequence of SEQ ID NO: 33 or 34, or has an amino acid sequence with at least 80%, 85%, 90%, 92%, 94%, 96%, 98%, 99% or 100% similarity to SEQ ID NO: 33 or 34; or
wherein the phytoene desaturase enzyme is capable of converting phytoene to neurosporene, and optionally is a phytoene desaturase enzyme having an amino acid sequence of SEQ ID NO: 36 or has an amino acid sequence with at least 80%, 85%, 90%, 92%, 94%, 96%, 98%, 99% or 100% similarity to SEQ ID NO: 36.

### R—Cell Free System

R1. A cell free phytoene production system comprising any one or more of:
a) a phytoene synthase enzyme according to any of embodiments A1-A7, optionally a phytoene synthase enzyme having an amino acid sequence of any of SEQ ID NO: 1-7, 51 or 52 or having an amino acid sequence with at least 80%, 85%, 90%, 92%, 94%, 96%, 98%, 99% or 100% similarity to any of SEQ ID NO: 1-7, 51 or 52, optionally a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
b) a GGPPS enzyme according to any of embodiments B1-B7, optionally a GGPPS enzyme having an amino acid sequence of any of SEQ ID NO: 8-16, 55, 56, 57 or 58 or having an amino acid sequence with at least 80%, 85%, 90%, 92%, 94%, 96%, 98%, 99% or 100% similarity to any of SEQ ID NO: 8-16, 55, 56, 57 or 58, optionally a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14; and/or
c) a HmgR enzyme (including engineered HmgR enzymes described herein) according to any of embodiments D1-D5, optionally a HmgR enzyme (including engineered HmgR enzymes described herein) that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
d) a MVK enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22.

R2. The cell free production system of embodiment R1 wherein the system further comprises any one or more of:
a) a HmgACT enzyme, optionally with an amino acid sequence of SEQ ID NO 17 or having a nucleic acid sequence that encodes a protein with an amino acid sequence with at least 80%, 85%, 90%, 92%, 94%, 96%, 98%, 99% or 100% similarity to SEQ ID NO: 17;
b) a HmgS enzyme, optionally with an amino acid sequence of SEQ ID NO: 18 or having a nucleic acid sequence that encodes a protein with an amino acid sequence with at least 80%, 85%, 90%, 92%, 94%, 96%, 98%, 99% or 100% similarity to SEQ ID NO: 18;
d) a PMK enzyme, optionally with an amino acid sequence of SEQ ID NO: 23 or having a nucleic acid sequence that encodes a protein with an amino acid sequence with at least 80%, 85%, 90%, 92%, 94%, 96%, 98%, 99% or 100% similarity to SEQ ID NO: 23;
e) a PMD enzyme, optionally with an amino acid sequence of SEQ ID NO: 24 or having a nucleic acid sequence that encodes a protein with an amino acid sequence with at least 80%, 85%, 90%, 92%, 94%, 96%, 98%, 99% or 100% similarity to SEQ ID NO: 24;
f) an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39; and/or
g) a Hmg-CoA synthase enzyme having an amino acid sequence of SEQ ID NO: 40 or having an amino acid sequence with at least 80%, 85%, 90%, 92%, 94%, 96%, 98%, 99% or 100% similarity to any of SEQ ID NO: 40.

R3. The cell free phytoene production system of any of embodiments R1 or R2 wherein the system comprises:
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
ii) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
iii) a HmgACT enzyme of SEQ ID NO 17 or having a nucleic acid sequence that encodes a protein with an amino acid sequence with at least 80%, 85%, 90%, 92%, 94%, 96%, 98%, 99% or 100% similarity to SEQ ID NO: 17;
iv) a HmgS enzyme of SEQ ID NO: 18 or having a nucleic acid sequence that encodes a protein with an amino acid sequence with at least 80%, 85%, 90%, 92%, 94%, 96%, 98%, 99% or 100% similarity to SEQ ID NO: 18;
v) a HmgR enzyme (including engineered HmgR enzymes described herein) that:
   has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
   has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
   comprises a Q236H substitution, a T188S substitution a G343D and/or a T102I substitution wherein said positions are numbered with reference to SEQ ID No: 19;
   comprises a K102I substitution, a G188S substitution, a W236H substitution and/or a P343D substitution wherein said positions are numbered with reference to SEQ ID NO: 20;
   comprises a F102I substitution, a Q188S substitution, a S236H substitution and/or a Q343D substitution wherein said positions are numbered with reference to SEQ ID NO: 21;
   comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
   is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
   is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
vi) a MVK enzyme that:
   has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
   comprises a E419D substitution, a K402V substitution, a E73G substitution, a G393V substitution, a L175V substitution, a D74E substitution, wherein said positions are numbered with reference to SEQ ID No: 22;
   has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
   comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
   has a nucleotide sequence that encodes a protein with an amino acid sequence of SEQ ID NO: 22;
   is from S. cerevisiae, optionally has an amino acid sequence of SEQ ID NO: 22;
vii) a PMK enzyme of SEQ ID NO: 23 or having a nucleic acid sequence that encodes a protein with an amino acid sequence with at least 80%, 85%, 90%, 92%, 94%, 96%, 98%, 99% or 100% similarity to SEQ ID NO: 23;
viii) a PMD enzyme of SEQ ID NO: 24 or having a nucleic acid sequence that encodes a protein with an amino acid sequence with at least 80%, 85%, 90%, 92%, 94%, 96%, 98%, 99% or 100% similarity to SEQ ID NO: 24;
ix) a CRTI enzyme having an amino acid sequence of SEQ ID NO: 33-38 or having an amino acid sequence with at least 80%, 85%, 90%, 92%, 94%, 96%, 98%, 99% or 100% similarity to any of SEQ ID NO: 33-38;
x) an Acetoacetyl-CoA thiolase enzyme having an amino acid sequence of SEQ ID NO: 39 or having an amino acid sequence with at least 80%, 85%, 90%, 92%, 94%, 96%, 98%, 99% or 100% similarity to any of SEQ ID NO: 39; and
xi) a Hmg-CoA synthase enzyme having an amino acid sequence of SEQ ID NO: 40 or having an amino acid sequence with at least 80%, 85%, 90%, 92%, 94%, 96%, 98%, 99% or 100% similarity to any of SEQ ID NO: 40.

R4. A cell free lycopene production system, wherein the system comprises the cell free phytoene production system of any of embodiments R1-R3 and further comprising a phytoene desaturase enzyme capable of converting phytoene to lycopene,
optionally wherein the phytoene desaturase enzyme has an amino acid sequence of SEQ ID NO: 33 or 34 or has an amino acid sequence with at least 80%, 85%, 90%, 92%, 94%, 96%, 98%, 99% or 100% similarity to any of SEQ ID NO: 33 or 34.

R5. A cell free neurosporene production system, wherein the system comprises the cell free phytoene production system of any of embodiments R1-R3 and further comprising a phytoene desaturase enzyme capable of converting phytoene to neurosporene,
optionally wherein the phytoene desaturase enzyme has an amino acid sequence of SEQ ID NO: 35 or has an amino acid sequence with at least 80%, 85%, 90%, 92%, 94%, 96%, 98%, 99% or 100% similarity to any of SEQ ID NO: 35.

R6. A cell free bisabolol production system.

### S—Methods

S1. A method for producing a carotenoid or terpenoid, optionally phytoene, wherein the method comprises culturing the cells according to any of embodiments Q1-Q8.

S2. A method for the production of lycopene, wherein the method comprises culturing the cells according to any of embodiments Q8, and where the phytoene desaturase enzyme is capable of converting phytoene to lycopene.

S3. A method for the production of neurosporene, wherein the method comprises culturing the cells according to any of embodiments Q8, and where the wherein the phytoene desaturase enzyme is capable of converting phytoene to neurosporene.

S4. The method of any of embodiments S1-S3 wherein expression of one or more of the enzymes or nucleic acids is under the control of an inducible promoter, the method further comprises culturing the cells in the presence of the corresponding inducing agent, optionally where the inducible promoter is a galactose inducible promoter and the inducing agent is galactose.

S5. The method according to any of embodiments S1-S4 wherein the cells are E. coli cells or are S. cerevisiae cells.

S6. The method according to any of embodiments S1-S5 wherein the cells are cultured using fermentation, optionally wherein the fermentation is a batch fermentation or a fed-batch fermentation or continuous culture.

S7. The method according to any of embodiments S1-S6 wherein the cells are cultured using:
a hexose sugar as a carbon source, optionally glucose;
a complex sugar as a carbon source;
crude glycerol as a carbon source; and/or
glycerol as a carbon source.

S8. A method for producing a carotenoid or terpenoid wherein the method comprises culturing a recombinant cell, wherein the cell

expresses or overexpresses one or more enzymes of the mevalonate pathway and/or methylerythritol pathway, and wherein said one or more enzymes of the mevalonate pathway and/or methylerythritol pathway includes at least 3-hydroxy-3-methylglutaryl-CoA reductase, wherein the 3-hydroxy-3-methylglutaryl-CoA reductase enzyme:
has at least 80%, 85%, 90%, 92%, 94%, 96%, 98%, 99% or 100% similarity to SEQ ID NO: 19, 20 or 21 provided that the enzyme comprises a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
   is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
   optionally wherein said expression or overexpression results in increased production of:
i) phytoene;
ii) carotenoids;
iii) bisabolol;
iv) terpenoids; and/or
v) isopentenyl pyrophosphate (IPP) and dimethylallyl pyrophosphate (DMAPP) relative to a cell that does not express and/or overexpress said one or more enzymes of the mevalonate pathway and/or methylerythritol pathway.

S9. A method for producing a carotenoid or terpenoid wherein the method comprises culturing a recombinant cell, wherein the cell

expresses or overexpresses one or more enzymes of the mevalonate pathway and/or methylerythritol pathway, and wherein said one or more enzymes of the mevalonate pathway and/or methylerythritol pathway includes at least mevalonate kinase, wherein the mevalonate kinase enzyme:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
has a nucleotide sequence that encodes a protein with an amino acid sequence of SEQ ID NO: 22;
is from S. cerevisiae, optionally has an amino acid sequence of SEQ ID NO: 22;
   optionally wherein said expression or overexpression
   results in increased production of:
i) phytoene;
ii) carotenoids;
iii) bisabolol;
iv) terpenoids; and/or
v) isopentenyl pyrophosphate (IPP) and dimethylallyl pyrophosphate (DMAPP)
relative to a cell that does not express and/or overexpress said one or more enzymes of the mevalonate pathway and/or methylerythritol pathway.

The listing or discussion of an apparently prior-published document in this specification should not necessarily be taken as an acknowledgement that the document is part of the state of the art or is common general knowledge. Preferences and options for a given aspect, feature or parameter of the invention should, unless the context indicates otherwise, be regarded as having been disclosed in combination with any and all preferences and options for all other aspects, features and parameters of the invention.

### BRIEF DESCRIPTION OF THE DRAWINGS

FIG. 1. The engineering of pathways and enzymes to produce terpenoids and carotenoids in E. coli including the genes in the MEP pathway (Module 2), mevalonate pathway (Module 1), and genes to produce various terpenoids and carotenoids including bisabolol, phytoene, lycopene, neurosporene, and β-carotene.

FIG. 2. An exemplary expression vector for overexpression of the MEP pathway in E. coli; SEQ ID NO: 45.

FIG. 3. An exemplary expression vector for overexpression of GGPPS and PYS for production of phytoene in E. coli; SEQ ID NO:46.

FIG. 4. Phytoene production using recombinant E. coli expressing various PYS diversity. Sample names represent the following genes: PY1G1 (PYS from Pantoea ananatis provided as SEQ ID NO: 1 and G1 from S. cerevisiae provided as SEQ ID NO: 8), PY2G1 (PYS from Daucus carota provided as SEQ ID NO: 2 and G1 from S. cerevisiae provided as SEQ ID NO: 8), PY3G1 (PYS from Enterobacter agglomerans provided as SEQ ID NO: 3 and G1 from S. cerevisiae provided as SEQ ID NO: 8), PY4G1 (PYS from Synechocystis sp. provided as SEQ ID NO: 4 and G1 from S. cerevisiae provided as SEQ ID NO: 8), PY5G1 (PYS from Arthrospira platensis provided as SEQ ID NO: 5 and G1 from S. cerevisiae provided as SEQ ID NO: 8), PY6G1 (PYS from Cucumis melo provided as SEQ ID NO: 6 and G1 from S. cerevisiae provided as SEQ ID NO: 8), and PY7G1 (PYS from Staphylococcus aureus provided as SEQ ID NO: 7 and G1 from S. cerevisiae provided as SEQ ID NO: 8).

FIG. 5. Phytoene production using recombinant E. coli expressing various GGPPS diversity. Sample names represent the following genes: PY3G1 (PYS from Enterobacter agglomerans provided as SEQ ID NO: 3 and G1 from S. cerevisiae provided as SEQ ID NO: 8), PY3G2 (PYS from Enterobacter agglomerans provided as SEQ ID NO: 3 and G2 from Capsicum annuum provided as SEQ ID NO: 9), PY3G3 (PYS from Enterobacter agglomerans provided as SEQ ID NO: 3 and G3 from Abies grandis provided as SEQ ID NO: 10), PY3G4 (PYS from Enterobacter agglomerans provided as SEQ ID NO: 3 and G4 from Penicillium paxilli provided as SEQ ID NO: 11), PY3G5 (PYS from Enterobacter agglomerans provided as SEQ ID NO: 3 and G5 from Mucor lusitanicus provided as SEQ ID NO: 12), PY3G6 (PYS from Enterobacter agglomerans provided as SEQ ID NO: 3 and G6 from Croton sublyratus provided as SEQ ID NO: 13), PY3G7 (PYS from Enterobacter agglomerans provided as SEQ ID NO: 3 and G7 from Hevea brasiliensis provided as SEQ ID NO: 14), PY3G8 (PYS from Enterobacter agglomerans provided as SEQ ID NO: 3 and G8 from Pseudescherichia vulneris provided as SEQ ID NO: 15), and PY3G9 (PYS from Enterobacter agglomerans provided as SEQ ID NO: 3 and G9 from Enterobacter agglomerans provided as SEQ ID NO: 16).

FIG. 6 An exemplary expression vector, pRSFDuet-tac-MM, for overexpression of key enzymes in the phytoene production pathway in E. coli including the expression cassettes IDI-MVK-PMK-PMD-Hmg, and HmgS-DXS.

FIG. 7. Phytoene production with engineered E. coli overexpressing MVA, MEP pathway genes, PYS, GGPPS, and HmgR mutants. Phytoene production is shown as a relative value with WT normalized to 100%. The WT sequence of S. pneumoniae HmgR is provided as SEQ ID NO: 19 and the identified improved mutations are as follows: Mut1 has the threonine at amino acid residue 188 mutated to serine (T188S); Mut2 has the glutamine at amino acid residue 236 mutated to histidine (Q236H); and Mut3, has the glycine at amino acid residue 343 mutated to aspartic acid (G343D).

FIG. 8. Relative HmgR activity in engineered E. coli overexpressing HmgR WT and HmgR1_Mut2. Mut2 has the glutamine at amino acid residue 236 mutated to histidine (Q236H).

FIG. 9. Qualitative measurement of lycopene and neurosporene production in E. coli strains with an active Crtl enzyme compared to the phytoene control strain. Lycopene production was confirmed by a dark pink coloured cell pellet whereas neurosporene production was confirmed by a yellowish orange coloured cell pellet compared to the control phytoene strain which is not coloured.

FIG. 10. Phytoene (mg/L) production using recombinant S. cerevisiae expressing different combinations of novel phytoene synthase (PYS) and Geranylgeranyl Diphosphate Synthase (GGPPS) genes found in a natural diversity library. PYS3 represents the PYS gene from Enterobacter agglomerans (SEQ ID NO: 3), PYS1 represents the PYS gene from Pantoea ananatis (SEQ ID NO: 1), GGPPS2 from Capsicum annuum (SEQ ID NO: 9), GGPPS6 represents the PYS gene from Croton sublyratus (SEQ ID NO: 13), and GGPPS7 from Hevea brasiliensis (SEQ ID NO: 14).

FIG. 11. Example of an exemplary expression vector for the overexpression of a PYS in S. cerevisiae to produce phytoene (SEQ ID NO: 59).

FIG. 12 Example of an exemplary expression vector for the overexpression of a GGPPS and truncated HMG1 in S. cerevisiae (SEQ ID NO: 60).

FIG. 13. Example of an exemplary expression vector for the overexpression of a BBS in S. cerevisiae to produce Bisabolol (SEQ ID NO: 61).

FIG. 14. Example of an exemplary expression vector for the overexpression of a BBS in E. coli to produce Bisabolol (SEQ ID NO: 62).

FIG. 15. Bisabolol production in E. coli from genes identified in a diversity search. Genes include: BBS-1 (from Cynara cardunculus; SEQ ID NO: 25), BBS-2 (from Artemisia annua; SEQ ID NO: 26), BBS-4 (from Eremanthus erythropappus; SEQ ID NO: 28).

FIG. 16. Bisabolol production in S. cerevisiae from genes identified in a diversity search. Genes include: McBBS (BBS6 from Matricaria chamomilla, SEQ ID NO:59) and CcBBS (BBS1 from Cynara cardunculus; SEQ ID NO: 25) with constitutive S. cerevisiae TEF1 promoter (McBBS-C and CcBBS-C) and inducible S. cerevisiae pGal1/10 promoter (McBBS-I and CcBBS-I)

FIG. 17. Relative MvaK activity in engineered E. coli overexpressing MvaK WT and MvaK_Mut1, MvaK_Mut2, MvaK_Mut3, MvaK_Mut4, MvaK_Mut5, MvaK_Mut6, and MvaK_Mut7. Mut1 has the glutamic acid at amino acid residue 419 mutated to aspartic acid (E419D), Mut2 has the lysine at amino acid residue 402 mutated to valine (K402V), Mut3 has the glutamic acid at amino acid residue 73 mutated to glycine (E73G), Mut4 has the glycine at amino acid residue 393 mutated to valine (G393V), Mut5 has the leucine at amino acid residue 175 mutated to valine (L175V), Mut6 has the aspartic acid at amino acid residue 74 mutated to glutamic acid (D74D).

### EXAMPLES

### Example 1. Generation of E. coli Strains Overexpressing the Methylerythritol Phosphate (MEP) Pathway

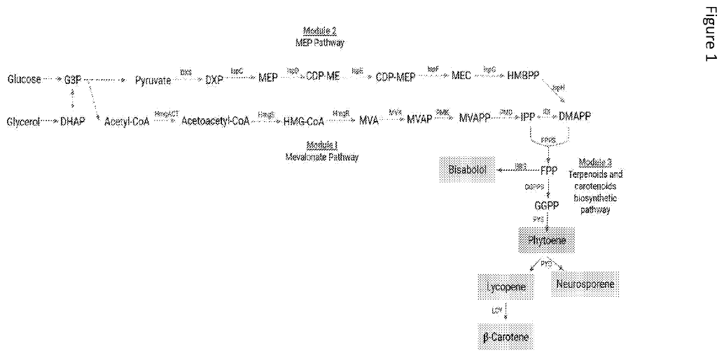

The biosynthetic pathway for phytoene was divided into three different modules (as shown in FIG. 1), Module 1 includes the, mevalonate pathway genes encoding HmgACT, HmgS, HmgR, MVK, PMK, PMD; Module 2, methylerythritol phosphate (MEP) pathway including DXS, IspC, IspD, IspE, IspF, IspG, IspH, IDI. To produce a parent strain for screening genes in Module 3 (PYS and, GGPPS), the enzymes of Module 2 (MEP) from E. coli were overexpressed from multiple expression vectors containing one or more of the genes required for a functional MEP pathway. Vectors including various antibiotic resistance genes were used for selection (including chloramphenicol, streptomycin and kanamycin). The expression vectors also contained the T7 or tac promoter to allow for which can provide inducible expression of the cloned genes with the chemical IPTG (Isopropyl ß-D-1-thiogalactopyranoside).

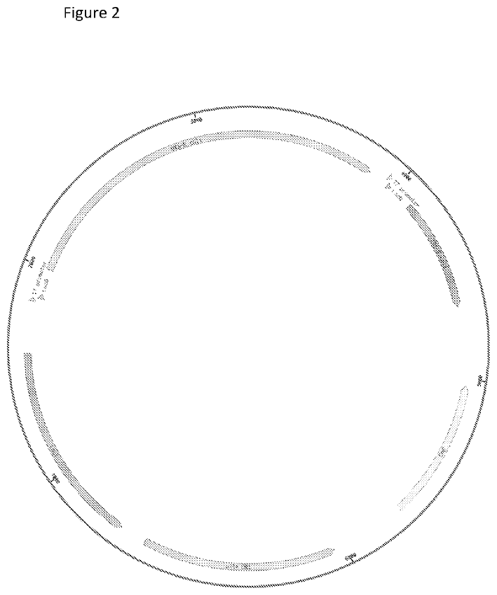

MEP pathway genes DXS (SEQ ID NO. 43) and IDI (SEQ ID NO. 44) were PCR-amplified from E. coli gDNA and cloned into the expression vectors using restriction cloning with various restriction enzymes using standard molecular biology techniques. Individual colonies from the transformation were confirmed for the presence of the cloned gene by using colony PCR with appropriate primers and the sequence was confirmed using Sanger sequencing. An example of exemplary expression vectors for the overexpression of Module 2 is provided in FIG. 2 as well as provided as SEQ ID NO. 45

The sequence-confirmed expression vectors were then transformed into the expression host E. coli using the standard molecular biology electroporation method. Transformations were plated on selective media and grown overnight at 37° C. Individual colonies were confirmed for the presence of the cloned gene using colony PCR and used for either in vivo or in vitro assessment of phytoene production.

### Example 2. Analytical Methods for Phytoene Extraction from E. coli

As phytoene accumulates inside E. coli, cell lysis and liquid-liquid extraction were required to quantify phytoene. Briefly, 20 OD₆₀₀ of cells were harvested in a 2 ml tube and resuspended in 800 μl 100% acetone. The 2 ml tube with resuspended cells was left in a chilled (8-10° C.) water bath sonicator with sonication ON for 30 min. The 2 mL tube was centrifuged at maximum speed (14000 rpm) for 1 min to pellet the cell debris. The cell lysate was transferred to a new tube and the pellets were washed twice with 800 μL ethyl acetate to enhance phytoene extraction. Each wash was transferred to the tube containing the cell lysate. Additional water (600 μL) was added to the cell lysate and the tube was vortexed vigorously for 30 seconds to ensure thorough mixing and allowing the phytoene to partition to the ethyl acetate. The tube was centrifuged at maximum speed (14000 rpm) for 1 min to separate the ethyl acetate and aqueous phases. The upper ethyl acetate phase (containing phytoene) was transferred to a new tube and the ethyl acetate evaporated at reduced pressure in a Concentrator plus (Eppendorf). To the dry phytoene, 100 μl 80% Methanol+20% Methyl tert-Butyl Ether was added to solubilize the phytoene and the concentration of phytoene in solution was quantified using the HPLC method described below in Example 3.

### Example 3. Analytical Methods for Quantification of Phytoene Produced in E. coli

The extracted phytoene from Example 2 was measured using an HPLC (LC-40D XS, Shimadzu Corporation) equipped with a photo diode array detector using UV absorbance at 360 nm (DAD) and SB-C18 column (Poroshell 120, 150×4.6 mm, 2.7 μm, Agilent) for separation. The mobile phase, 80% Methanol+20% Methyl tert-Butyl Ether, was used as an eluent with a flow rate of 1.2 mL/min. The column temperature was maintained at 20° C. Phytoene (mixture of isomers) was purchased from Sigma-Aldrich (Product No. 78903) and used as an external standard for phytoene peak detection and quantification. The retention time of phytoene was 18.5 min for the above described method. Comparing the peak area from prepared dilution standards (a standard curve) containing phytoene we were able to quantify the amount of Phytoene extracted from the E. coli extracted samples.

### Example 4. Shake Flask Production of Phytoene in E. coli

The recombinant E. coli strains with the expression vectors described below in Examples 5 and 6 (containing the essential genes for phytoene production) were inoculated from glycerol stock into 5 ml of Luria-Bertani (LB) broth containing the respective antibiotics to ensure plasmid retention and grown shaking at 37° C., 250 RPM for 6 h. To induce production, 20 mL of modified M9 media (described below) containing the respective antibiotics were inoculated with from the LB seed culture to obtain an initial OD₆₀₀ of 0.1. The composition of modified M9 media is as follows (per liter): Na2HPO4·7H2O, 11.3 g; KH2PO4, 3.0 g; NaCl, 0.5 g; NH4Cl, 1.0 g; yeast extract, 6.0 g; Glycerol, 20.0 g, MgSO4, 1 mM; CaCl2, 0.1 mM. The production flasks were incubated at 37° C., 250 RPM for 2 h after which Isopropyl β-D-1-thiogalactopyranoside (IPTG) was added to a final concentration of 0.5 mM to induce expression of the recombinant plasmid containing genes and allow for phytoene production. During induction, the growth temperature was brought down to 30° C. and maintained at 250 RPM for 24-48 h. At this point, 20 OD₆₀₀% of cells were harvested and used for phytoene extraction and quantification as described above in Examples 2 and 3.

### Example 5. Identification of Novel Proteins with Phytoene Synthase (PYS) Activity from a Natural Diversity Library Screen in E. coli

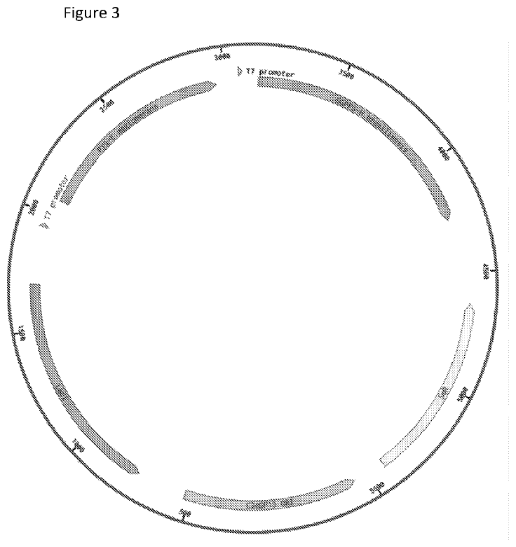

To construct a strain for in vivo phytoene synthase (PYS) testing, several novel phytoene synthases were identified from natural diversity, codon optimised and synthesised by Genescript (provided as SEQ ID NO: 1, NO: 2, NO: 3, NO: 4 NO: 5, NO: 6, and NO: 7). To screen PYS enzymes, we used a plasmid similar to that described above in Example 1 overexpressing DXS and IDI along with a second plasmid carrying a fixed codon optimised Geranylgeranyl Diphosphate Synthase (GGPPS) from S. cerevisiae (SEQ ID NO: 8) and novel GGPPS enzymes. To produce a single expression vector containing the fixed GGPPS and novel PYS enzymes the individual genes were initially cloned into either the MCS-1 (Multiple Cloning Site 1) or MCS-2 (Multiple Cloning Site 2) in the plasmid pCDFDuet-1 (Sequence available at https://www.addgene.org/vector-database/5878/) and called “pCDFDuet-1-PYxG1” where the “x” represents the specific PYS gene used and “G1” represents the S. cerevisiae GGPPS. An example of an exemplary expression vector for the overexpression of PYS and GGPPS is shown in FIG. 3 and provided as SEQ ID NO: 46

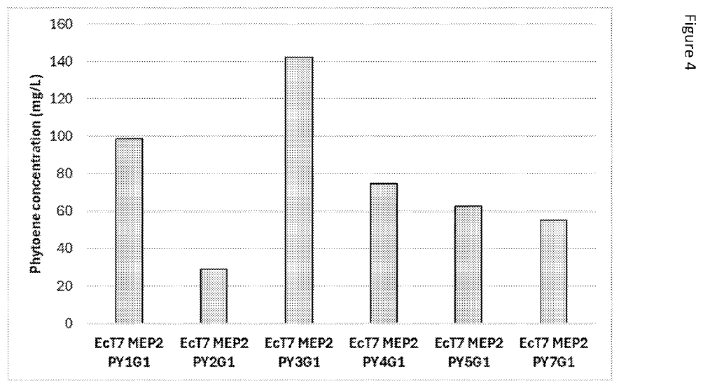

To screen the various pCDFDuet-1-PYxG1 vectors, each was transformed into the E. coli strain with the resulting strains named “EcT7 MEP2 PYxG1” where again “x” denotes the reference number of PYS and “G1” denotes that the S. cerevisiae GGPPS was used. The data in FIG. 4 shows data from six different PYS genes screened using the methods described in Examples 2, 3 and 4 above, the seventh gene is not included as it did not function and therefore served as built in control with no phytoene production. Strain “EcT7 MEP2 PY3G1” accumulated 142.1 mg/L phytoene (the most for all strains) in shake flasks and was selected as the parent enzyme for further screening of GGPPS enzymes as shown below in Example 6. The best PYS gene (called “PY3”) was found in the E. agglomerans genome and is provided as SEQ ID NO: 3.

### Example 6. Identification of Novel Proteins with Geranylgeranyl Diphosphate

Synthase (GGPPS) activity from a natural diversity library screen in E. coli To construct a strain for in vivo Geranylgeranyl Diphosphate Synthase (GGPPS) testing, several novel GGPPS enzymes were identified from natural diversity, codon optimised and synthesised by Genescript (provided as SEQ ID NO: 8, NO: 9, NO: 10, NO: 11, NO: 12, NO: 13, NO: 14, NO: 15, NO: 16). To screen GGPPS enzymes, we used a plasmid similar to that described above in Example 1 overexpressing DXS and IDI along with a second plasmid carrying the codon optimised PYS gene discovered in Example 5 (PY3; provided as SEQ ID NO. 3) and novel GGPPS enzymes cloned into either the MCS-1 (Multiple Cloning

Site 1) or MCS-2 (Multiple Cloning Site 2) in the plasmid pCDFDuet-1 (Sequence available at https://www.addgene.org/vector-database/5878/) and called “pCDFDuet-1-PY3Gx” where “PY3” represents the fixed E. agglomerans PYS gene used and “Gx” represents the novel GGPPS. An example of an exemplary expression vector for the overexpression of PYS and GGPPS is shown in FIG. 3 and provided as SEQ ID NO: 46

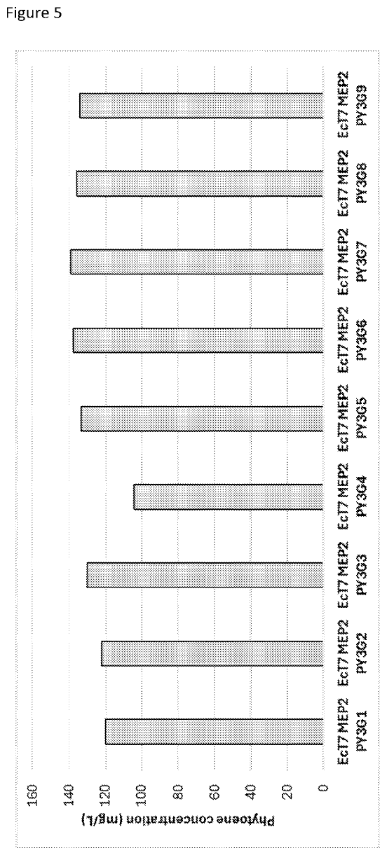

The activity of the novel GGPPS enzymes were screened using the same methods described in Examples 2, 3 and 4 above. The data in FIG. 5 shows data from 9 different GGPPS enzymes tested. Strain “EcT7 MEP2 PY3G7” accumulated 139.1 mg/L phytoene (the most for all strains) in shake flasks. This titer represents a 20% increase over the control strain expressing S. cerevisiae GGPPS (G1). This best GGPPS gene (“G7”) was found in the H. brasiliensis genome and provided as SEQ ID NO: 14.

To simplify the testing of multiple parameters only PY3 from E. agglomerans (Seq ID no: 3) and G7 from H. brasiliensis (Seq ID no: 14) were used in the following Examples.

### Example 7. Generation of a Recombinant E. coli Strain Overexpressing Both the MEP and Mevalonate Pathway for Improved IPP and DMAPP Production

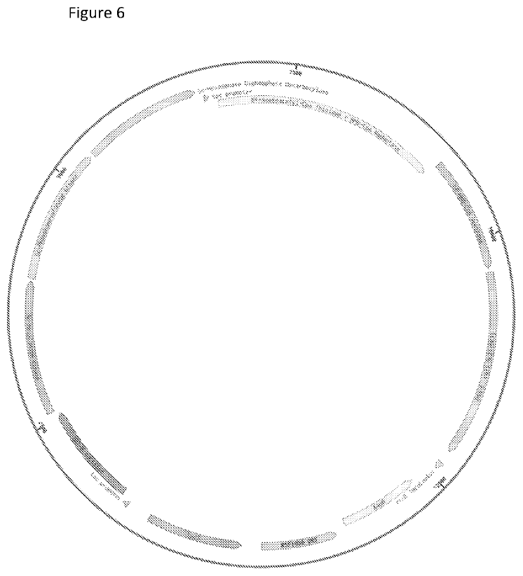

To further improve the precursors' (IPP and DMAPP) availability and therefore increase phytoene production, heterologous overexpression of the mevalonate pathway (Shown in FIG. 1 as Module 1) was added to a strain similar to “EcT7 MEP2 PY3G7” as described above in Example 6. The enzymes used in E. coli to overexpress the Mevalonate pathway are: E. faecalis Acetoacetyl-CoA thiolase (HmgACT; SEQ ID NO: 17), E. faecalis Hydroxymethylglutaryl-CoA synthase (HmgS; SEQ ID NO: 18), S. pneumoniae Hydroxymethylglutaryl-CoA reductase (HmgR; SEQ ID NO: 19), S. cerevisiae Mevalonate kinase (MVK; SEQ ID NO: 22), S. cerevisiae Phosphomevalonate kinase (PMK; SEQ ID NO: 23), S. cerevisiae Phosphomevalonate decarboxylase (PMD; SEQ ID NO:24), and either E. coli Isopentenyl-diphosphate delta isomerase (IDI; SEQ ID NO: 41) or B. subtilis Isopentenyl-diphosphate delta isomerase (IDI; SEQ ID NO:31). To make the expression vectors all of the enzymes for the mevalonate pathway were cloned into pRSFDuet-1 and the recombinant plasmid was named as pRSFDuet-1-MM. The promoter was changed from T7 to tac and the plasmid was renamed as pRSFDuet-1-tac-MM (Shown in FIG. 6, SEQ ID NO: 47). Described briefly, to construct pRSFDuet-1-tac-MM, gene IDI was amplified, digested with NcoI/BamHI, and cloned in the MCS-1 of pRSFDuet-1. Then the fragment MVK-PMK-PMD was codon optimised, synthesised, and cloned into the MCS-1 using EcoRI/NotI. The HmgACT, HmgR, and HmgS genes were cloned into the BglII and KpnI sites in the MCS-2 using overlapping PCR from codon optimised and synthesised genes. Finally, the DXS gene was amplified from codon optimised and synthesised template and cloned into the KpnI and XhoI sites in the MCS-2. The T7 promoter was swapped with a tac promoter using an In-Fusion® Snap Assembly kit to complete the pRSFDuet-1-tac-MM plasmid. To make sure promoters match in all plasmids, the T7 promoter from pCDFDuet-1-PY3G7 was also swapped with a tac promoter using an In-Fusion® Snap Assembly kit resulting in the plasmid pCDFDuet-1-tac-PY3G7.

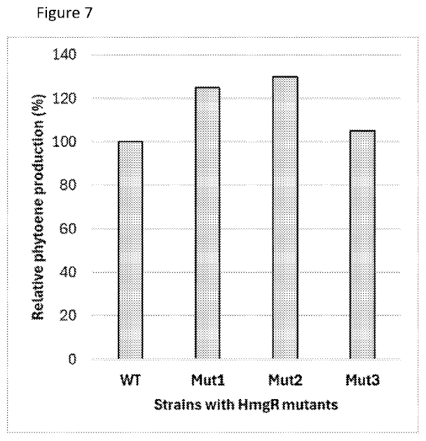

pRSFDuet-1-tac-MM containing and pCDFDuet-1-tac-PY3G7 was co-transformed into E. coli T7 resulting in the WT strain shown in FIG. 7.

### Example 8. Optimization of Phytoene Production Through Modification of Copy Number in E. coli

In order to study gene dosage, we developed vectors that are expected to result in varying copy numbers in the strain. In this case we wanted to compare pRSFDuet-1 (>100 copy number), pCDFDuet-1 (20-40 copy number), and pCOLADuet-1 (20-40 copy number; sequence available at https://www.addgene.org/vector-database/2260/). While pCDFDuet-1 contains the antibiotic resistance marker for ampicillin and is compatible with either pRSFDuet-1 or pCOLADuet-1 based vectors, both pRSFDuet-1 or pCOLADuet-1 contain the antibiotic resistance marker for kanamycin and so are therefore not compatible to use together. Therefore, the genes and tac promoter from pRSFDuet-1-tac-MM were subcloned into pCDFDuet-1 and pCOLADuet-1 to construct plasmids with relatively lower copy numbers that express the genes required for the mevalonate (Module 1 in FIG. 1) pathway. In addition, the genes and tac promoter from pCDFDuet-1-tac-P3G7 were cloned into both pCOLADuet-1 and pRSFDuet-1 to construct plasmids with relatively lower copy numbers that express the genes for PYS and GGPPS.

Creating the E. coli strains with 4 combinations of plasmids (shown in Table 1) we found that by balancing the copy number of plasmids we could change the amount of phytoene produced. The strain expressing both lower copy numbers of the genes for the mevalonate pathway and the GGPPS/PYS genes produced the least amount of phytoene (75 mg/L; combination 2), while a strain that expressed higher copy numbers of mevalonate pathway and lower copy numbers of the GGPPS/PYS genes produced twice the amount of phytoene (154 mg/L; combination 3). Finally, a strain that expressed a lower copy numbers of the genes for the mevalonate pathway with high copy numbers of the GGPPS/PYS genes produced 682 mg/L phytoene (combination 4) which represents more than 9 times the amount of phytoene produced in combination 2 (both lower copy plasmids). These results indicate that properly balancing the gene dosage between the mevalonate pathway (Module 2; precursors of IPP and DMAPP) and the GGPPS/PYS downstream portion of the pathway can play a significant effect on the amount of phytoene produced by E. coli.

TABLE 1
Phytoene production using recombinant E. coli expressing
PYS and GGPPS from different copy number plasmids

| | MM | PYGP | Phytoene |
|---|---|---|---|
| Combination | Plasmid | Plasmid | Production (mg/L) |
| 1 | pCDFDuet-1 | pRSFDuet-1 | 101.16 |
| 2 | pCDFDuet-1 | pCOLADuet-1 | 75.19 |
| 3 | pRSFDuet-1 | pCDFDuet-1 | 154.1 |
| 4 | pCOLADuet-1 | pCDFDuet-1 | 682 |

### Example 9. Generation of a Strain for Bisabolol Production in E. coli

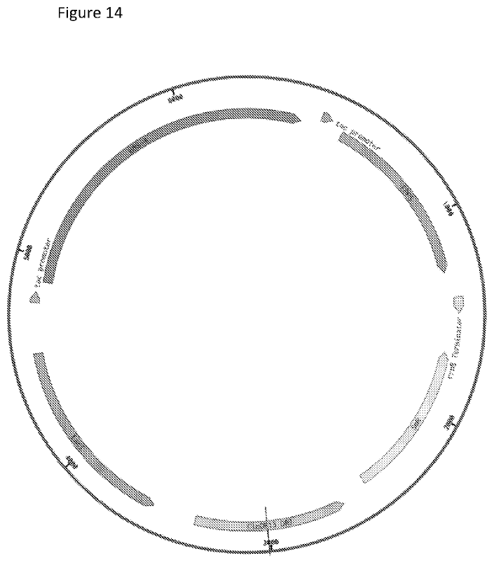

The phytoene production pathway in E. coli was modified for the production of bisabolol by replacing geranylgeranyl Diphosphate Synthase (GGPPS) and phytoene synthase (PYS) with farnesyl diphosphate synthase (FPPS) and bisabolol synthase (BBS), respectively. Homologous overexpression was performed by amplifying FPPS (SEQ ID NO: 42) from the E. coli genome and cloned into a pCDFDuet-1-tac vector to construct pCDFDuet-1-tac-FP. Genes coding six different BBS enzymes were synthesised; the BBS enzymes from Cynara cardunculus (BBS1; SEQ ID NO: 25), Artemisia annua (BBS2; SEQ ID NO: 26), Artemisia annua (BBS3; SEQ ID NO: 27), Eremanthus erythropappus (BBS4; SEQ ID NO: 28), Helianthus annuus (BBS5; SEQ ID NO: 29) and Matricaria recutita (BBS6; SEQ ID NO: 30). The genes were cloned into a pCDFDuet-1-tac-FP vector and pCDFDuet-1-tac-FPBS overexpressing different BBS were obtained; an example of an exemplary sequence is shown in FIG. 14 and provided as SEQ ID NO: 62. This vector was co-transformed into E. coli with pRSFDuet-1-tac-MM (described in Example 7) to construct the bisabolol producing E. coli strains.

### Example 10. Shake Flask Production of Bisabolol in E. coli

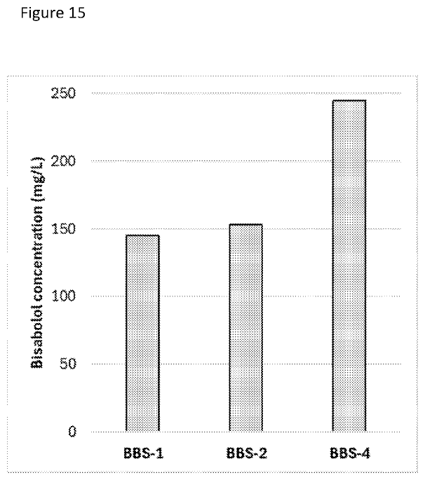

The recombinant E. coli strains with the expression vectors described above in Example 9 were inoculated from glycerol stock into 5 ml of Luria-Bertani (LB) broth containing the respective antibiotics to ensure plasmid retention and grown shaking at 37° C., 250 RPM for 6 h. To induce production, 20 ml of modified M9 media (described below) containing the respective antibiotics were inoculated with from the LB seed culture to obtain an initial OD₆₀₀ of 0.1. The composition of modified M9 media is as follows (per liter): Na2HPO4.7H2O, 11.3 g; KH2PO4, 3.0 g; NaCl, 0.5 g; NH4Cl, 1.0 g; yeast extract, 6.0 g; Glycerol, 20.0 g, MgSO4, 1 mM; CaCl2, 0.1 mM. The production flasks were incubated at 30° C., 250 RPM for 2 h after which Isopropyl β-D-1-thiogalactopyranoside (IPTG) was added to a final concentration of 0.5 mM to induce expression of the recombinant plasmid containing genes and allow for bisabolol production. During induction, 2 mL of dodecane was added to the flask to perform in situ extraction of bisabolol. The fermentation was continued at 250 RPM for 24-48 h. At the end of fermentation, dodecane was collected from the flasks and analysed for bisabolol concentration in GC as described in Example 17. The bisabolol titers from the recombinant strain were presented in FIG. 15.

### Example 11. Assay for the In Vitro Quantification of HmgR Activity from E. coli

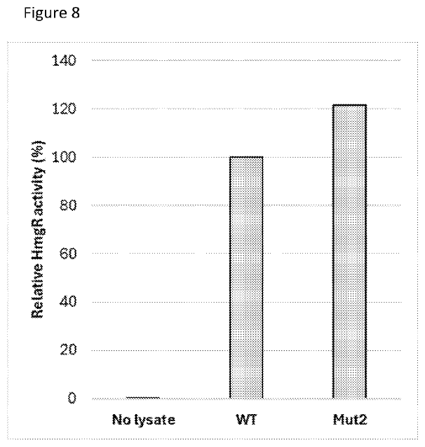

To measure the activity of improved variants of HmgR, a spectrophotometric assay was developed. In vivo, the HmgR enzyme oxidises NADPH to NADP+ to convert Hmg-CoA to mevalonate. As NADPH can be measured by absorbance at 340 nm, the activity HmgR can be indirectly measured by monitoring the change in absorbance using a spectrophotometer to monitor the oxidation of NADPH to NADP+. To confirm that this method would work as expected, E. coli cells expressing the wild type S. pneumoniae HmgR were constructed by cloning the HmgR gene into pET-28a (+) vector at NcoI/XhoI under the control of T7 promoter. The HmgR protein was expressed by growing the recombinant strain in LB medium and induced with 0.1 mM IPTG. The cells were grown for 16 h at 25° C. and lysed by mechanical lysis using glass beads. The lysate was centrifuged at 10,000×g for 30 min and the clear supernatant was used for the HmgR activity assay. The activities were compared with negative buffer control and E. coli cell lysate with HmgR overexpression, and rapid decrease in NADPH absorbance was only observed in the cell lysate with HmgR overexpression as seen in FIG. 8.

### Example 12. Identification of S. pneumoniae Variant HmgR Proteins with Improved HmgR Activity and Phytoene Production Using Site Saturation Mutagenesis

The WT HmgR of S. pneumoniae was scanned for beneficial mutations by mutating >80% of residues through single site saturation mutagenesis or combinatorial saturation mutagenesis using standard molecular biology techniques. The enzyme library was expressed similar to the wild type control in Example 11. After 16 h of enzyme expression, single cells were encapsulated in droplets and lysed and screened using a microfluidics screening platform which detects the amount of NADPH present inside the droplets (as described in the previous patent US 2021/0318315 A1). The droplets with low NADPH presence were sorted and pooled and sequenced. The identified mutations were introduced into the wild type HmgR gene present into both strains that express only HmgR and the full pathway containing, phytoene producing strain. The improved activity of HmgR (measured in the spectrophotometric assay) is shown in FIG. 8 and phytoene production was quantified and shown in FIG. 7. Mut2 had both an increase in HmgR activity (25%) as well as phytoene production (25%). The WT sequence of S. pneumoniae HmgR is provided as SEQ ID NO: 19 and the identified improved mutations are as follows: Mut1 has the threonine at amino acid residue 188 mutated to serine (T188S); Mut2 has the glutamine at amino acid residue 236 mutated to histidine (Q236H); and Mut3, has the glycine at amino acid residue 343 mutated to aspartic acid (G343D)

### Example 13. Generation of a Strain for Lycopene and Neurosporene Production in E. coli

The phytoene production pathway in E. coli was extended for the production of both lycopene and neurosporene by the addition of phytoene desaturase (CrtI) to the existing enzyme cascade. Five different CrtI enzymes were obtained and tested; the CrtI enzymes from Pantoea ananatis (CrtI1; SEQ ID NO: 33), Paracoccus sp. (CrtI2; SEQ ID NO: 34) Pseudescherichia vulneris (CrtI3; SEQ ID NO: 35) Synechocystis sp. (CrtI5; SEQ ID NO: 37) and Streptomyces griseus (CrtI6; SEQ ID NO: 38) are expected to be lycopene-forming and the CrtI from Rhodobacter capsulatus (CrtI4; SEQ ID NO: 36) is expected to be neurosporene-forming The genes were codon optimised for expression in E. coli, synthesised, and cloned into the pCDFDuet-1-tac-PYGP vector described in example 8. This vector was co-transformed into E. coli with pRSFDuet-1-tac-MM to construct the lycopene and neurosporene producing E. coli strains.

### Example 14. Qualitative Measurement of Lycopene and Neurosporene Production in E. coli Strains with an Active CrtI Enzyme

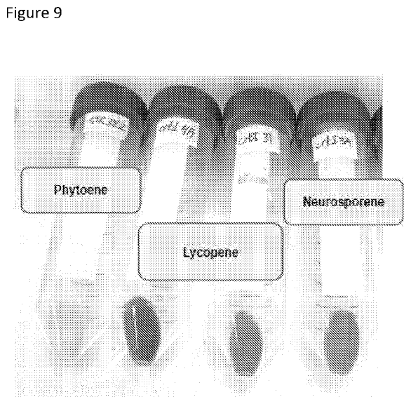

The recombinant E. coli strains produced in Example 13 containing the genes for the lycopene or neurosporene production were grown using the same method described in Example 4. In Brief, strains were inoculated from a glycerol stock into 5 ml of Luria-Bertani (LB) broth containing antibiotics and grown at 37° C., 250 RPM for 6 h. To initiate the shake flask production, 20 ml of modified M9 media containing antibiotics were inoculated from the seed culture to bring the initial OD₆₀₀ in flask to 0.1. After incubation for 2 hours at 37° C. and 250 RPM strains were induced with the addition of 0.5 mM IPTG. At the end of the fermentation, the culture was harvested in a 50 ml tube and centrifuged at 5,000×g for 10 min. The liquid was discarded and the cell pellet was resuspended with 20 mL of 100 mM Tris buffer pH 8 to dilute any residual media. The centrifugation was repeated and the liquid was discarded. The colour of the cell pellets were observed for confirmation of product production. Shown in FIG. 9, Lycopene production was confirmed by a dark pink coloured cell pellet whereas neurosporene production was confirmed by a yellowish orange coloured cell pellet. The colour of lycopene and neurosporene is known to individuals in the field and referenced in publications including: Maoka T. (2020). Carotenoids as natural functional pigments. Journal of natural medicines, 74(1), 1-16. The results of this experiment indicate that E. coli strains with an active CrtI enzyme can efficiently produce either lycopene or neurosporene depending on the source of the enzyme.

### Example 15. Generation of S. cerevisiae Strains Overexpressing the Mevalonate Pathway

To produce a S. cerevisiae parent strain overexpressing the mevalonate pathway (shown as Module 1 in FIG. 1), we integrated overexpressed copies of genes required to increase expression to IPP and DMAPP. All S. cerevisiae, promoters, terminators and upstream/downstream regions for integration into the genome were amplified directly from S. cerevisiae gDNA using the sequences provided by Saccharomyces Genome Database (available at: https://www.yeastgenome.org) and heterologous genes were codon optimised and synthesised for expression in S. cerevisiae by Genescript. Sc.Erg20F96W-N127W was described previously (Wu, T., et al. Microbial cell factories, 17(1), 85). Primers used contained overlapping fragments and assembly was performed by transforming all required parts into S. cerevisiae for homologous recombination into the yeast shuttle vector pRS425 vector (https://www.addgene.org/vector-database/3988/). Integration into the parent strain resulted in deletion of the open reading frame of the integration site. The resulting strain had the genotype as described in Table 2.

TABLE 2
Genotype of S. cerevisiae parent strain overexpressing the mevalonate pathway.

| Locus | Promoter 1 | Gene 1 | Terminator 1 | Promoter 2 | Gene 2 | Terminator 2 |
|---|---|---|---|---|---|---|
| LPP1 | pGPM1 | MVK | tHXT7 | pPYK1 | PMD | tPGK1 |
| | | (SEQ ID NO: 48) | | | (SEQ ID NO: 49) | |
| DPP1 | pTEF1 | Sc.IDI1 | tADH2 | pTPI1 | Sc.Erg20 | tHXT7 |
| ALG1 | pENO2 | HmgR3 | tADH1 | pTPI1 | HmgS | tPGI2 |
| | | (SEQ ID NO: 21) | | | (SEQ ID NO: 18) | |
| MGA1 | pGPM1 | MVK | tHXT7 | pHSP104 | PMD | tPGK1 |
| | | (SEQ ID NO: 48) | | | (SEQ ID NO: 49) | |
| YCT1 | pHSP26 | Sc.IDI1 | tADH2 | pSSA1 | Sc.Erg20F96W- | tHXT7 |
| | | | | | N127W | |
| NSIX 9491- | pENO2 | HmgR3 | tADH1 | pTPI1 | MVK | tPGI2 |
| 11219 | | (SEQ ID NO: 21) | | | (SEQ ID NO: 48) | |
| NSVIII-1 | pGPM1 | HmgR3 | tHXT7 | pPYK1 | MVK | tPGK1 |
| | | (SEQ ID NO: 21) | | | (SEQ ID NO: 48) | |
| ROX1 | pENO2 | HmgR3 | tADH1 | pTPI1 | HmgS | tPGI2 |
| | | (SEQ ID NO: 21) | | | (SEQ ID NO: 18) | |
| URA3 | pTEF1 | tHMGR1 | tADH1 | | | |
| | | (SEQ ID NO: 20) | | | | |

The cassettes described above in Example 15 were transformed into S. cerevisiae using a standard lithium acetate (LiAC) mediated protocol. Briefly, single colonies from a yeast extract peptone dextrose agar plate (YPD Agar, 1% yeast extract, 2% peptone, 2% dextrose, in distilled water) were inoculated into 5 ml yeast extract peptone dextrose broth (YPD, 1% yeast extract, 2% peptone, 2% dextrose, in distilled water) and grown overnight at 30° C. at 200 RPM. The next day, the culture was diluted to an OD600 of 0.2 and grown at 30° C., 200 RPM, until the culture reached OD600 of 0.8-1. The cells were harvested by centrifugation at 2500 RPM for 5 minutes. The cell pellet was resuspended in 1 ml of 1 M lithium acetate (LiAc) and centrifuged again at 2500 RPM for five minutes. The supernatant was discarded and the pellet was resuspended in 100 μl of 1 M LiAc. For each transformation reaction 24 μl cells were added to 90 μl 50% PEG 3350, 8 μl boiled salmon sperm, 6 μl PCR product and 2 μl plasmid.

The mixture was vortexed to mix and incubated for 30 minutes at 25° C. 12 μl of 60% glycerol was added, mixed by vortexing, and again incubated at 25° C. for 30 minutes.

The transformation was briefly mixed again by vortexing and incubated at 42° C. for 20 minutes. Cells are recovered in 3 ml YPD media for 3 to 5 hours at 30° C. or overnight at 25° C. before 300 μl of the cells were plated on YPD agar containing appropriate antibiotics.

All DNA integrations were confirmed by colony PCR using primers specific to the integrations using a Phire Plant Direct PCR Master mix (Cat #F160L, Thermo Fisher) kit.

### Example 16. Analytical Methods for Phytoene and Bisabolol Extraction from S. cerevisiae

As phytoene accumulates inside S. cerevisiae, cell lysis and liquid-liquid extraction were required to quantify phytoene. Briefly, 20 OD₆₀₀ of cells were harvested in a 1.5 mL tube by pelleting at 10,000×g for one minute. The cells were washed once with 1 ml water and then resuspended in 300 μl 3N HCl. Following pelleting at 10,000×g for one minute the cells were resuspended in 200 μl 3N HCl. A 80° C. preheated Thermomixer (Eppendorf) was used to mix the cells at 1000 rpm for 7 minutes. The tube was then moved to an ice-water bath to cool for 3 mins. Mixing with the Thermomixer was repeated an additional time. In the meantime, bead beating tubes were prepared containing about ¼ tube of 1 mm beads. The heated mixed cell/HCl mixture was transferred to the bead beating tubes and 800 μl acetone added on top. The tubes were transferred to a bead beater (FASTPREP 24 5G; MB Biomedical) and a twenty second cell lysis protocol was run 3 times with 1 minute cooling between bead beating. Following bead beating the tubes were centrifuged at max speed for 1 minute and 500 μl of the supernatant was transferred to a new 2 ml tube along with 500 μl ethyl acetate. The tube was vortexed at max speed for 30 seconds to mix. 500 μl (half) of the mixture was transferred to 2 new 1.5 mL tubes and 500 μl water added to each tube. The tubes were again mixed by vortexing at max speed for 30 seconds to mix the organic and aqueous layer. The layers were separated by centrifugation for 1 minute at max speed and 125 μl of the top organic layer from each tube is added to a single 1.5 mL tube (250 μl total) to recombine the two samples. Samples were evaporated using a SpeedVac (Concentrator plus; Eppendorf) without heating for 30 minutes to evaporate the ethyl acetate. The pellet (containing phytoene) was dissolved in 100 μl 80% Methanol+20% Methyl tert-Butyl Ether (HPLC mobile phase) and quantified as described in Example 3.

Bisabolol can be extracted from cultures using a 10% ethyl oleate overlay. After growth and induction the culture is spun down at 10,000×g for 10 minutes to remove cell debris and separate the organic layer. The top organic layer was diluted ten times in a diluent containing 90% ethyl acetate and 10% 1,3,5-triisopropylbenzene and analysed by GC-FID.

### Example 17. Analytical Methods for Quantification of Bisabolol Produced in S. cerevisiae

The bisabolol was quantified using Gas Chromatography with flame ionisation detector (FID) and a HP-5 column (Part number: 19091J-413, 30 m×0.32 mm×0.25 μm, Agilent, SC, USA). Helium was used as the carrier gas at a flow rate of 1.4 mL/min with injector and FID temperatures set at 240° C. and 300° C., respectively. The oven temperature gradient was set as follows: Hold at 60° C. for 2 minutes; increase at 20° C./min to 300° C.; hold at 300° C. for 2 minutes. 1 μl of the diluted sample was injected into the column for analysis. The retention times for bisabolol and ethyl oleate are 10.4 min and 12.9 min respectively.

### Example 18. Shake Flask Production of Bisabolol or Phytoene in S. cerevisiae

Transformed yeast strains expressing bisabolol were inoculated into 5 ml of YPD containing the appropriate antibiotics (zeocin) in a corning 50 ml mini bio reactor tube (Cat #CLS431720, Sigma) and grown overnight at 30° C. with shaking at 200 rpm. A 250 mL baffled flask was filled with 25 ml YPD and inoculated to OD_{6O0} with the cells from the overnight culture. Cells were grown for 6 hours at 30° C. with shaking at 200 rpm. 10% volume ethyl oleate was added to the flask and allowed to continue shaking at 200 rpm for 72 hours. The cultures were harvested and extracted and quantified as described above in Example 16 and 18 above.

Transformed yeast strains expressing Phytoene were inoculated into 5 mL YPD and allowed to grow overnight. In the morning a 250 ml baffled flask was inoculated with 25 mL fresh YPD and cells added to OD₆₀₀. After incubating the flask for 22 hours at 30° C. with shaking at 200 rpm the OD₆₀₀ was approximately 15 for all strains. The cells were spun down at 2000×g for 5 minutes and cells were resuspended in 25 ml YPGalactose (YPGalactose, 1% yeast extract, 2% peptone, 2% galactose) and allowed to grow in inducing conditions for another 48 hours. 20 OD600 of cells were harvested. Phytoene was extracted and quantified as described above in Examples 2 and 3 above.

### Example 19. Identification of Novel Proteins with Phytoene Synthase (PYS) and Geranyigeranyl Diphosphate Synthase (GGPPS) Activity from a Natural Diversity Library Screen in S. cerevisiae

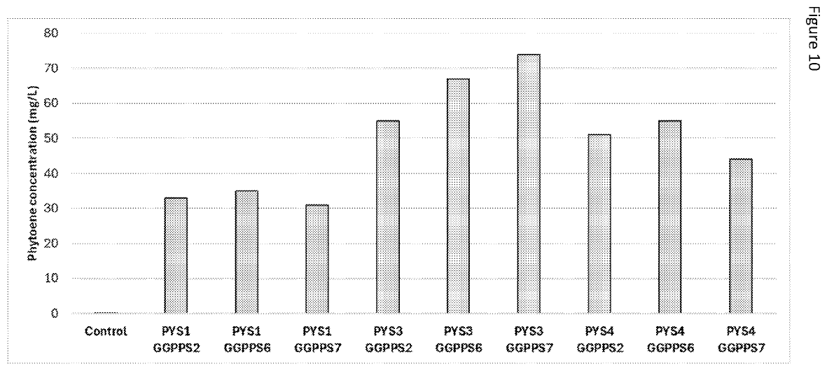

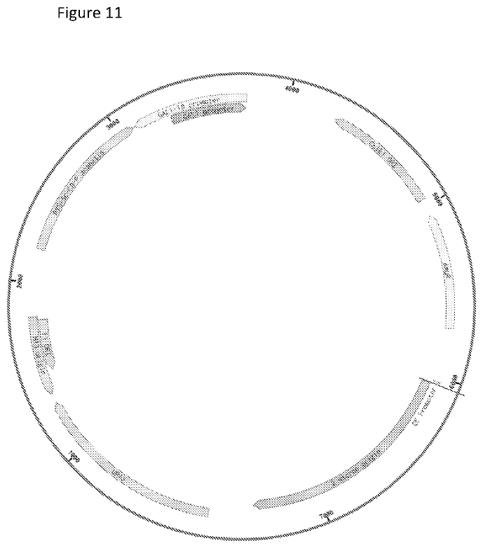

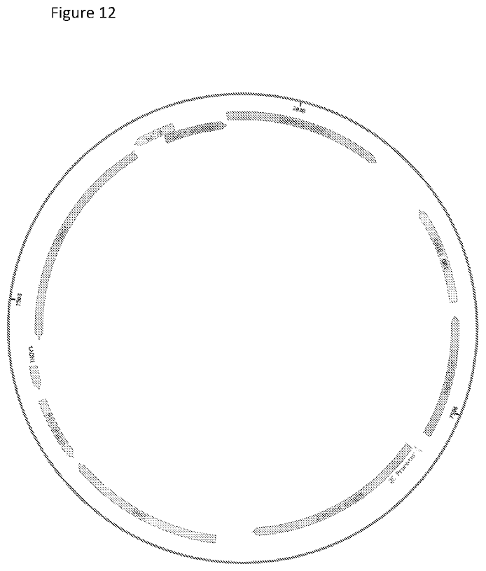

Similar to the work performed in E. coli, we also tested the activity of different phytoene synthase (PYS) and Geranylgeranyl Diphosphate Synthase (GGPPS) genes from a diversity library. To produce the library we then overexpressed the following PYS genes in a yeast episomal plasmid with HIS3 as the selectable marker (FIG. 11; an example of an exemplary sequence is found in SEQ ID NO: 59). GGPPS and a truncated version of native yeast HMG1 (SEQ ID NO: 20) are expressed using a yeast episomal plasmid with URA3 as the selectable marker (FIG. 12; an example of an exemplary sequence is found in SEQ ID NO: 60). PYS, GGPPS, and tHMG1 are under the pGal1/10 promoter. Both plasmids are transformed into un-modified S. cerevisiae BY4741 strain. The titers of phytoene produced by these strains are shown in FIG. 10 where PYS3 represents the PYS gene from Enterobacter agglomerans (SEQ ID NO: 3), PYS1 represents the PYS gene from Pantoea ananatis (SEQ ID NO: 1), GGPPS2 from Capsicum annuum (SEQ ID NO: 9), GGPPS6 represents the GGPPS from Croton sublyratus (SEQ ID NO: 13), and GGPPS7 from Hevea brasiliensis (SEQ ID NO: 14). Shown in FIG. 10, we see that the combination of PYS3 (Enterobacter agglomerans; SEQ ID NO: 3) and GGPPS7 (Hevea brasiliensis; SEQ ID NO: 14) result in a strain producing the most phytoene at 75 mg/L and the second best strain expressing the same PYS3 (Enterobacter agglomerans; SEQ ID NO: 3) with GGPPS6 (Croton sublyratus; SEQ ID NO: 13) at 68 mg/L.

### Example 20. Promoter Titration of Strains Expressing the Genes Phytoene Synthase (PYS) and Geranylgeranyl Diphosphate Synthase (GGPPS) to Produce Phytoene in S. cerevisiae

Because some of the intermediates produced can be toxic and it is common that different expression promoters can result in varying amounts of product produced, we tested several promoters to identify the optimal combination. The parent strain described above in Example 15 was transformed with the codon optimised PYS from E. agglomerans (PYS3; SEQ ID NO: 3) and GGPPS from H. brasiliensis (GGPPS7; SEQ ID NO: 14) using different promoters from S. cerevisiae. We tested four different promoter designs and two integration loci, FCY1 and NSIX. Strains were transformed using the method described in Example 15, grown using the method described in Example 18, extracted using the method described in Example 16, and quantified using the method described in Example 3. In addition to the amount of phytoene produced, we also quantified the dry cell weight (DCW) of the biomass using standard methods and the data in Table 3 is in phytoene as a % gDCW.

TABLE 3
Optimization of promoters for the expression of PYS from E. agglomerans (PYS3;
SEQ ID NO: 3) and GGPPS from H. brasiliensis (GGPPS7; SEQ ID NO: 14).

| | Integration | terminator | | Promoter | Promoter | | terminator | Phytoene |
|---|---|---|---|---|---|---|---|---|
| Strain | Locus | 1 | Gene 1 | 1 | 2 | Gene 2 | 2 | (% gDCW) |
| Parent | | | | | | | | 0 |
| PI0001 | FCY1 | tCYC1 | PYS3 | pGal1 | pGal10 | GGPPS7 | tDIT1 | 0.094 |
| PI0002 | FCY1 | tCYC1 | PYS3 | pGal7 | pGal3 | GGPPS7 | tDIT1 | 0.037 |
| PI0003 | FCY1 | tCYC1 | PYS3 | pGal3 | pGal7 | GGPPS7 | tDIT1 | 0.03 |
| PI0004 | FCY1 | tADH1 | PYS3 | pTDH1 | pENO2 | GGPPS7 | tDIT1 | 0.018 |
| PI0005 | NSIX | tCYC1 | PYS3 | pGal1 | pGal10 | GGPPS7 | tDIT1 | 0.052 |
| PI0006 | NSIX | tCYC1 | PYS3 | pGal7 | pGal3 | GGPPS7 | tDIT1 | 0.103 |
| PI0007 | NSIX | tCYC1 | PYS3 | pGal3 | pGal7 | GGPPS7 | tDIT1 | 0.092 |
| PI0008 | NSIX | tADH1 | PYS3 | pTDH1 | pENO2 | GGPPS7 | tDIT1 | 0 |

Based on the results shown in Table 3, among the eight designs, galactose inducible promoters resulted in higher phytoene production than the constitutive promoters. Therefore, we focused on using a strong bidirectional galactose promoter, pGal1/10, or weaker galactose promoters pGal3 and pGal7 in the later experiments.

### Example 21. Identification of Novel Bacterial Proteins with Phytoene Synthase (CrtB), or a Bifunctional Lycopene Cyclase/Phytoene Synthase (CrtYB) and Geranylgeranyl Diphosphate Synthase (CrtE) to Produce Phytoene in S. cerevisiae

In order to expand the diversity of genes, we screened novel bacterial proteins that have phytoene synthase (CrtB), or a bifunctional lycopene cyclase/phytoene synthase (CrtYB) and Geranylgeranyl Diphosphate Synthase (CrtE) activity for production of phytoene in yeast. All genes were codon optimised for yeast and synthesised by Genscript. In this case we transformed all DNA into a single locus NSIX using different pGal promoters to drive expression. Similar to Example 20 we tested several promoters to identify the optimal combination and toxic ensure intermediates were not building up in the strains. Strains were transformed using the method described in Example 15, grown using the method described in Example 18, extracted using the method described in Example 16, and quantified using the method described in Example 3. In addition to the amount of phytoene produced, we also quantified the dry cell weight (DCW) of the biomass using standard methods and the data in Table 4 is in phytoene as a % gDCW.

TABLE 4
Optimization of promoters and screening bacterial proteins with phytoene synthase (CrtB), or a bifunctional
lycopene cyclase/phytoene synthase (CrtYB) and Geranylgeranyl Diphosphate Synthase (CrtE)

| | Integration | terminator | | Promoter | Promoter | | terminator | Phytoene |
|---|---|---|---|---|---|---|---|---|
| Strain | Locus | 1 | Gene 1 | 1 | 2 | Gene 2 | 2 | (% gDCW) |
| Parent | | | | | | | | 0 |
| PI0009 | NSIX | tCyc1 | CrtB_Aa | pGal3 | pGal7 | CrtE_Af | tDit1 | 0.176 |
| | | | (SEQ ID | | | (SEQ ID | | |
| | | | NO: 51) | | | NO: 55) | | |
| PI00015 | NSIX | tCyc1 | CrtB_Aa | pGal7 | pGal3 | CrtE_Af | tDit1 | 0.105 |
| | | | (SEQ ID | | | (SEQ ID | | |
| | | | NO: 51) | | | NO: 55) | | |
| PI00018 | NSIX | tCyc1 | CrtB_Pa | pGal3 | pGal7 | CrtE_Af | tDit1 | 1.143 |
| | | | (SEQ ID | | | (SEQ ID | | |
| | | | NO: 52) | | | NO: 55) | | |
| PI00020 | NSIX | tCyc1 | CrtB_Aa | pGal1 | pGal10 | CrtE_Af | tDit1 | 0.452 |
| | | | (SEQ ID | | | (SEQ ID | | |
| | | | NO: 51) | | | NO: 55) | | |
| PI00021 | NSIX | tCyc1 | CrtB_Pa | pGal1 | pGal10 | CrtE_Af | tDit1 | 0.52 |
| | | | (SEQ ID | | | (SEQ ID | | |
| | | | NO: 52) | | | NO: 55) | | |
| PI00024 | NSIX | tCyc1 | CrtB_Pa | pGal3 | pGal7 | CrtE_Txm | tDit1 | 0.405 |
| | | | (SEQ ID | | | (SEQ ID | | |
| | | | NO: 52) | | | NO: 56) | | |
| PI00025 | NSIX | tCyc1 | CrtB_Pa | pGal3 | pGal7 | CrtE_Xd | tDit1 | 0.133 |
| | | | (SEQ ID | | | (SEQ ID | | |
| | | | NO: 52) | | | NO: 57) | | |
| PI00026 | NSIX | tCyc1 | CrtB_Pa | pGal3 | pGal7 | CrtE_Bt | tDit1 | 0.309 |
| | | | (SEQ ID | | | (SEQ ID | | |
| | | | NO: 52) | | | NO: 58) | | |
| PI00027 | NSIX | tCyc1 | CrtYB_MI | pGal3 | pGal7 | CrtE_Af | tDit1 | 0.531 |
| | | | (SEQ ID | | | (SEQ ID | | |
| | | | NO: 53) | | | NO: 55) | | |
| PI00028 | NSIX | tCyc1 | CrtYB_MI | pGal3 | pGal7 | CrtE_Txm | tDit1 | 0.119 |
| | | | (SEQ ID | | | (SEQ ID | | |
| | | | NO: 53) | | | NO: 56) | | |
| PI00030 | NSIX | tCyc1 | CrtYB_MI | pGal3 | pGal7 | CrtE_Bt | tDit1 | 0.084 |
| | | | (SEQ ID | | | (SEQ ID | | |
| | | | NO: 53) | | | NO: 58) | | |
| PI00031 | NSIX | tCyc1 | CrtYB_Xd | pGal3 | pGal7 | CrtE_Af | tDit1 | 0.116 |
| | | | (SEQ ID | | | (SEQ ID | | |
| | | | NO: 57) | | | NO: 55) | | |
| PI00032 | NSIX | tCyc1 | CrtYB_Xd | pGal3 | pGal7 | CrtE_Txm | tDit1 | 0.223 |
| | | | (SEQ ID | | | (SEQ ID | | |
| | | | NO: 57) | | | NO: 56) | | |
| PI00033 | NSIX | tCyc1 | CrtYB_Xd | pGal3 | pGal7 | CrtE_Xd | tDit1 | 0.096 |
| | | | (SEQ ID | | | (SEQ ID | | |
| | | | NO: 57) | | | NO: 57) | | |
| PI00034 | NSIX | tCyc1 | CrtYB_Xd | pGal3 | pGal7 | CrtE_Bt | tDit1 | 0.261 |
| | | | (SEQ ID | | | (SEQ ID | | |
| | | | NO: 57) | | | NO: 58) | | |

As seen in Table 4, we screened the following bacterial CrtB, CrtYB and CrtE genes: CrtB_Aa from Agrobacterium aurantiacum (SEQ ID NO: 51), CrtB_Pa from Pantoea agglomerans (SEQ ID NO: 52), CrtYB_MI from Mucor lusitanicus (SEQ ID NO: 53), CrtYB_Xd from Xanthophyllomyces dendrorhous (SEQ ID NO: 54), CrtE_Af from Archaeoglobus fulgidus (SEQ ID NO: 55), CrtE_Txm from Taxus x media (SEQ ID NO: 56), CrtE_Xd from Xanthophyllomyces dendrorhous (SEQ ID NO: 57), and CrtE_Bt from Blakeslea trispora (SEQ ID NO: 58). The highest phytoene producer used the lowest expression promoter, pGal3 to drive CrtB_Pa from Pantoea agglomerans (SEQ ID NO: 52) and pGal7 the middle expression promoter to drive CrtE_Af from Archaeoglobus fulgidus (SEQ ID NO: 55). These results indicate that while pGal1 and pGal10 are much more highly expressed, the production of phytoene requires the right balance of enzymes expressed. In summary, between experiments described above in Example 20 and here in Example 21, the highest phytoene producer, PI0018, is producing ~40-fold higher phytoene than a low producer, PI003.

### Example 22. Production of Bisabolol in S. cerevisiae Using Novel Bisabolol Synthase (BBS) Genes from a Novel Diversity Library

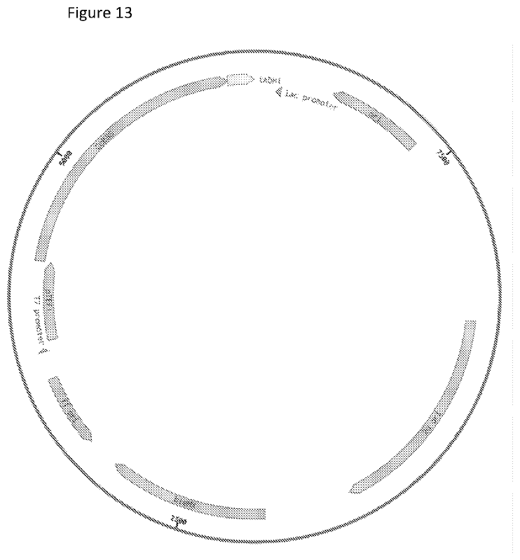

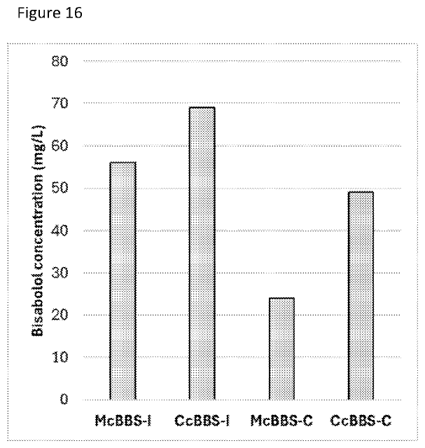

Similar to the work done in E. coli, we also tested the expression of novel bisabolol synthase (BBS) genes to assess if S. cerevisiae could produce bisabolol. In this case we used the same parent strain described in Example 15 and transformed various BBS genes using a protocol similar to that described above in Example 15 above. In this case, the BBS genes were expressed from a plasmid instead of integrated into the genome. An exemplary example of this plasmid is shown in FIG. 13 and in SEQ ID NO: 61. Briefly, this plasmid is a yeast shuttle vector based on pPRS425 also used in Example 15. Each BBS gene was codon optimised for yeast and synthesised by Genescript. They are driven by the native S. cerevisiae TEF1 promoter (amplified from gDNA) and end with the native S. cerevisiae ADH1 terminator (amplified from gDNA). In addition, this plasmid contains the bleMX gene allowing for resulting transformants to be resistant to phleomycin. Strains were transformed using the method described in Example 15, grown using the method described in Example 18, extracted using the method described in Example 16, and quantified using the method described in Example 17. We tested McBBS (BBS6 from Matricaria chamomilla, SEQ ID NO: 30) and CcBBS (BBS1 from Cynara cardunculus; SEQ ID NO: 25) with constitutive S. cerevisiae TEF1 promoter and inducible S. cerevisiae pGal1/10 promoter using the pGal1 to drive the BBS gene. The data for this experiment is shown in FIG. 16. The most bisabolol produced in S. cerevisiae was 70 mg/L was observed with the pGal inducible CcBBS (BBS1 from Cynara cardunculus; SEQ ID NO: 25).

### Example 23. Analytical Methods for In Vitro Quantification of Mevalonate Kinase (MVK) Activity Expressed in E. coli

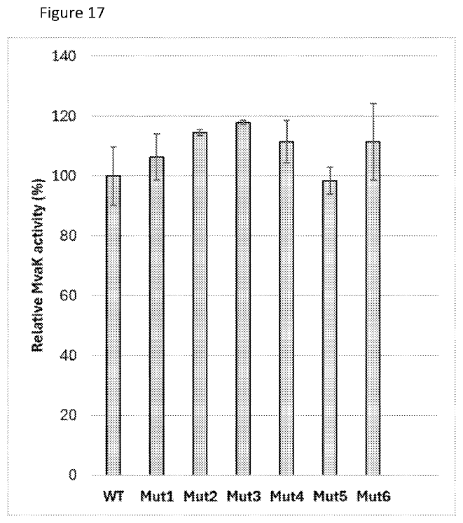

To measure the activity of improved variants of Mevalonate Kinase (MVK), a fluorimetric assay was developed. In vivo, the MVK enzyme phosphorylates mevalonate, converting ATP to ADP in the process. A universal kinase assay that produces a fluorescent surrogate marker for kinase activity was used to measure kinase activity across different MVK enzyme mutants. E. coli cells expressing the wild type S. cerevisiae MVK (SEQ ID NO: 22) were constructed by cloning the ERG12 gene from S. cerevisiae into pCDFDuet vector under the control of T7 promoter. The MVK protein was expressed by growing the recombinant strain in LB medium supplemented with glucose and expression induced by addition of 0.4 mM IPTG. The cells were grown for 16 h at 25° C. and then added to a mixture containing mevalonate, ATP, and an assay buffer. Cells were incubated for 1 h at 30° C., at which point a sensor buffer was added to the mixture to convert kinase activity into a fluorescent surrogate marker. Fluorescence intensity was measured for individual clones. FIG. 17 shows the activity of WT S. cerevisiae MVK, expressed in E. coli.

### Example 24. Identification of S. cerevisiae Variant MVK Proteins with Improved MVK Activity Using Site Saturation Mutagenesis

The WT MVK of S. cerevisiae was scanned for beneficial mutations by mutating >20% of residues through single site saturation mutagenesis using standard molecular biology techniques. The library was expressed using the protocol described in Example 23. After 16 h of enzyme expression, single cells were encapsulated in droplets and screened using a microfluidics screening platform in which kinase activity is converted to a fluorescent marker, and the fluorescence of every individual droplet is detected (as described in the previous patent US 2021/0318315 A1). Droplets were sorted into Bright and Dark pools based on their individual fluorescence values. The collection of MVK enzyme mutants within each pool was then sequenced to identify mutations enriched in Bright pools of droplets. To confirm that the single mutations are causal for improvement, each identified mutation was introduced into the plasmid described in Example 23 (containing the pCDFDuet with the T7 promoter driving the MVK gene) and MVK expression was induced as described in Example 23. The improved activity of mutant MVK enzymes (measured in the fluorometric assay) is shown in FIG. 17. The top mutation, Mut3 showed an increase of 18% activity compared to the WT MVK activity (SEQ ID NO: 22). The Identified improved mutations are shown in FIG. 17 and described as follows: Mut1 has the glutamic acid at amino acid residue 419 mutated to aspartic acid (E419D), Mut2 has the lysine at amino acid residue 402 mutated to valine (K402V), Mut3 has the glutamic acid at amino acid residue 73 mutated to glycine (E73G), Mut4 has the glycine at amino acid residue 393 mutated to valine (G393V), Mut5 has the leucine at amino acid residue 175 mutated to valine (L175V), Mut6 has the aspartic acid at amino acid residue 74 mutated to glutamic acid (D74D).

1. A method for producing a carotenoid or terpenoid wherein the method comprises culturing a recombinant cell, wherein the cell expresses or overexpresses one or more enzymes of the mevalonate pathway and/or methylerythritol pathway, and wherein said one or more enzymes of the mevalonate pathway and/or methylerythritol pathway includes at least 3-hydroxy-3-methylglutaryl-CoA reductase, wherein the 3-hydroxy-3-methylglutaryl-CoA reductase enzyme:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
wherein said expression or overexpression results in increased production of:
i) phytoene;
ii) carotenoids;
iii) bisabolol;
iv) terpenoids; and/or
v) isopentenyl pyrophosphate (IPP) and dimethylallyl pyrophosphate (DMAPP)
relative to a cell that does not express and/or overexpress said one or more enzymes of the mevalonate pathway and/or methylerythritol pathway,
optionally relative to a cell that is able to produce
i) phytoene;
ii) carotenoids;
iii) bisabolol;
iv) terpenoids; and/or
v) isopentenyl pyrophosphate (IPP) and dimethylallyl pyrophosphate (DMAPP)
but that does not express and/or overexpress said one or more enzymes of the mevalonate pathway and/or methylerythritol pathway.

2. The method according to claim 1 wherein the method is for producing a carotenoid and wherein the cell further expresses or overexpresses:
a) phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 1-7, 51 or 52; and
b) a GGPPS enzyme having an amino acid sequence of any of SEQ ID NO: 14, 8-13, 15, 16, 55, 56, 57 or 58 or having an amino acid sequence with at least 80%, 85%, 90%, 92%, 94%, 96%, 98%, 99% or 100% similarity to any of SEQ ID NO: 14, 8-13, 15, 16, 55, 56, 57 or 58.

3. The method according to claim 1 or 2 wherein the carotenoid is phytoene, or any carotenoid derived from phytoene as a precursor; and/or the terpenoid is bisabolol or any other terpenoid derived from IPP and DMAPP as precursors.

4. The method according to any of claims 1-3 wherein the carotenoid is lycopene and the cell further comprises a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38.

5. The method according to any of claims 1-3 wherein the carotenoid is neurosporene and the cell further comprises a phytoene desaturase enzyme that converts phytoene to neurosporene, optionally wherein the phytoene desaturase enzyme has an amino acid sequence of SEQ ID NO: 36, or has an amino acid sequence with at least 80%, 85%, 90%, 92%, 94%, 96%, 98%, 99% or 100% similarity to SEQ ID NO: 36.

6. The method according to any of claims 1-3 wherein the method is for the production of Bisabolol and the cell comprises a Bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30.

7. The method according to any of claims 1-6 wherein the cell comprises an MVA pathway that comprises one or more or all of the following enzymes:
i) a HmgACT enzyme, optionally of SEQ ID NO: 17 or a protein with an amino acid sequence with at least 80%, 85%, 90%, 92%, 94%, 96%, 98%, 99% or 100% similarity to said sequence;
ii) a HmgS enzyme, optionally of SEQ ID NO: 18 or a protein with an amino acid sequence with at least 80%, 85%, 90%, 92%, 94%, 96%, 98%, 99% or 100% similarity to said sequence;
iii) the HmgR enzyme as set out in claim 1;
iv) a MVK enzyme, optionally:
of SEQ ID NO: 22 or a protein with an amino acid sequence with at least 80%, 85%, 90%, 92%, 94%, 96%, 98%, 99% or 100% similarity to said sequence; has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
that comprises a E419D substitution, a K402V substitution, a E73G substitution, a G393V substitution, a L175V substitution, a D74E substitution, wherein said positions are numbered with reference to SEQ ID No: 22;
that has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
that comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74; or
that has an amino acid sequence of SEQ ID NO: 22;
v) a PMK enzyme, optionally of SEQ ID NO: 23 or a protein with an amino acid sequence with at least 80%, 85%, 90%, 92%, 94%, 96%, 98%, 99% or 100% similarity to said sequence;
vi) a PMD enzyme, optionally of SEQ ID NO: 24 or a protein with an amino acid sequence with at least 80%, 85%, 90%, 92%, 94%, 96%, 98%, 99% or 100% similarity to said sequence; and
vii) an isopentenyl-diphosphate delta isomerase 1 enzyme, optionally with an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41.

8. The method of any of claims 1-7 wherein the cell comprises an MEP pathway that comprises expression or overexpression of one or more of all of the following enzymes:
an isopentenyl-diphosphate delta isomerase 1 enzyme, optionally an isopentenyl-diphosphate delta isomerase enzyme from B. subtilis or E. coli, optionally an isopentenyl-diphosphate delta isomerase enzyme with an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
a 1-deoxy-D-xylulose-5-phosphate synthase enzyme, optionally a 1-deoxy-D-xylulose-5-phosphate synthase enzyme from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

9. The method of any of claims 1-8 wherein the cell is a prokaryotic cell, optionally wherein the cell is an E. coli cell, Corynebacterium glutamicum cell, or a Bacillus sp cell.

10. The method of any of claims 1-8 wherein the cell is a eukaryotic cell, optionally wherein the cell is an yeast cell, optionally a S. cerevisiae cell or a Yarrowia lipolytica cell.

11. The method of any of claims 1-10 wherein the method produces:
at least 0.1%, 0.5%, 1%, 2%, 5%, 10%, 15%, 20%, 25%, 30%, 35%, 40%, 45%, 50%, 55%, 60%, 70%, 80%, 90%, 100% or more phytoene from a cell that expresses or overexpresses the 3-hydroxy-3-methylglutaryl-CoA reductase set out in claim 1 than the amount of phytoene produced from the same cell but which does not express or overexpress the 3-hydroxy-3-methylglutaryl-CoA reductase enzyme set out in claim 1.

12. The method of any of claims 1-10 wherein the method produces:
at least 0.1%, 0.5%, 1%, 2%, 5%, 10%, 15%, 20%, 25%, 30%, 35%, 40%, 45%, 50%, 55%, 60%, 70%, 80%, 90%, 100% or more phytoene from a cell that expresses or overexpresses:
a) the 3-hydroxy-3-methylglutaryl-CoA reductase set out in claim 1;
b) the phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 1-7, 51 or 52; and
c) the GGPPS enzyme having an amino acid sequence of any of SEQ ID NO: 14, 8-13, 15, 16, 55, 56, 57 or 58 or having an amino acid sequence with at least 80%, 85%, 90%, 92%, 94%, 96%, 98%, 99% or 100% similarity to any of SEQ ID NO: 14, 8-13, 15, 16, 55, 56, 57 or 58
compared to the amount of phytoene produced by a native or wild type cell that does not express and/or overexpress any of the above enzymes, optionally comprised to the amount of phytoene produced by a native or wild type cell that is able to produce phytoene but that does not express and/or overexpress any of the above enzymes.

13. An engineered carotenoid production cell, wherein the engineered carotenoid production cell comprises:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 3, 1, 2, 4-7, 51 or 52; and
c) a polynucleotide encoding a GGPPS enzyme having an amino acid sequence of any of SEQ ID NO: 14, 8-13, 15, 16, 55, 56, 57, or 58 or having an amino acid sequence with at least 80%, 85%, 90%, 92%, 94%, 96%, 98%, 99% or 100% similarity to any of SEQ ID NO: 14, 8-13, 15, 16, 55, 56, 57, or 58;
d) a polynucleotide encoding a mevalonate kinase enzyme that:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.

14. The engineered carotenoid production cell of claim 13 wherein the:
a) phytoene synthase has an amino acid of sequence of SEQ ID NO: 3, optionally an amino acid sequence with at least 80%, 85%, 90%, 92%, 94%, 96%, 98%, 99% or 100% similarity to SEQ ID NO 3; and/or
b) GGPPS enzyme has an amino acid sequence of SEQ ID NO: 14, optionally an amino acid sequence with at least 80%, 85%, 90%, 92%, 94%, 96%, 98%, 99% or 100% similarity to SEQ ID NO: 14.

15. The engineered carotenoid production cell of any of claim 13 or 14 wherein the carotenoid is phytoene.

16. The engineered carotenoid production cell of any of claim 13 or 14 wherein the carotenoid is lycopene, optionally wherein the cell further comprises a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38.

17. The engineered carotenoid production cell of any of claim 13 or 14 wherein the carotenoid is neurosporene, optionally wherein cell further comprises a phytoene desaturase enzyme that converts phytoene to neurosporene, optionally wherein the phytoene desaturase enzyme has an amino acid sequence of SEQ ID NO: 36, or has an amino acid sequence with at least 80%, 85%, 90%, 92%, 94%, 96%, 98%, 99% or 100% similarity to SEQ ID NO: 36.

18. An engineered terpenoid production cell wherein the cell comprises:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
and/or
b) a polynucleotide encoding a mevalonate kinase enzyme that:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.

19. The engineered terpenoid production cell of claim 18 wherein the terpenoid is bisabolol and the cell further comprises a bisabolol synthase enzyme with an amino acid sequence of any of SEQ ID NO: 25-30 or with an amino acid sequence with at least 80%, 85%, 90%, 92%, 94%, 96%, 98%, 99% or 100% similarity to any of SEQ ID NO: 25-30, optionally has an amino acid sequence of SEQ ID NO: 29 or 25 or with at least 80%, 85%, 90%, 92%, 94%, 96%, 98%, 99% or 100% similarity to any of SEQ ID NO: 29 or 25.

20. The engineered carotenoid production cell of any of claims 13-17 or the engineered terpenoid production cell of any of claim 18 or 19 wherein, where present, the polynucleotides encoding the HmgR, phytoene synthase enzyme, GGPPS, phytoene desaturase or bisabolol synthase are all present on the same nucleic acid molecule, optionally are both present on the same plasmid.

21. The engineered carotenoid production cell of any of claims 13-17 or the engineered terpenoid production cell of any of claim 18 or 19 wherein, where present, the polynucleotides encoding the HmgR, phytoene synthase enzyme, GGPPS, phytoene desaturase or bisabolol synthase are all integrated into the genome of the phytoene production cell.

22. The engineered carotenoid production cell of any of claims 13-17, 20 and 21 or the engineered terpenoid production cell of any of claims 18-2 further comprising a functional MVA pathway, wherein the MVA pathway comprises expression or overexpression of one or more or all of the following enzymes:
a HmgACT enzyme, optionally of SEQ ID NO: 17 or a protein with an amino acid sequence with at least 80%, 85%, 90%, 92%, 94%, 96%, 98%, 99% or 100% similarity to said sequence;
a HmgS enzyme, optionally of SEQ ID NO: 18 or a protein with an amino acid sequence with at least 80%, 85%, 90%, 92%, 94%, 96%, 98%, 99% or 100% similarity to said sequence;
the said HmgR enzyme,
a MVK enzyme, optionally that
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
a PMK enzyme, optionally of SEQ ID NO: 23 or a protein with an amino acid sequence with at least 80%, 85%, 90%, 92%, 94%, 96%, 98%, 99% or 100% similarity to said sequence;
a PMD enzyme, optionally of SEQ ID NO: 24 or a protein with an amino acid sequence with at least 80%, 85%, 90%, 92%, 94%, 96%, 98%, 99% or 100% similarity to said sequence; and
an isopentenyl-diphosphate delta isomerase 1 enzyme, optionally with an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41.

23. The engineered carotenoid production cell of any of claims 13-17, and 20-22 or the engineered terpenoid production cell of any of claims 18-22 wherein the cell comprises a functional MEP pathway, and wherein the MEP pathway includes the expression or overexpression of any one or more or all of the following enzymes:
an isopentenyl-diphosphate delta isomerase 1 enzyme, optionally an isopentenyl-diphosphate delta isomerase enzyme from B. subtilis or E. coli, optionally an isopentenyl-diphosphate delta isomerase enzyme with an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
a 1-deoxy-D-xylulose-5-phosphate synthase enzyme, optionally a 1-deoxy-D-xylulose-5-phosphate synthase enzyme from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

24. The engineered carotenoid production cell of any of claims 13-17, and 20-23 or the engineered terpenoid production cell of any of claims 18-23 wherein the cell is a prokaryotic cell, optionally wherein the cell is an E. coli cell, Corynebacterium glutamicum cell, or a Bacillus sp cell.

25. The engineered carotenoid production cell of any of claims 13-17, and 20-23 or the engineered terpenoid production cell of any of claims 18-23 wherein the cell is a eukaryotic cell, optionally wherein the cell is an yeast cell, optionally a S. cerevisiae cell or a Yarrowia lipolytica cell.

26. The engineered carotenoid production cell of any of claims 13-17 and 20-25 wherein the engineered cell is able to produce at least 0.1%, 0.5%, 1%, 2%, 5%, 10%, 15%, 20%, 25%, 30%, 35%, 40%, 45%, 50%, 55%, 60%, 70%, 80%, 90%, 100% or more phytoene, neurosporene and/or lycopene relative to a cell of the same species and genetic background that does not comprise any one or more or all of the following:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 3, 1, 2, 4-7, 51 or 52;
c) a polynucleotide encoding a GGPPS enzyme having an amino acid sequence of any of SEQ ID NO: 14, 8-13, 15, 16, 55, 56, 57 or 58 or having an amino acid sequence with at least 80%, 85%, 90%, 92%, 94%, 96%, 98%, 99% or 100% similarity to any of SEQ ID NO: 14, 8-13, 15, 16, 55, 56, 57 or 58; or
d) a polynucleotide encoding a phytoene synthase has an amino acid of sequence of SEQ ID NO: 3, optionally an amino acid sequence with at least 80%, 85%, 90%, 92%, 94%, 96%, 98%, 99% or 100% similarity to SEQ ID NO 3;
e) a polynucleotide encoding a GGPPS enzyme has an amino acid sequence of SEQ ID NO: 14, optionally an amino acid sequence with at least 80%, 85%, 90%, 92%, 94%, 96%, 98%, 99% or 100% similarity to SEQ ID NO: 14; and/or
f) a polynucleotide encoding a mevalonate kinase enzyme that:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
optionally wherein the phytoene production cell is capable of producing at least 0.1%, 0.5%, 1%, 2%, 5%, 10%, 15%, 20%, 25%, 30%, 35%, 40%, 45%, 50%, 55%, 60%, 70%, 80%, 90%, 100% or more phytoene than a cell that does not comprise one or more or all of the above polynucleotides.

27. The engineered terpenoid production cell of any of claims 18-25 wherein the engineered cell is able to produce at least 0.1%, 0.5%, 1%, 2%, 5%, 10%, 15%, 20%, 25%, 30%, 35%, 40%, 45%, 50%, 55%, 60%, 70%, 80%, 90%, 100% or more bisabolol relative to a cell of the same species and genetic background that does not comprise any one or more or all of the following:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a bisabolol synthase enzyme with an amino acid sequence of any of SEQ ID NO: 25-30 or with an amino acid sequence with at least 80%, 85%, 90%, 92%, 94%, 96%, 98% 99% or 100% similarity to any of SEQ ID NO: 25-30, optionally has an amino acid sequence of SEQ ID NO: 29 or 25 or with at least 80%, 85%, 90%, 92%, 94%, 96%, 98%, 99% or 100% similarity to any of SEQ ID NO: 29 or 25; and/or
c) a polynucleotide encoding a mevalonate kinase enzyme that:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.

28. A method for the production of phytoene wherein the method comprises culturing an engineered carotenoid production cell according to any of claims 13-17 and 20-25.

29. A method for the production of lycopene wherein the method comprises culturing an engineered carotenoid production cell according to any of claims 13-17 and 20-25, optionally the cell according to any of claims 16, 17 and 20-25.

30. A method for the production of neurosporene wherein the method comprises an engineered carotenoid production cell according to any of claims 13-17 and 20-25 optionally the cell according to any of claims 17 and 20-25.

31. A method for the production of bisabolol wherein the method comprises culturing an engineered terpenoid production cell according to any of claims 18-25, optionally the cell according to any of claims 19-25.

32. An engineered 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence.

33. A plasmid comprising one or more polynucleotides encoding any one or more of:
a) a phytoene synthase enzyme as defined herein;
b) a phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 3, 1, 2, 4-7, 51 or 52;
c) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3;
d) a GGPPS enzyme as defined herein;
e) a GGPPS enzyme having an amino acid sequence of any of SEQ ID NO: 14, 8-13, 15, 16, 55, 56, 57 or 58 or having an amino acid sequence with at least 80%, 85%, 90%, 92%, 94%, 96%, 98%, 99% or 100% similarity to any of SEQ ID NO: 14, 8-13, 15, 16, 55, 56, 57 or 58;
f) a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
g) a phytoene synthase enzyme as defined herein and a GGPPS enzyme as defined herein;
h) a phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 3, 1, 2, 4-7, 51 or 52 and a GGPPS enzyme having an amino acid sequence of any of SEQ ID NO: 14, 8-13, 15, 16, 55, 56, 57 or 58 or having an amino acid sequence with at least 80%, 85%, 90%, 92%, 94%, 96%, 98%, 99% or 100% similarity to any of SEQ ID NO: 14, 8-13, 15, 16, 55, 56, 57 or 58;
i) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 14 or with an amino acid sequence with at least 80%, 85%, 90%, 92%, 94%, 96%, 98%, 99% or 100% similarity to SEQ ID NO: 14;
j) a phytoene synthase enzyme as defined herein and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
k) a phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 3, 1, 2, 4-7, 51 or 52 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
l) a phytoene synthase enzyme with an amino acid sequence of SEQ ID NO: 3 or with an amino acid sequence with at least 80%, 85%, 90%, 92%, 94%, 96%, 98%, 99% or 100% similarity to SEQ ID NO: 3 and a GGPPS enzyme having an amino acid sequence of SEQ ID NO: 8 or with an amino acid sequence with at least 80%, 85%, 90%, 92%, 94%, 96%, 98%, 99% or 100% similarity to SEQ ID NO: 8;
m) the mevalonate pathway expression system according to any of claim 22;
n) a HmgACT enzyme, for example of SEQ ID NO: 17 or a protein with an amino acid sequence with at least 80%, 85%, 90%, 92%, 94%, 96%, 98%, 99% or 100% similarity to said sequence;
o) a HmgS enzyme, for example of SEQ ID NO: 18 or a protein with an amino acid sequence with at least 80%, 85%, 90%, 92%, 94%, 96%, 98%, 99% or 100% similarity to said sequence;
p) a HmgR enzyme, for example of SEQ ID NO; 19, 20, 21 or a protein with an amino acid sequence with at least 80%, 85%, 90%, 92%, 94%, 96%, 98%, 99% or 100% similarity to said sequence; or that is selected from:
q) An engineered 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
q) a MVK enzyme, optionally that:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74;
r) a PMK enzyme, for example of SEQ ID NO: 23 or a protein with an amino acid sequence with at least 80%, 85%, 90%, 92%, 94%, 96%, 98%, 99% or 100% similarity to said sequence;
s) a PMD enzyme, for example of SEQ ID NO: 24 or a protein with an amino acid sequence with at least 80%, 85%, 90%, 92%, 94%, 96%, 98%, 99% or 100% similarity to said sequence;
t) a HmgACT enzyme, for example of SEQ ID NO: 17 or a protein with an amino acid sequence with at least 80%, 85%, 90%, 92%, 94%, 96%, 98%, 99% or 100% similarity to said sequence;
a HmgS enzyme, for example of SEQ ID NO: 18 or a protein with an amino acid sequence with at least 80%, 85%, 90%, 92%, 94%, 96%, 98%, 99% or 100% similarity to said sequence;
a HmgR enzyme, for example that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
an isopentenyl-diphosphate delta isomerase 1 enzyme as described herein, for example wherein the IDI enzyme is from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41;
u) a phytoene desaturase enzyme with an amino acid sequence of any of SEQ ID NO: 33-38 or has an amino acid sequence with at least 80%, 85%, 90%, 92% 94%, 96%, 98%, 99% or 100% similarity to any of SEQ ID NO: 33-38;
v) a bisabolol synthase enzyme having an amino acid sequence of SEQ ID NO: 25-29 or having an amino acid sequence with at least 80%, 85%, 90%, 92%, 94%, 96%, 98%, 99% or 100% similarity to any of SEQ ID NO: 25-30;
w) an isopentenyl-diphosphate delta isomerase 1 enzyme from B. subtilis or E. coli, for example wherein the isopentenyl-diphosphate delta isomerase enzyme has an amino acid sequence of SEQ ID NO: 31, or 41, or an amino acid sequence with at least 80%, 85%, 90%, 92%, 94%, 96%, 98%, 99% or 100% similarity to SEQ ID NO: 31 or 41; and/or
x) a 1-deoxy-D-xylulose-5-phosphate synthase enzyme from E. coli, optionally wherein the 1-deoxy-D-xylulose-5-phosphate synthase enzyme has an amino acid sequence of SEQ ID NO: 32, or an amino acid sequence with at least 80%, 85%, 90%, 92%, 94%, 96%, 98%, 99% or 100% similarity to SEQ ID NO: 32.

34. A method for producing retinol wherein the method comprises culturing a recombinant cell, wherein the cell expresses or overexpresses one or more enzymes of the mevalonate pathway and/or methylerythritol pathway, and wherein said one or more enzymes of the mevalonate pathway and/or methylerythritol pathway includes at least 3-hydroxy-3-methylglutaryl-CoA reductase, wherein the 3-hydroxy-3-methylglutaryl-CoA reductase enzyme:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
wherein said expression or overexpression results in increased production of:
i) phytoene;
ii) carotenoids;
iii) bisabolol;
iv) terpenoids; and/or
v) isopentenyl pyrophosphate (IPP) and dimethylallyl pyrophosphate (DMAPP)
relative to a cell that does not express and/or overexpress said one or more enzymes of the mevalonate pathway and/or methylerythritol pathway,
optionally relative to a cell that is able to produce
i) phytoene;
ii) carotenoids;
iii) bisabolol;
iv) terpenoids; and/or
v) isopentenyl pyrophosphate (IPP) and dimethylallyl pyrophosphate (DMAPP)
but that does not express and/or overexpress said one or more enzymes of the mevalonate pathway and/or methylerythritol pathway.

35. The method of claim 34 wherein the cell further comprises a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38.

36. An engineered retinol production cell, wherein the engineered retinol production cell comprises:
a) a polynucleotide encoding a 3-hydroxy-3-methylglutaryl-CoA reductase enzyme that:
has at least 80%, 85%, 90%, 92%, 94%, 96%, 98%, 99% or 100% similarity to SEQ ID NO: 19, 20 or 21, a substitution at any one or more of position 236, 188, 343 and/or 102, or has at least 80%, 85%, 90%, 92%, 94%, 96%, 98%, 99% or 100% to any of SEQ ID NO: 19, 20 or 21 provided that the enzyme comprises a substitution at any one or more of position 236, 188, 343 and/or 102;
has at least 80%, 85%, 90%, 92%, 94%, 96%, 98%, 99% or 100% similarity to SEQ ID NO: 19, 20 or 21 provided that the enzyme comprises at least one of the following: a histidine at residue 236, a serine at residue 188, an aspartic acid at residue 343, and/or an isoleucine at position 102; or
comprises a Q236H substitution, a T188S substitution a G343D and/or a T102I substitution wherein said positions are numbered with reference to SEQ ID No: 19;
comprises a K102I substitution, a G188S substitution, a W236H substitution and/or a P343D substitution wherein said positions are numbered with reference to SEQ ID NO: 20;
comprises a F102I substitution, a Q188S substitution, a S236H substitution and/or a Q343D substitution wherein said positions are numbered with reference to SEQ ID NO: 21;
comprises or consists of a sequence of SEQ ID NO: 64, 63, 65, 76, 72, 73, 74, 75, 76, 77, 78, 79, or 80, or that comprises or consists of a sequence that has at least 80%, 85%, 90%, 92%, 94%, 96%, 98%, 99% or 100% similarity to SEQ ID N: 64, 63, 65, 76, 72, 73, 74, 75, 76, 77, 78, 79, or 80;
is from S. pneumoniae, optionally has a nucleotide sequence that encodes a protein with an amino acid sequence of SEQ ID NO: 19 with at least 80%, 85%, 90%, 92%, 94%, 96%, 98%, 99% or 100% similarity to said sequence;
is from S. cerevisiae, optionally has a nucleotide sequence that encodes a protein with an amino acid sequence of SEQ ID NO: 20 with at least 80%, 85%, 90%, 92%, 94%, 96%, 98%, 99% or 100% similarity to said sequence; or
is from Enterococcus faecalis, optionally has a nucleotide sequence that encodes a protein with an amino acid sequence of SEQ ID NO: 21 with at least 80%, 85%, 90%, 92%, 94%, 96%, 98%, 99% or 100% similarity to said sequence;
b) a polynucleotide encoding a phytoene synthase enzyme having an amino acid sequence of any of SEQ ID NO: 3, 1, 2, 4-7, 51 or 52 or having an amino acid sequence with at least 80%, 85%, 90%, 92%, 94%, 96%, 98%, 99% or 100% similarity to any of SEQ ID NO: 3, 1, 2, 4-7, 51 or 52; and
c) a polynucleotide encoding a GGPPS enzyme having an amino acid sequence of any of SEQ ID NO: 14, 8-13, 15, 16, 55, 56, 57 or 58 or having an amino acid sequence with at least 80%, 85%, 90%, 92%, 94%, 96%, 98%, 99% or 100% similarity to any of SEQ ID NO: 14, 8-13, 15, 16, 55, 56, 57 or 58,
optionally wherein:
the phytoene synthase has an amino acid of sequence of SEQ ID NO: 3, optionally an amino acid sequence with at least 80%, 85%, 90%, 92%, 94%, 96%, 98%, 99% or 100% similarity to SEQ ID NO 3; and/or
the GGPPS enzyme has an amino acid sequence of SEQ ID NO: 14, optionally an amino acid sequence with at least 80%, 85%, 90%, 92%, 94%, 96%, 98%, 99% or 100% similarity to SEQ ID NO: 14.

37. The engineered retinol production cell of claim 36 wherein the cell further comprises a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38.

38. A method for the production of retinol wherein the method comprises culturing an engineered retinol production cell according to any of claim 36 or 37.

39. A method for producing a carotenoid or terpenoid wherein the method comprises culturing a recombinant cell, wherein the cell expresses or overexpresses one or more enzymes of the mevalonate pathway and/or methylerythritol pathway, and wherein said one or more enzymes of the mevalonate pathway and/or methylerythritol pathway includes at least mevalonate kinase, wherein the mevalonate kinase enzyme:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.

40. A method for producing retinol wherein the method comprises culturing a recombinant cell, wherein the cell
expresses or overexpresses one or more enzymes of the mevalonate pathway and/or methylerythritol pathway, and wherein said one or more enzymes of the mevalonate pathway and/or methylerythritol pathway includes at least mevalonate kinase, wherein the mevalonate kinase enzyme:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.

41. The method of claim 40 wherein the cell further comprises a polynucleotide encoding a phytoene desaturase enzyme that converts phytoene to lycopene, optionally wherein the phytoene desaturase enzyme is selected from any of a phytoene desaturase with:
an amino acid sequence of SEQ ID NO: 33, 34, 35, 37 or 38, or with an amino acid sequence with at least 80%, 85%, 90%, 92%, 94%, 96%, 98%, 99% or 100% similarity to SEQ ID NO: 33, 34, 35, 37 or 38.

42. An engineered retinol production cell, wherein the engineered retinol production cell comprises:
a) a polynucleotide encoding a mevalonate kinase enzyme that:
has an amino acid sequence of SEQ ID NO: 22, or an amino acid sequence with at least 80%, 85%, 90%, 92%, 94%, 96%, 98%, 99% or 100% similarity to said sequence;
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.

43. An engineered mevalonate kinase enzyme that:
has at least 80%, 85%, 90%, 92%, 94%, 96%, 98%, 99% or 100% similarity to SEQ ID NO: 22, provided that the enzyme comprises at least one of the following: a aspartic acid at residue 419, a valine at residue 402, a glycine at residue 73, a valine at residue 393, a valine at residue 175, a glutamic acid at residue 74; or
comprises a E419D substitution, a K402V substitution, a E73G substitution, a G393V substitution, a L175V substitution, a D74E substitution, wherein said positions are numbered with reference to SEQ ID No: 22;
has at least 80%, 85%, 90%, 92%, 94%, 96%, 98%, 99% or 100% similarity to SEQ ID NO: 22, provided that the enzyme comprises a substitution at one or more positions selected from positions 419, 402, 73, 393, 175, 74, wherein said positions are numbered with reference to SEQ ID No: 22;
comprises or consists of a sequence of any of SEQ ID NO: 66-71 or that has at least 80%, 85%, 90%, 92%, 94%, 96%, 98%, 99% or 100% similarity to SEQ ID NO: 66-71 provided that the enzyme comprises at least one of the following: a aspartic acid at residue 419, a valine at residue 402, a glycine at residue 73, a valine at residue 393, a valine at residue 175, a glutamic acid at residue 74.


SEQUENCE LISTING (80 sequences)

SEQ ID NO: 1 - protein, 296 aa, Pantoea ananatis
MAVGSKSFATASKLFDAKTRRSVLMLYAWCRHCDDVIDDQTLGFQARQPALQTPEQRLMQLEMKTRQAYAGSQMHEPAFAAFQEVAMAHDIAPAYAFDHLEGFAMDVREAQYSQLDDTLRYCYHVAGVVGLMMAQIMGVRDKATLDRACDLGLAFQLTNIARDIVDDAHAGRCYLPASWLEHEGLNKENYAAPENRQALSRIARRLVQEAEPYYLSATAGLARLPLRSAWAIATAKQVYRKIGVKVEQAGQQAWDQRQSTTTPEKLTLLLAASGQALTSRMRAHPPRPAHLWQRPL

SEQ ID NO: 2 - protein, 398 aa, Daucus carota
MACNFAVRVIYYPKEIHGVSVLNTNRSRKSRFSCRVMKLSTGVSAVAANPVRTSEERVYEVVLKQAALVREEKRSSRGLCLDTKRTGSKSFDKSENDDAGMKSWNLLNEAYDRCGEVCAEYAKTFYLGTLLMTPERRRAVWAIYVWCRRTDELVDGPNASHITPKALDRWEKRLNDLFDGQPYDMYDAALADTVSTYPVDIQPFKDMIDGMRMDLKKSRYQTFDELYLYCYYVAGTVGLMSVPVMGIAPESKATTESVYSAALALGIANQLTNILRDVGEDARRGRIYLPQEELKLAGITPEYIFKGKVTDKWRSFMKGQIKRARMFFDEAEKGVAELSSASRWPVWASLLLYKQILDAIEANDYDNFTKRAYVGKAKKLVSLPLAYSRALFAPSTVR

SEQ ID NO: 3 - protein, 296 aa, Enterobacter agglomerans
MEVGSKSFATASKLFDAKTRRSVLMLYAWCRHCDDVIDDQVLGFSNDTPSLQSAEQRLAQLEMKTRQAYAGSQMHEPAFAAFQEVAMAHDILPAYAFDHLAGFAMDVHETRYQTLDDTLRYCYHVAGVVGLMMAQIMGVRDNATLDRACDLGLAFQLTNIARDIVEDAEAGRCYLPAAWLAEEGLTRENLADPQNRKALSRVARRLVETAEPYYRSASAGLPGLPLRSAWAIATAQQVYRKIGMKVVQAGSQAWEQRQSTSTPEKLALLVAASGQAVTSRVARHAPRSADLWQRPV

SEQ ID NO: 4 - protein, 337 aa, Synechocystis sp.
MANGQISPQRAVTKPQSWWLTSEPRPSLMLQLPKPSPSAKPCASVEEAYEICRQVTAQYAKTFYLGTLLMPPAKRRAIWAIYLWCRRTDELVDGPQAATTTPETLDHWERRLEGIFAGQPQDDADVALVDTLETFPLDIQPFRDMIAGQRMDLYRSRYQTFEELDLYCYRVAGTVGLMSSAVLGVDTGNGQAPWQPDAVYIPQEEAIALGVANQLTNILRDVGEDVERGRIYLPLEDLERFNYSEQDLLNGVNDDRWRSLMKFEIDRARHYFEDAERGIRALNRDARWPVWTALMLYKGILDVIEANNYNVFNRRAYVPTPKKLLYLPVAWLRAQVL

SEQ ID NO: 5 - protein, 309 aa, Arthrospira platensis
MLQLSKSPCMRTPATRSDSYELCRQITAQYAKTFYLGTQLMPLAKRQAIWAIYVWCRRTDELVDGPMASSTTLETLDHWEEQLESIFAGHPIEPVDVALVDTLGRFPLDIQPFRDMIAGQRMDLSRNRYNTFDELNLYCYRVAGTVGLMSLAVMGTAEPDLSVPWNRDQSIYYPKEEAIALGIANQLTNILRDVGEDARRGRIYLPLDDLALFNYTEADLLNGKVDERWRELMRFQIQRARKFYTLAEEGIAALHPDIRWPVWTALMLYRQILDEIERNEYDVFNQRAYVPTWKKMMCLPLAQLRARVL

SEQ ID NO: 6 - protein, 422 aa, Cucumis melo
MSLASSLVVSSNVELSPSSFGFLDSVRDGPQIPDSFRFSSRNRVPNLINKKQKWGNHSHSTELKYPILHESGYGSVIVASMVANPAGEIAVSAEQKVYNVVMKQAALVKRQLRTAGELDVKPDIVLPGTLSLLNEAYDRCGEVCAEYAKTFYLGTMLMTPERQKAIWAIYVWCRRTDELVDGPNASHITPTALDRWEARLEELFQGRPFDMLDAALADTVTKFPVDIQPFKDMIEGMRMDLRKSRYKNFDELYLYCYYVAGTVGLMSVPVMGIAPESQASTESVYNAALALGIANQAPPNILRDVGEDARRGRIYLPQDELAQAGLSDEDIFAGRVTDKWRNFMKNQIKRARMFFDEAEKGVLELNKASRWPVWASLLLYRQILDEIEANDYDNFTKRAYVSKAKKILALPMAYGRALLGPS

SEQ ID NO: 7 - protein, 287 aa, Staphylococcus aureus
MTMMDMNFKYCHKIMKKHSKSFSYAFDLLPEDQRKAVWAIYAVCRKIDDSIDVYGDIQFLNQIKEDIQSIEKYPYEHHHFQSDRRIMMALQHVAQHKNIAFQSFYNLIDTVYKDQHFTMFETDAELFGYCYGVAGTVGEVLTPILSDHETHQTYDVARRLGESLQLINILRDVGEDFENERIYFSKQRLKQYEVDIAEVYQNGVNNHYIDLWEYYAAIAEKDFRDVMDQIKVFSIEAQPIIELAARIYIEILDEVRQASYTLHERVFVYKRKKAKLFHEINSKYHRI

SEQ ID NO: 8 - protein, 335 aa, Saccharomyces cerevisiae [sequence omitted]

SEQ ID NO: 9 - protein, 369 aa, Capsicum annuum
MRSMNLVDLWAQQACLVFNQTLSYKSFNGFMKIPLKNSKINPKLNKKRPFSPLTVSAIATTKEDERIEAAQTEEPFNFKIYVTEKAISVNKALDEAIIVKEPHVIHEAMRYSLLAGGKRVRPMLCLAACELVGGNQENAMAAACAVEMIHTMSLIHDDLPCMDNDDLRRGKPTNHKIYGEDVAVLAGDSLLAFAFEHIVNSTAGVTPSRIVGAVAELAKSIGTEGLVAGQVADIKCTGNASVSLETLEFIHVHKTAALLESSVVLGAILGGGTNVEVEKLRRFARCIGLLFQVVDDILDVTKSSEELGKTAGKDLVVDKTTYPKLLGLEKAKEFAAELNREAKQQLEGFDSRKAAPLIALADYIAYRDN

SEQ ID NO: 10 - protein, 383 aa, Abies grandis
MAYSGMATSYHGLHFMNIATQECNLKRLSIPSRRFHGVSPSLWASNGFQGHLKRELSANSFLVSSSRYSNTIAKFTNLPEKVKEKVIEFDFKEYLRSKAMAVNEALDRAVPLRYPERIHEAMRYSLLAGGKRVRPVLCISACELVGGTEEVAMPTACAMEMIHTMSLIHDDLPCMDNDDFRRGKPTNHKVFGEGTAILAGDALLSFAFEHIAVSTSKSVGTDRILRVVSELGRTIGSQGLVGGQVADITSEGDASVDLDTLEWIHIHKTAVLLECSVMCGAIISGASDNEIERIQRYARSVGLLFQVVDDILDVTKSSKELGKTAGKDLISDKATYPKLMGLEKAKQFASDLLIRAKEDLSCFDPMKAAPLLGLADYIAFRQN

SEQ ID NO: 11 - protein, 371 aa, Penicillium paxilli
MSYILAEALNFVRRGISHLNYWGASHSLSADNYWESNFQGFPRLLSDSSKAPSTIRTVQVLEDDVDDIAIQYNKIVRGPLDYLLAIPGKDIRSKLIDSFNIWLQLPEEKLSIVKDIINLLHTASLLIDDIQDASRLRRGKPVAHDVYGVAQTINSANYAYYLQQARLKEIGDPRAFEIFTRSLLDLHLGQGMDLYWRDMVVCPTEEEYTRMVMYKTGGLFNLALDLMRIQSRKNTDFSKLVELLGVIFQIRDDYMNLQSGLYAEKKGLMEDLTEGKFSYPIIHSIRASPESSELLDILKQRTEDEAVKIRAVKIMESTGSFQYTRETLSRLSAEARGYVKKLETSLGPNPGIHKILDLLEVEYPTNEKGRV

SEQ ID NO: 12 - protein, 303 aa, Mucor lusitanicus
MLNSHNRTEERSTEDIILEPYTYLISQPGKDIRAKLISAFDLWLHVPKDVLCVINKIIGMLHNASLMIDDVQDDSDLRRGVPVAHHIYGVPQTINTANYVIFLALQEVMKLNIPSMMQVCTEELINLHRGQGIELYWRDSLTCPTEEEYIDMVNNKTSGLLRLAVRLMQAASESDIDYTPLVNIIGIHFQVRDDYMNLQSTSYTNNKGFCEDLTEGKFSFPIIHAIRKDPSNRQLLNIISQKPTSIEVKKYALEVIRKAGSFEYVREFLRQKEAESLKEIKRLGGNPLLEKYIETIRVEATND

SEQ ID NO: 13 - protein, 368 aa, Croton sublyratus
MSSVNLGSWVHTSSVISQATRSRSKSKPLSFSPVSIPLFYRNSKRSVSYVSAIVTKDEETIQEEQNKNSSSSLGFDFKSYMVQKASAINQALEAAVSLREPLKIHESMRYSLLAGGKRVRPALCLAACELVGGDESMAMPAACAVEMIHTMSLIHDDLPCMDNDDLRRGKPTNHIVFGENVAVLAGDALLAFAFEHIAVSTLNVSPVRIVRGVGELAKAIGAEGLVAGQVVDICSEGLSEVDLEKLEFIHIHKTAKLLEGAVVLGAIMGGGTDEEVEKLRKYARDIGLLFQVVDDILDVTKSSQELGKTAGKDLVADKVTYPKLMGIEKSREFAEKLNKEAQEQLAGFDPEKAAPLIALANYIAYRQN

SEQ ID NO: 14 - protein, 370 aa, Hevea brasiliensis
MSSVNLGSWVHTSYVLNQATRSRSKSKSFSLPFNPLKSLAISFAYRKSERPISSVSAIITKEEETLQEEQNNPPPSFDFKSYMLQKGNSINQALEAAIPLQEPAKIHESMRYSLLAGGKRVRPALCLAACELVGGNDSMAMPAACAVEMIHTMSLIHDDLPCMDNDDLRRGKPTNHIVFGEDVAVLAGDALLAFAFEHIAVSTLNVSSARIVRAVGELAKAIGAEGLVAGQVVDINSEGSSEVDLEKLEFIHIHKTAKLLEGAVVLGAILGGGTDEEVEKLRKYARDIGLLFQVVDDILDVTKSSQELGKTAGKDLVADKVTYPKLLGIEKSREFAEKLNKEAQEQLAGFDPEKAAPLIALANYIAHRQN

SEQ ID NO: 15 - protein, 307 aa, Pseudescherichia vulneris
MVSGSKAGVSPHREIEVMRQSIDDHLAGLLPETDSQDIVSLAMREGVMAPGKRIRPLLMLLAARDLRYQGSMPTLLDLACAVELTHTASLMLDDMPCMDNAELRRGQPTTHKKFGESVAILASVGLLSKAFGLIAATGDLPGERRAQAVNELSTAVGVQGLVLGQFRDLNDAALDRTPDAILSTNHLKTGILFSAMLQIVAIASASSPSTRETLHAFALDFGQAFQLLDDLRDDHPETGKDRNKDAGKSTLVNRLGADAARQKLREHIDSADKHLTFACPQGGAIRQFMHLWFGHHLADWSPVMKIA

SEQ ID NO: 16 - protein, 302 aa, Enterobacter agglomerans
MTVCAEQHVNFIHSDAASLLNDIEQRLDQLLPVESERDLVGAAMRDGALAPGKRIRPLLLLLAARDLGCNATSAGLLDLACAVEMVHAASLILDDMPCMDDAQLRRGRPTIHCQYGEHVAILAAVALLSKAFGVVAAAEGLTATARADAVAELSHAVGMQGLVQGQFKDLSEGDKPRSADAILMTNHYKTSTLFCASMQMASIVAEASGEAREQLHRFSLNLGQAFQLLDDLTDGMADTGKDAHQDDGKSTLVNLLGPQAVETRLRDHLRCASEHLLSACQDGYATHHFVQAWFEKKLAAVS

SEQ ID NO: 17 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVSQQRLMRGQIVFYNVADPESLIDKLQVREAEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 18 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEGCYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 19 - protein, 424 aa, Streptococcus pneumoniae
MKISWNGFSKKSYQERLELLKAQALLSPERQASLEKDEQMSVTVADQLSENVVGTFSLPYSLVPEVLVNGQEYTVPYVTEEPSVVAAASYASKIIKRAGGFTAQVHQRQMIGQVALYQVANPKLAQEKIASKKAELLELANQAYPSIVKRGGGARDLHVEQIKGEPDFLVVYIHVDTQEAMGANMLNTMLEALKPVLEELSQGQSLMGILSNYATDSLVTASCRIAFRYLSRQKDQGREIAEKIALASQFAQADPYRAATHNKGIFNGIDAILIATGNDWRAIEAGAHAFASRDGRYQGLSCWTLDLEREELVGEMTLPMPVATKGGSIGLNPRVALSHDLLGNPSARELAQIIVSIGLAQNFAALKALVSTGIQQGHMKLQAKSLALLAGASESEVAPLVERLISDKTFNLETAQRYLENLRS

SEQ ID NO: 20 - protein, 527 aa, Saccharomyces cerevisiae
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 21 - protein, 407 aa, Enterococcus faecalis
MSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 22 - protein, 443 aa, Saccharomyces cerevisiae
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 23 - protein, 451 aa, Saccharomyces cerevisiae
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDK

SEQ ID NO: 24 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 25 - protein, 569 aa, Cynara cardunculus
MSNFLVSTCSSPLALDENNSTKQDHVIRNTVTFHSSIWGDQFLTYDEKDDLVAEKQLAEELIEETRKELIITTSSHEPIQHMKLIQLIDAVQRLGVAYHFEKEIEDALQHVYRTYGHQGIHNNNDLQSISLWFRILRQQGFNVSSEIFKNHMDEKGNLFSNDVQSMLALYEASYMRVEGEKVLDDALEFTKTHLAIIAKDPSCDSSLRTQIQDALRQPLRKRLPRLEAVRYIPIYQQQSSHNQILLKLAKLDFNMLQTMHKKELSEICKWWKDLDMQNKLPFVRDRLIEGYFWILGIYFEPHHSRSRMFLIKSCMWLVVIDDTFDNYGTYEELEIFTEAVERWSINCLDMLPEYMKLIYKELVIVHQEMEETLEKEGKAYHIHHVKELAKECTRSLLVEAKWLKEGYMPTLDEYISNSLITCAYAVMIARSYVGGDDKLVNEDSFKWVATHPPLVKASCLILRLMDDIATHKEEQERGHVASSIECYIKETGATEEEAREHFSKQVEDAWKVVNRESLRPTAVAFPLVMPAINLARMCDALYKGNHDGYNHAGKEVIQYIKSLLVHPLI

SEQ ID NO: 26 - protein, 572 aa, Artemisia annua
MSTLSVSSPSFSSFPLSSADNTHSTKKDVIRNSVIFHDSIWGDQFLEHDEKFNVATEKQLIEELKEEVRKELMIRASNEASQDIKLIQLIDEVQRLGVAYHFEKEIEESLQHIYVTYGDNWIGNNIESVSLWFQLLRQHGFNISSDIFKNYKDEKGKIQESLCDDPQGMLALYEATYLRVEGEEVLDDANEFTKINLGIMSKDPFCDSSLRTEIQQALKQPLRRRLPRLEAVRYIPIYQQKASHNEVLLKLAKLDFNVLQDMHKHELSLICKWWKDLDIRNKLPFVRDRLIEGYFWIMGIYFEPQHSRTRIFLMKTCMWLVVLDDTFDNYGTYEELEMFTQAVERWSITCLDELPEYMKLIYQEQLSVHQEMEESLEKEGKAYQIHYIKEMAKEGTRSLLLEAKWLREGYMPTLEEYISNSLVTCGYAVMTARSYVARDDDIVSEDAFKWVATHPPLVKATCKILRLMDDIATHKEEQERGHIASSIECYRKETGASEEEACEFFSKQVEDAWKVINRESLRPTDVPFPLLIPAINLARVSDTLYKYNDGYNHAGKEVISHIKSIFVNPMIV

SEQ ID NO: 27 - protein, 574 aa, Artemisia annua
MSTLPISSVSSSSSTSPIVVDDKDSTKPDVIRHTTNFNASIWGDQFLTYDEPEDLVMKKQLVEELKEEVKKELITIKGSNEPMQHVKLIELIDAVQRLGIAYHFEEEIEEALQHIHVTYGEQWVDKENLQSISLWFRLLRQQGFNVSSGVFKDFMDEKGKFKESLCNDAQGILALYEAAFMRVEDETILDNALEFSKVHLDIIAKDPSCDSSLRTQIHQALKQPLRRRLARIEALHYMPIYQQETSHDEVLLKLAKLDFSVLQSMHKKELSHICKWWKDLDLQNKLPFVRDRVVEGYFWILSIYYEPQHARTRMFLMKSCMWLVVLDDTFDNYGTYEELEIFTQAVEKWSISCLDMLPEYMKLIYQELVNLHVEMEESLEKEGKAYQIHYVKEMAKELVRNYLVEARWLKEGYMPTLEEYMSVSMVTGTYGLMTARSYVGRGDIVNEDTFKWVSSYPPIVKASCVIIRLMDDIVSHKEEQERGHVASSIECYSKESGASEEEACEYISRKVEDAWKVINRESLRPTAVPFPLLMPAINLARMCEVLYSVNDGFTHAEGDMKSYMKSFFVHPMVV

SEQ ID NO: 28 - protein, 574 aa, Eremanthus erythropappus
MSTAFPVAALVSASASFSSSEPLPSPPPTTTATKQRNTTNHHSSIWGDQFLTYHESEDILKEKLLVDELKEQVRKELISASVDDHLELIDAIQRLGVAYYYENEIEEALQHIYVTYGDHWIHTDNLQSISLWFRLLRQQGFNVSSGIFKNHMNDKGNFKESISNDVQGMLELYEAAYMRVEGEEILDYALEFTKTNLGILANDPSCDSSLRSQIQQALKQPLRKRLPRLEAVRYIPIYKQQACHNDVLLKLAKLDFNVLQSMHKKELSHICKWWKDLNMQEKLPYVRDRLVEGYFWILGIYFEPHHARTRMFLIKTCMWLVVIDDTFDNYGAYEELEQFTEAVERWSISCLDELPEYMKLVYQELVNVHQEMEESLEKEGKAFQIHYVKEMAKECTRSLLLEAKWLKDGYMPTLEEYLSNSLITCAYAVMVARSYVGREDVMIREETFKWVATHPPLVKASCLILRLMDDIATHKEEQERGHVASSIECYIRENVGATEEEACELFSKQIEDAWKVINRECLRPMDVPFLLVMPAINLARICDALYSKGNDGFNHAGEEVINYIKSLVVHPLVV

SEQ ID NO: 29 - protein, 575 aa, Helianthus annuus
MSNFQLSSVSFSSSILPSAVDDNSDTKLDVIRNTMNFSASIWGDQFLTFEEPDDLEMEKKVVEVLKEEVRKELVIKGSSNESLQHMKLIELIDVVQRLGVAYHFEEEIEEALKHIYVTYGEKWVDLNNLHNLSLWFRLLRQQGFNVSSGIFKYHMCEKGNFKESLCEDAQGLLALYEASYMRVEGEKVLDDALEFTKTHLAIIAKDPSCDSLLRAQIQEALKQPLRKRLPRLEAVRYIPIYQQDVSHNEVLLKLAKSDFNVLQSMHKEELSQICKWWKDLDMQKKLPYVRDRLIEGYFWILGVYFEPQHSHTRIFLMKTSMWLIVLDDTYDNYGTYEELKIFTDAVQRWSMSCLDLLPEYMKLIFQELLNHHQEMEESLEKEGKTYQIHYVIEMAKEVLENNLVEAKWLKEGYMPTLEEYMSVSMKTCTYGLMIARSFVGRVDNMVTEDTFKWVATYPPIVKAACLVLRLMDDITTHKEEQERGHVASSIECYQKETGASEKEACEFISNMVEDAWKVINRESLRPTDIPFPLLPSTINFARACDVIYKVNDSYTHARKEMINHIKSLLVHPLAI

SEQ ID NO: 30 - protein, 572 aa, Matricaria chamomilla
  source 1..572: note = var. recutita
MSTLSVSTPSFSSSPLSSVNKNSTKQHVTRNSVIFHDSIWGDQFLEYKEKFNVATEKQLIEELKEEVRNELMIRACNEASRYIKLIQLIDVVERLGLAYHFEKEIEESLQHIYVTYGHKWTNYNNIESLSLWFRLLRQNGFNVSSDIFENHIDEKGNFQESLCNDPQGMLALYEAAYMRVEGEIILDKALEFTKLHLGIISNDPSCDSSLRTEIKQALKQPLRRRLPRLEAVRYIAIYQQKASHSEVLLKLAKLDFNVLQEMHKDELSQICKWWKDLDIRNKLPYVRDRLIEGYFWILGIYFEPQHSRTRMFLMKTCMWLIVLDDTFDNYGTYEELEIFTQAVERWSITCLDELPEYMKLIYHEQFRVHQEMEESLEKEGKAYQIHYIKEMAKEGTRSLLLEAKWLKEGYMPTLDEYLSNSLVTCGYALMTARSYVARDDGIVTEDAFKWVATHPPIVKAACKILRLMDDIATHKEEQERGHIASSIECYRKETGASEEEACMDFLKQVEDGWKVINQESLMPTDVPFPLLIPAINLARVSDTLYKDNDGYNHADKEVIGYIKSLFVHPMIV

SEQ ID NO: 31 - protein, 349 aa, Bacillus subtilis
MTRAERKRQHINHALSIGQKRETGLDDITFVHVSLPDLALEQVDISTKIGELSSSSPIFINAMTGGGGKLTYEINKSLARAASQAGIPLAVGSQMSALKDPSERLSYEIVRKENPNGLIFANLGSEATAAQAKEAVEMIGANALQIHLNVIQEIVMPEGDRSFSGALKRIEQICSRVSVPVIVKEVGFGMSKASAGKLYEAGAAAVDIGGYGGTNFSKIENLRRQRQISFFNSWGISTAASLAEIRSEFPASTMIASGGLQDALDVAKAIALGASCTGMAGHFLKALTDSGEEGLLEEIQLILEELKLIMTVLGARTIADLQKAPLVIKGETHHWLTERGVNTSSYSVR

SEQ ID NO: 32 - protein, 620 aa, Escherichia coli
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKNRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVLGLITTLKNMRDLKGPQFLHIMTKKGRGYEPAEKDPITFHAVPKFDPSSGCLPKSSGGLPSYSKIFGDWLCETAAKDNKLMAITPAMREGSGMVEFSRKFPDRYFDVAIAEQHAVTFAAGLAIGGYKPIVAIYSTFLQRAYDQVLHDVAIQKLPVLFAIDRAGIVGADGQTHQGAFDLSYLRCIPEMVIMTPSDENECRQMLYTGYHYNDGPSAVRYPRGNAVGVELTPLEKLPIGKGIVKRRGEKLAILNFGTLMPEAAKVAESLNATLVDMRFVKPLDEALILEMAASHEALVTVEENAIMGGAGSGVNEVLMAHRKPVPVLNIGLPDFFIPQGTQEEMRAELGLDAAGMEAKIKAWLA

SEQ ID NO: 33 - protein, 492 aa, Pantoea ananatis
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFAYLEQHYMPGLRSQLVTHRMFTPFDFRDQLNAYHGSAFSVEPVLTQSAWFRPHNRDKTITNLYLVGAGTHPGAGIPGVIGSAKATAGLMLEDLI

SEQ ID NO: 34 - protein, 501 aa, Paracoccus sp.
MNAHSPAAKTAIVIGAGFGGLALAIRLQSAGIATTLVEARDKPGGRAYVWHDQGHLFDAGPTVITDPDALKELWALTGQDMARDVTLMPVSPFYRLMWPGGKVFDYVNEADQLERQIAQFNPDDLEGYRRFRDYAEEVYQEGYVKLGTVPFLKLGQMLKAAPALMKLEAYKSVHAKVATFIKDPYLRQAFSYHTLLVGGNPFSTSSIYALIHALERRGGVWFAKGGTNQLVAGMVALFERLGGQMLLNAKVARIDTDGPRATGVTLADGRALTADMVASNGDVMHNYRDLLGHTARGQSRAKSLNAKRWSMSLFVLHFGLREAPKDVAHHTILFGPRYKELVNEIFKGPKLAEDFSLYLHSPCTTDPEMAPPGMSTHYVLAPVPHLGRADIDWAVEGPRYADRILASLEERLIPNLRANLTTTRIFTPSDFASELNAHHGSAFSVEPILTQSAWFRPHNRDKTIRNFYLVGAGTHPGAGIPGVVGSAKATAQVMLSDLASA

SEQ ID NO: 35 - protein, 492 aa, Pseudescherichia vulneris
MKKTVVIGAGFGGLALAIRLQAAGIPTVLLEQRDKPGGRAYVWHDQGFTFDAGPTVITDPTALEALFTLAGRRMEDYVRLLPVKPFYRLCWESGKTLDYANDSAELEAQITQFNPRDVEGYRRFLAYSQAVFQEGYLRLGSVPFLSFRDMLRAGPQLLKLQAWQSVYQSVSRFIEDEHLRQAFSFHSLLVGGNPFTTSSIYTLIHALEREWGVWFPEGGTGALVNGMVKLFTDLGGEIELNARVEELVVADNRVSQVRLADGRIFDTDAVASNADVVNTYKKLLGHHPVGQKRAAALERKSMSNSLFVLYFGLNQPHSQLAHHTICFGPRYRELIDEIFTGSALADDFSLYLHSPCVTDPSLAPPGCASFYVLAPVPHLGNAPLDWAQEGPKLRDRIFDYLEERYMPGLRSQLVTQRIFTPADFHDTLDAHLGSAFSIEPLLTQSAWFRPHNRDSDIANLYLVGAGTHPGAGIPGVVASAKATASLMIEDLQ

SEQ ID NO: 36 - protein, 524 aa, Rhodobacter capsulatus
MSKNTEGMGRAVVIGAGLGGLAAAMRLGAKGYKVTVVDRLDRPGGRGSSITKGGHRFDLGPTIVTVPDRLRELWADCGRDFDKDVSLVPMEPFYTIDFPDGEKYTAYGDDAKVKAEVARISPGDVEGFRHFMWDAKARYEFGYENLGRKPMSKLWDLIKVLPTFGWLRADRSVYGHAKKMVKDDHLRFALSFHPLFIGGDPFHVTSMYILVSQLEKKFGVHYAIGGVQAIADAMAKVITDQGGEMRLNTEVDEILVSRDGKATGIRLMDGTELPAQVVVSNADAGHTYKRLLRNRDRWRWTDEKLDKKRWSMGLFVWYFGTKGTAKMWKDVGHHTVVVGPRYKEHVQDIFIKGELAEDMSLYVHRPSVTDPTAAPKGDDTFYVLSPVPNLGFDNGVDWSVEAEKYKAKVLKVIEERLLPGVAEKITEEVVFTPETFRDRYLSPLGAGFSLEPRILQSAWFRPHNASEEVDGLYLVGAGTHPGAGVPSVIGSGELVAQMIPDAPKPETPAAAAPKARTPRAKAAQ

SEQ ID NO: 37 - protein, 532 aa, Synechocystis sp.
MLPAVGVDRPVGRRLQLHARVQQGIGVRSIAWSRVQAVALLAVVRDRQAAVQTPRVGQHQAVVQRNRRRHVRRARQGVADGRQHLGHARRAERDDLCVQEGGGDIEIQALVRAFEQDGVECELDPLALGLADVSAAVGVIGQTGRRLQLLEFIGEDVHRQAKTVRRPTQPGLIGGDGFGIERQGLGLGEDRGAVEAAAAIAGPRRDEDVQVRGKGVFGRDPTRHVVVLEGAVPDEGIGRGEGLGEGRDAPRVQVVVAPAEGRLVAGVTQTARQGQLVGQVIVDLAEHGEAVARLLVQVVVLGPGRIAGPVGVEVDIVRDVEGAHDPVQTTVVVVAQTHFQRGLVQRLIGIEARGRIGRAVAVALHRASRALDVADHRQIQVIAQTMLQRRRHAIDVDAMGRILRRIELVRVVVLARIGDIAVQRRRDTVVLARVAPVAAAEQLHGQVGVRREIEGDSGPAGEQILVAETFTAARIDDGRNLGLGHAGDDRLEARQVARARPGHEAAPCSASTASRTPTVSLRGPPTAASIAR

SEQ ID NO: 38 - protein, 507 aa, Streptomyces griseus
MITVKGPVDHVVVVGAGLAGLAAALHLLGAGRRVTVVEREDVPGGRAGLLESGGFRIDTGPTVLTMPDLVEDAFAAVGERMADRLELIRLAPAYRARFADGSQLDVHTDGAAMEAAVEEFAGARQAVGYRRLRIWLERLYRVQMRRFIDTNFDSPLQLAHPDLARLAALGGFGRLDARIGHFVSDERLRRVFSFQALYAGVPPARALAAYAVIAYMDTVAGVYFPRGGMHALPRAMADAASDAGAVLRYGQRVTRLERSGDRVTAVVTDQEHIPCDAVVLTPDLPVSYRLLGRTPHRPLPLRHSPSAVILHTGTDRTWPDLAHHTISFGAAWKNTFHELTRTGRLMSDPSLLITRPTATDPSLAPPGKHLHYVLAPCPNTEVGPGVREWRELGPRYRDELLAELERREMPGLGAAIEEEGLVTPVDWTAQGHAAGTPFSVAHTFPQTGPFRPRNLVRGTVNAVLAGCGTTPGVGVPTVLISGKLAAERITGPRIARAPRSAPGGSSA

SEQ ID NO: 39 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 40 - protein, 491 aa, Saccharomyces cerevisiae
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 41 - protein, 182 aa, Escherichia coli
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 42 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAVLAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 43 - DNA, 1863 bp, Escherichia coli
  source 1..1863: mol_type = genomic DNA
atgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataa

SEQ ID NO: 44 - DNA, 549 bp, Escherichia coli
  source 1..549: mol_type = genomic DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 45 - DNA, 6463 bp, synthetic construct
  source 1..6463: mol_type = other DNA
accggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatccgaattcgagctcaccaaacgccaaaggagcatccagcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccgaaaaaatcaaaaggagcatcgcctatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggttt

SEQ ID NO: 46 - DNA, 5781 bp, synthetic construct
  source 1..5781: mol_type = other DNA
gatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatccgatggaagtgggtagcaagagcttcgcgaccgcgagcaagctgtttgatgcgaaaacccgtcgtagcgttctgatgctgtacgcgtggtgccgtcactgcgacgatgtgatcgacgatcaagttctgggtttcagcaacgataccccgagcctgcagagcgcggagcaacgtctggcgcagctggaaatgaaaacccgtcaagcgtatgcgggcagccagatgcatgagccggcgttcgcggcgtttcaagaagtggcgatggcgcatgacatcctgccggcgtacgcgtttgatcacctggcgggctttgcgatggacgttcacgagacccgttatcagaccctggacgataccctgcgttactgctatcacgtggcgggtgtggttggcctgatgatggcgcaaattatgggtgttcgtgataacgcgaccctggaccgtgcgtgcgatctgggcctggcgtttcagctgaccaacatcgcgcgtgacattgtggaggatgcggaagcgggtcgttgctacctgccggcggcgtggctggcggaggaaggcctgacccgtgaaaacctggcggacccgcagaaccgtaaggcgctgagccgtgtggcgcgtcgtctggttgagaccgcggaaccgtactatcgtagcgcgagcgcgggtctgccgggtctgccgctgcgtagcgcgtgggcgatcgcgaccgcgcagcaagtttatcgtaagattggtatgaaagtggtgcaggcgggcagccaggcgtgggagcagcgtcaaagcaccagcaccccggaaaaactggcgctgctggttgcggcgagcggtcaagcggttaccagccgtgttgcgcgtcatgcgccgcgtagcgcggatctgtggcagcgtccggtttaagaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagcagcgtgaacctgggcagctgggtgcacaccagctacgttctgaaccaggcgacccgtagccgtagcaagagcaagagcttcagcctgccgtttaacccgctgaagagcctggcgattagcttcgcgtatcgtaaaagcgagcgtccgatcagcagcgttagcgcgatcattaccaaggaagaggagaccctgcaggaagagcaaaacaacccgccgccgagcttcgactttaagagctacatgctgcagaagggtaacagcatcaaccaagcgctggaagcggcgattccgctgcaagaaccggcgaagatccacgagagcatgcgttatagcctgctggcgggtggcaaacgtgtgcgtccggcgctgtgcctggcggcgtgcgaactggttggtggcaacgacagcatggcgatgccggcggcgtgcgcggtggagatgatccacaccatgagcctgattcacgacgatctgccgtgcatggataacgacgatctgcgtcgtggcaaaccgaccaaccacattgtttttggcgaagacgttgcggttctggcgggtgatgcgctgctggcgtttgcgtttgagcacatcgctgtgagcaccctgaacgttagcagcgcgcgtattgtgcgtgcggttggtgaactggcgaaagcgattggtgcggagggtctggtggcgggccaggtggttgacattaacagcgaaggtagcagcgaggttgatctggaaaaactggagttcatccacattcacaagaccgcgaaactgctggaaggcgcggtggttctgggtgcgatcctgggtggtggcaccgacgaagaggtggagaagctgcgtaaatacgcgcgtgatattggtctgctgtttcaggtggttgacgacatcctggacgttaccaagagcagccaagaactgggcaagaccgcgggtaaagacctggtggcggataaggttacctatccgaaactgctgggcatcgagaagagccgtgagttcgcggagaagctgaacaaagaagcgcaggagcaactggcgggttttgatccggaaaaagcggcgccgctgattgcgctggcgaactacattgcgcaccgtcaaaactaaagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaa

SEQ ID NO: 47 - DNA, 14356 bp, synthetic construct
  source 1..14356: mol_type = other DNA
atttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaatttgacaattaatcatcggctcgtataatgtgtggaattgtgagcgctcacaattcccctgtagaaataattttgtttaactttaataaggagatataccatggcaatgacccgtgcggagcgtaagcgtcagcacatcaaccacgcgctgagcattggtcaaaaacgtgagaccggcctggacgacatcaccttcgtgcacgttagcctgccggacctggcgctggaacaggtggacatcagcaccaagattggcgagctgagcagcagcagcccgatctttattaacgcgatgaccggtggcggtggcaagctgacctacgaaatcaacaagagcctggcgcgtgcggcgagccaggcgggtattccgctggcggtgggtagccaaatgagcgcgctgaaggacccgagcgagcgtctgagctatgaaatcgttcgtaaagagaacccgaacggtctgattttcgcgaacctgggtagcgaggcgaccgcggcgcaggcgaaagaggcggttgaaatgatcggtgcgaacgcgctgcaaattcacctgaacgtgatccaagaaattgttatgccggagggtgaccgtagcttcagcggcgcgctgaagcgtatcgaacaaatttgcagccgtgtgagcgttccggtgatcgttaaagaggtgggtttcggcatgagcaaggcgagcgcgggtaaactgtatgaagcgggtgcggcggcggtggacatcggtggttacggtggcaccaacttcagcaagattgagaacctgcgtcgtcagcgtcaaatcagcttctttaacagctggggtattagcaccgcggcgagcctggcggagatccgtagcgaatttccggcgagcaccatgattgcgagcggtggcctgcaagacgcgctggatgtggcgaaagcgattgcgctgggtgcgagctgcaccggtatggcgggccactttctgaaagcgctgaccgacagcggcgaggaaggcctgctggaggaaatccagctgattctggaggaactgaaactgatcatgaccgtgctgggtgcgcgtaccattgcggatctgcaaaaggcgccgctggttattaaaggtgaaacccaccactggctgaccgagcgtggcgtgaacaccagcagctacagcgttcgttaaggatccgaattcgaagtcagattaaaggaggatcgcgcatgagcctgccgttcctgaccagcgcgccgggcaaagttatcatttttggcgagcacagcgcggtgtacaacaaaccggcggttgcggcgagcgtgagcgcgctgcgtacctatctgctgatcagcgagagcagcgcgccggacaccatcgaactggacttcccggatattagctttaaccacaaatggagcatcaacgactttaacgcgattaccgaagatcaggttaacagccaaaaactggcgaaggcgcagcaagcgaccgacggtctgagccaagagctggtgagcctgctggacccgctgctggcgcaactgagcgagagcttccactaccacgcggcgttctgctttctgtatatgtttgtttgcctgtgcccgcacgcgaaaaacatcaagttcagcctgaaaagcaccctgccgattggtgcgggtctgggtagcagcgcgagcatcagcgtgagcctggcgctggcgatggcgtacctgggtggcctgattggcagcaacgacctggagaaactgagcgaaaacgataagcacatcgttaaccagtgggcgtttatcggcgagaagtgcattcacggcaccccgagcggtattgacaacgcggtggcgacctacggcaacgcgctgctgtttgaaaaagatagccacaacggcaccatcaacaccaacaacttcaagtttctggacgatttcccggcgatcccgatgattctgacctatacccgtattccgcgtagcaccaaagacctggttgcgcgtgtgcgtgttctggtgaccgagaaattcccggaagtgatgaagccgattctggatgcgatgggcgagtgcgcgctgcagggtctggaaatcatgaccaagctgagcaaatgcaagggcaccgacgatgaggcggttgaaaccaacaacgagctgtacgaacaactgctggagctgatccgtattaaccacggtctgctggttagcatcggcgtgagccacccgggtctggaactgatcaaaaacctgagcgacgatctgcgtattggcagcaccaagctgaccggtgcgggtggcggtggctgcagcctgaccctgctgcgtcgtgacatcacccaggagcaaattgatagcttcaagaaaaagctgcaggacgatttcagctatgagacctttgaaaccgacctgggtggcaccggttgctgcctgctgagcgcgaaaaacctgaacaaggatctgaaaattaagagcctggtgttccaactgtttgaaaacaaaaccaccaccaagcagcaaatcgacgatctgctgctgccgggtaacaccaacctgccgtggaccagctaacaatgacgttaaaggagaatctgtcatgagcgagctgcgtgcgtttagcgcgccgggtaaagcgctgctggcgggtggctacctggttctggacaccaagtatgaagcgtttgtggttggtctgagcgcgcgtatgcatgcggttgcgcacccgtacggtagcctgcagggcagcgacaagttcgaagtgcgtgttaagagcaagcaattcaaggatggtgaatggctgtatcacatcagcccgaagagcggcttcatcccggttagcattggtggcagcaagaacccgtttatcgagaaagttattgcgaacgtgttcagctactttaaaccgaacatggacgattattgcaaccgtaacctgttcgttatcgacatttttagcgacgatgcgtaccacagccaggaagacagcgtgaccgaacaccgtggtaaccgtcgtctgagcttccacagccaccgtatcgaggaagttccgaaaaccggtctgggcagcagcgcgggtctggtgaccgttctgaccaccgcgctggcgagcttctttgtgagcgacctggagaacaacgttgataaataccgtgaagtgattcacaacctggcgcaagttgcgcactgccaggcgcaaggcaagattggtagcggttttgatgtggcggcggcggcgtacggtagcattcgttatcgtcgttttccgccggcgctgatcagcaacctgccggatattggtagcgcgacctatggcagcaaactggcgcacctggtggacgaggaagattggaacatcaccattaagagcaaccacctgccgagcggtctgaccctgtggatgggtgacatcaagaacggcagcgagaccgtgaaactggttcagaaggtgaaaaactggtacgacagccacatgccggagagcctgaaaatctacaccgaactggatcacgcgaacagccgtttcatggacggcctgagcaagctggatcgtctgcacgaaacccacgacgattacagcgaccagattttcgagagcctggaacgtaacgattgcacctgccaaaaatatccggagattaccgaagttcgtgacgcggtggcgaccatccgtcgtagcttccgtaagattaccaaagagagcggtgcggacatcgaaccgccggttcagaccagcctgctggacgattgccaaaccctgaaaggtgtgctgacctgcctgattccgggtgcgggtggctacgatgcgattgcggttattaccaagcaggacgtggatctgcgtgcgcaaaccgcgaacgataagcgttttagcaaagttcagtggctggacgtgacccaagcggattggggtgttcgtaaggagaaagacccggaaacctatctggataagtaacctatcgcttaaaggagcatcacgaatgaccgtttacaccgcgagcgtgaccgcgccggttaacatcgcgaccctgaagtattggggtaaacgtgataccaagctgaacctgccgaccaacagcagcattagcgtgaccctgagccaggacgatctgcgtaccctgaccagcgcggcgaccgcgccggagttcgaacgtgataccctgtggctgaacggcgagccgcacagcatcgacaacgaacgtacccagaactgcctgcgtgatctgcgtcaactgcgtaaagagatggaaagcaaagatgcgagcctgccgaccctgagccaatggaaactgcacatcgttagcgagaacaacttcccgaccgcggcgggtctggcgagcagcgcggcgggctttgcggcgctggttagcgcgattgcgaaactgtaccagctgccgcaaagcaccagcgaaatcagccgtattgcgcgtaagggtagcggcagcgcgtgccgtagcctgtttggtggctatgttgcgtgggagatgggtaaagcggaagacggccacgatagcatggcggtgcagatcgcggacagcagcgattggccgcaaatgaaggcgtgcgttctggtggtgtctgacattaagaaagatgttagcagcacccagggtatgcaactgaccgtggcgaccagcgagctgttcaaagagcgtatcgaacacgtggttccgaagcgttttgaggtgatgcgtaaagcgattgttgaaaaggatttcgcgacctttgcgaaagaaaccatgatggacagcaacagcttccacgcgacctgcctggatagcttcccgccgatcttttacatgaacgacaccagcaagcgtatcattagctggtgccacaccatcaaccagttctacggcgagaccattgttgcgtatacctttgatgcgggcccgaacgcggtgctgtactatctggcggagaacgaaagcaaactgttcgcgtttatttataagctgttcggtagcgttccgggctgggacaagaaatttaccaccgagcagctggaagcgttcaaccaccaatttgagagcagcaactttaccgcgcgtgagctggacctggaactgcagaaggatgtggcgcgtgttatcctgacccaagtgggtagcggcccgcaagagaccaacgaaagcctgattgacgcgaaaaccggcctgccgaaggaataaaagcttgcggccgcataatgcttaagtcgaacagaaagtaatcgtattgtacacggccgcataatcgaaatttgacaattaatcatcggctcgtataatgtgtggaattgtgagcgctcacaattccccatcttagtatattagttaagtataagaaggagatatacatatggcagatctacaacacaattaaaggagcatcttgtatgaagaccgtggttatcattgatgcgctgcgtaccccgatcggcaagtacaaaggtagcctgagccaagtgagcgcggttgacctgggcacccacgttaccacccagctgctgaaacgtcacagcaccattagcgaggaaatcgatcaagtgattttcggcaacgttctgcaagcgggtaacggtcaaaacccggcgcgtcagatcgcgattaacagcggtctgagccacgagatcccggcgatgaccgtgaacgaagtttgcggcagcggtatgaaggcggttatcctggcgaaacagctgattcaactgggtgaagcggaagtgctgattgcgggtggcattgagaacatgagccaagcgccgaagctgcagcgtttcaactacgagaccgaaagctatgatgcgccgtttagcagcatgatgtacgacggtctgaccgatgcgttcagcggccaagcgatgggtctgaccgcggagaacgtggcggaaaaatatcacgttacccgtgaggaacaggaccaattcagcgttcacagccaactgaaggcggcgcaggcgcaagcggaaggcatctttgcggatgagattgcgccgctggaagtgagcggcaccctggttgagaaagacgaaggtatccgtccgaacagcagcgtggagaagctgggcaccctgaagaccgtgtttaaagaagatggcaccgttaccgcgggtaacgcgagcaccattaacgacggcgcgagcgcgctgatcattgcgagccaggagtacgcggaagcgcacggtctgccgtatctggcgatcattcgtgacagcgttgaggtgggtatcgatccggcgtacatgggtatcagcccgattaaggcgatccaaaaactgctggcgcgtaaccagctgaccaccgaggaaatcgacctgtatgagattaacgaagcgttcgcggcgaccagcatcgtggttcagcgtgaactggcgctgccggaggaaaaggttaacatctacggtggcggtattagcctgggtcatgcgattggtgcgaccggtgcgcgtctgctgaccagcctgagctaccagctgaaccaaaaagagaagaaatatggtgtggcgagcctgtgcatcggcggtggcctgggtctggcgatgctgctggaacgtccgcagcaaaagaaaaacagccgtttctatcaaatgagcccggaggaacgtctggcgagcctgctgaacgagggtcagatcagcgcggataccaagaaagagtttgaaaacaccgcgctgagcagccaaattgcgaaccacatgatcgagaaccagattagcgagaccgaagtgccgatgggcgttggtctgcacctgaccgtggacgaaaccgattacctggttccgatggcgaccgaggaaccgagcgttatcgcggcgctgagcaacggcgcgaagattgcgcaaggtttcaaaaccgtgaaccagcaacgtctgatgcgtggtcagatcgtgttttatgacgttgcggatgcggagagcctgatcgacgaactgcaagttcgtgagaccgaaattttccagcaagcggagctgagctacccgagcattgtgaaacgtggtggcggtctgcgtgacctgcagtatcgtgcgttcgatgagagcttcgtgagcgttgactttctggtggacgttaaggatgcgatgggcgcgaacatcgtgaacgcgatgctggagggtgttgcggaactgttccgtgagtggtttgcggaacagaaaatcctgttcagcattctgagcaactacgcgaccgagagcgtggttaccatgaagaccgcgattccggtgagccgtctgagcaaaggcagcaacggtcgtgagatcgcggaaaagattgttctggcgagccgttatgcgagcctggacccgtatcgtgcggtgacccacaacaagggcatcatgaacggtattgaggcggtggttctggcgaccggtaacgatacccgtgcggttagcgcgagctgccatgcgtttgcggttaaagaaggccgttatcaaggtctgaccagctggaccctggacggcgagcagctgattggtgaaatcagcgttccgctggcgctggcgaccgtgggcggtgcgaccaaggttctgccgaaaagccaggcggcggcggacctgctggcggtgaccgatgcgaaggagctgagccgtgtggttgcggcggttggtctggcgcagaacctggcggcgctgcgtgcgctggttagcgaaggcatccaaaaaggtcacatggcgctgcaagcgcgtagcctggcgatgaccgtgggtgcgaccggtaaagaggtggaagcggttgcgcagcaactgaagcgtcaaaaaaccatgaaccaggatcgtgcgctggcgatcctgaacgacctgcgtaaacagtaaacatgtcatgaaaggagcatcttccatgaccatcggtattgataagatcagcttctttgtgccgccgtactatattgatatgaccgcgctggcggaagcgcgtaacgttgacccgggcaaattccacatcggtattggccaggatcaaatggcggtgaacccgatcagccaggacattgttaccttcgcggcgaacgcggcggaggcgatcctgaccaaggaagataaagaggcgatcgacatggtgattgttggcaccgaaagcagcattgacgagagcaaggctgcggcggtggttctgcaccgtctgatgggtatccagccgttcgcgcgtagctttgaaattaaagagggttgctatggtgcgaccgcgggtctgcaactggcgaaaaaccacgtggcgctgcacccggataagaaagttctggtggttgcggcggacatcgcgaagtatggtctgaacagcggtggcgaaccgacccagggtgcgggtgcggtggcgatgctggttgcgagcgaaccgcgtatcctggcgctgaaagaggataacgtgatgctgacccaagatatttacgacttctggcgtccgaccggtcacccgtatccgatggtggacggtccgctgagcaacgaaacctacatccagagctttgcgcaagtttgggatgagcacaagaaacgtaccggcctggacttcgcggactacgatgcgctggcgtttcacatcccgtataccaagatgggtaagaaagcgctgctggcgaaaatcagcgatcagaccgaggcggaacaagagcgtattctggcgcgttacgaggaaagcatcatttatagccgtcgtgttggtaacctgtacaccggcagcctgtatctgggtctgatcagcctgctggaaaacgcgaccaccctgaccgcgggtaaccagattggcctgttcagctacggtagcggtgcggtggcggagttcttcaccggcgagctggttgcgggttatcagaaccacctgcaaaaggaaacccacctggcgctgctggataaccgtaccgagctgagcatcgcggaatacgaggcgatgtttgcggagaccctggacaccgatattgaccaaaccctggaagacgagctgaaatacagcatcagcgcgattaacaacaccgtgcgtagctatcgtaactaagacgtcggtaccaccaaacgccaaaggagcatccagcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgagaaagaaggcaaaaatcgccgcaccgtctgtgtaatcggcgatggtgcgattaccgcaggcatggcgtttgaagcgatgaatcacgcgggcgatatccgtcctgatatgctggtggttctcaacgacaatgaaatgtcgatttccgaaaatgtcggcgcgctcaacaaccatctggcgcagctgctttccggtaagctttactcttcactgcgcgaaggcgggaaaaaagttttctctggcgttccgccaattaaagagctgctcaaacgcaccgaagaacatattaaaggcatggtagtgcctggcacgttgtttgaagagctgggctttaactacatcggcccggttgacggtcacgatgtgctggggcttatcaccacgcttaagaacatgcgcgacctgaaaggcccgcagttcctgcatatcatgaccaaaaaaggtcgtggttatgaaccggcagaaaaagacccaatcactttccacgccgtgcctaaatttgatccctccagcggttgtttgccgaaaagtagcggcggtttgccgagctattcaaaaatctttggcgactggttgtgtgaaacggcagcgaaagacaacaagctgatggcgattactccggcaatgcgtgaaggttccggcatggtcgagttttcacgtaaattcccggatcgctacttcgacgtggcaattgccgagcaacacgcagtgacctttgccgccggtctggcgattggtgggtacaaacccattgtcgcgatttactccactttcctgcaacgcgcctatgatcaggtgctgcatgacgtggcgattcaaaagctaccggtcctgttcgccattgaccgcgcgggcattgttggtgctgacggtcaaactcaccagggcgcttttgatctctcttacctgcgctgcataccagaaatggtcattatgaccccgagcgatgaaaacgaatgtcgccagatgctctataccggctatcactataacgatggcccgtccgcggtgcgctacccgcgcggcaacgcggttggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccaccagccatgaagcgctggtcaccgtagaagaaaacgccattatgggcggcgcaggtagcggagtgaacgaagtgctgatggcccatcgtaaaccagtacccgtgctgaacattggtctgccggacttctttattccgcaaggaactcaggaagaaatgcgcgccgaactcggcctcgatgccgccggtatggaagccaaaatcaaggcctggctggcataactcgagtctggtaaagaaaccgctgctgcgaaatttgaacgccagcacatggactcgtctactagcgcagcttaattaacctaggctgctgccaccgctgagcaataactagcataaccccttggggccaggcatcaaataaaacgaaaggctcagtcgaaagactgggcctttcgttttatctgttgtttgtcggtgaacgctctccctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctag

SEQ ID NO: 48 - protein, 368 aa, Enterococcus faecalis
MIEVTTPGKLFIAGEYAVVEPGHPAIIVAVDQFVTVTVEETTDEGSIQSAQYSSLPIRWTRRNGELVLDIRENPFHYVLAAIHLTEKYAQEQNKELSFYHLKVTSELDSSNGRKYGLGSSGAVTVGTVKALNIFYDLGLENEEIFKLSALAHLAVQGNGSCGDIAASCYGGWIAFSTFDHDWVNQKVATETLTDLLAMDWPELMIFPLKVPKQLRLLIGWTGSPASTSDLVDQVHQSKEEKQAAYEQFLMKSRLCVETMINGFNTGKISVIQKQITKNRQLLAELSSLTGVVIETEALKNLCDLAESYTGAAKSSGAGGGDCGIVIFRQKSGILPLMTAWEKDGITPLPLHVYTYGQKECKEKHESKR

SEQ ID NO: 49 - protein, 331 aa, Enterococcus faecalis
MLSGKARAHTNIALIKYWGKANEEYILPMNSSLSLTLDAFYTETTVIFDAHYSEDVFILDGILQNEKQTKKVKEFLNLVRQQADCTWFAKVESQNFVPTAAGLASSASGLAALAGACNVALGLNLSAKDLSRLARRGSGSACRSIFGGFAQWNKGHSDETSFAENIPANNWENELAMLFILINDGEKDVSSRDGMKRTVETSSFYQGWLDNVEKDLSQVHEAIKTKDFPRLGEIIEANGLRMHGTTLGAVPPFTYWSPGSLQAMALVRQARAKGIPCYFTMDAGPNVKVLVEKKNLEALKTFLSEHFSKEQLVPAFAGPGIELFETKGMDK

SEQ ID NO: 50 - skipped

SEQ ID NO: 51 - protein, 304 aa, Agrobacterium aurantiacum
MSDLVLTSTEAITQGSQSFATAAKLMPPGIRDDTVMLYAWCRHADDVIDGQALGSRPEAVNDPQARLDGLRADTLAALQGDGPVTPPFAALRAVARRHDFPQAWPMDLIEGFAMDVEARDYRTLDDVLEYSYHVAGIVGVMMARVMGVRDDPVLDRACDLGLAFQLTNIARDVIDDARIGRCYLPGDWLDQAGARVDGPVPSPELYTVILRLLDAAELYYASARVGLADLPPRCAWSIAAALRIYRAIGLRIRKGGPEAYRQRISTSKAAKIGLLGIGGWDVARSRLPGAGVSRQGLWTRPHHA

SEQ ID NO: 52 - protein, 309 aa, Pantoea agglomerans
MSQPPLLDHATQTMANGSKSFATAAKLFDPATRRSVLMLYTWCRHCDDVIDDQTHGFASEAAAEEEATQRLARLRTLTLAAFEGAEMQDPAFAAFQEVALTHGITPRMALDHLDGFAMDVAQTRYVTFEDTLRYCYHVAGVVGLMMARVMGVRDERVLDRACDLGLAFQLTNIARDIIDDAAIDRCYLPAEWLQDAGLTPENYAARENRAALARVAERLIDAAEPYYISSQAGLHDLPPRCAWAIATARSVYREIGIKVKAAGGSAWDRRQHTSKGEKIAMLMAAPGQVIRAKTTRVTPRPAGLWQRPV

SEQ ID NO: 53 - protein, 614 aa, Mucor lusitanicus
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 54 - protein, 673 aa, Xanthophyllomyces dendrorhous
MTALAYYQIHLIYTLPILGLLGLLTSPILTKFDIYKISILVFIAFSATTPWDSWIIRNGAWTYPSAESGQGVFGTFLDVPYEEYAFFVIQTVITGLVYVLATRHLLPSLALPKTRSSALSLALKALIPLPIIYLFTAHPSPSPDPLVTDHYFYMRALSLLITPPTMLLAALSGEYAFDWKSGRAKSTIAAIMIPTVYLIWVDYVAVGQDSWSINDEKIVGWRLGGVLPIEEAMFFLLTNLMIVLGLSACDHTQALYLLHGRTIYGNKKMPSSFPLITPPVLSLFFSSRPYSSQPKRDLELAVKLLEEKSRSFFVASAGFPSEVRERLVGLYAFCRVTDDLIDSPEVSSNPHATIDMVSDFLTLLFGPPLHPSQPDKILSSPLLPPSHPSRPTGMYPLPPPPSLSPAELVQFLTERVPVQYHFAFRLLAKLQGLIPRYPLDELLRGYTTDLIFPLSTEAVQARKTPIETTADLLDYGLCVAGSVAELLVYVSWASAPSQVPATIEEREAVLVASREMGTALQLVNIARDIKGDATEGRFYLPLSFFGLRDESKLAIPTDWTEPRPQDFDKLLSLSPSSTLPSSNASESFRFEWKTYSLPLVAYAEDLAKHSYKGIDRLPTEVQAGMRAACASYLLIGREIKVVWKGDVGERRTVAGWRRVRKVLSVVMSGWEGQ

SEQ ID NO: 55 - protein, 317 aa, Archaeoglobus fulgidus
MLKEEIAKRAEIINKAIEELLPEREPIGLYKAARHLIKAGGKRLRPVISLLAVEALGKDYRKIIPAAVSIETIHNFTLVHDDIMDRDEMRRGVPTVHRVYGEATAILAGDTLFAEAFKLLTKCDVESEGIRKATEMLSDVCIKICEGQYYDMSFEKKESVSEEEYLRMVELKTGVLIAASAALPAVLFGESEEIVKALWDYGVLSGIGFQIQDDLLDLTEETGKDWGSDLLKGKKTLIVIKAFEKGVKLKTFGKEKADVSEIRDDIEKLRECGAIDYAASMARKMAEEAKRKLEVLPESKAKETLLELTDFLVTRKK

SEQ ID NO: 56 - protein, 393 aa, Taxus X media
MAYTAMAAGTQSLQLRTVASYQECNSMRSCFKLTPFKSFHGVNFNVPSLGAANCEIMGHLKLGSLPYKQCSVSSKSTKTMAQLVDLAETEKAEGKDIEFDFNEYMKSKAVAVDAALDKAIPLEYPEKIHESMRYSLLAGGKRVRPALCIAACELVGGSQDLAMPTACAMEMIHTMSLIHDDLPCMDNDDFRRGKPTNHKVFGEDTAVLAGDALLSFAFEHIAVATSKTVPSDRTLRVISELGKTIGSQGLVGGQVVDITSEGDANVDLKTLEWIHIHKTAVLLECSVVSGGILGGATEDEIARIRRYARCVGLLFQVVDDILDVTKSSEELGKTAGKDLLTDKATYPKLMGLEKAKEFAAELATRAKEELSSFDQIKAAPLLGLADYIAFRQN

SEQ ID NO: 57 - protein, 376 aa, Xanthophyllomyces dendrorhous
MDYANILTAIPLEFTPQDDIVLLEPYHYLGKNPGKEIRSQLIEAFNYWLDVKKEDLEVIQNVVGMLHTASLLMDDVEDSSVLRRGSPVAHLIYGIPQTINTANYVYFLAYQEIFKLRPTPIPMPVIPPSSASLQSSVSSASSSSSASSENGGTSTPNSQIPFSKDTYLDKVITDEMLSLHRGQGLELFWRDSLTCPSEEEYVKMVLGKTGGLFRIAVRLMMAKSECDIDFVQLVNLISIYFQIRDDYMNLQSSEYAHNKNFAEDLTEGKFSFPTIHSIHANPSSRLVINTLQKKSTSPEILHHCVNYMRTETHSFEYTQEVLNTLSGALERELGRLQGEFAEANSKIDLGDVESEGRTGKNVKLEAILKKLADIPL

SEQ ID NO: 58 - protein, 320 aa, Blakeslea trispora
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 59 - DNA, 7522 bp, synthetic construct
  source 1..7522: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccaccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcttataatggtctttgccacaaatgagctggtcttggtggatgtgctctcattcttgaagtcaatgcttgaccagatgcagccaacaacaatgtcaatttttctggagttgtagtagattgtctttgatcccatgcttgttgacctgcttgttcaactttaacaccgatctttctgtaaacttgcttagctgttgcgatagcccatgctgatctcaatggtaatctagctaaaccagcagttgcagacaagtaatatggttctgcttcttgaactaatcttctagcaattcttgacaatgcttgtctattttctggtgcagcgtagttttccttattcaaaccttcatgttccaaccaagaagctggcaaataacatctaccagcatgtgcatcatcaacaatatctctagcgatgtttgtcaattggaatgccaaacccaaatcacaagctctatccaaagttgctttatctctaacacccataatttgagccatcattaaaccaacaacacctgcaacatggtaacagtatctcaaagtatcatccaattgtgagtattgagcttctctaacatccattgcaaaaccttccaaatgatcaaaagcataagctggagcaatatcatgtgccatagcaacttcttgaaatgcagcaaaagctggttcatgcatttgagaaccagcataggcttgtcttgtcttcatttccaattgcatcaatctttgttctggagtttgtaaagctggttgtcttgcttgaaaacccaatgtttgatcatcgataacatcatcacaatgtctacaccaagcatacaacatcaaaactgatcttctagtctttgcatcgaacaatttagatgctgtagcaaatgatttagaaccaacagccatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 60 - DNA, 8209 bp, synthetic construct
  source 1..8209: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcttaggatttaatgcaggtgacggacccatctttcaaacgatttatatcagtggcgtccaaattgttaggttttgttggttcagcaggtttcctgttgtgggtcatatgactttgaaccaaatggccggctgctagggcagcacataaggataattcacctgccaagacggcacaggcaactattcttgctaattgacgtgcgttggtaccaggagcggtagcatgcgggcctcttacacctaataagtccaacatggcaccttgtggttctagaacagtaccaccaccgatggtacctacttcgatggatggcatggatacggaaattctcaaatcaccgtccacttctttcatcaatgttatacagttggaactttcaacattttgtgcaggatcttgtcctaatgccaagaaaacagctgtcactaaattagctgcatgtgcgttaaatccaccaacagacccagccattgcagatccaaccaaattcttagcaatgttcaactcaaccaatgcggaaacatcactttttaacacttttctgacaacatcaccaggaatagtagcttctgcgacgacactcttaccacgaccttcgatccagttgatggcagctggttttttgtcggtacagtagttaccagaaacggagacaacctccatatcttcccagccatactcttctaccatttgctttaatgagtattcgacacctttagaaatcatattcatacccattgcgtcaccagtagttgttctaaatctcatgaagagtaaatctcctgctagacaagtttgaatatgttgcagacgtgcaaatcttgatgtagagttaaaagcttttttaattgcgttttgtccctcttctgagtctaaccatatcttacaggcaccagatcttttcaaagttgggaaacggactactgggcctcttgtcataccatccttagttaaaacagttgttgcaccaccgccagcattgattgccttacagccacgcatggcagaagctaccaaacaaccctctgtagttgccattggtatatgataagatgtaccatcgataaccaaggggcctataacaccaacgggcaaaggcatgtaacctataacattttcacaacaagcgccaaatacgcggtcgtagtcataatttttatatggtaaacgatcagatgctaatacaggagcttctgccaaaattgaaagagccttcctacgtaccgcaaccgctctcgtagtatcacctaattttttctccaaagcgtacaaaggtaacttaccgtgaataaccaaggcagcgacctctttgttcttcaattgttttgtatttccactacttaataatgcttctaattcttctaaaggacgtattttcttatccaagctttcaatatcgcgggaatcatcttcctcactagatgatgaaggtcctgatgagctcgattgcgcagatgataaacttttgactttcgatccagaaatgactgttttattggttaaaactggtgtagaagccttttgtacaggagcagtaaaagacttcttggtgacttcagttttcaccaattggtccatggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 61 - DNA, 8129 bp, synthetic construct
  source 1..8129: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccccccagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatgtcaactttgtcagtttctacaccatctttttcttcttctccattgtcttctgttaacaaaaattcaactaagcaacatgttacaagaaattcagttatcttccatgattctatttggggtgatcaattcttggaatacaaggaaaagttcaatgttgctactgaaaaacaattaattgaagaattgaaagaagaagttagaaatgaattaatgatcagagcttgtaacgaagcatcaagatacatcaagttgatccaattgatcgatgttgttgaaagattgggtttggcataccatttcgaaaaggaaatcgaagaatctttgcaacatatctacgttacttacggtcataagtggacaaactacaacaacatcgaatcattgtctttgtggttcagattgttgagacaaaatggtttcaacgtttcatctgatattttcgaaaatcatattgatgaaaagggtaattttcaagaatctttgtgtaatgatccacaaggcatgttagctttgtatgaagctgcatacatgagagttgaaggtgaaatcatcttggataaggcattggaatttactaagttgcatttgggtatcatctcaaacgatccatcttgtgattcatctttgagaacagaaatcaagcaagctttgaaacaaccattgagaagaagattgccaagattggaagctgttagatacatcgcaatctaccaacaaaaagcttcacattctgaagttttgttgaagttggcaaagttggatttcaatgttttgcaagaaatgcataaggatgaattatcacaaatctgtaagtggtggaaggatttggatattagaaacaagttgccatacgttagagatagattgatcgaaggttacttctggatcttgggtatctacttcgaaccacaacattctagaactagaatgttcttgatgaagacatgtatgtggttgatcgttttggatgatactttcgataactatggtacatacgaagaattagaaatcttcactcaagctgttgaaagatggtctatcacatgtttggatgaattgccagaatacatgaagttgatctaccatgaacaattcagagttcatcaagaaatggaagaatcattggaaaaagaaggtaaagcttaccaaatccattacatcaaggaaatggcaaaagaaggtactagatctttgttattggaagctaaatggttgaaagaaggttatatgccaacattggatgaatacttgtcaaattctttggttacttgtggttacgctttaatgacagcaagatcatacgttgcaagagatgatggtatcgttactgaagatgctttcaagtgggttgcaacacatccaccaatcgttaaggctgcttgtaagatcttgagattgatggatgatattgctactcataaagaagaacaagaaagaggtcatatcgcatcatctatcgaatgttacagaaaagaaacaggtgcttctgaagaagaagcttgtatggatttcttgaaacaagttgaagatggttggaaggttatcaaccaagaatcattgatgccaactgatgttccattcccattgttgatcccagctatcaacttggcaagagtttctgatacattgtacaaggataatgatggttacaatcatgctgataaagaagttattggttacattaaatctttatttgttcatccaatgattgtttaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctgatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 62 - DNA, 6439 bp, synthetic construct
  source 1..6439: mol_type = other DNA
tgcttgcgccctatggatgtaccttttctgcttgtgatgccagccattaatctggcacgtatctgtgacgctttgtattcgaaaggtaatgatgggttcaaccacgctggagaagaagtgatcaactacatcaaaagcctcgtagtccatccgctcgtggtgtaagagctcggcgcgcctgcaggtcgacaagcttgcggccgcataatgcttaagtcgaacagaaagtaatcgtattgtacacggccgcataatcgaaatttgacaattaatcatcggctcgtataatgtgtggaattgtgagcgctcacaattccccatcttagtatattagttaagtataagaaggagatatacatatggcagatctcaattggatatcgatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataactcgagtctggtaaagaaaccgctgctgcgaaatttgaacgccagcacatggactcgtctactagcgcagcttaattaacctaggctgctgccaccgctgagcaataactagcataaccccttggggccaggcatcaaataaaacgaaaggctcagtcgaaagactgggcctttcgttttatctgttgtttgtcggtgaacgctctccctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaatttgacaattaatcatcggctcgtataatgtgtggaattgtgagcgctcacaattcccctgtagaaataattttgtttaactttaataaggagatataccatggcaatgtcaaccgcttttccggtggcggcattagtaagtgccagcgcatcattttcctcttcagaaccccttccttctccaccaccgactacgactgcgacgaaacaacgcaacactacaaaccatcacagttctatctggggtgatcagtttctgacctaccacgaaagtgaagacatcctgaaagaaaaactgctggttgacgaactgaaagaacaggtccgtaaagagctgatttcggcgtctgttgacgatcatctggaactgattgatgcgattcagagactgggggtggcttactattatgaaaatgagatcgaggaggctctacagcacatctatgtaacctacggtgatcattggatacacactgacaacctgcaaagcatcagtctatggtttcgtttgttacgccagcaaggcttcaatgtttcgtcgggcatctttaagaaccatatgaacgacaaggggaacttcaaggagtctattagcaatgacgtccagggcatgttggaactgtatgaagccgcctatatgcgcgtcgagggtgaagaaatcttggactacgctctggaattcaccaaaaccaacttaggcattttagcgaacgatccgtcatgcgatagcagtttacgttcgcagatccaacaagcactgaaacaaccactgcgaaaaagattgccgcgtttggaagccgtcaggtatatcccgatatataaacaacaggcgtgtcacaacgacgttctgctgaaactggcaaaacttgatttcaacgtgctccagtccatgcacaaaaaggagctttcccacatctgcaaatggtggaaggacttaaacatgcaggaaaaactgccgtatgtgcgtgatcgactggtggaaggctacttctggatcttaggtatctactttgaaccccatcatgcgcgcacgcgcatgtttctgatcaaaacctgtatgtggctcgtagttatcgatgatacgttcgacaattacggtgcctatgaagaactggaacagtttaccgaggcggtagaacgttggtcgatttcttgcttggatgaactcccggagtatatgaaactggtttatcaggagctggtcaacgttcatcaagagatggaggagagcctcgaaaaggaaggcaaggcgtttcagattcactacgtgaaggaaatggcgaaagaatgcacccgcagcttactgctggaagcgaaatggctgaaagatggctatatgccgacgctggaagaatacctcagcaatagcctgattacatgcgcctatgcagttatggtggcgcggtcatatgtgggacgtgaagatgttatgattcgtgaggagacgtttaaatgggtggcaacacatccgccgcttgtcaaagccagttgcctaattctgcgcctgatggatgatattgcaacccataaagaagagcaggaacgcggccatgtggccagctccattgaatgttacattcgcgagaatgtcggtgcgaccgaagaagaagcgtgtgaacttttctccaaacagatcgaggatgcctggaaagttatcaatcgggaa

SEQ ID NO: 63 - protein, 424 aa, synthetic construct
MKISWNGFSKKSYQERLELLKAQALLSPERQASLEKDEQMSVTVADQLSENVVGTFSLPYSLVPEVLVNGQEYTVPYVTEEPSVVAAASYASKIIKRAGGFTAQVHQRQMIGQVALYQVANPKLAQEKIASKKAELLELANQAYPSIVKRGGGARDLHVEQIKGEPDFLVVYIHVDTQEAMGANMLNSMLEALKPVLEELSQGQSLMGILSNYATDSLVTASCRIAFRYLSRQKDQGREIAEKIALASQFAQADPYRAATHNKGIFNGIDAILIATGNDWRAIEAGAHAFASRDGRYQGLSCWTLDLEREELVGEMTLPMPVATKGGSIGLNPRVALSHDLLGNPSARELAQIIVSIGLAQNFAALKALVSTGIQQGHMKLQAKSLALLAGASESEVAPLVERLISDKTFNLETAQRYLENLRS

SEQ ID NO: 64 - protein, 424 aa, synthetic construct
MKISWNGFSKKSYQERLELLKAQALLSPERQASLEKDEQMSVTVADQLSENVVGTFSLPYSLVPEVLVNGQEYTVPYVTEEPSVVAAASYASKIIKRAGGFTAQVHQRQMIGQVALYQVANPKLAQEKIASKKAELLELANQAYPSIVKRGGGARDLHVEQIKGEPDFLVVYIHVDTQEAMGANMLNTMLEALKPVLEELSQGQSLMGILSNYATDSLVTASCRIAFRYLSRQKDHGREIAEKIALASQFAQADPYRAATHNKGIFNGIDAILIATGNDWRAIEAGAHAFASRDGRYQGLSCWTLDLEREELVGEMTLPMPVATKGGSIGLNPRVALSHDLLGNPSARELAQIIVSIGLAQNFAALKALVSTGIQQGHMKLQAKSLALLAGASESEVAPLVERLISDKTFNLETAQRYLENLRS

SEQ ID NO: 65 - protein, 424 aa, synthetic construct
MKISWNGFSKKSYQERLELLKAQALLSPERQASLEKDEQMSVTVADQLSENVVGTFSLPYSLVPEVLVNGQEYTVPYVTEEPSVVAAASYASKIIKRAGGFTAQVHQRQMIGQVALYQVANPKLAQEKIASKKAELLELANQAYPSIVKRGGGARDLHVEQIKGEPDFLVVYIHVDTQEAMGANMLNTMLEALKPVLEELSQGQSLMGILSNYATDSLVTASCRIAFRYLSRQKDQGREIAEKIALASQFAQADPYRAATHNKGIFNGIDAILIATGNDWRAIEAGAHAFASRDGRYQGLSCWTLDLEREELVGEMTLPMPVATKGGSIGLNPRVALSHDLLDNPSARELAQIIVSIGLAQNFAALKALVSTGIQQGHMKLQAKSLALLAGASESEVAPLVERLISDKTFNLETAQRYLENLRS

SEQ ID NO: 66 - protein, 443 aa, synthetic construct
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFDNKTTTKQQIDDLLLPGNTNLPWTS

SEQ ID NO: 67 - protein, 443 aa, synthetic construct
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAVNLNKDLKIKSLVFQLFENKTTTKQQIDDLLLPGNTNLPWTS

SEQ ID NO: 68 - protein, 443 aa, synthetic construct
MSLPFLTSAPGKVIIFGEHSAVYNKPAVAASVSALRTYLLISESSAPDTIELDFPDISFNHKWSINDFNAITGDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 69 - protein, 443 aa, synthetic construct
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGVTGCCLLSAKNLNKDLKIKSLVFQLFENKTTTKQQIDDLLLPGNTNLPWTS

SEQ ID NO: 70 - protein, 443 aa, synthetic construct
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKVSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 71 - protein, 443 aa, synthetic construct
MSLPFLTSAPGKVIIFGEHSAVYNKPAVAASVSALRTYLLISESSAPDTIELDFPDISFNHKWSINDFNAITEEQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 72 - protein, 407 aa, synthetic construct
MSPEERLASLLNEGQISADTKKEFENTALSSQIANHMIENQISETEVPMGVGLHLTVDETDYLVPMATEEPSVIAALSNGAKIAQGFKTVNQQRLMRGQIVIYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 73 - protein, 407 aa, synthetic construct
MSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAESKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 74 - protein, 407 aa, synthetic construct
MSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYAHLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 75 - protein, 407 aa, synthetic construct
MSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLADNLAALRALVSEGIQKGHMALQARSLAMTVGATGKEVEAVAQQLKRQKTMNQDRALAILNDLRKQ

SEQ ID NO: 76 - protein, 424 aa, synthetic construct
MKISWNGFSKKSYQERLELLKAQALLSPERQASLEKDEQMSVTVADQLSENVVGTFSLPYSLVPEVLVNGQEYTVPYVTEEPSVVAAASYASKIIKRAGGFIAQVHQRQMIGQVALYQVANPKLAQEKIASKKAELLELANQAYPSIVKRGGGARDLHVEQIKGEPDFLVVYIHVDTQEAMGANMLNTMLEALKPVLEELSQGQSLMGILSNYATDSLVTASCRIAFRYLSRQKDQGREIAEKIALASQFAQADPYRAATHNKGIFNGIDAILIATGNDWRAIEAGAHAFASRDGRYQGLSCWTLDLEREELVGEMTLPMPVATKGGSIGLNPRVALSHDLLGNPSARELAQIIVSIGLAQNFAALKALVSTGIQQGHMKLQAKSLALLAGASESEVAPLVERLISDKTFNLETAQRYLENLRS

SEQ ID NO: 77 - protein, 527 aa, synthetic construct
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGILPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 78 - protein, 527 aa, synthetic construct
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTESCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 79 - protein, 527 aa, synthetic construct
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIHLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 80 - protein, 527 aa, synthetic construct
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIDGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS